(19) United States
(12) Patent Application Publication
(10) Pub. No.: US 2022/0356174 A1
(43) Pub. Date: Nov. 10, 2022

(54) PYRIDIN-2-ONE COMPOUNDS USEFUL AS SMARCA2 ANTAGONISTS

(71) Applicant: Epizyme, Inc., Cambridge, MA (US)

(72) Inventors: Oscar Moradei, Burlington, MA (US); John W. Lampe, Norfolk, MA (US); Darren Martin Harvey, Acton, MA (US); John Emmerson Campbell, Cambridge, MA (US); Kenneth William Duncan, Westwood, MA (US); Michael John Munchhof, Corvallis, MT (US)

(73) Assignee: Epizyme, Inc., Cambridge, MA (US)

(21) Appl. No.: 17/262,395

(22) PCT Filed: Jul. 24, 2019
(86) PCT No.: PCT/US2019/043274

§ 371 Date: Jan. 22, 2021

Related U.S. Application Data
(60) Provisional application No. 62/702,481, filed on Jul. 24, 2018.
(60) Provisional application No. 62/815,208, filed on Mar. 7, 2019.

(51) Int. Cl. C07D 409/12; A61P 35/00; C07D 401/12; C07D 405/12; C07D 417/12; C07D 413/12; C07D 409/14; C07D 413/14; C07D 417/14; C07D 401/14; C07D 491/056; C07D 471/04; C07D 487/04; C07D 513/04; C07D 213/75; C07D 495/04; C07D 498/04

(52) U.S. Cl. CPC C07D 409/12; C07D 498/04; C07D 401/12; C07D 405/12; C07D 417/12; C07D 413/12; C07D 409/14; C07D 413/14; C07D 417/14; C07D 401/14; C07D 491/056; C07D 471/04; C07D 487/04; C07D 513/04; C07D 213/75; C07D 495/04; A61P 35/00

(57) ABSTRACT

This disclosure generally relates to pyridine-2-one compounds of formula (I) and methods of using them in the treatment of a disorder, such as cancer or a SMARCA2-associated disorder, including as antagonists (e.g., inhibitors) of SMARCA2. The present disclosure provides treatment modalities, e.g., strategies, treatment methods, patient stratification methods, compounds, combinations, and compositions that are useful for the treatment of disorders, e.g., proliferative disorders, such as certain cancers. Some aspects of this disclosure provide treatment modalities, methods, strategies, compounds, compositions, combinations, and dosage forms for the treatment of cell proliferative disorders, e.g., cancers with decreased activity or function, or loss of function, of SMARCA4 with a SMARCA2 antagonist.

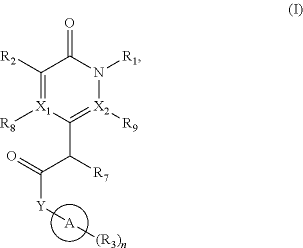

### CROSS-REFERENCE TO RELATED APPLICATIONS

This application claims the benefit of priority to U.S. Provisional Patent Application No. 62/702,481, filed Jul. 24, 2018, and U.S. Provisional Patent Application No. U.S. 62/815,208, filed Mar. 7, 2019, each of which is incorporated by reference herein in its entirety.

### REFERENCE TO SEQUENCE LISTING SUBMITTED ELECTRONICALLY

This application incorporates by reference a Computer Readable Form (CRF) of a Sequence Listing in ASCII text format submitted with this application, entitled “13015-025-228_ST25.txt”, was created on Jul. 22, 2019, and is 14 kilobytes in size.

### FIELD OF DISCLOSURE

This disclosure generally relates to pyridine-2-one compounds and methods of using them in the treatment of a disorder, such as cancer or a SMARCA2-associated disorder, including as antagonists (e.g., inhibitors) of SMARCA2.

### SUMMARY

In some aspects, the present disclosure features a compound of Formula (I):

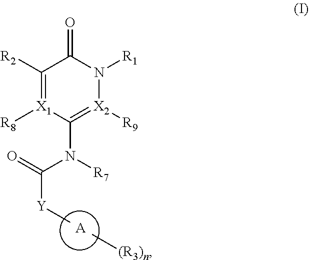

or a pharmaceutically acceptable salt thereof, wherein

A is a heteroaryl, heterocycloalkyl, aryl, or a cycloalkyl;

X₁ and X₂ are each independently selected from —CH and N;

Y is selected from the group consisting of a bond, —NH, —C(O), C₁-C₆ alkyl, —C(CH₃)₂—O—, and —CH₂—NH—CH₂—;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, —OR₅, —C(O)NH₂, —NO₂;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

each R₅ is independently selected from the group consisting of H, cyano, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₆ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, C₃-C₆ cycloalkyl, C₃-C₆ heterocycloalkyl or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

R₈ and R_{9′} are each independently selected from the group consisting of H, halo, and C₁-C₃ alkyl;

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

In some aspects, the present disclosure features a compound of Formula (IA):

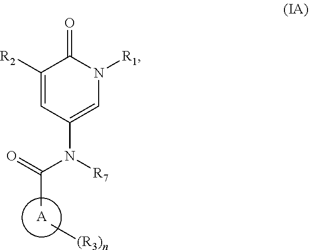

or a pharmaceutically acceptable salt thereof, wherein

A is a heteroaryl, heterocycloalkyl, aryl, or a cycloalkyl;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, C₃-C₆ cycloalkyl, C₃-C₆ heterocycloalkyl or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

In some embodiments, for a compound Formula (IA) or a pharmaceutically acceptable salt thereof,

A is a heteroaryl, heterocycloalkyl, aryl, or a cycloalkyl;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅, wherein Q is C₁-C₃ alkyl, C₂-C₆ alkenyl, or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

In some aspects, the present disclosure features a compound of Formula (IB):

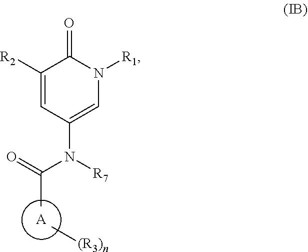

or a pharmaceutically acceptable salt thereof, wherein

A is a heteroaryl, heterocycloalkyl, aryl, or a cycloalkyl;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, C₃-C₆ cycloalkyl, C₃-C₆ heterocycloalkyl or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H and C₁-C₆ alkyl;

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted

In some embodiments, for a compound Formula (IB) or a pharmaceutically acceptable salt thereof,

A is a heteroaryl, heterocycloalkyl, aryl, or a cycloalkyl;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H and C₁-C₆ alkyl;

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted

In some aspects, the present disclosure features a compound of Formula (IC):

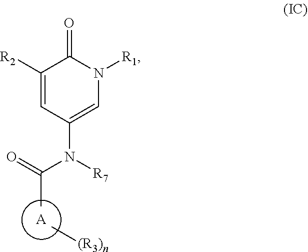

or a pharmaceutically acceptable salt thereof, wherein

A is a 5- or 6-membered heteroaryl having 1 to 4 heteroatoms selected from N, O, and S;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, C₃-C₆ cycloalkyl, C₃-C₆ heterocycloalkyl or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

In some embodiments, for a compound Formula (IC) or a pharmaceutically acceptable salt thereof,

A is a 5- or 6-membered heteroaryl having 1 to 4 heteroatoms selected from N, O, and S;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅, wherein Q is C₁-C₃ alkyl, C₂-C₆ alkenyl, or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

In some aspects, the present disclosure features a compound of Formula (ID):

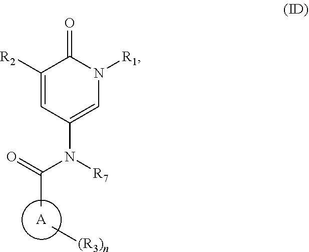

or a pharmaceutically acceptable salt thereof, wherein

A is a heteroaryl, heterocycloalkyl, aryl, or a cycloalkyl;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅;

each Q is independently selected from the group consisting of C₁-C₃ alkyl, C₂-C₆ alkenyl, C₃-C₆ cycloalkyl, C₃-C₆ heterocycloalkyl, and C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted;

provided that at least one R₃ is QR₆, wherein Q is C₂-C₆ alkynyl.

In some aspects, the present disclosure features a compound of Formula (IE)

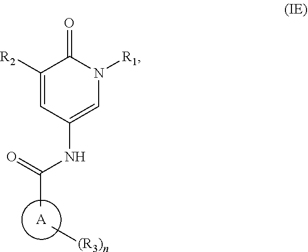

or a pharmaceutically acceptable salt thereof, wherein

A is a 5-membered heteroaryl having 1 to 4 heteroatoms selected from N, O, and S;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅, wherein Q is C₁-C₃ alkyl, C₂-C₆ alkenyl, or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

In some embodiments, one or more of the compounds described herein, or a pharmaceutically acceptable salt thereof may be used in the treatment of a disorder, such as cancer or a SMARCA2-associated disorder.

In some embodiments, one or more of the compounds disclosed herein are antagonists (e.g., inhibitors) of SMARCA2. In some embodiments, one or more of the compounds disclosed herein inhibit SMARCA2 with an enzyme inhibition IC₅₀ value of about 50 μM or less, 1 μM or less, about 500 nM or less, about 200 nM or less, about 100 nM or less, about 50 nM or less, or about 10 nM or less.

Also provided herein are pharmaceutical compositions comprising one or more pharmaceutically acceptable carriers and one or more compounds of Formula (I), (IA), (IB), (IC), (ID), or (E) or a pharmaceutically acceptable salt thereof, described herein.

Some aspects of this disclosure provide methods comprising modulating (e.g., inhibiting) a SMARCA2 activity in a cell or subject. In some embodiments, this disclosure provides methods comprising modulating (e.g., inhibiting) a SMARCA2 activity in a cell or subject exhibiting a decreased activity or function of SMARCA4 (e.g., a loss of function of SMARCA4).

Some aspects of this disclosure provide methods of treating cancer in a subject in need thereof, comprising administering a therapeutically effective amount of a SMARCA2 antagonist (e.g., a SMARCA2 inhibitor, e.g., a compound of Formula (I), (IA), (IB), (IC), (ID), or (E) or a pharmaceutically acceptable salt thereof) to the subject or a cell of the subject. In some embodiments, the subject or cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

Some aspects of the disclosure relate to a SMARCA2 antagonist (e.g., a SMARCA2 inhibitor, e.g., a compound of Formula (I), (IA), (IB), (IC), (ID), or (E) or a pharmaceutically acceptable salt thereof) for use in the treatment of cancer in a cell or subject. In some embodiments, the subject or cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

Some aspects of the disclosure relate to a SMARCA2 antagonist (e.g., a SMARCA2 inhibitor, e.g., a compound of Formula (I), (IA), (IB), (IC), (ID), or (IE) or a pharmaceutically acceptable salt thereof) for use as a medicament for the treatment of cancer in a cell or subject. In some embodiments, the subject or cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

Some aspects of the disclosure relate to the use of a SMARCA2 antagonist (e.g., a SMARCA2 inhibitor, e.g., a compound of Formula (I), (IA), (IB), (IC), (ID), or (IE) or a pharmaceutically acceptable salt thereof) in the manufacture of a medicament for the treatment of cancer in a cell or subject. In some embodiments, the subject or cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

Some aspects of this disclosure provide methods of modulating (e.g., inhibiting) an activity of SMARCA2, comprising contacting SMARCA2 enzyme with a SMARCA2 antagonist (e.g., a SMARCA2 inhibitor, e.g., a compound of Formula (I), (IA), (IB), (IC), (ID), or (IE) or a pharmaceutically acceptable salt thereof). In some embodiments, the SMARCA2 enzyme is within a cell, e.g., a cancer cell, and the method comprises contacting the cell with a compound of Formula (I), (IA), (IB), (IC), (ID), or (IE) or a pharmaceutically acceptable salt thereof, wherein the cell comprises a biomarker of sensitivity to the SMARCA2 antagonist (e.g. a compound of Formula (I), (IA), (IB), (IC), (ID), or (IE) or a pharmaceutically acceptable salt thereof).

Some aspects of this disclosure provide a SMARCA2 antagonist (e.g., a SMARCA2 inhibitor, e.g., a compound of Formula (I), (IA), (IB), (IC), (ID), or (IE) or a pharmaceutically acceptable salt thereof) for use in inhibiting an activity of SMARCA2, wherein the SMARCA2 antagonist (e.g., a SMARCA2 inhibitor, e.g., a compound of Formula (I), (IA), (IB), (IC), (ID), or (IE) or a pharmaceutically acceptable salt thereof) is contacted with a SMARCA2 enzyme. In some embodiments, the SMARCA2 enzyme is within a cell, e.g., a cancer cell, wherein the cell comprises a biomarker of sensitivity to a SMARCA2 antagonist (e.g., a compound of Formula (I), (IA), (IB), (IC), (ID), or (IE) or a pharmaceutically acceptable salt thereof).

Some aspects of this disclosure provide a SMARCA2 antagonist (e.g., a SMARCA2 inhibitor, e.g., a compound of Formula (I), (IA), (IB), (IC), (ID), or (IE) or a pharmaceutically acceptable salt thereof) for use as a medicament for inhibiting an activity of SMARCA2, wherein the medicament is contacted with a SMARCA2 enzyme. In some embodiments, the SMARCA2 enzyme is within a cell, e.g., a cancer cell, wherein the cell comprises a biomarker of sensitivity to a SMARCA2 antagonist (e.g., a compound of Formula (I), (IA), (IB), (IC), (ID), or (IE) or a pharmaceutically acceptable salt thereof).

Some aspects of this disclosure provide the use of a SMARCA2 antagonist (e.g., a SMARCA2 inhibitor, e.g., a compound of Formula (I), (IA), (IB), (IC), (ID), or (IE) or a pharmaceutically acceptable salt thereof) in the manufacture of a medicament for inhibiting an activity of SMARCA2, wherein the medicament is to be contacted with a SMARCA2 enzyme. In some embodiments, the SMARCA2 enzyme is within a cell, e.g., a cancer cell, wherein the cell comprises a biomarker of sensitivity to a SMARCA2 antagonist (e.g., a compound of Formula (I), (IA), (IB), (IC), (ID), or (IE) or a pharmaceutically acceptable salt thereof).

Some aspects of this disclosure provide methods of treating cancer in a subject in need thereof, comprising administering to the subject a therapeutically effective amount of a SMARCA2 antagonist (e.g., a SMARCA2 inhibitor, e.g., a compound of Formula (I), (IA), (IB), (IC), (ID), or (IE) or a pharmaceutically acceptable salt thereof), wherein the subject or a cell of the subject comprises a biomarker of sensitivity to a SMARCA2 antagonist (e.g., a compound of Formula (I), (IA), (IB), (IC), (ID), or (IE) or a pharmaceutically acceptable salt thereof).

Some aspects of this disclosure provide a SMARCA2 antagonist (e.g., a SMARCA2 inhibitor, e.g., a compound of Formula (I), (IA), (IB), (IC), (ID), or (IE) or a pharmaceutically acceptable salt thereof) for use in treating cancer in a subject in need thereof, wherein the subject or a cell of the subject comprises a biomarker of sensitivity to a SMARCA2 antagonist (e.g., a compound of Formula (I), (IA), (IB), (IC), (ID), or (IE) or a pharmaceutically acceptable salt thereof).

Some aspects of this disclosure provide a SMARCA2 antagonist (e.g., a SMARCA2 inhibitor, e.g., a compound of Formula (I), (IA), (IB), (IC), (ID), or (IE) or a pharmaceutically acceptable salt thereof) for use as a medicament for treating cancer in a subject in need thereof, wherein the subject or a cell of the subject comprises a biomarker of sensitivity to a SMARCA2 antagonist (e.g., a compound of Formula (I), (IA), (IB), (IC), (ID), or (IE) or a pharmaceutically acceptable salt thereof).

Some aspects of this disclosure provide the use of a SMARCA2 antagonist (e.g., a SMARCA2 inhibitor, e.g., a compound of Formula (I), (IA), (IB), (IC), (ID), or (IE) or a pharmaceutically acceptable salt thereof) in the manufacture of a medicament for treating cancer in a subject in need thereof, wherein the subject or a cell of the subject comprises a biomarker of sensitivity to a SMARCA2 antagonist (e.g., a compound of Formula (I), (IA), (IB), (IC), (ID), or (IE) or a pharmaceutically acceptable salt thereof).

In some embodiments, the biomarker is a decreased activity or function of SMARCA4. In certain embodiments, the biomarker is loss of function of SMARCA4.

Some aspects of this disclosure provide methods of identifying a subject sensitive to treatment with a SMARCA2 antagonist (e.g., a compound of Formula (I), (IA), (IB), (IC), (ID), or (IE) or a pharmaceutically acceptable salt thereof), comprising detecting a decreased activity or function of SMARCA4 compared to a control level of the activity or the function of SMARCA4 in the subject and administering a SMARCA2 antagonist (e.g., a SMARCA2 inhibitor, e.g., a compound of Formula (I), (IA), (IB), (IC), (ID), or (IE) or a pharmaceutically acceptable salt thereof) to the subject, wherein the subject has a cancer and wherein an improvement in a sign or symptom of the cancer indicates a sensitivity of the subject or of a cancer cell of the subject for SMARCA2 antagonist (e.g., a compound of Formula (I), (IA), (IB), (IC), (ID), or (IE) or a pharmaceutically acceptable salt thereof).

In some embodiments, the control level is the level of activity of SMARCA4 in a subject that does not have cancer.

In some embodiments, the subject is a participant in a clinical trial. In some embodiments, a criterion for participation of a subject in the clinical trial is a decreased activity or function of SMARCA4, or loss of function of SMARCA4, in said subject or a cell of said subject.

In some embodiments, the present disclosure features a method comprising inhibiting a SMARCA2 activity in a cell exhibiting loss of function of SMARCA4, comprising contacting the cell with a SMARCA2 antagonist (e.g., a SMARCA2 inhibitor, e.g., a compound of Formula (I), (IA), (IB), (IC), (ID), or (IE) or a pharmaceutically acceptable salt thereof).

In certain embodiments of the methods disclosed herein, the cell is in a subject, and the method comprises administering a SMARCA2 antagonist (e.g., a SMARCA2 inhibitor, e.g., a compound of Formula (I), (IA), (IB), (IC), (ID), or (IE) or a pharmaceutically acceptable salt thereof) to the subject.

In some aspects, this present disclosure features methods of treating cancer, comprising inhibiting a SMARCA2 activity in a subject in need thereof, wherein the subject has a cancer characterized by loss of function of SMARCA4.

In some aspects, this present disclosure features methods of treating cancer, comprising inhibiting a SMARCA2 activity, e.g., a SMARCA2 helicase activity or a SMARCA2 ATPase activity, in a subject in need thereof, wherein the subject has a cancer characterized by loss of function of SMARCA4.

Some aspects of this disclosure provide methods comprising modulating (e.g., inhibiting) a SMARCA2 activity in a cell or subject. In some embodiments this disclosure provides methods comprising modulating (e.g., inhibiting) a SMARCA2 activity in a cell or subject Some aspects of this disclosure provide methods comprising modulating a SMARCA2 activity in a cell exhibiting a decreased activity or function of SMARCA4. In some embodiments, the cell is in vivo, ex vivo, in vitro, or in situ. In some embodiments, the cell is in a subject, and the method comprises administering a SMARCA2 antagonist (e.g., a SMARCA2 inhibitor, e.g., a compound of Formula (I), (IA), (IB), (IC), (ID), or (IE) or a pharmaceutically acceptable salt thereof) to the subject. In some embodiments, the cell is ex vivo or in vitro, and wherein the cell is isolated or derived from a subject that has a tumor. In some embodiments, the tumor is malignant. In some embodiments, the tumor is metastatic.

Some aspects of this disclosure provide methods of treating cancer in a subject in need thereof, comprising administering a therapeutically effective amount of a SMARCA2 antagonist (e.g., a SMARCA2 inhibitor, e.g., a compound of Formula (I), (IA), (IB), (IC), (ID), or (IE) or a pharmaceutically acceptable salt thereof) to the subject or a cell of the subject, wherein said subject or cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

Some aspects of this disclosure provide a compound of Formula (I), (IA), (IB), (IC), (ID), or (IE) or a pharmaceutically acceptable salt thereof for use in treating cancer in a subject in need thereof.

Some aspects of this disclosure provide a SMARCA2 antagonist (e.g., a SMARCA2 inhibitor, e.g., a compound of Formula (I), (IA), (IB), (IC), (ID), or (IE) or a pharmaceutically acceptable salt thereof) for use in treating cancer in a subject in need thereof, wherein said subject or a cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

Some aspects of this disclosure provide a compound of Formula (I), (IA), (IB), (IC), (ID), or (IE) or a pharmaceutically acceptable salt thereof as a medicament for treating cancer in a subject in need thereof.

Some aspects of this disclosure provide a SMARCA2 antagonist (e.g., a SMARCA2 inhibitor, e.g., a compound of Formula (I), (IA), (IB), (IC), (ID), or (IE) or a pharmaceutically acceptable salt thereof) as a medicament for treating cancer in a subject in need thereof, wherein said subject or a cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

Some aspects of this disclosure provide the use of a compound of Formula (I), (IA), (IB), (IC), (ID), or (IE) or a pharmaceutically acceptable salt thereof in the manufacture of a medicament for treating cancer in a subject in need thereof.

Some aspects of this disclosure provide the use of a SMARCA2 antagonist (e.g., a SMARCA2 inhibitor, e.g., a compound of Formula (I), (IA), (IB), (IC), (ID), or (IE) or a pharmaceutically acceptable salt thereof) in the manufacture of a medicament for treating cancer in a subject in need thereof, wherein said subject or a cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

In some embodiments, the control level is the level of activity or function of SMARCA4 in a subject that does not have cancer. In some embodiments, the method comprises administering the SMARCA2 antagonist (e.g., a SMARCA2 inhibitor, e.g., a compound of Formula (I), (IA), (IB), (IC), (ID), or (IE) or a pharmaceutically acceptable salt thereof) to the cell or the subject based on the decreased activity or function of SMARCA4 in the cell or the subject.

Some aspects of this disclosure provide methods of identifying a subject having a cancer as a candidate for treatment with a SMARCA2 antagonist (e.g., a SMARCA2 inhibitor, e.g., a compound of Formula (I), (IA), (IB), (IC), (ID), or (IE) or a pharmaceutically acceptable salt thereof), comprising detecting a level of activity or function of SMARCA4 in a cancer cell in the subject, comparing the level of activity or function of SMARCA4 detected in the cancer cell to a control or reference level, wherein the subject is identified as a candidate for treatment with a SMARCA2 antagonist (e.g., a SMARCA2 inhibitor, e.g., a compound of Formula (I), (IA), (IB), (IC), (ID), or (IE) or a pharmaceutically acceptable salt thereof), if the level of activity or function of SMARCA4 in the cancer cell is decreased as compared to the control or reference level. In some embodiments, the method comprises obtaining a sample comprising a cancer cell from the subject.

Some aspects of this disclosure provide methods of identifying a cancer cell as sensitive to treatment with a SMARCA2 antagonist (e.g., a SMARCA2 inhibitor, e.g., a compound of Formula (I), (IA), (IB), (IC), (ID), or (IE) or a pharmaceutically acceptable salt thereof), comprising detecting a level of activity or function of SMARCA4 in the cancer cell, comparing the level of activity or function of SMARCA4 detected in the cancer to a control or reference level, wherein the cell is identified as sensitive to treatment with a SMARCA2 antagonist (e.g., a SMARCA2 inhibitor, e.g., a compound of Formula (I), (IA), (IB), (IC), (ID), or (IE) or a pharmaceutically acceptable salt thereof), if the level of activity or function of SMARCA4 is decreased as compared to the control or reference level. In some embodiments, the control or reference level of SMARCA4 activity or function is a level of SMARCA4 observed or expected in a healthy cell of the same origin as the cancer cell.

Some aspects of this disclosure provide methods of treating cancer, comprising inhibiting a SMARCA2 activity in a subject in need thereof, wherein the subject has a cancer characterized by decreased activity of SMARCA4. Some aspects of this disclosure provide methods of treating cancer, comprising inhibiting a SMARCA2 activity in a subject in need thereof, wherein the subject has a cancer characterized by loss of function of SMARCA4.

In some embodiments, the methods of the disclosure comprise contacting a cell with a SMARCA2 antagonist (e.g., a SMARCA2 inhibitor, e.g., a compound of Formula (I), (IA), (IB), (IC), (ID), or (IE) or a pharmaceutically acceptable salt thereof). In certain embodiments, the cell is in vivo, ex vivo, in vitro, or in situ. In certain embodiments of the methods disclosed herein, the cell is in a subject. In some embodiments, the methods of the disclosure comprise administering a SMARCA2 antagonist (e.g., a SMARCA2 inhibitor, e.g., a compound of Formula (I), (IA), (IB), (IC), (ID), or (IE) or a pharmaceutically acceptable salt thereof) to the subject.

In some embodiments, the SMARCA2 antagonist is a SMARCA2 inhibitor. In some embodiments, the SMARCA2 antagonist is a compound of Formula (I), (IA), (IB), (IC), (ID), or (IE) or a pharmaceutically acceptable salt thereof. In some embodiments, the SMARCA2 antagonist is a compound of Table 2, 2a, 2b, 2c, or 2d. In some embodiments, the SMARCA2 inhibitor is a compound of Formula (I), (IA), (IB), (IC), (ID), or (IE) or a pharmaceutically acceptable salt thereof. In some embodiments, the SMARCA2 inhibitor is a compound of Table 2, 2a, 2b, 2c, or 2d.

In some embodiments, the SMARCA2 antagonist (e.g., a SMARCA2 inhibitor, e.g., a compound of Formula (I), (IA), (IB), (IC), (ID), or (IE) or a pharmaceutically acceptable salt thereof) inhibits SMARCA2 helicase activity by at least 10%, at least 20%, at least 30%, at least 40%, at least 50%, at least 60%, at least 70%, at least 80%, at least 90%, at least 95%, at least 98%, or at least 99%, or abolishes SMARCA2 activity. In some embodiments, the SMARCA2 antagonist (e.g., a SMARCA2 inhibitor, e.g., a compound of Formula (I), (IA), (IB), (IC), (ID), or (IE) or a pharmaceutically acceptable salt thereof) inhibits SMARCA2 ATPase activity by at least 10%, at least 20%, at least 30%, at least 40%, at least 50%, at least 60%, at least 70%, at least 80%, at least 90%, at least 95%, at least 98%, or at least 99%, or abolishes SMARCA2 activity.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 illustrates the inhibition of SMARCA2 (IC₅₀) by Compound 139 in the lung cancer cell lines of Table 5. The figure shows that cell lines comprising a loss or absence of SMARCA4 were more sensitive to inhibition of SMARCA2 by Compound 139 than cell lines in which the SMARCA4 protein was present.

FIG. 2 illustrates the results of body weight change of mice (RCBW %) in a Compound 82c efficacy study in a A549 subQ model.

FIG. 3 illustrates mice tumor volume change (%) in a Compound 82c efficacy study in a A549 subQ model.

FIG. 4 illustrates tumor weights (g) in a Compound 82c efficacy study in a A549 subQ model.

FIG. 5 illustrates Day 21 plasma PK (ng/mL) in a Compound 82c efficacy study in a A549 subQ model. The (x) axis represents the vehicle po BIDx21; each set of 4 bars for each time period (pre or post dose), from left to right, represent: (1) Compound 82c 5 mg/kg, po, BIDx21; (2) Compound 82c 12.5 mg/kg, po, BIDx10, QDx11; (3) Compounds 82c 25 mg/kg, po, BIDx7, QDx14; (4) Compound 82c 50 mg/kg, po, QDx10, 3 day soff, 30 mg/kg, po, QDx8.

### DETAILED DESCRIPTION

The present disclosure provides compounds, methods, strategies, compositions, combinations, and dosage forms for the treatment of cell proliferative disorders, e.g., cancers, associated with decreased activity or function of SMARCA4 (e.g., loss of function of SMARCA4).

Some aspects of this disclosure provide methods comprising modulating (e.g., inhibiting) a SMARCA2 activity in a cell or subject. In some embodiments, this disclosure provides methods comprising modulating (e.g., inhibiting) a SMARCA2 activity in a cell or subject.

Some aspects of this disclosure provide methods of treating cancer in a subject in need thereof, comprising administering a therapeutically effective amount of a compound of Formula (I), (IA), (IB), (IC), (ID), or (IE) or a pharmaceutically acceptable salt thereof to the subject or a cell of the subject. In some embodiments, the subject or cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

While many of the embodiments herein describe compounds of Formula (I), (IA), (IB), (IC), (ID), or (IE), it should be understood that such reference also include any pharmaceutically acceptable salts of Formula (I), (IA), (IB), (IC), (ID), or (IE), however, such language has not been included for conciseness.

Some aspects of the disclosure relate to a compound of Formula (I), (IA), (IB), (IC), (ID), or (IE) or a pharmaceutically acceptable salt thereof (e.g., a compound of Table 2, 2a, 2b, 2c, or 2d) for use in the treatment of cancer in a cell or subject. In some embodiments, the subject or cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

Some aspects of the disclosure relate to a compound of Formula (I), (IA), (IB), (IC), (ID), or (IE) or a pharmaceutically acceptable salt thereof (e.g., a compound of Table 2, 2a, 2b, 2c, or 2d) for use as a medicament for the treatment of cancer in a cell or subject. In some embodiments, the subject or cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

Some aspects of the disclosure relate to the use of a compound of Formula (I), (IA), (IB), (IC), (ID), or (IE) or a pharmaceutically acceptable salt thereof (e.g., a compound of Table 2, 2a, 2b, 2c, or 2d) in the manufacture of a medicament for the treatment of cancer in a cell or subject. In some embodiments, the subject or cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

Some aspects of this disclosure provide methods of modulating (e.g., inhibiting) an activity of SMARCA2, comprising contacting SMARCA2 enzyme with a compound of Formula (I), (IA), (IB), (IC), (ID), or (IE) or a pharmaceutically acceptable salt thereof (e.g., a compound of Table 2, 2a, 2b, 2c, or 2d). In some embodiments, the SMARCA2 enzyme is within a cell, e.g., a cancer cell, and the method comprises contacting the cell with a compound of Formula (I), (IA), (IB), (IC), (ID), or (IE) or a pharmaceutically acceptable salt thereof (e.g., a compound of Table 2, 2a, 2b, 2c, or 2d), wherein the cell comprises a biomarker of sensitivity to the SMARCA2 antagonist (e.g. a compound of Formula (I), (IA), (IB), (IC), (ID), or (IE) or a pharmaceutically acceptable salt thereof, e.g., a compound of Table 2, 2a, 2b, 2c, or 2d).

Some aspects of this disclosure provide a compound of Formula (I), (IA), (IB), (IC), (ID), or (IE) or a pharmaceutically acceptable salt thereof (e.g., a compound of Table 2, 2a, 2b, 2c, or 2d) for use in inhibiting an activity of SMARCA2, wherein the compound of Formula (I), (IA), (IB), (IC), (ID), or (IE) or a pharmaceutically acceptable salt thereof (e.g., a compound of Table 2, 2a, 2b, 2c, or 2d) is contacted with a SMARCA2 enzyme. In some embodiments, the SMARCA2 enzyme is within a cell, e.g., a cancer cell, wherein the cell comprises a biomarker of sensitivity to a SMARCA2 antagonist (e.g., a compound of Formula (I), (IA), (IB), (IC), (ID), or (IE) or a pharmaceutically acceptable salt thereof, e.g., a compound of Table 2, 2a, 2b, 2c, or 2d).

Some aspects of this disclosure provide a compound of Formula (I), (IA), (IB), (IC), (ID), or (IE) or a pharmaceutically acceptable salt thereof (e.g., a compound of Table 2, 2a, 2b, 2c, or 2d) for use as a medicament for inhibiting an activity of SMARCA2, wherein the medicament is contacted with a SMARCA2 enzyme. In some embodiments, the SMARCA2 enzyme is within a cell, e.g., a cancer cell, wherein the cell comprises a biomarker of sensitivity to a SMARCA2 antagonist (e.g., a compound of Formula (I), (IA), (IB), (IC), (ID), or (IE) or a pharmaceutically acceptable salt thereof, e.g., a compound of Table 2, 2a, 2b, 2c, or 2d).

Some aspects of this disclosure provide the use of a compound of Formula (I), (IA), (IB), (IC), (ID), or (IE) or a pharmaceutically acceptable salt thereof (e.g., a compound of Table 2, 2a, 2b, 2c, or 2d) in the manufacture of a medicament for inhibiting an activity of SMARCA2, wherein the medicament is to be contacted with a SMARCA2 enzyme. In some embodiments, the SMARCA2 enzyme is within a cell, e.g., a cancer cell, wherein the cell comprises a biomarker of sensitivity to a SMARCA2 antagonist (e.g., a compound of Formula (I), (IA), (IB), (IC), (ID), or (IE) or a pharmaceutically acceptable salt thereof, e.g., a compound of Table 2, 2a, 2b, 2c, or 2d).

Some aspects of this disclosure provide methods of treating cancer in a subject in need thereof, comprising administering to the subject a therapeutically effective amount of a compound of Formula (I), (IA), (IB), (IC), (ID), or (IE) or a pharmaceutically acceptable salt thereof (e.g., a compound of Table 2, 2a, 2b, 2c, or 2d), wherein the subject or a cell of the subject comprises a biomarker of sensitivity to a SMARCA2 antagonist (e.g., a compound of Formula (I), (IA), (IB), (IC), (ID), or (IE) or a pharmaceutically acceptable salt thereof, e.g., a compound of Table 2, 2a, 2b, 2c, or 2d).

Some aspects of this disclosure provide a compound of Formula (I), (IA), (IB), (IC), (ID), or (IE) or a pharmaceutically acceptable salt thereof (e.g., a compound of Table 2, 2a, 2b, 2c, or 2d) for use in treating cancer in a subject in need thereof, wherein the subject or a cell of the subject comprises a biomarker of sensitivity to a SMARCA2 antagonist (e.g., a compound of Formula (I), (IA), (IB), (IC), (ID), or (IE) or a pharmaceutically acceptable salt thereof, e.g., a compound of Table 2, 2a, 2b, 2c, or 2d).

Some aspects of this disclosure provide a compound of Formula (I), (IA), (IB), (IC), (ID), or (IE) or a pharmaceutically acceptable salt thereof (e.g., a compound of Table 2, 2a, 2b, 2c, or 2d) for use as a medicament for treating cancer in a subject in need thereof, wherein the subject or a cell of the subject comprises a biomarker of sensitivity to a SMARCA2 antagonist (e.g., a compound of Formula (I), (IA), (IB), (IC), (ID), or (IE) or a pharmaceutically acceptable salt thereof, e.g., a compound of Table 2, 2a, 2b, 2c, or 2d).

Some aspects of this disclosure provide the use of a compound of Formula (I), (IA), (IB), (IC), (ID), or (IE) or a pharmaceutically acceptable salt thereof (e.g., a compound of Table 2, 2a, 2b, 2c, or 2d) in the manufacture of a medicament for treating cancer in a subject in need thereof, wherein the subject or a cell of the subject comprises a biomarker of sensitivity to a SMARCA2 antagonist (e.g., a compound of Formula (I), (IA), (IB), (IC), (ID), or (IE) or a pharmaceutically acceptable salt thereof, e.g., a compound of Table 2, 2a, 2b, 2c, or 2d).

In some embodiments, the biomarker is a decreased activity or function of SMARCA4. In certain embodiments, the biomarker is loss of function of SMARCA4.

Some aspects of this disclosure provide methods of identifying a subject sensitive to treatment with a SMARCA2 antagonist (e.g., a compound of Formula (I), (IA), (IB), (IC), (ID), or (IE) or a pharmaceutically acceptable salt thereof, e.g., a compound of Table 2, 2a, 2b, 2c, or 2d), comprising detecting a decreased activity or function of SMARCA4 compared to a control level of the activity or the function of SMARCA4 in the subject and administering a compound of Formula (I), (IA), (IB), (IC), (ID), or (IE) or a pharmaceutically acceptable salt thereof (e.g., a compound of Table 2, 2a, 2b, 2c, or 2d) to the subject, wherein the subject has a cancer and wherein an improvement in a sign or symptom of the cancer indicates a sensitivity of the subject or of a cancer cell of the subject for the compound of Formula (I), (IA), (IB), (IC), (ID), or (IE) or a pharmaceutically acceptable salt thereof (e.g., a compound of Table 2, 2a, 2b, 2c, or 2d).

In some embodiments, the control level is the level of activity of SMARCA4 in a subject that does not have cancer.

In some embodiments, the subject is a participant in a clinical trial. In some embodiments, a criterion for participation of a subject in the clinical trial is a decreased activity or function of SMARCA4, or loss of function of SMARCA4, in said subject or a cell of said subject.

In some embodiments, the present disclosure features a method comprising inhibiting a SMARCA2 activity in a cell exhibiting loss of function of SMARCA4, comprising contacting the cell with a compound of Formula (I), (IA), (IB), (IC), (ID), or (IE) or a pharmaceutically acceptable salt thereof (e.g., a compound of Table 2, 2a, 2b, 2c, or 2d).

In certain embodiments of the methods disclosed herein, the cell is in a subject, and the method comprises administering a compound of Formula (I), (IA), (IB), (IC), (ID), or (IE) or a pharmaceutically acceptable salt thereof (e.g., a compound of Table 2, 2a, 2b, 2c, or 2d) to the subject.

In some aspects, this present disclosure features methods of treating cancer, comprising inhibiting a SMARCA2 activity in a subject in need thereof, wherein the subject has a cancer characterized by loss of function of SMARCA4.

In some embodiments, a compound of Formula (I), (IA), (IB), (IC), (ID), or (IE) or a pharmaceutically acceptable salt thereof is a SMARCA2 inhibitor.

In some aspects, this present disclosure features methods of treating cancer, comprising inhibiting a SMARCA2 activity, e.g., a SMARCA2 helicase activity or a SMARCA2 ATPase activity, in a subject in need thereof, wherein the subject has a cancer characterized by loss of function of SMARCA4.

Some aspects of this disclosure provide methods comprising modulating (e.g., inhibiting) a SMARCA2 activity in a cell or subject. In some embodiments, this disclosure provides methods comprising modulating a SMARCA2 activity in a cell exhibiting a decreased activity or function of SMARCA4. In some embodiments, the cell is in vivo, ex vivo, in vitro, or in situ. In some embodiments, the cell is in a subject, and the method comprises administering a compound of Formula (I), (IA), (IB), (IC), (ID), or (IE) or a pharmaceutically acceptable salt thereof (e.g., a compound of Table 2, 2a, 2b, 2c, or 2d) to the subject. In some embodiments, the cell is ex vivo or in vitro, and wherein the cell is isolated or derived from a subject that has a tumor. In some embodiments, the tumor is malignant. In some embodiments, the tumor is metastatic.

Some aspects of this disclosure provide methods of treating cancer in a subject in need thereof, comprising administering a therapeutically effective amount of a compound of Formula (I), (IA), (IB), (IC), (ID), or (IE) or a pharmaceutically acceptable salt thereof (e.g., a compound of Table 2, 2a, 2b, 2c, or 2d) to the subject or a cell of the subject, wherein said subject or cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

Some aspects of this disclosure provide a compound of Formula (I), (IA), (IB), (IC), (ID), or (IE) or a pharmaceutically acceptable salt thereof (e.g., a compound of Table 2, 2a, 2b, 2c, or 2d) for use in treating cancer in a subject in need thereof, wherein said subject or a cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

Some aspects of this disclosure provide a compound of Formula (I), (IA), (IB), (IC), (ID), or (IE) or a pharmaceutically acceptable salt thereof (e.g., a compound of Table 2, 2a, 2b, 2c, or 2d) as a medicament for treating cancer in a subject in need thereof, wherein said subject or a cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

Some aspects of this disclosure provide the use of a SMARCA2 antagonist in the manufacture of a medicament for treating cancer in a subject in need thereof, wherein said subject or a cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

In some embodiments, the control level is the level of activity or function of SMARCA4 in a subject that does not have cancer. In some embodiments, the method comprises administering the SMARCA2 antagonist to the cell or the subject based on the decreased activity or function of SMARCA4 in the cell or the subject. In some embodiments, the subject or cell of the subject exhibits a SMARCA4 mutation as compared to wild-type SMARCA4.

Some aspects of this disclosure provide methods of identifying a subject having a cancer as a candidate for treatment with a compound of Formula (I), (IA), (IB), (IC), (ID), or (IE) or a pharmaceutically acceptable salt thereof (e.g., a compound of Table 2, 2a, 2b, 2c, or 2d), comprising detecting a level of activity or function of SMARCA4 in a cancer cell in the subject, comparing the level of activity or function of SMARCA4 detected in the cancer cell to a control or reference level, wherein the subject is identified as a candidate for treatment with a compound of Formula (I), (IA), (IB), (IC), (ID), or (IE) or a pharmaceutically acceptable salt thereof (e.g., a compound of Table 2, 2a, 2b, 2c, or 2d), if the level of activity or function of SMARCA4 in the cancer cell is decreased as compared to the control or reference level. In some embodiments, the method comprises obtaining a sample comprising a cancer cell from the subject.

Some aspects of this disclosure provide methods of identifying a cancer cell as sensitive to treatment with a compound of Formula (I), (IA), (IB), (IC), (ID), or (IE) or a pharmaceutically acceptable salt thereof (e.g., a compound of Table 2, 2a, 2b, 2c, or 2d), comprising detecting a level of activity or function of SMARCA4 in the cancer cell, comparing the level of activity or function of SMARCA4 detected in the cancer to a control or reference level, wherein the cell is identified as sensitive to treatment with a compound of Formula (I), (IA), (IB), (IC), (ID), or (IE) or a pharmaceutically acceptable salt thereof (e.g., a compound of Table 2, 2a, 2b, 2c, or 2d), if the level of activity or function of SMARCA4 is decreased as compared to the control or reference level. In some embodiments, the control or reference level of SMARCA4 activity or function is a level of SMARCA4 observed or expected in a healthy cell of the same origin as the cancer cell.

Some aspects of this disclosure provide methods of treating cancer, comprising inhibiting a SMARCA2 activity in a subject in need thereof, wherein the subject has a cancer characterized by decreased activity of SMARCA4. Some aspects of this disclosure provide methods of treating cancer, comprising inhibiting a SMARCA2 activity in a subject in need thereof, wherein the subject has a cancer characterized by loss of function of SMARCA4.

In some embodiments, the methods of the disclosure comprise contacting a cell with a compound of Formula (I), (IA), (IB), (IC), (ID), or (IE) or a pharmaceutically acceptable salt thereof (e.g., a compound of Table 2, 2a, 2b, 2c, or 2d). In certain embodiments, the cell is in vivo, ex vivo, in vitro, or in situ. In certain embodiments of the methods disclosed herein, the cell is in a subject. In some embodiments, the methods of the disclosure comprise administering a SMARCA2 antagonist to the subject.

In some embodiments, the cell is ex vivo or in vitro. In further embodiments, the cell is isolated or derived from a subject that has a tumor.

In some embodiments, the tumor is malignant. In some embodiments, the tumor is metastatic.

In some embodiments of the disclosure, a compound of Formula (I), (IA), (IB), (IC), (ID), or (IE) or a pharmaceutically acceptable salt thereof (e.g., a compound of Table 2, 2a, 2b, 2c, or 2d) targets an ATPase domain of SMARCA2. In certain embodiments of the methods disclosed herein, the SMARCA2 inhibitor inhibits an ATPase activity of SMARCA2.

In some embodiments of the disclosure, a compound of Formula (I), (IA), (IB), (IC), (ID), or (IE) or a pharmaceutically acceptable salt thereof (e.g., a compound of Table 2, 2a, 2b, 2c, or 2d) does not target a bromodomain activity of SMARCA2.

In some embodiments, the SMARCA2 antagonist (e.g. a compound of Formula (I), (IA), (IB), (IC), (ID), or (IE) or a pharmaceutically acceptable salt thereof, e.g., a compound of Table 2, 2a, 2b, 2c, or 2d) is a SMARCA2 inhibitor.

In certain embodiments of the methods disclosed herein, the SMARCA2 activity is an ATPase activity.

In certain embodiments of the methods, uses, or medicaments disclosed herein, the SMARCA2 activity is not a bromodomain activity.

In certain embodiments of the disclosure, the SMARCA2 inhibitor inhibits an ATPase activity of SMARCA2.

In some embodiments of the disclosure, the decreased activity of SMARCA4 is caused by a genetic mutation.

In some embodiments of the disclosure, the decreased activity of SMARCA4 is caused by an epigenetic alteration.

In some embodiments of the disclosure, the decreased activity of SMARCA4 is caused by a decrease in SMARCA4 gene transcription, SMARCA4 gene transcript translation, or a combination thereof.

In some embodiments of the disclosure, the decreased activity of SMARCA4 is caused by an epigenetic process, e.g., silencing of a SMARCA4 gene, post-transcriptional or post-translational modulation of the half-life of a SMARCA4 gene product, e.g., inhibition of translation of a SMARCA4 transcript into SMARCA4 protein, or increased turnover of a SMARCA4 protein.

In some embodiments of the disclosure, the decreased activity of SMARCA4 is caused by a decrease in SMARCA4 gene transcription, SMARCA4 gene transcript translation, or a combination thereof.

In some embodiments, the compound of Formula (I), (IA), (IB), (IC), (ID), or (IE) or a pharmaceutically acceptable salt thereof (e.g., a compound of Table 2, 2a, 2b, 2c, or 2d) inhibits SMARCA2 helicase activity by at least 10%, at least 20%, at least 30%, at least 40%, at least 50%, at least 60%, at least 70%, at least 80%, at least 90%, at least 95%, at least 98%, or at least 99%, or abolishes SMARCA2 activity. In some embodiments, the compound of Formula (I), (IA), (IB), (IC), (ID), or (IE) or a pharmaceutically acceptable salt thereof (e.g., a compound of Table 2, 2a, 2b, 2c, or 2d) inhibits SMARCA2 ATPase activity by at least 10%, at least 20%, at least 30%, at least 40%, at least 50%, at least 60%, at least 70%, at least 80%, at least 90%, at least 95%, at least 98%, or at least 99%, or abolishes SMARCA2 activity.

### SMARCA2/SMARCA4

Some aspects of this disclosure are based on the recognition that SMARCA2 is a synthetic lethal target in SMARCA4-mutated cancers or cancers associated with decrease or loss of activity or a function of SMARCA4. Some aspects of this disclosure thus provide methods or medicaments for decreasing or abolishing survival and/or proliferation of cancer cells that exhibit a loss of SMARCA4 function by inhibiting SMARCA2 in such cells.

SMARCA2 and SMARCA4 are SWI/SNF related, matrix associated, actin dependent regulators of chromatin and mutually exclusive paralogs in the SWF/SNF complex. SWF/SNF complexes regulate many cell processes by direct modulation of nucleosomal structure. The catalytic subunits SMARCA2 and SMARCA4 have ATP-dependent helicase activity that repositions nucleosomes.

SWI/SNF complex members are mutated in about 20% of human cancers (Kardoch et al. Nat. Genet., 2013, 45(6), 592-601, incorporated herein by reference in its entirety). For example SMARCA4 mutations occur across a diverse range of cancer types with varying population size and clinical need.

Table 1 below provides a summary of the frequency of SMARCA4 mutations in certain cancer types.

TABLE 1
SMARCA4 mutations in certain cancers

| | | | | Estimated |
|---|---|---|---|---|
| | SMARCA4 | US | 5 Year | SMARCA4-Mutant |
| Cancer Type | Mutations (%) | Cases/Year | Survival (%) | Patients/Year |
| Ovary - SCCOHT | >95% | <300 | 33% | <300 |
| Bladder | 8% | 75,000 | 77% | 6000 |
| Stomach | 6% | 22,000 | 28% | 1320 |
| Lung | 4-5% (NSCLC) | 220,000 | 17% | ~10,000 |
| Glioma/GBM | 2-5% | 20,000 | Variable | ~360 |
| Head and Neck | 4% | 36,000 | 56% | 1440 |
| Kidney | 3-4% (Clear cell, | 64,000 | 72% | ~2000 |
| | Papillary) | | | |
| Uterine/Cervical | 3-4% | 12,000 | 68% | ~400 |
| Pancreas | 3% | 46,000 | 7% | 1380 |

However, SMARCA4 expression can also be regulated by post-transcriptional and post-translational mechanisms. As such, an analysis of mutation frequencies only is likely to underestimate protein loss, and observing only mutations of SMARCA4 may underestimate decrease or loss of activity or a function of SMARCA4 in a patient. Decrease or loss of activity or a function of SMARCA4 can appear in patients who have no mutation of SMARCA4. These patients can by identified by methods such as mRNA or protein assays. In some embodiments of the present disclosure, methods comprising detecting a loss of activity or function of SMARCA4 in a cell or tissue comprise assaying SMARCA4 protein expression levels by a suitable method, such as, e.g., antibody-based assays allowing for quantification of expressed protein in the cell or tissue (e.g., western blot, immunohistochemistry, ELISA, etc.).

Exemplary sequences for SMARCA2 and SMARCA4 are provided herein.

Exemplary sequences for SMARCA2:

mRNA sequence of human SWI/SNF related, matrix associated, actin dependent regulator of chromatin, subfamily a, member 2 (SMARCA2), transcript variant 3 (GenBank Accession No. NM_001289396.1)

mRNA sequence of human SWI/SNF related, matrix associated, actin dependent regulator of chromatin, subfamily a, member 2 (SMARCA2), transcript variant 2 (GenBank Accession No. NM_139045.3)

mRNA sequence of human SWI/SNF related, matrix associated, actin dependent regulator of chromatin, subfamily a, member 2 (SMARCA2), transcript variant 4 (GenBank Accession No. NM_001289397.1)

mRNA sequence of human SWI/SNF related, matrix associated, actin dependent regulator of chromatin, subfamily a, member 2 (SMARCA2), transcript variant 5 (GenBank Accession No. NM_001289398.1)

Protein sequence of human probable global transcription activator SNF2L2 isoform a (GenBank Accession No. NP_001276325.1)

Protein sequence of human probable global transcription activator SNF2L2 isoform b (GenBank Accession No. NP_620614.2)

Protein sequence of human probable global transcription activator SNF2L2 isoform c (GenBank Accession No. NP_001276326.1)

Protein sequence of human probable global transcription activator SNF2L2 isoform d (GenBank Accession No. NP_001276327.1)

Exemplary Sequences for SMARCA4:

mRNA sequence of human SWI/SNF related, matrix associated, actin dependent regulator of chromatin, subfamily a, member 4 (SMARCA4), transcript variant 1 (GenBank Accession No. NM_001128849.1)

mRNA sequence of human SWI/SNF related, matrix associated, actin dependent regulator of chromatin, subfamily a, member 4 (SMARCA4), transcript variant 2 (GenBank Accession No. NM_001128844.1)

mRNA sequence of human SWI/SNF related, matrix associated, actin dependent regulator of chromatin, subfamily a, member 4 (SMARCA4), transcript variant 4 (GenBank Accession No. NM_001128845.1)

mRNA sequence of human SWI/SNF related, matrix associated, actin dependent regulator of chromatin, subfamily a, member 4 (SMARCA4), transcript variant 5 (GenBank Accession No. NM_001128846.1)

mRNA sequence of human SWI/SNF related, matrix associated, actin dependent regulator of chromatin, subfamily a, member 4 (SMARCA4), transcript variant 6 (GenBank Accession No. NM_001128847.1)

mRNA sequence of human SWI/SNF related, matrix associated, actin dependent regulator of chromatin, subfamily a, member 4 (SMARCA4), transcript variant 7 (GenBank Accession No. NM_001128848.1)

Protein sequence of human transcription activator BRG1 isoform A (GenBank Accession No. NP_001122321.1)

Protein sequence of human transcription activator BRG1 isoform B (GenBank Accession No. NP_001122316.1)

Protein sequence of human transcription activator BRG1 isoform C (GenBank Accession No. NP_001122317.1)

Protein sequence of human transcription activator BRG1 isoform D (GenBank Accession No. NP_001122318.1)

Protein sequence of human transcription activator BRG1 isoform E (GenBank Accession No. NP_001122319.1)

Protein sequence of human transcription activator BRG1 isoform F (GenBank Accession No. NP_001122320.1

### SMARCA2 Antagonists

In some embodiments, reduced expression or function, or loss of function, of SMARCA4 confers sensitivity of said cell to inhibition of SMARCA2.

In certain aspects of the disclosure, the inhibitor or antagonist targets the helicase domain of SMARCA2. In some embodiments, the inhibitor or antagonist targets the ATP domain of SMARCA2. In some embodiments, the inhibitor or antagonist does not target the bromodomain of SMARCA2. In some embodiments, the inhibitor or antagonist targets the bromodomain of SMARCA2.

In some aspects, a SMARCA2 antagonist (e.g., a SMARCA2 inhibitor) inhibits SMARCA2 helicase activity. In some embodiments, a SMARCA2 antagonist (e.g., a SMARCA2 inhibitor) inhibits SMARCA2 helicase activity by at least 10%. In some embodiments, a SMARCA2 antagonist (e.g., a SMARCA2 inhibitor) inhibits SMARCA2 helicase activity by at least 20%. In some embodiments, a SMARCA2 antagonist (e.g., a SMARCA2 inhibitor) inhibits SMARCA2 helicase activity by at least 30%. In some embodiments, a SMARCA2 antagonist (e.g., a SMARCA2 inhibitor) inhibits SMARCA2 helicase activity by at least 40%. In some embodiments, a SMARCA2 antagonist (e.g., a SMARCA2 inhibitor) inhibits SMARCA2 helicase activity by at least 50%. In some embodiments, a SMARCA2 antagonist (e.g., a SMARCA2 inhibitor) inhibits SMARCA2 helicase activity by at least 60%. In some embodiments, a SMARCA2 antagonist (e.g., a SMARCA2 inhibitor) inhibits SMARCA2 helicase activity by at least 70%. In some embodiments, a SMARCA2 antagonist (e.g., a SMARCA2 inhibitor) inhibits SMARCA2 helicase activity by at least 80%. In some embodiments, a SMARCA2 antagonist (e.g., a SMARCA2 inhibitor) inhibits SMARCA2 helicase activity by at least 90%. In some embodiments, a SMARCA2 antagonist (e.g., a SMARCA2 inhibitor) inhibits SMARCA2 helicase activity by at least 95%. In some embodiments, a SMARCA2 antagonist (e.g., a SMARCA2 inhibitor) inhibits SMARCA2 helicase activity by at least 98%. In some embodiments, a SMARCA2 antagonist (e.g., a SMARCA2 inhibitor) inhibits SMARCA2 helicase activity by or at least 99%. In some embodiments, a SMARCA2 antagonist (e.g., a SMARCA2 inhibitor) inhibits SMARCA2 helicase activity and abolishes SMARCA2 activity.

In some aspects, a SMARCA2 antagonist (e.g., a SMARCA2 inhibitor) inhibits SMARCA2 ATPase activity. In some embodiments, a SMARCA2 antagonist (e.g., a SMARCA2 inhibitor) inhibits SMARCA2 ATPase activity by at least 10%. In some embodiments, a SMARCA2 antagonist (e.g., a SMARCA2 inhibitor) inhibits SMARCA2 ATPase activity by at least 20%. In some embodiments, a SMARCA2 antagonist (e.g., a SMARCA2 inhibitor) inhibits SMARCA2 ATPase activity by at least 30%. In some embodiments, a SMARCA2 antagonist (e.g., a SMARCA2 inhibitor) inhibits SMARCA2 ATPase activity by at least 40%. In some embodiments, a SMARCA2 antagonist (e.g., a SMARCA2 inhibitor) inhibits SMARCA2 ATPase activity by at least 50%. In some embodiments, a SMARCA2 antagonist (e.g., a SMARCA2 inhibitor) inhibits SMARCA2 ATPase activity by at least 60%. In some embodiments, a SMARCA2 antagonist (e.g., a SMARCA2 inhibitor) inhibits SMARCA2 ATPase activity by at least 70%. In some embodiments, a SMARCA2 antagonist (e.g., a SMARCA2 inhibitor) inhibits SMARCA2 ATPase activity by at least 80%. In some embodiments, a SMARCA2 antagonist (e.g., a SMARCA2 inhibitor) inhibits SMARCA2 ATPase activity by at least 90%. In some embodiments, a SMARCA2 antagonist (e.g., a SMARCA2 inhibitor) inhibits SMARCA2 ATPase activity by at least 95%. In some embodiments, a SMARCA2 antagonist (e.g., a SMARCA2 inhibitor) inhibits SMARCA2 ATPase activity by at least 98%. In some embodiments, a SMARCA2 antagonist (e.g., a SMARCA2 inhibitor) inhibits SMARCA2 ATPase activity by or at least 99%. In some embodiments, a SMARCA2 antagonist (e.g., a SMARCA2 inhibitor) inhibits SMARCA2 ATPase activity and abolishes SMARCA2 activity

In certain aspects of the disclosure, the SMARCA2 antagonist or inhibitor inhibits SMARCA2 activity. Inhibition of SMARCA2 activity can be detected using any suitable method. The inhibition can be measured, for example, either in terms of rate of SMARCA2 activity or as product of SMARCA2 activity.

The inhibition is a measurable inhibition compared to a suitable control. In some embodiments, inhibition is at least 10 percent inhibition compared to a suitable control. That is, the rate of enzymatic activity or the amount of product with the inhibitor is less than or equal to 90 percent of the corresponding rate or amount made without the inhibitor. In some embodiments, inhibition is at least 20, 25, 30, 40, 50, 60, 70, 75, 80, 90, or 95 percent inhibition compared to a suitable control. In some embodiments, inhibition is at least 99 percent inhibition compared to a suitable control. That is, the rate of enzymatic activity or the amount of product with the inhibitor is less than or equal to 1 percent of the corresponding rate or amount made without the inhibitor.

In some embodiments, the SMARCA2 antagonist is a compound of Formula (I):

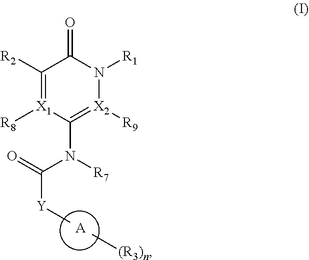

or a pharmaceutically acceptable salt thereof, wherein

A is a heteroaryl, heterocycloalkyl, aryl, or a cycloalkyl;

X₁ and X₂ are each independently selected from —CH and N;

Y is selected from the group consisting of a bond, —NH, —C(O), C₁-C₆ alkyl, —C(CH₃)₂—O—, and —CH₂—NH—CH₂—;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, —OR₅, —C(O)NH₂, —NO₂;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

each R₅ is independently selected from the group consisting of H, cyano, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅, wherein Q is C₁-C₃ alkyl, C₂-C₆ alkenyl, C₃-C₆ cycloalkyl, C₃-C₆ heterocycloalkyl or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

R₈ and R_{9′} are each independently selected from the group consisting of H, halo, and C₁-C₃ alkyl;

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

In some aspects, the present disclosure features a compound of Formula (IA) (e.g., a compound of Table 2, 2a, 2b, 2c, or 2d):

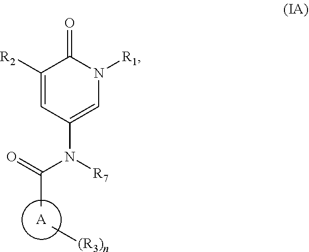

or a pharmaceutically acceptable salt thereof, wherein

A is a heteroaryl, heterocycloalkyl, aryl, or a cycloalkyl;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆—C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, C₃-C₆ cycloalkyl, C₃-C₆ heterocycloalkyl or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

In some embodiments, for a compound Formula (IA) or a pharmaceutically acceptable salt thereof,

A is a heteroaryl, heterocycloalkyl, aryl, or a cycloalkyl;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

In some aspects, the present disclosure features a compound of Formula (IB):

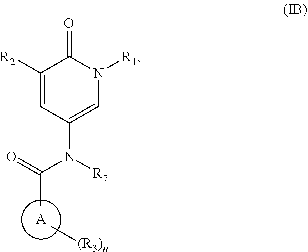

or a pharmaceutically acceptable salt thereof, wherein

A is a heteroaryl, heterocycloalkyl, aryl, or a cycloalkyl;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, C₃-C₆ cycloalkyl, C₃-C₆ heterocycloalkyl or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H and C₁-C₆ alkyl;

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted

In some embodiments, for a compound Formula (IB) or a pharmaceutically acceptable salt thereof,

A is a heteroaryl, heterocycloalkyl, aryl, or a cycloalkyl;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H and C₁-C₆ alkyl;

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

In some aspects, the present disclosure features a compound of Formula (IC):

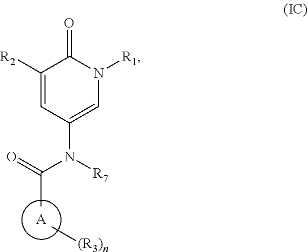

or a pharmaceutically acceptable salt thereof, wherein

A is a 5- or 6-membered heteroaryl having 1 to 4 heteroatoms selected from N, O, and S;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆—C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, C₃-C₆ cycloalkyl, C₃-C₆ heterocycloalkyl or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

In some embodiments, for a compound Formula (IC) or a pharmaceutically acceptable salt thereof,

A is a 5- or 6-membered heteroaryl having 1 to 4 heteroatoms selected from N, O, and S;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

In some aspects, the present disclosure features a compound of Formula (ID) (e.g., a compound of Table 2, 2a, 2b, 2c, or 2d):

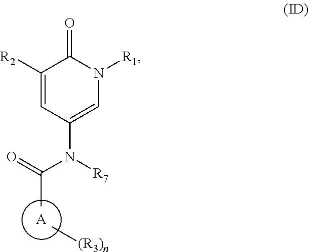

or a pharmaceutically acceptable salt thereof, wherein

A is a heteroaryl, heterocycloalkyl, aryl, or a cycloalkyl;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅;

each Q is independently selected from the group consisting of C₁-C₃ alkyl, C₂-C₆ alkenyl, C₃-C₆ cycloalkyl, C₃-C₆ heterocycloalkyl, and C₂-C₆ alkynyl; each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

provided that at least one R₃ is QR₆, wherein Q is C₂-C₆ alkynyl.

In some embodiments, the SMARCA2 antagonist is a compound of Formula (IE):

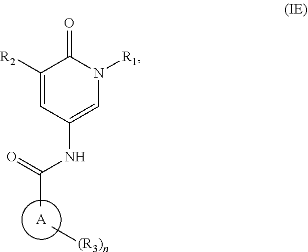

or a pharmaceutically acceptable salt thereof, wherein

A is a 5-membered heteroaryl having 1 to 4 heteroatoms selected from N, O, and S;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆—C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

In some embodiments, each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted with one or more substituents selected from the group consisting of an alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino, alkylamino, dialkylamino, arylamino, diarylamino and alkylarylamino, acylamino, alkylcarbonylamino, arylcarbonylamino, carbamoyl and ureido, amidino, imino, sulfhydryl, alkylthio, arylthio, thiocarboxylate, sulfates, alkylsulfinyl, sulfonato, aminosulfonyl, alkylsulfonyl, sulfonamido, nitro, trifluoromethyl, cyano, azido, cycloalkyl, heterocyclyl, alkylaryl, aromatic and heteroaromatic substituent.

In some embodiments, each alkyl, alkoxyl, alkenyl, alkynyl, alkylcarbonyl, or alkylsulfonyl is unsubstituted or substituted with one or more substituents from the group consisting of halo, amino, alkoxyl, cycloalkyl, aryl, heterocycloalkyl, and heteroaryl.

In some embodiments, each cycloalkyl, aryl, aryloxyl, heterocycloalkyl, or heteroaryl is unsubstituted or substituted with one or more substituents from the group consisting of halo, alkyl, haloalkyl, alkoxyl, cycloalkyl, aryl, heterocycloalkyl, and heteroaryl. In some embodiments, each cycloalkyl, aryl, aryloxyl, heterocycloalkyl, or heteroaryl is unsubstituted or substituted with one or more substituents from the group consisting of halo, alkyl, haloalkyl, and alkoxyl.

In some embodiments, each aminocarbonyl, or aminosulfonyl is unsubstituted or substituted with one or more substituents from the group consisting of halo, alkyl, alkoxyl, cycloalkyl, aryl, heterocycloalkyl, and heteroaryl.

In some embodiments, each cycloalkyl is independently a C₃-C₁₄ cycloalkyl. In some embodiments, each cycloalkyl is independently a C₃-C₈ cycloalkyl.

In some embodiments, each aryl is independently a C₆-C₁₀ aryl.

In some embodiments, each heteroaryl is independently a 5 to 6 membered heteroaryl.

In some embodiments, each heterocycloalkyl is independently a 3 to 8-membered heterocycloalkyl or a 7 to 12-membered heterocycloalkyl.

In some embodiments, A is a 6 membered heteroaryl. In some embodiments, A is a 7-12 membered heteroaryl.

In some embodiments, A is a 3 to 8-membered heterocycloalkyl having 1 to 4 heteroatoms selected from N, O, and S. In some embodiments, A is a 7 to 12-membered heterocycloalkyl having 1 to 4 heteroatoms selected from N, O, and S. In some embodiments, A is a 10-membered heterocycloalkyl having 1 to 4 heteroatoms selected from N, O, and S. In some embodiments, A is a monocyclic heterocycloalkyl. In some embodiments, A is a bicyclic heterocycloalkyl.

In some embodiments, A is C₃-C₁₄ cycloalkyl. In some embodiments, A is C₃-C₈ cycloalkyl. For example, in some embodiments, A is a C₃ cycloalkyl. For example, in some embodiments, A is a C₄ cycloalkyl. For example, in some embodiments, A is a C₅ cycloalkyl. For example, in some embodiments, A is a C₆ cycloalkyl. In some embodiments, A is cyclopropyl.

In some embodiments, A is selected from thiazolyl, isothiazolyl, thiazol-2-onyl, thiophenyl, pyrrolyl, pyrazolyl, imidazolyl, 1,2,3-thiadiazolyl, 1,2,4-thiadiazolyl, furanyl, oxazolyl, isoxazolyl, 1,2,4-triazolyl, and 1,2,3-triazolyl.

In some embodiments, A is selected from the group consisting of thiazolyl, thiophenyl, pyrrolyl, and pyrazolyl. In some embodiments, A is selected from thiazolyl and thiophenyl.

In some embodiments, A is thiazolyl.

In some embodiments, A is isothiazolyl.

In some embodiments, A is thiazol-2-onyl.

In some embodiments, A is thiophenyl.

In some embodiments, A is pyrrolyl.

In some embodiments, A is pyrazolyl.

In some embodiments, A is pyridinyl.

In some embodiments, A is pyrrolidinyl.

In some embodiments, A is imidazolyl.

In some embodiments, A is 1,2,3-thiadiazolyl.

In some embodiments, A is 1,2,4-thiadiazolyl.

In some embodiments, A is benzothiophenyl.

In some embodiments, A is furanyl.

In some embodiments, A is tetrahydrofuranyl.

In some embodiments, A is oxazolyl.

In some embodiments, A is isoxazolyl.

In some embodiments, A is 1,2,4-triazolyl.

In some embodiments, A is 1,2,3-triazolyl.

In some embodiments, A is N-substituted pyrrolyl.

In some embodiments, A is

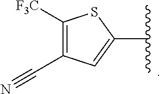 [SMILES: *c1cc(C#N)c(C(F)(F)F)s1]

In some embodiments, A is

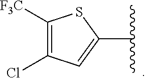 [SMILES: *c1cc(Cl)c(C(F)(F)F)s1]

In some embodiments, Y is a bond.

In some embodiments, Y is —NH.

In some embodiments, Y is —C(O).

In some embodiments, Y is C₁-C₆ alkyl.

In some embodiments, Y is —CH₃.

In some embodiments, Y is CH₂CH₃.

In some embodiments, Y is —C(CH₃)₂—O—.

In some embodiments, Y is —CH₂—NH—CH₂.

In some embodiments, X₁ is —CH.

In some embodiments, X₁ is N.

In some embodiments, X₂ is —CH.

In some embodiments, X₂ is —N.

In some embodiments, R₁ is selected from the group consisting of H, C₁-C₆ alkyl, C₁-C₆ haloalkyl, C₆-C₁₀ aryl, C₃-C₈ cycloalkyl, and —(CH₂)ₘR₄.

In some embodiments, R₁ is selected from the group consisting of H, C₁-C₆ alkyl, or C₁-C₆ haloalkyl.

In some embodiments, R₁ is H.

In some embodiments, R₁ is C₁-C₆ alkyl. For example, in some embodiments, R₁ is methyl, ethyl, n-propyl, i-propyl, n-butyl, i-butyl, tert-butyl, pentyl, or hexyl.

In some embodiments, R₁ is C₁-C₆ haloalkyl. For example, in some embodiments, R₁ is fluoromethyl, fluoroethyl, fluoropropyl, difluoromethyl, difluoroethyl, difluoropropyl, trifluoromethyl, trifluoroethyl, trifluoropropyl, chloromethyl, chloroethyl, chloropropyl, dichloromethyl, dichloroethyl, dichloropropyl, trichloromethyl, trichloroethyl, trichloropropyl, bromomethyl, bromoethyl, bromopropyl, dibromomethyl, dibromoethyl, dibromopropyl, tribromomethyl, tribromoethyl, tribromopropyl, iodomethyl, iodoethyl, iodopropyl, diiodomethyl, diiodoethyl, diiodopropyl, triiodomethyl, triiodoethyl, or triiodopropyl.

In some embodiments, R₁ is methyl, ethyl, halomethyl or haloethyl.

In some embodiments, R₁ is C₁-C₆ fluoroalkyl. In some embodiments, R₁ is selected from the group consisting of fluoromethyl, difluoromethyl, trifluoromethyl, fluoroethyl, difluoroethyl, and trifluoroethyl.

In some embodiments, R₁ is 1,1-difluoroethyl, 1,2-difluoroethyl, 2,1-difluoroethyl, 2,2-difluoroethyl, 1,1,2-trifluoroethyl, 1,2,2-trifluoroethyl, 2,2,1-trifluoroethyl, or 2,2,2-trifluoroethyl.

In some embodiments, R₁ is difluoromethyl.

In some embodiments, R₁ is difluoroethyl.

In some embodiments, R₁ is 2,2-difluoroethyl

In some embodiments, R₁ is C₃-C₈ cycloalkyl. For example, in some embodiments, R₁ is a C₃ cycloalkyl. For example, in some embodiments, R₁ is a C₅ cycloalkyl. For example, in some embodiments, R₁ is a C₆ cycloalkyl. In some embodiments, R₁ is cyclopropyl.

In some embodiments, R₁ is C₆-C₁₀ aryl. For example, in some embodiments, R₁ is phenyl.

In some embodiments, R₁ is —(CH₂)ₘR₄. In some embodiments where R₁ is —(CH₂)ₘR₄, R₄ is selected from the group consisting of C₁-C₆ alkoxyl, mono-C₁-C₆ alkylamino, and di-C₁-C₆ alkylamino.

In some embodiments where R₁ is —(CH₂)ₘR₄, R₄ is hydroxyl.

In some embodiments where R₁ is —(CH₂)ₘR₄, R₄ is C₁-C₆ alkoxyl. For example, in some embodiments, R₄ is methoxyl, ethoxyl, or propyloxyl. In some embodiments, R₄ is methoxyl.

In some embodiments where R₁ is —(CH₂)ₘR₄, R₄ is mono-C₁-C₆ alkylamino. For example, in some embodiments R₄ is methylamino, ethylamino, or propylamino. In some embodiments, R₄ is methylamino.

In some embodiments where R₁ is —(CH₂)ₘR₄, R₄ is di-C₁-C₆ alkylamino. For example, in some embodiments R₄ is dimethylamino, diethylamino, or dipropylamino. For example, in some embodiments R₄ is methylethylamino, methylpropylamino, or ethylpropylamino. In some embodiments, R₄ is dimethylamino.

In some embodiments where R₁ is —(CH₂)ₘR₄, R₄ is C₆-C₁₀ aryl. For example, in some embodiments, R₄ is phenyl.

In some embodiments where R₁ is —(CH₂)ₘR₄, R₄ is C₃-C₈ cycloalkyl. For example, in some embodiments, R₄ is a C₃ cycloalkyl. For example, in some embodiments, R₄ is a C₅ cycloalkyl. For example, in some embodiments, R₄ is a C₆ cycloalkyl. For example, in some embodiments, R₄ is cyclopropyl.

In some embodiments where R₁ is —(CH₂)ₘR₄, R₄ is a 5-membered heteroaryl. For example, in some embodiments, R₄ is pyrazolyl. For example, in some embodiments, R₄ is imidazolyl.

In some embodiments, R₄ is 5-membered a heterocycloalkyl. For example, in some embodiments, R₄ is pyrrolidinyl.

In some embodiments where R₁ is —(CH₂)ₘR₄, m is 1. In some embodiments where R₁ is —(CH₂)ₘR₄, m is 2. In some embodiments where R₁ is —(CH₂)ₘR₄, m is 3, 4, 5, or 6.

In some embodiments, R₂ is selected from the group consisting of H, halo, cyano, C₁-C₆ alkyl, —(CH₂)ₘR_{4′}, —NR₅R_{5′}, and —OR₅.

In some embodiments, R₂ is H.

In some embodiments, R₂ is cyano.

In some embodiments, R₂ is halo. For example, in some embodiments, R₂ is fluoro, chloro, or bromo. In some embodiments, R₂ is fluoro.

In some embodiments, R₂ is C₁-C₆ alkyl. For example, in some embodiments, R₂ is methyl, ethyl, n-propyl, i-propyl, n-butyl, i-butyl, tert-butyl, pentyl, or hexyl. In some embodiments, R₂ is methyl, ethyl, or propyl (e.g., n-propyl, or i-propyl).

In some embodiments, R₂ is —(CH₂)ₘR₄.

In some embodiments wherein R₂ is —(CH₂)ₘR₄, m is 1 or 2. In some embodiments wherein R₂ is —(CH₂)ₘR₄, R₄ is C₁-C₆ aryl. For example, in some embodiments, R₄ is phenyl. In some embodiments wherein R₂ is —(CH₂)ₘR₄, R₄ is a 5-membered heteroaryl. For example, in some embodiments, R₄ is 1-methyl-pyrazolyl.

In some embodiments, R₂ is —NR₅R_{5′.}

In some embodiments where R₂ is —NR₅R_{5′}, R₅ is H and R_{5′} is C₁-C₆ alkyl. For example, in some embodiments, R_{5′} is methyl. For example, in some embodiments, R₂ is methylamino.

In some embodiments where R₂ is —NR₅R_{5′}, R₅ and R_{5′} are both C₁-C₆ alkyl. For example, in some embodiments, R₅ is methyl and R_{5′} is methyl. For example, in some embodiments, R₂ is dimethylamino.

In some embodiments where R₂ is —NR₅R_{5′}, R₅ is H and R_{5′}—(CH₂)ₘR_{4′}. In some embodiments, R_{4′} is C₁-C₆ alkoxyl. For example, in some embodiments, R_{4′} is methoxyl. In some embodiments, R_{4′} is di-C₁-C₆ alkylamino. For example, in some embodiments, R_{4′} is dimethylamino. In some embodiments, R_{4′} is a 6-membered heteroaryl. For example, in some embodiments, R_{4′} is pyridinyl. In some embodiments, R_{4′} is a 6-membered heterocycloalkyl. For example, in some embodiments, R_{4′} is morpholinyl. In some embodiments, R_{4′} is a 5-membered heteroaryl. For example, in some embodiments, R_{4′} is 1-methylpyrazolyl. For example, in some embodiments, R_{4′} is imidazolyl. In some embodiments, R_{4′} is a 5-membered heterocyclyl. For example, in some embodiments, R_{4′} is pyrrolidinyl.

In some embodiments, R₂ is —OR₅. In some embodiments, R₂ is —OR₅ and R₅ is —(CH₂)ₘR_{4′}.

In some embodiments where R₂ is —OR₅ and R₅ is —(CH₂)ₘR_{4′}, R_{4′} is selected from the group consisting of C₁-C₆ alkoxyl, mono-C₁-C₆ alkylamino, and di-C₁-C₆ alkylamino.

In some embodiments where R₂ is —OR₅ and R₅ is —(CH₂)ₘR_{4′}, R_{4′} is C₁-C₆ alkoxyl. For example, in some embodiments, R_{4′} is methoxyl, ethoxyl, or propyloxyl. In some embodiments, R_{4′} is methoxyl.

In some embodiments where R₂ is —OR₅ and R₅ is —(CH₂)ₘR_{4′}, R_{4′} is mono-C₁-C₆ alkylamino. For example, in some embodiments R_{4′} is methylamino, ethylamino, or propylamino. In some embodiments, R_{4′} is methylamino.

In some embodiments R₂ is —C(O)NH₂.

In some embodiments R₂ is —NO₂.

In some embodiments where R₂ is —OR₅ and R₅ is —(CH₂)ₘR_{4′}, R_{4′} is di-C₁-C₆ alkylamino. For example, in some embodiments R_{4′} is dimethylamino, diethylamino, or dipropylamino. For example, in some embodiments R_{4′} is methylethylamino, methylpropylamino, or ethylpropylamino. In some embodiments R_{4′} is dimethylamino.

In some embodiments where R₂ is —OR₅ and R₅ is —(CH₂)ₘR_{4′}, R_{4′} is a 6-membered heterocycloalkyl. For example, in some embodiments, R_{4′} is 1-methylpiperazine or morpholinyl.

In some embodiments wherein R₂ is —OR_{5′} or —NR₅R_{5′} and R₅ is —(CH₂)ₘR₄, m is 1. In some embodiments wherein R₂ is —OR_{5′} or —NR₅R_{5′} and R₅ is —(CH₂)ₘR₄, m is 2.

In some embodiments, m is 1 or 2. In some embodiments, m is 2, 3, 4, 5, or 6.

In some embodiments, m is 1. In some embodiments, m is 2. In some embodiments, m is 3. In some embodiments, m is 4. In some embodiments, m is 5. In some embodiments, m is 6.

In some embodiments, R₄ is halo, COOH, or cyano.

In some embodiments, R₄ is C₂-C₆ alkenyl, or C₂-C₆ alkynyl.

In some embodiments, R₄ is hydroxyl.

In some embodiments, R₄ is C₁-C₆ alkoxyl. For example, in some embodiments, R₄ is methoxyl, ethoxyl, or propyloxyl. In some embodiments, R₄ is methoxyl. In some embodiments, R₄ is ethoxyl.

In some embodiments, R₄ is C₃-C₈ cycloalkyl. For example, in some embodiments, R₄ is a C₃ cycloalkyl. For example, in some embodiments, R₄ is a C₅ cycloalkyl. For example, in some embodiments, R₄ is a C₆ cycloalkyl. For example, R₄ is cyclopropyl.

In some embodiments, R₄ is C₆-C₁₀ aryl, or C₆-C₁₀ aryloxyl. In some embodiments, R₄ is C₆-C₁₀ aryl. For example, in some embodiments R₄ is phenyl.

In some embodiments, R₄ is 3 to 8-membered heterocycloalkyl or a 7 to 12-membered heterocycloalkyl. In some embodiments, R₄ is a 5-membered heterocycloalkyl. For example, in some embodiments, R₄ is pyrrolidinyl. In some embodiments, R₄ is a 6-membered heterocycloalkyl. For example, in some embodiments, R₄ is morpholinyl. For example, in some embodiments, R₄ is methylpiperazinyl. For example, in some embodiments, R₄ is pyrrolidinyl.

In some embodiments, R₄ is 5 to 6-membered heteroaryl. In some embodiments, R₄ is a 5-membered heteroaryl. For example, in some embodiments, R₄ is 1-methylpyrazolyl. In some embodiments, R₄ is a 6-membered heteroaryl. For example, in some embodiments, R₄ is pyridinyl. For example, in some embodiments, R₄ is pyrazolyl. For example, in some embodiments, R₄ is imidazolyl.

In some embodiments, R₄ is mono-C₁-C₆ alkylamino. For example, in some embodiments, R₄ is methylamino, ethylamino, or propylamino. In some embodiments, R₄ is methylamino.

In some embodiments, R₄ is di-C₁-C₆ alkylamino. In some embodiments, R₄ is dimethylamino, diethylamino, or dipropylamino. For example, in some embodiments, R₄ is methylethylamino, methylpropylamino, or ethylpropylamino. In some embodiments, R₄ is dimethylamino.

In some embodiments, R_{4′} is halo, COOH, or cyano.

In some embodiments, R_{4′} is C₂-C₆ alkenyl, or C₂-C₆ alkynyl.

In some embodiments, R_{4′} is hydroxyl.

In some embodiments, R_{4′} is C₁-C₆ alkoxyl. For example, in some embodiments, R_{4′} is methoxyl. For example, in some embodiments, R_{4′} is ethoxyl. For example, in some embodiments, R_{4′} is methoxyl, ethoxyl, or propyloxyl. In some embodiments, R_{4′} is methoxyl.

In some embodiments, R_{4′} is C₃-C₈ cycloalkyl. For example, in some embodiments, R_{4′} is a C₃ cycloalkyl. For example, in some embodiments, R_{4′} is a C₅ cycloalkyl. For example, in some embodiments, R_{4′} is a C₆ cycloalkyl.

In some embodiments, R_{4′} is C₆-C₁₀ aryl, or C₆-C₁₀ aryloxyl. In some embodiments, R_{4′} is C₆-C₁₀ aryl.

In some embodiments, R_{4′} is mono-C₁-C₆ alkylamino. For example, in some embodiments, R_{4′} is methylamino, ethylamino, or propylamino. In some embodiments, R_{4′} is methylamino

In some embodiments, R_{4′} is di-C₁-C₆ alkylamino. For example, in some embodiments R_{4′} is dimethylamino, diethylamino, or dipropylamino. For example, in some embodiments, R_{4′} is methylethylamino, methylpropylamino, or ethylpropylamino. In some embodiments, R_{4′} is dimethylamino.

In some embodiments, R_{4′} is a 3 to 8-membered heterocycloalkyl or a 7 to 12-membered heterocycloalkyl. In some embodiments, R_{4′} is a 5-membered heterocycloalkyl. For example, in some embodiments, R_{4′} is pyrrolidinyl. In some embodiments, R_{4′} is a 6-membered heterocycloalkyl. For example, in some embodiments, R_{4′} is morpholinyl. For example, in some embodiments, R_{4′} is methylpiperazinyl.

In some embodiments, R_{4′} is a 5 to 6-membered heteroaryl. In some embodiments, R_{4′} is a 5-membered heteroaryl. For example, in some embodiments, R_{4′} is 1-methylpyrazolyl. For example, in some embodiments, R_{4′} is imidazolyl. In some embodiments, R_{4′} is a 6-membered heteroaryl. For example, in some embodiments, R_{4′} is pyridinyl.

In some embodiments, R₄ and R_{4′} are each independently selected from the group consisting of hydroxyl, C₁-C₆ alkoxyl, C₃-C₈ cycloalkyl, C₆-C₁₀ aryl, 3 to 8-membered heterocycloalkyl, a 7 to 12-membered heterocycloalkyl, mono-C₁-C₆ alkylamino, and di-C₁-C₆ alkylamino. For example, in some embodiments, R₄ and R_{4′} are each independently selected from the group consisting of methoxyl, cyclopropyl, phenyl, morpholino, methylpiperazinyl, methylamino, and di-methylamino.

In some embodiments, R₅ is H.

In some embodiments, R₅ is cyano.

In some embodiments, R₅ is C₁-C₆ alkyl, C₂-C₆ alkenyl, or C₂-C₆ alkynyl. For example, in some embodiments, R₅ is methyl, ethyl, n-propyl, i-propyl, n-butyl, i-butyl, tert-butyl, pentyl, or hexyl. In some embodiments, R₅ is methyl. In some embodiments, R₅ is i-propyl.

In some embodiments, R₅ is C₁-C₆ alkoxyl. For example, in some embodiments, R₅ is methoxyl, ethoxyl, or propyloxyl.

In some embodiments, R₅ is C₁-C₆ alkylcarbonyl. For example, in some embodiments, R₅ is methanoyl, ethanonyl, or propanoyl. In some embodiments, R₅ is ethanonyl.

In some embodiments, R₅ is C₃-C₈ cycloalkyl. For example, R₅ is a C₃ cycloalkyl. For example, R₅ is a C₅ cycloalkyl. For example, R₅ is a C₆ cycloalkyl. For example, R_{5′} is cyclopentyl.

In some embodiments, R₅ is C₆-C₁₀ aryl, or C₆-C₁₀ aryloxyl. For example, R₅ is phenyl. For example, in some embodiments, R₅ is phenyloxy.

In some embodiments, R₅ is a 3 to 8-membered heterocycloalkyl or a 7 to 12-membered heterocycloalkyl.

In some embodiments, R₅ is 5 to 6-membered heteroaryl.

In some embodiments, R₅ is —(CH₂)ₘR₄.

In some embodiments, R_{5′} is H.

In some embodiments, R_{5′} is C₁-C₆ alkyl, C₂-C₆ alkenyl, or C₂-C₆ alkynyl. For example, in some embodiments, R_{5′} is methyl, ethyl, n-propyl, i-propyl, n-butyl, i-butyl, tert-butyl, pentyl, or hexyl. In some embodiments, R_{5′} is methyl.

In some embodiments, R_{5′} is C₃-C₈ cycloalkyl. For example, in some embodiments, R_{5′} is a C₃ cycloalkyl. For example, in some embodiments, R_{5′} is a C₅ cycloalkyl. For example, in some embodiments, R_{5′} is a C₆ cycloalkyl. For example, R_{5′} is cyclopentyl. In some embodiments, R₅ is i-propyl.

In some embodiments, R_{5′} is C₆-C₁₀ aryl.

In some embodiments, R_{5′} is C₁-C₆ alkylcarbonyl. For example, in some embodiments, R_{5′} is methanoyl, ethanonyl, or propanoyl. In some embodiments, R_{5′} is ethanonyl.

In some embodiments, R_{5′} is a 3 to 8-membered heterocycloalkyl or a 7 to 12-membered heterocycloalkyl.

In some embodiments, R_{5′} is 5 to 6-membered heteroaryl.

In some embodiments, R_{5′} is —(CH₂)ₘR₄.

In some embodiments, R₅ is H and R_{5′} is C₁-C₆ alkyl. For example, R_{5′} is methyl, ethyl, n-propyl, i-propyl, n-butyl, i-butyl, tert-butyl, pentyl, or hexyl.

In some embodiments, R₅ is H and R_{5′} is —(CH₂)ₘR₄.

In some embodiments, R_{5′} is H and R₅ is —(CH₂)ₘR₄.

In some embodiments, where R₅ is —(CH₂)ₘR_{4′}, R_{4′} is selected from hydroxyl, C₁-C₆ alkoxyl, 3 to 8-membered heterocycloalkyl, 7 to 12-membered heterocycloalkyl, mono-C₁-C₆ alkylamino, and di-C₁-C₆ alkylamino.

In some embodiments, R_{4′} is C₁-C₆ alkoxyl. For example, in some embodiments, —(CH₂)ₘR_{4′} is methoxyl, ethoxyl, or propyloxyl. In some embodiments, R_{4′} is methoxyl. For example, in some embodiments, R_{4′} is methylamino, ethylamino, or propylamino. For example, in some embodiments R_{4′} is dimethylamino, diethylamino, or dipropylamino. For example, in some embodiments R_{4′} is methylethylamino, methylpropylamino, or ethylpropylamino. In some embodiments, R_{4′} is dimethylamino. In some embodiments, m is 1 or 2.

In some embodiments, where R_{5′} is —(CH₂)ₘR_{4′}, R_{4′} is selected from hydroxyl, C₁-C₆ alkoxyl, 3 to 8-membered heterocycloalkyl or 7 to 12-membered heterocycloalkyl, mono-C₁-C₆ alkylamino, and di-C₁-C₆ alkylamino. In some embodiments, R_{4′} is C₁-C₆ alkoxyl. For example, in some embodiments, R_{4′} is methoxyl, ethoxyl, or propyloxyl. In some embodiments, R_{4′} is methoxyl. For example, in some embodiments R_{4′} is methylamino, ethylamino, or propylamino. For example, in some embodiments R_{4′} is dimethylamino, diethylamino, or dipropylamino. For example, in some embodiments R_{4′} is methylethylamino, methylpropylamino, or ethylpropylamino. In some embodiments, R_{4′} is dimethylamino. In some embodiments, m is 1 or 2.

In some embodiments, where R_{5′} is —(CH₂)ₘR₄, R₄ is C₁-C₆ aryl. For example, in some embodiments R₄ is phenyl.

In some embodiments, where R₅ is —(CH₂)ₘR₄, R₄ is C₁-C₆ aryl. For example, in some embodiments R₄ is phenyl.

In some embodiments, each R₃ is selected from the group consisting of halo, hydroxyl, COOH, cyano, nitro, oxo, C₁-C₆ alkyl, C₂-C₆ alkenyl, C₂-C₆ alkynyl, C₁-C₆ haloalkyl, C₃-C₈ cycloalkyl, 3 to 8-membered heterocycloalkyl, 7 to 12-membered heterocycloalkyl, aminocarbonyl, mono-C₁-C₆ alkylaminocarbonyl, di-C₁-C₆ alkylaminocarbonyl, C₁-C₆ alkylcarbonylamino, C₁-C₆ alkylsulfonyl, aminosulfonyl, QR₆, —(CH₂)ₘR₆, —NR₅R_{5′}, and —OR₅.

In some embodiments, each R₃ is selected from the group consisting of halo, hydroxyl, COOH, cyano, nitro, C₁-C₆ alkyl, C₂-C₆ alkenyl, C₂-C₆ alkynyl, C₁-C₆ haloalkyl, C₃-C₈ cycloalkyl, C₆-C₁₀ aryl, C₆-C₁₀ aryloxyl, heterocycloalkyl, heteroaryl, aminocarbonyl, mono-C₁-C₆ alkylaminocarbonyl, di-C₁-C₆ alkylaminocarbonyl, C₁-C₆ alkylsulfonyl, aminosulfonyl, QR₆, —(CH₂)ₘR₆, —NR₅R_{5′}, and —OR₅.

In some embodiments, each R₃ is selected from the group consisting of halo, cyano, nitro, oxo, C₁-C₆ alkyl, C₁-C₆ alkenyl, C₁-C₆ haloalkyl, C₃-C₈ cycloalkyl, C₆-C₁₀ aryl, heteroaryl, heterocycloalkyl, aminocarbonyl, mono-C₁-C₆ alkylaminocarbonyl, di-C₁-C₆ alkylaminocarbonyl, C₁-C₆ alkylsulfonyl, aminosulfonyl, -QR₆, —(CH₂)ₘR₆, —NR₅R_{5′}, and —OR₅.

In some embodiments, each R₃ is selected from the group consisting of halo, cyano, nitro, oxo, C₁-C₆ alkyl, C₁-C₆ alkenyl, C₁-C₆ haloalkyl, C₃-C₈ cycloalkyl, heterocycloalkyl, aminocarbonyl, mono-C₁-C₆ alkylaminocarbonyl, di-C₁-C₆ alkylaminocarbonyl, C₁-C₆ alkylsulfonyl, aminosulfonyl, -QR₆, —(CH₂)ₘR₆, —NR₅R_{5′}, and —OR₅.

In some embodiments, R₃ is halo. For example, in some embodiments R₃ is chloro, fluoro, or bromo. In some embodiments R₃ is chloro or fluoro.

In some embodiments, R₃ is hydroxyl or COOH.

In some embodiments, R₃ is cyano.

In some embodiments, R₃ is nitro.

In some embodiments, R₃ is oxo.

In some embodiments, one R₃ is halo and the other R₃ is cyano.

In some embodiments, one R₃ is fluoro and the other R₃ is cyano.

In some embodiments, one R₃ is trifluoromethyl and the other R₃ is cyano.

In some embodiments, one R₃ is C₁-C₆ haloalkyl and the other R₃ is cyano.

In some embodiments, one R₃ is C₁-C₆ trifluoroalkyl and the other R₃ is cyano.

In some embodiments, R₃ is C₁-C₆ alkyl. For example, R₃ is methyl, ethyl, n-propyl, i-propyl, n-butyl, i-butyl, tert-butyl, pentyl, or hexyl.

In some embodiments, R₃ is C₂-C₆ alkenyl or C₂-C₆ alkynyl. For example, in some embodiments, R₃ is C₃ alkenyl. For example, in some embodiments, R₃ is C₃ alkynyl.

In some embodiments, R₃ is C₁-C₆ haloalkyl. For example, in some embodiments, R₃ is fluoromethyl, fluoroethyl, fluoropropyl, difluoromethyl, difluoroethyl, difluoropropyl, trifluoromethyl, trifluoroethyl, trifluoropropyl, chloromethyl, chloroethyl, chloropropyl, dichloromethyl, dichloroethyl, dichloropropyl, trichloromethyl, trichloroethyl, trichloropropyl, bromomethyl, bromoethyl, bromopropyl, dibromomethyl, dibromoethyl, dibromopropyl, tribromomethyl, tribromoethyl, tribromopropyl, iodomethyl, iodoethyl, iodopropyl, diiodomethyl, diiodoethyl, diiodopropyl, triiodomethyl, triiodoethyl, or triiodopropyl. In some embodiments, R₃ is trifluoromethyl.

In some embodiments, R₃ is C₃-C₈ cycloalkyl. For example, in some embodiments, R₃ is a C₃ cycloalkyl. For example, in some embodiments, R₃ is a C₅ cycloalkyl. For example, in some embodiments, R₃ is a C₆ cycloalkyl. In some embodiments, R₃ is cyclopropyl.

In some embodiments, R₃ is aminocarbonyl.

In some embodiments, R₃ is mono-C₁-C₆ alkylaminocarbonyl or di-C₁-C₆ alkylaminocarbonyl. For example, in some embodiments, R₃ is methylaminocarbonyl. For example, in some embodiments, R₃ is dimethylaminocarbonyl.

In some embodiments, R₃ is C₁-C₆ alkylsulfonyl. For example, in some embodiments, R₃ is methylsulfonyl.

In some embodiments, R₃ is aminosulfonyl.

In some embodiments, R₃ is C₆-C₁₀ aryl. For example, in some embodiments, R₃ is phenyl. In some embodiments, C₆-C₁₀ aryl is substituted with one or more groups selected from halo, C₁-C₆ alkyl, and C₁-C₆ alkoxyl. For example, in some embodiments, R₃ is C₆-C₁₀ aryl substituted with Cl, F, Br, or I. For example, in some embodiments, R₃ is C₆-C₁₀ aryl substituted with methyl. For example, in some embodiments, R₃ is C₆-C₁₀ aryl substituted with methoxyl. For example, in some embodiments, R₃ is chlorophenyl. For example, in some embodiments, R₃ is fluorophenyl. For example, in some embodiments, R₃ is bromophenyl. For example, in some embodiments, R₃ is iodophenyl. For example, in some embodiments, R₃ is toluyl. For example, in some embodiments, R₃ is methoxyphenyl.

In some embodiments, R₃ is C₆-C₁₀ aryloxyl.

In some embodiments, R₃ is a 3 to 8-membered heterocycloalkyl or a 7 to 12-membered heterocycloalkyl.

In some embodiments, R₃ is a 5 to 6-membered heteroaryl. For example, in some embodiments, R₃ is selected from oxazolyl, pyridinyl, furanyl, thiazolyl, pyrrolyl, imidazolyl, and pyrazolyl. In some embodiments, the 5 to 6-membered heteroaryl is substituted with one or more methyl. For example, in some embodiments, R₃ is selected from the group consisting of 2-methylthiazolyl, 1,2-dimethyl-pyrrolyl, 1-methyl-imidazolyl, and 1-methyl-pyrazolyl. In some embodiments, the 5 to 6-membered heteroaryl is substituted with one or more C₁-C₆ haloalkyl. For example, in some embodiments, the 5 to 6-membered heteroaryl is substituted with one or more trifluoromethyl. For example, in some embodiments, R₃ is 2-(trifluoromethyl)-2H-imidazolyl.

In some embodiments, R₃ is a 7 to 12-membered heterocycloalkyl. For example, in some embodiments, R₃ is 2,3-dihydrobenzofuranyl.

In some embodiments, R₃ is —(CH₂)ₘR₆. In some embodiments, R₃ is —(CH₂)ₘR₆ and m is 1. In some embodiments, R₃ is —(CH₂)ₘR₆ and m is 2. In some embodiments, R₃ is —(CH₂)ₘR₆ and m is 3, 4, 5, or 6.

In some embodiments, where R₃ is —(CH₂)ₘR₆, R₆ is C₆-C₁₀ aryl. For example, in some embodiments, R₆ is phenyl.

In some embodiments, where R₃ is —(CH₂)ₘR₆, R₆ is C₆-C₁₀ aryl substituted with C₁-C₆ alkoxyl. In some embodiments, R₆ is phenyl substituted with C₁-C₆ alkoxyl. For example, in some embodiments, R₆ is methoxyphenyl.

In some embodiments, where R₃ is —(CH₂)ₘR₆, R₆ is di-C₁-C₆ alkylamino. For example, in some embodiments R₆ is dimethylamino, diethylamino, or dipropylamino. For example, in some embodiments R₆ is methylethylamino, methylpropylamino, or ethylpropylamino. In some embodiments, R₆ is dimethylamino.

In some embodiments, where R₃ is —(CH₂)ₘR₆, R₆ is hydroxyl.

In some embodiments, R₃ is QR₆.

In some embodiments, at least one R₃ is QR₆.

In some embodiments, at least one R₃ is QR₆, wherein Q is C₂-C₆ alkynyl.

In some embodiments, Q is C₂-C₆ alkynyl. For example, in some embodiments, Q is prop-1-ynyl.

In some embodiments, Q is a C₁-C₃ alkyl. For example, in some embodiments, Q is methyl.

In some embodiments, Q is substituted with halogen or hydroxyl. For example, in some embodiments, Q is substituted with hydroxyl. For example, in some embodiments, Q is methanolyl. In some embodiments, Q is substituted with halo. For example, in some embodiments, Q is substituted with fluoro, chloro, iodo, or bromo. In some embodiments, Q is 1,1-difluoropropanyl.

In some embodiments, wherein R₃ is QR₆, R₆ is a 5-membered heterocycloalkyl. For example, in some embodiments, R₆ is pyrrolidine.

In some embodiments, wherein R₃ is QR₆, R₆ is a 6-membered heteroaryl. For example, in some embodiments, R₆ is pyridinyl.

In some embodiments, wherein R₃ is QR₆, R₆ is amino.

In some embodiments, wherein R₃ is QR₆, R₆ is di-C₁-C₆ alkylamino. For example, in some embodiments, R₆ is dimethylamino. In some embodiments, wherein R₃ is QR₆, R₆ is hydroxyl.

In some embodiments, wherein R₃ is QR₆, R₆ is C₁-C₆ haloalkyl. For example, in some embodiments, R₆ is trifluoromethyl.

In some embodiments, R₃ is —NR₅R_{5′}.

In some embodiments where R₃ is —NR₅R_{5′}, R₅ is H and R_{5′} is C₃-C₈ cycloalkyl. For example, in some embodiments, R_{5′} is a C₃ cycloalkyl. For example, in some embodiments, R_{5′} is a C₅ cycloalkyl. For example, in some embodiments, R_{5′} is a C₆ cycloalkyl. In some embodiments, R_{5′} is cyclopentyl.

In some embodiments where R₃ is —NR₅R_{5′}, R₅ is H and R_{5′} is C₁-C₆ alkyl. For example, R_{5′} is methyl, ethyl, n-propyl, i-propyl, n-butyl, i-butyl, tert-butyl, pentyl, or hexyl. In some embodiments, R_{5′} is methyl. In some embodiments, R_{5′} is i-propyl.

In some embodiments where R₃ is —NR₅R_{5′}, R₅ is H and R_{5′} is C₁-C₆ alkenyl or C₁-C₆ haloalkyl. For example, in some embodiments, R₅ is C₃ alkenyl.

In some embodiments where R₃ is —NR₅R_{5′}, R₅ is H and R_{5′} is C₁-C₆ alkylcarbonyl. For example, in some embodiments, R_{5′} is ethanoyl.

In some embodiments, R₃ is —OR₅.

In some embodiments where R₃ is —OR₅, R₅ is C₁-C₆ alkyl. For example, in some embodiments, R₅ is methyl, ethyl, n-propyl, i-propyl, n-butyl, i-butyl, tert-butyl, pentyl, or hexyl. In some embodiments, R₅ is methyl.

In some embodiments where R₃ is —OR₅, R₅ is C₁-C₆ alkenyl or C₁-C₆ alkynyl.

In some embodiments where R₃ is —OR₅, R₅ is C₁-C₆ haloalkyl.

In some embodiments, n is 1. In some embodiments, n is 2. In some embodiments, n is 3. In some embodiments, n is 4.

In some embodiments, n is 1 and R₃ is cyano. In some embodiments, n is 1 or 2 and R₃ is halo. In some embodiments, n is 2, one R₃ is halo and the other R₃ is cyano. In some embodiments, halo is selected from Cl, Br, and I. For example, in some embodiments, n is 2, one R₃ is Cl and the other R₃ is cyano. For example, in some embodiments, n is 2, one R₃ is Br and the other R₃ is cyano. For example, in some embodiments, n is 2, one R₃ is I and the other R₃ is cyano.

In some embodiments, R₆ is selected from the group consisting of halo, hydroxyl, COOH, cyano, C₂-C₆ alkenyl, C₂-C₆ alkynyl, C₁-C₆ alkoxyl, heterocycloalkyl, amino, mono-C₁-C₆ alkylamino, and di-C₁-C₆ alkylamino.

In some embodiments, R₆ is halo, hydroxyl, COOH, or cyano.

In some embodiments, R₆ is C₂-C₆ alkenyl, C₂-C₆ alkynyl.

In some embodiments, R₆ is C₁-C₆ alkoxyl. For example, in some embodiments, R₆ is methoxyl, ethoxyl, or propyloxyl.

In some embodiments, R₆ is C₃-C₈ cycloalkyl. For example, in some embodiments, R₆ is a C₃ cycloalkyl. For example, in some embodiments, R₆ is a C₅ cycloalkyl. For example, in some embodiments, R₆ is a C₆ cycloalkyl. In some embodiments, R₆ is cyclopropyl.

In some embodiments, R₆ is C₆-C₁₀ aryl or C₆-C₁₀ aryloxyl.

In some embodiments, R₆ is a 3 to 8-membered heterocycloalkyl.

In some embodiments, R₆ is a 4-membered heterocycloalkyl. For example, R₆ is oxetanyl.

In some embodiments, R₆ is a 5-membered heterocycloalkyl. For example, in some embodiments, R₆ is pyrrolidinyl or morpholinyl.

In some embodiments, R₆ is 5 to 6-membered heteroaryl. For example, in some embodiments, R₆ is pyridinyl, pyrimidinyl, furanyl, thiazolyl, imidazolyl, or pyrrolyl. In some embodiments, the 5 to 6-membered heteroaryl is substituted with one or more methyl. For example, in some embodiments, R₆ is 2-methylthiazolyl or 1,2-dimethyl-1H-pyrrolyl.

In some embodiments, R₆ is selected from the group consisting of halo, hydroxyl, COOH, cyano, C₂-C₆ alkenyl, C₂-C₆ alkynyl, C₁-C₆ alkoxyl, 3 to 8-membered heterocycloalkyl, amino, mono-C₁-C₆ alkylamino, and di-C₁-C₆ alkylamino.

In some embodiments, R₆ is amino, mono-C₁-C₆ alkylamino, or di-C₁-C₆ alkylamino.

In some embodiments, each amino, mono-C₁-C₆ alkylamino, or di-C₁-C₆ alkylamino is unsubstituted or substituted. In some embodiments, each amino, mono-C₁-C₆ alkylamino, or di-C₁-C₆ alkylamino is unsubstituted.

In some embodiments, R₈ is H.

In some embodiments, R₈ is halo.

In some embodiments, R₈ is F.

In some embodiments, R₈ is Cl.

In some embodiments, R₈ is C₁-C₃ alkyl

In some embodiments, R₈ is CH₃.

In some embodiments, R₈ is CH₂CH₃.

In some embodiments, R₉ is H.

In some embodiments, R₉ is halo.

In some embodiments, R₉ is F.

In some embodiments, R₉ is Cl.

In some embodiments, R₉ is C₁-C₃ alkyl

In some embodiments, R₉ is CH₃.

In some embodiments, R₉ is CH₂CH₃.

In some embodiments, at least one R₃ is QR₆.

In some embodiments, R₃ is QR₆.

In some embodiments, R₃ is QR₆ and Q is selected from the group consisting of C₃-C₆ cycloalkyl, C₃-C₆ heterocycloalkyl or C₂-C₆ alkynyl.

In some embodiments, R₃ is QR₆ and Q is C₃-C₆ cycloalkyl.

In some embodiments, R₃ is QR₆ and Q is a C₃-C₆ cycloalkyl selected from the group consisting of cyclopropyl, cyclopentyl, and cyclohexyl.

In some embodiments, R₃ is QR₆ and Q is C₃-C₆ heterocycloalkyl.

In some embodiments, R₃ is QR₆ and Q is a C₃-C₆ heterocycloalkyl selected from the group consisting of azetidinyl, oxtanyl, pyrrolidinyl, piperidinyl, piperazinyl, and tetrahydropyranyl.

In some embodiments, R₃ is QR₆ and Q is C₂-C₆ alkynyl.

In some embodiments, R₃ is

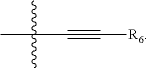

In some embodiments, R₃ is

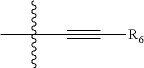

and R₆ is selected from the group consisting of oxetanyl, azetidinyl, piperidinyl, tetrahydropyranyl, phenyl, pyridinyl, pyridazinyl, pyrimidinyl, pyrazinyl, tetrahydropyranyl, tetrahydrofuranyl, pyrrolyl, pyrazolyl, imidazolyl, 1,2,3-triazolyl, tetrazolyl, 1,2,5-thiadiazolyl, 1,3,4-thiadiazolyl, 1,2,3-oxadiazolyl, 1,2,5-oxadiazolyl, thiazolyl, isothiazolyl, isoxazolyl, oxazolyl, and thiophenyl.

In some embodiments, R₃ is

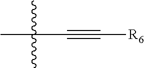

and R₆ is unsubstituted or substituted with an alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, aminosulfonyl, alkylsulfonyl, sulfonamido, nitro, trifluoromethyl, cyano, azido, cycloalkyl, heterocyclyl, alkylaryl, or an aromatic or heteroaromatic moiety.

In some embodiments, R₃ is

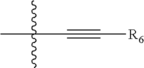

and R₆ is unsubstituted or substituted with halogen or hydroxyl. For example, in some embodiments, R₆ is substituted with hydroxyl. In some embodiments, R₆ is substituted with halo. For example, in some embodiments, R₆ is substituted with fluoro, chloro, iodo, or bromo.

In some embodiments, at least one R₃ is QR₆ and Q is selected from the group consisting of C₃-C₆ cycloalkyl, C₃-C₆ heterocycloalkyl or C₂-C₆ alkynyl.

In some embodiments, at least one R₃ is QR₆ and Q is C₃-C₆ cycloalkyl.

In some embodiments, at least one R₃ is QR₆ and Q is a C₃-C₆ cycloalkyl selected from the group consisting of cyclopropyl, cyclopentyl, and cyclohexyl.

In some embodiments, at least one R₃ is QR₆ and Q is C₃-C₆ heterocycloalkyl.

In some embodiments, at least one R₃ is QR₆ and Q is a C₃-C₆ heterocycloalkyl selected from the group consisting of azetidinyl, oxtanyl, pyrrolidinyl, piperidinyl, piperazinyl, and tetrahydropyranyl.

In some embodiments, at least one R₃ is QR₆ and Q is C₂-C₆ alkynyl.

In some embodiments, at least one R₃ is

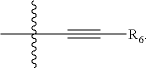

In some embodiments, at least one R₃ is

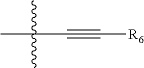

and R₆ is selected from the group consisting of oxetanyl, azetidinyl, piperidinyl, tetrahydropyranyl, phenyl, pyridinyl, pyridazinyl, pyrimidinyl, pyrazinyl, tetrahydropyranyl, tetrahydrofuranyl, pyrrolyl, pyrazolyl, imidazolyl, 1,2,3-triazolyl, tetrazolyl, 1,2,5-thiadiazolyl, 1,3,4-thiadiazolyl, 1,2,3-oxadiazolyl, 1,2,5-oxadiazolyl, thiazolyl, isothiazolyl, isoxazolyl, oxazolyl, and thiophenyl.

In some embodiments, at least one R₃ is

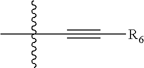

and R₆ is unsubstituted or substituted with an alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, aminosulfonyl, alkylsulfonyl, sulfonamido, nitro, trifluoromethyl, cyano, azido, cycloalkyl, heterocyclyl, alkylaryl, or an aromatic or heteroaromatic moiety.

In some embodiments, at least one R₃ is

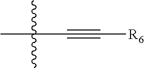

and R₆ is unsubstituted or substituted with halogen or hydroxyl. For example, in some embodiments, R₆ is substituted with hydroxyl. In some embodiments, R₆ is substituted with halo. For example, in some embodiments, R₆ is substituted with fluoro, chloro, iodo, or bromo.

In some embodiments,

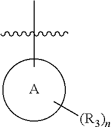

is selected from:

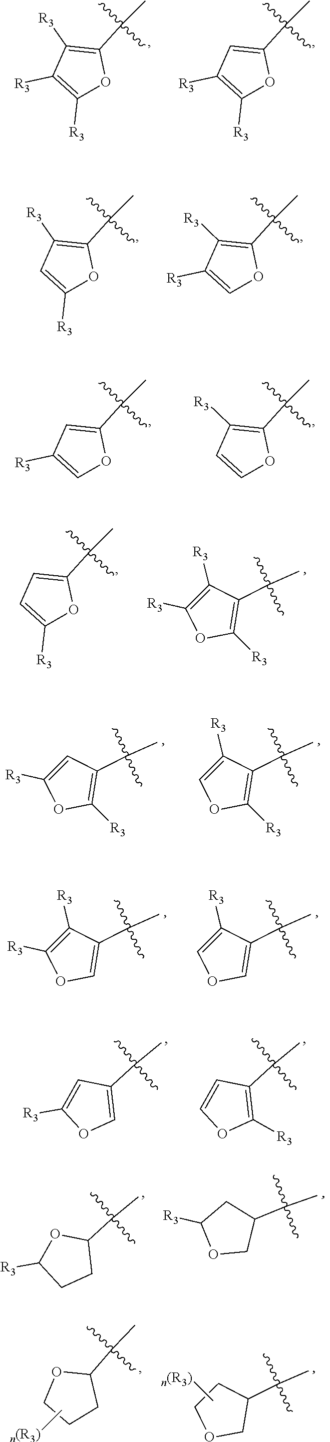

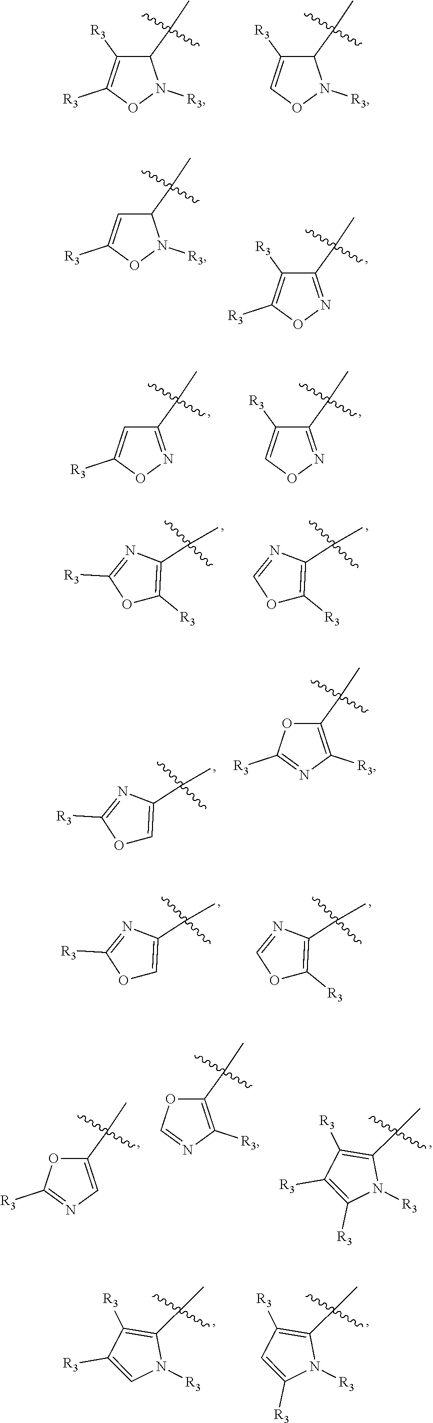

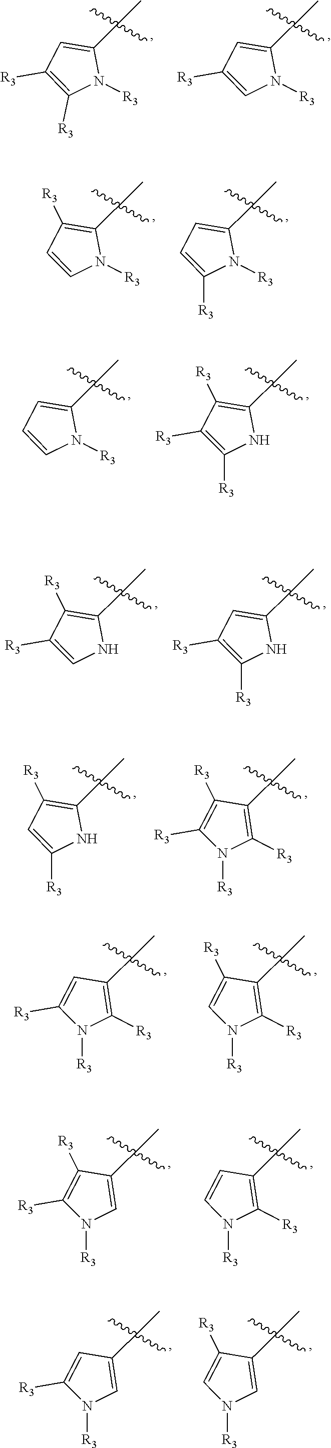

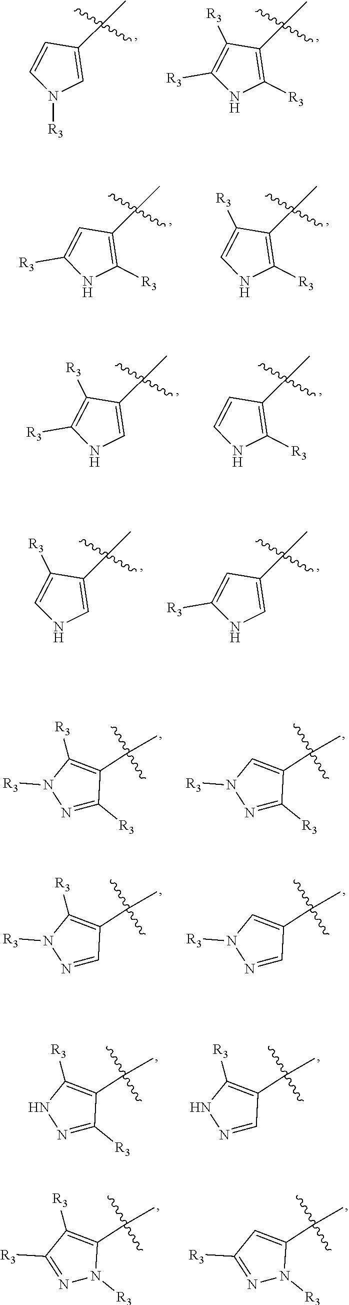

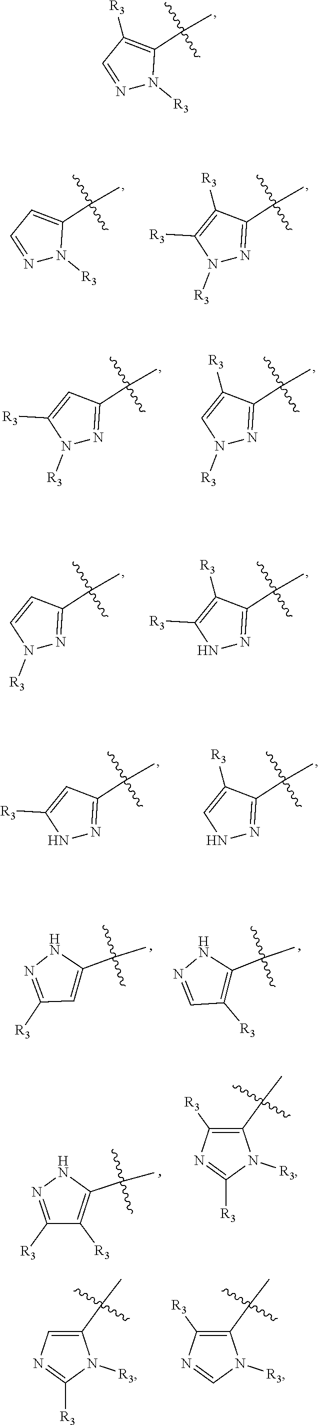

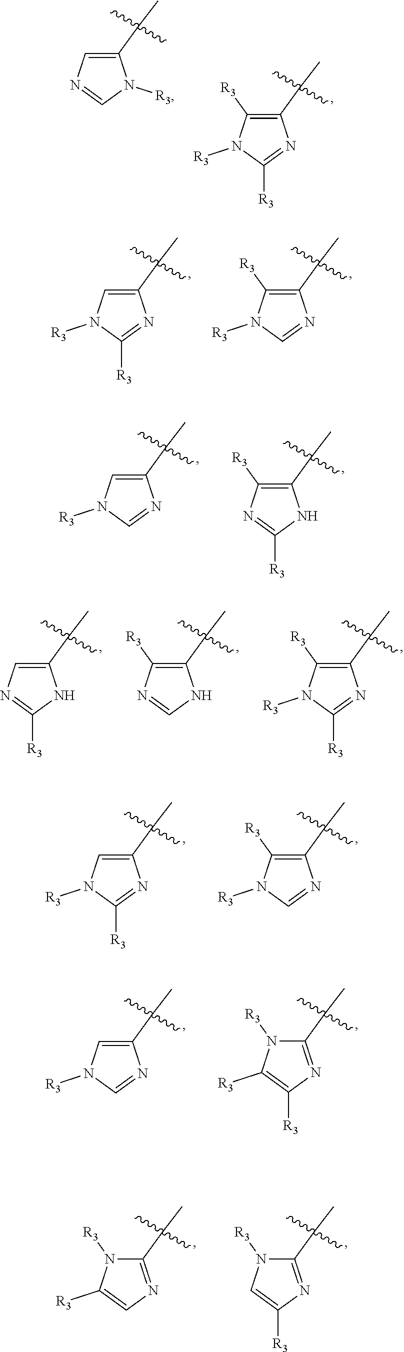

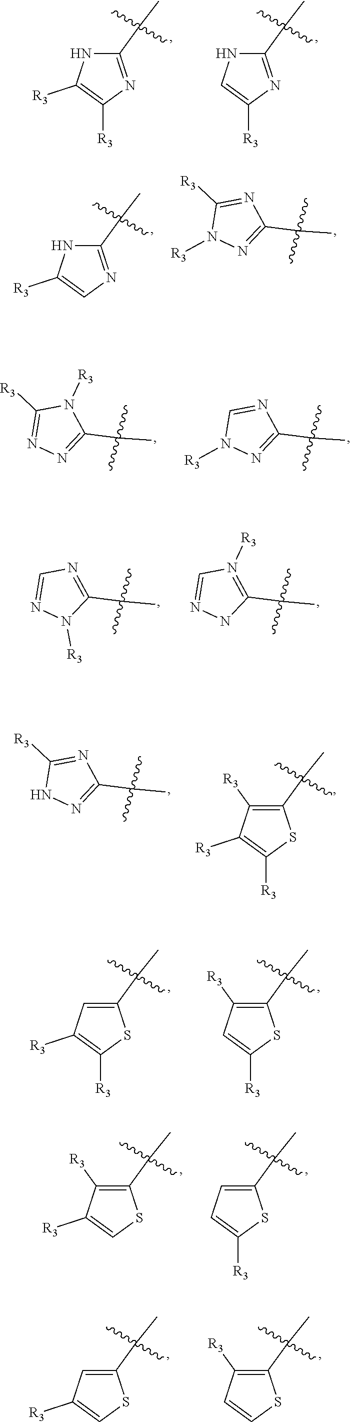

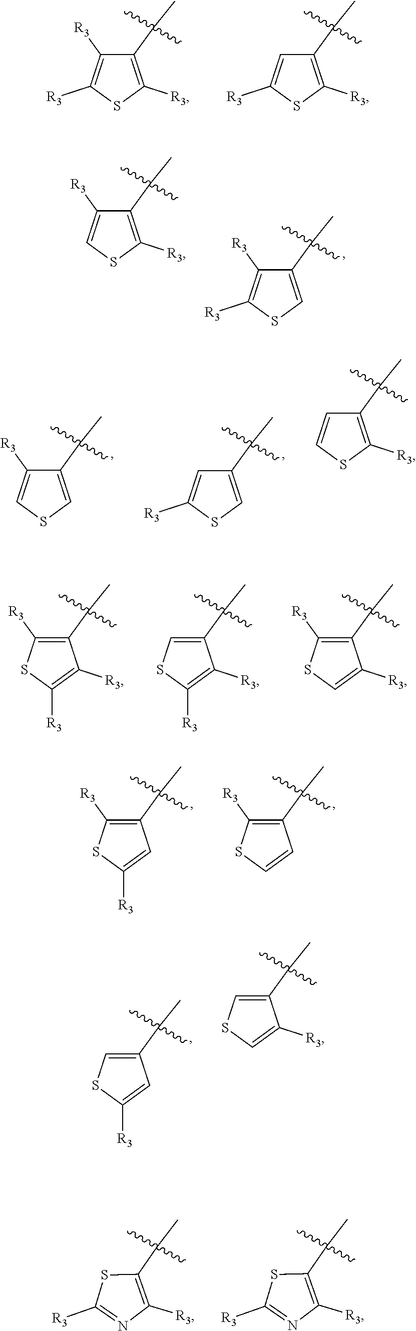

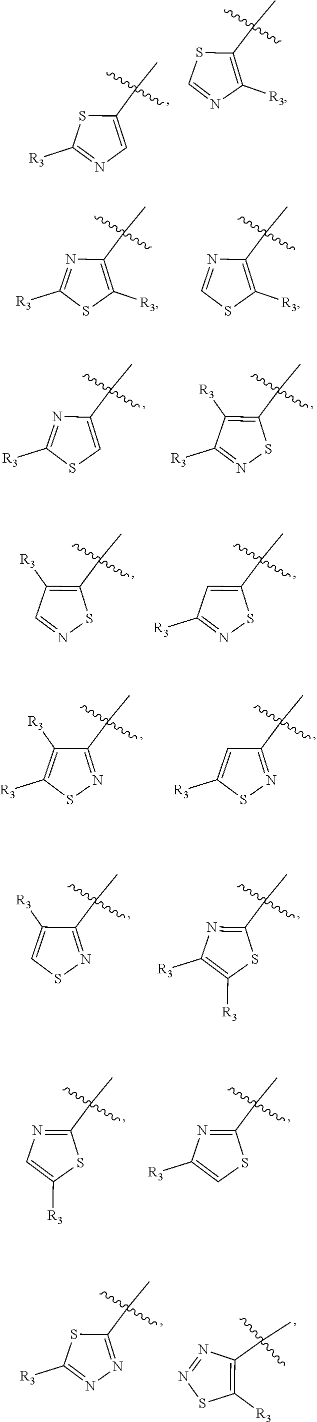

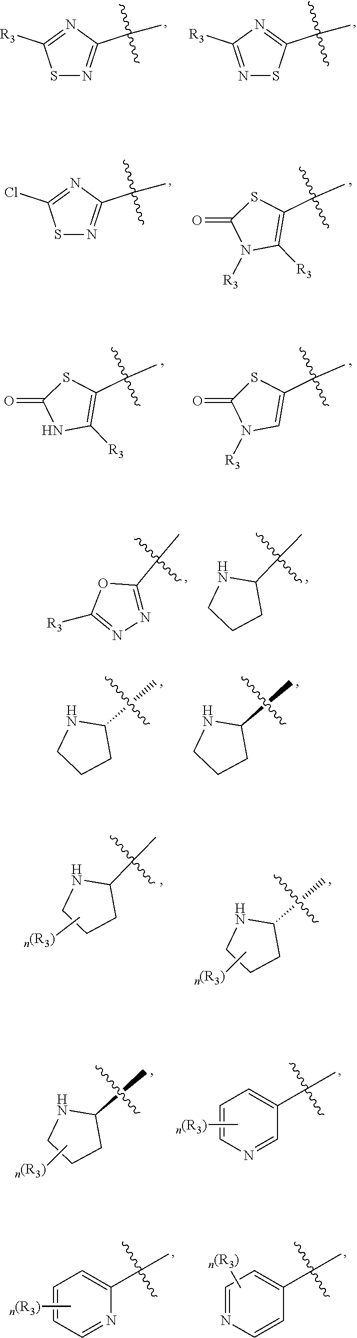

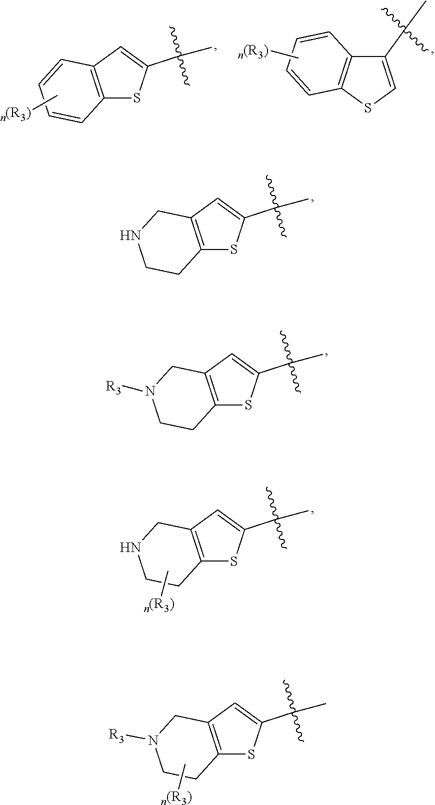

and tautomers thereof.

In some embodiments

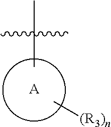

is selected from:

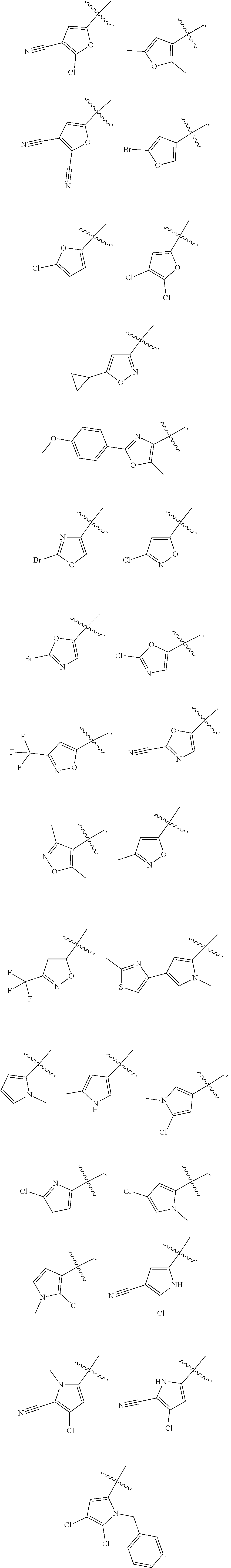

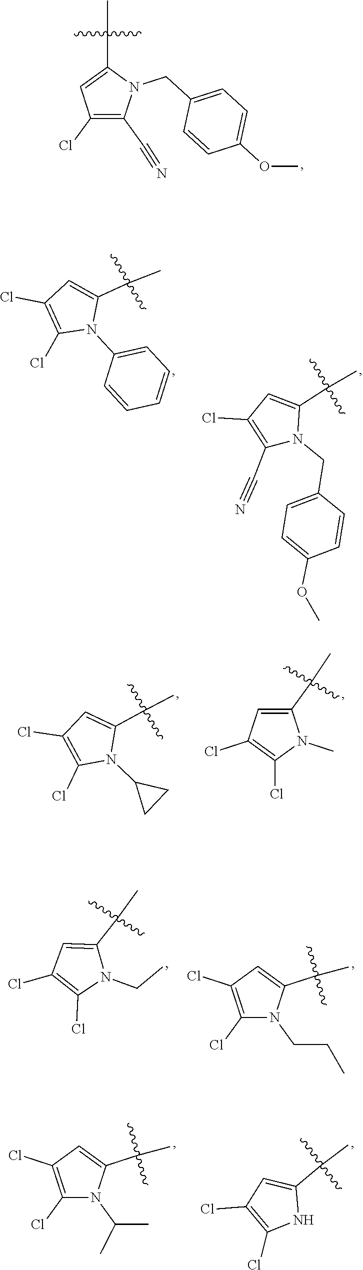

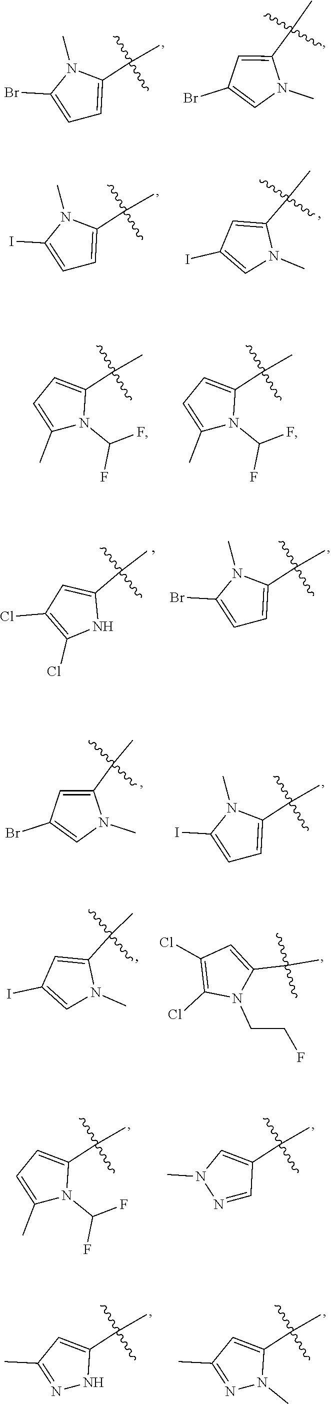

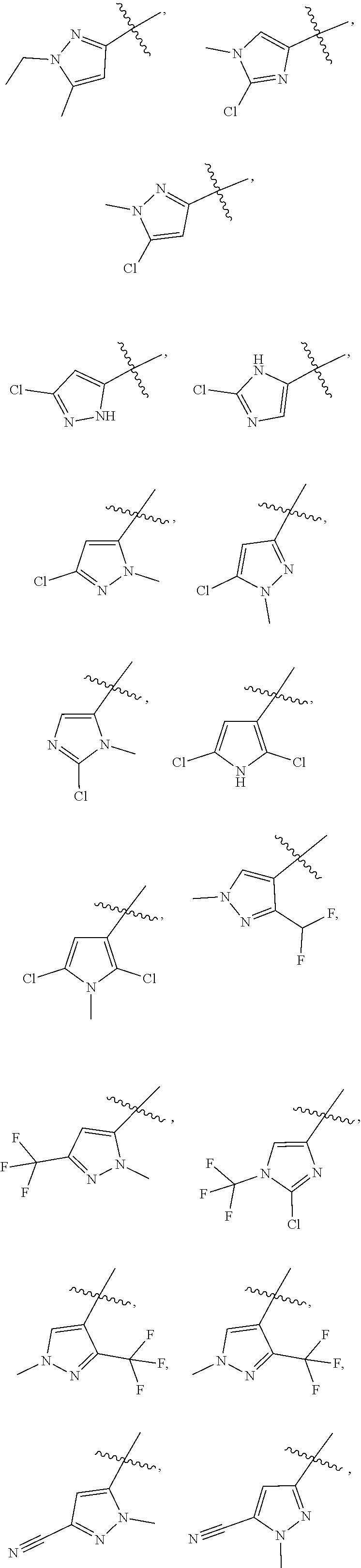

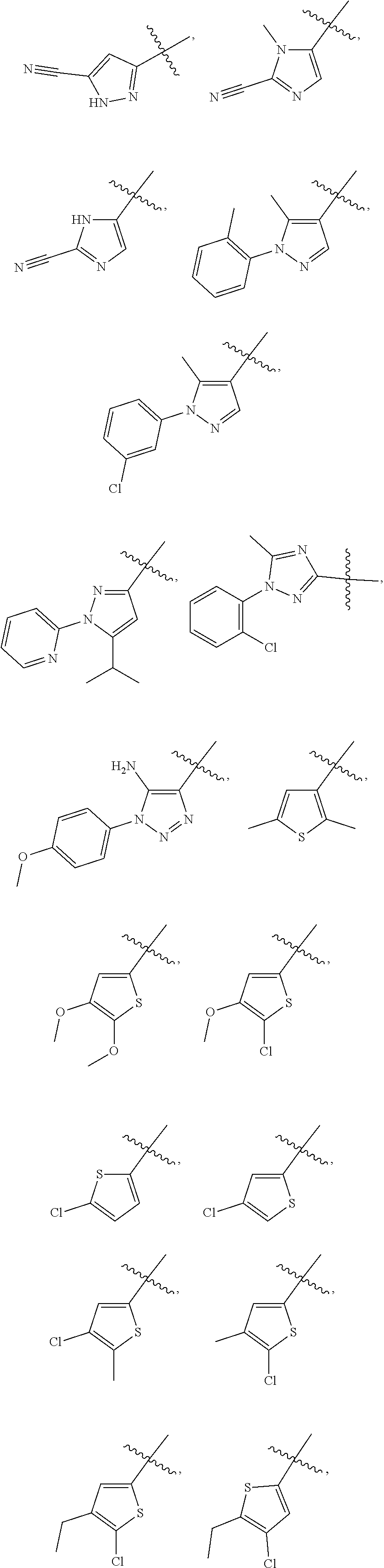

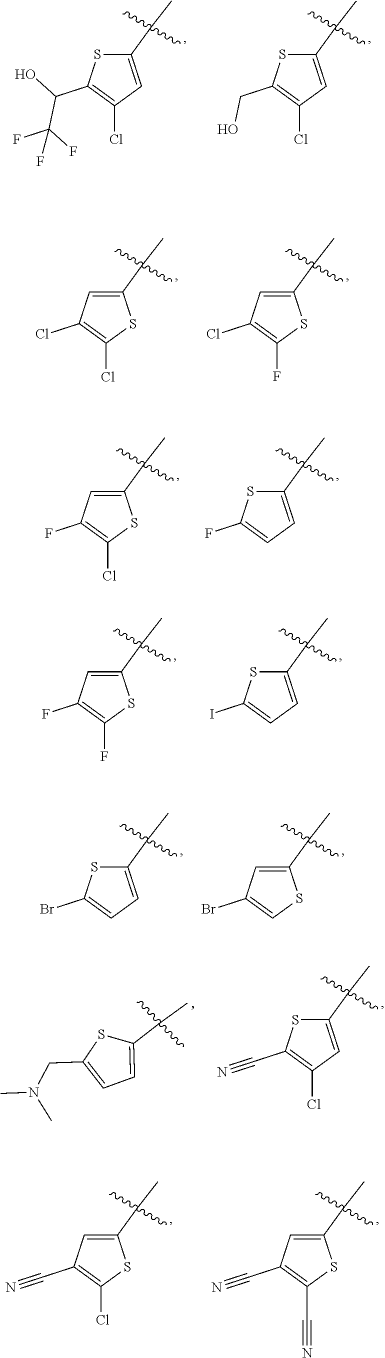

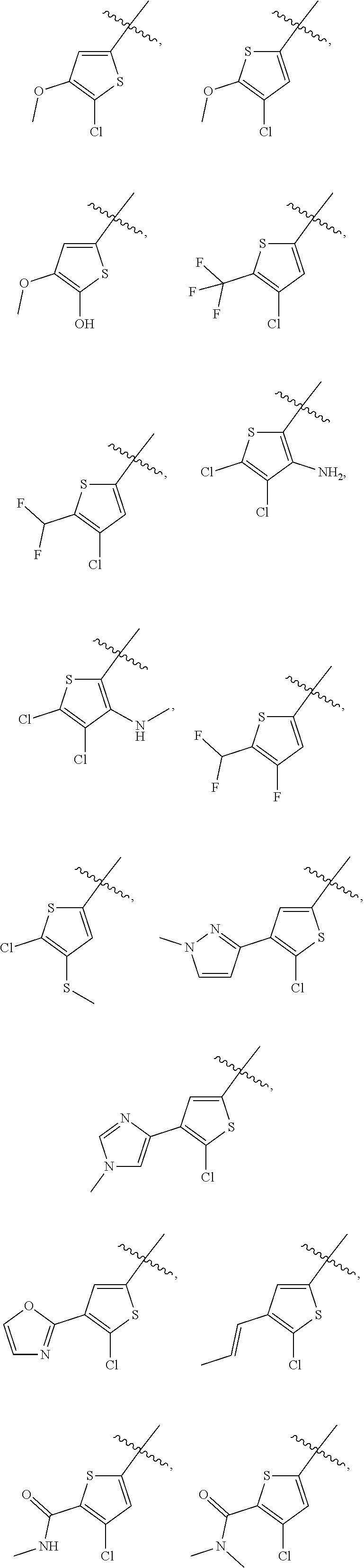

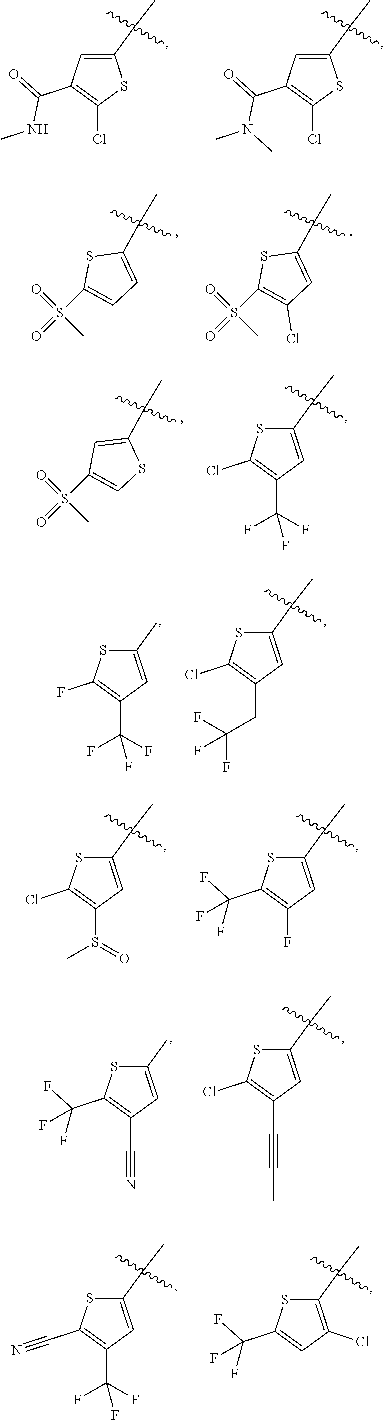

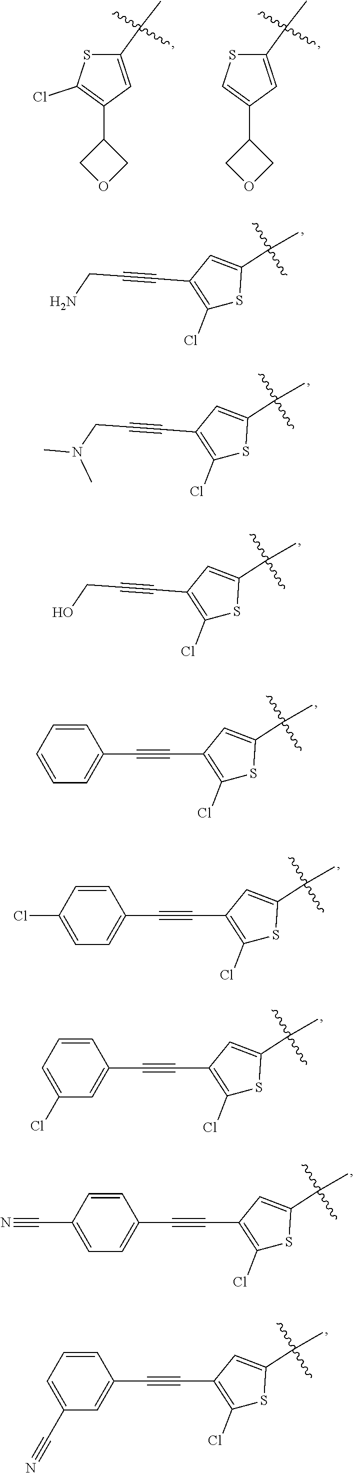

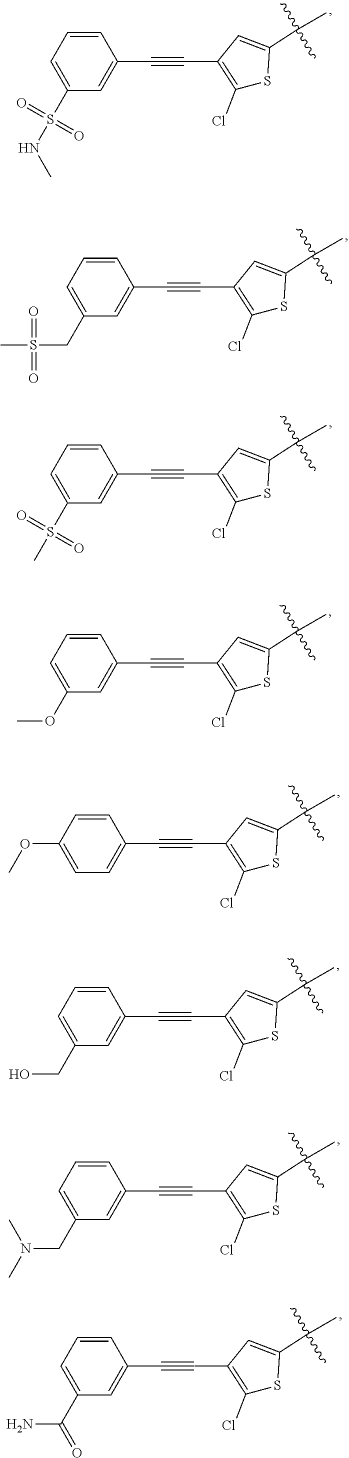

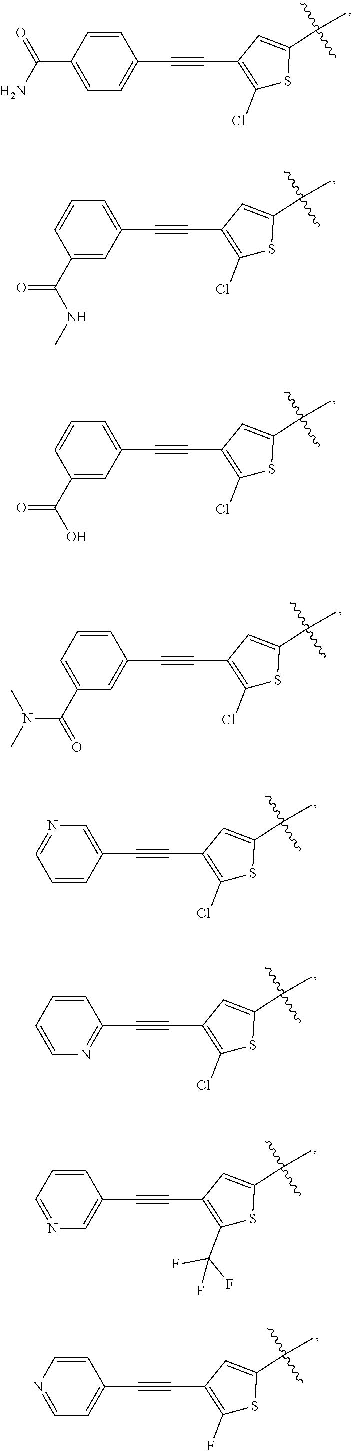

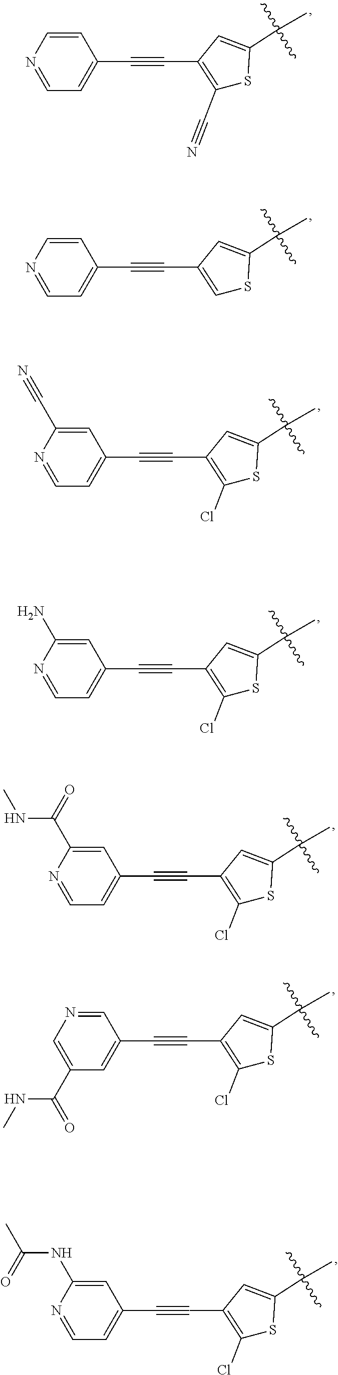

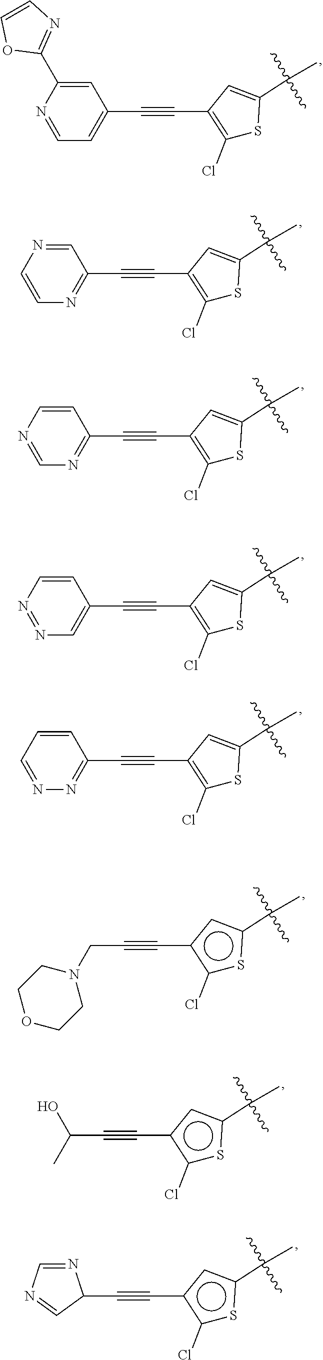

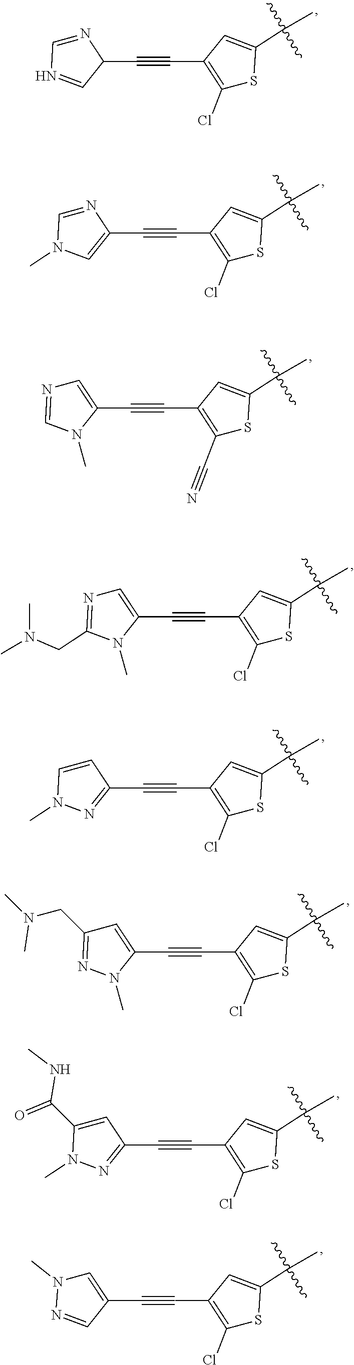

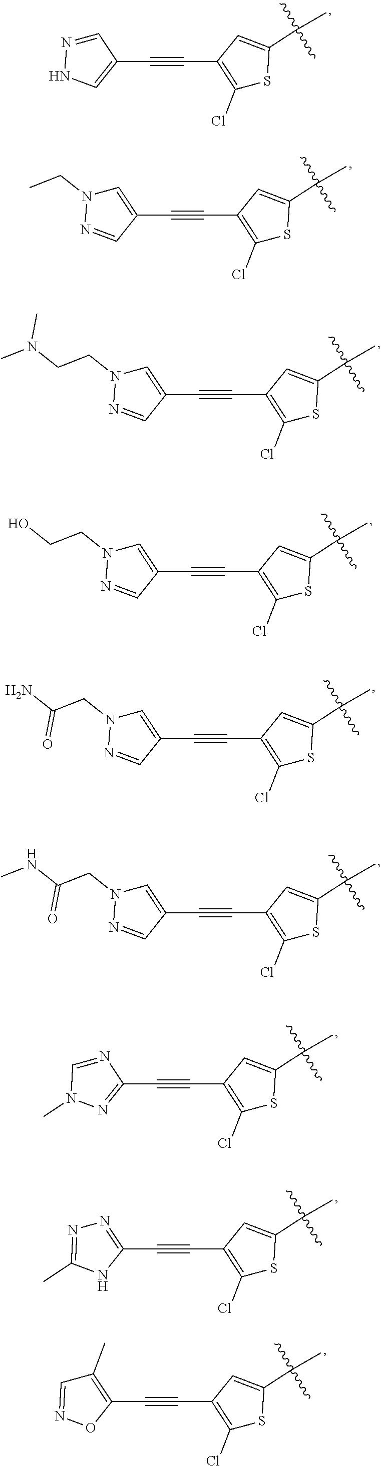

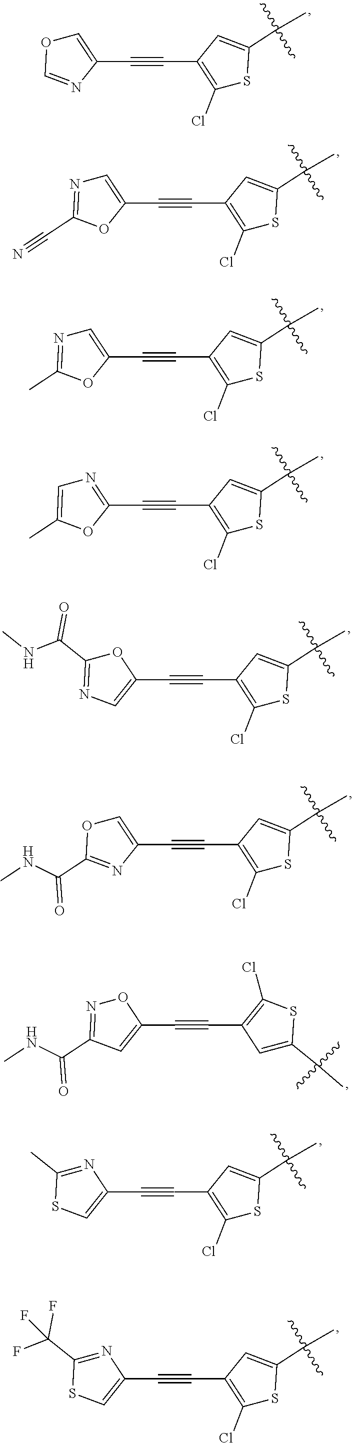

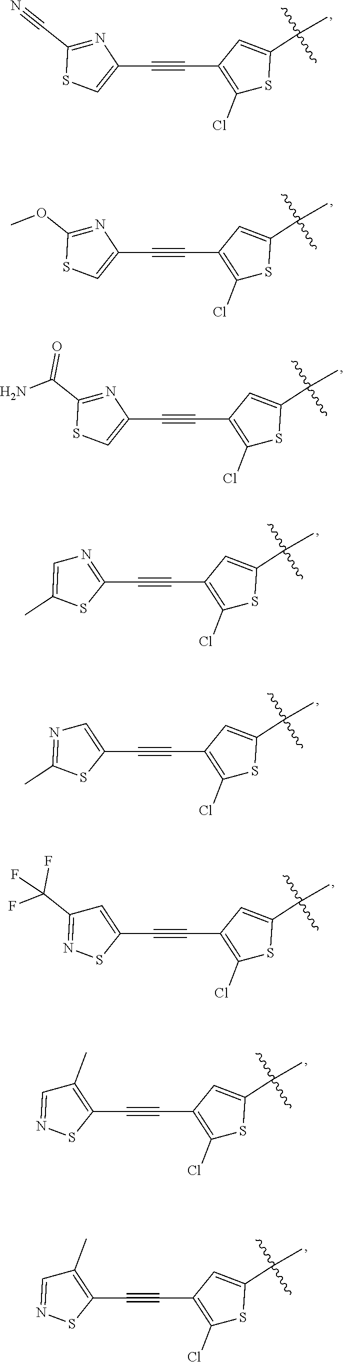

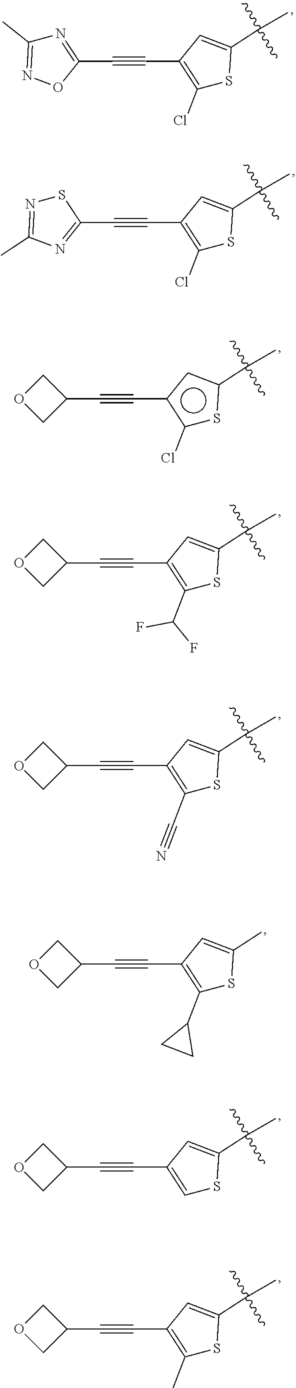

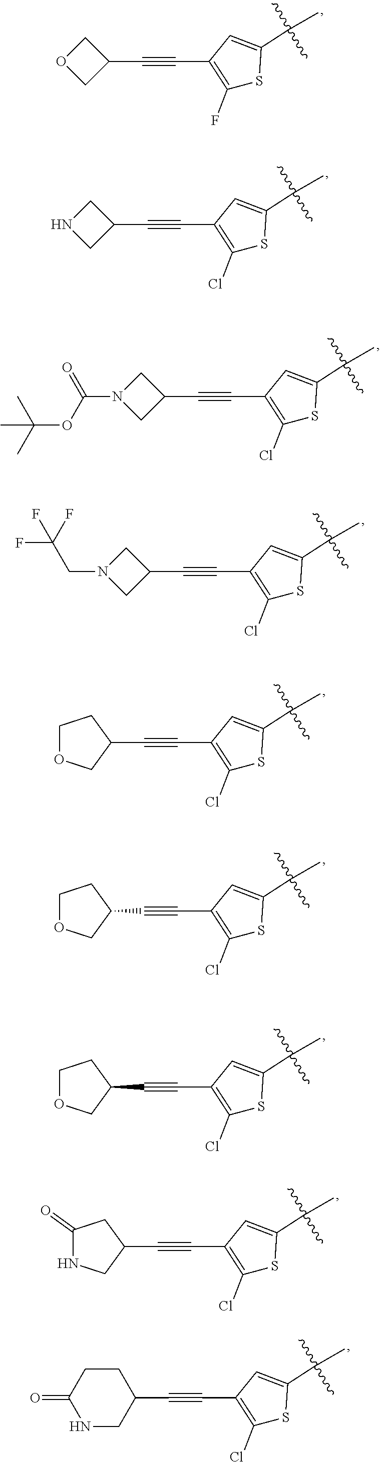

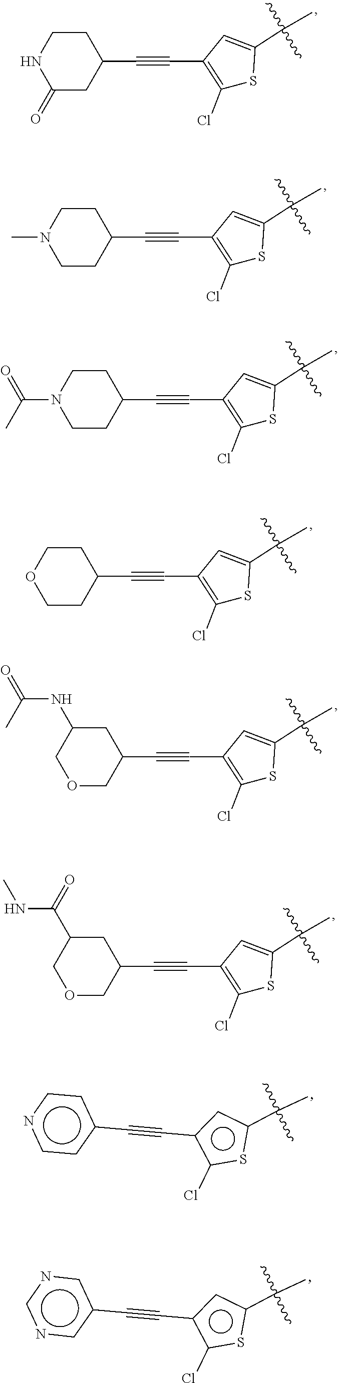

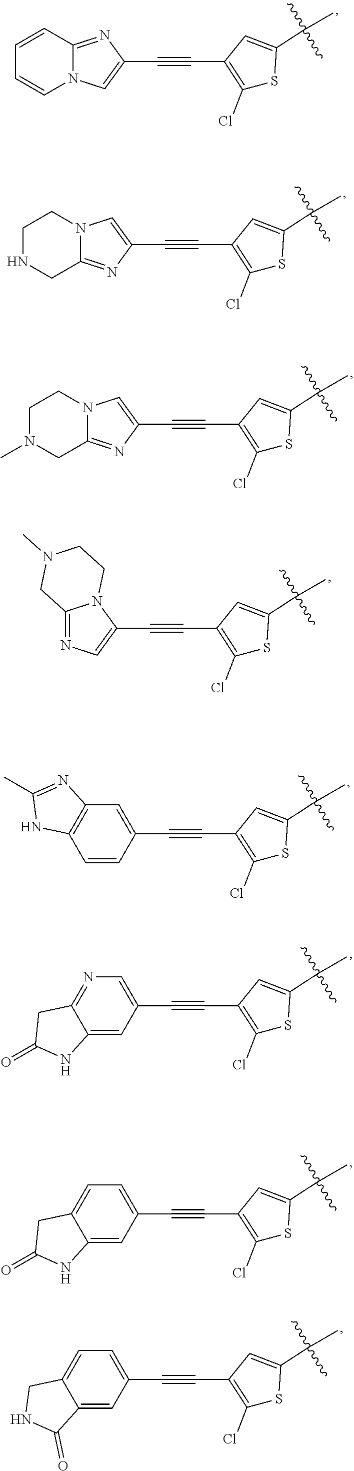

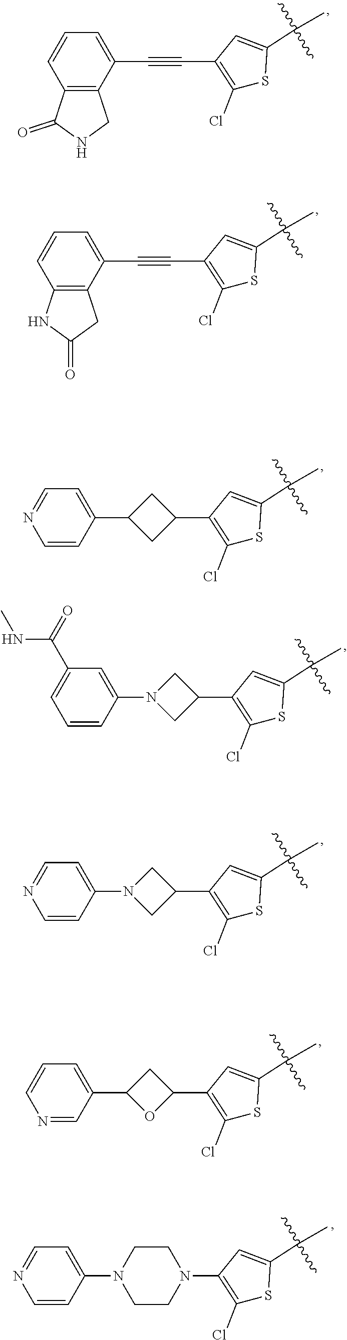

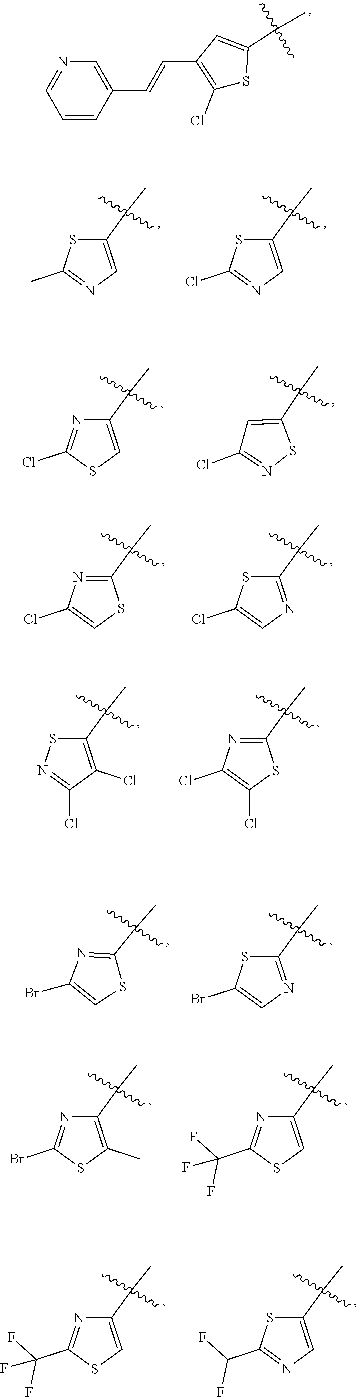

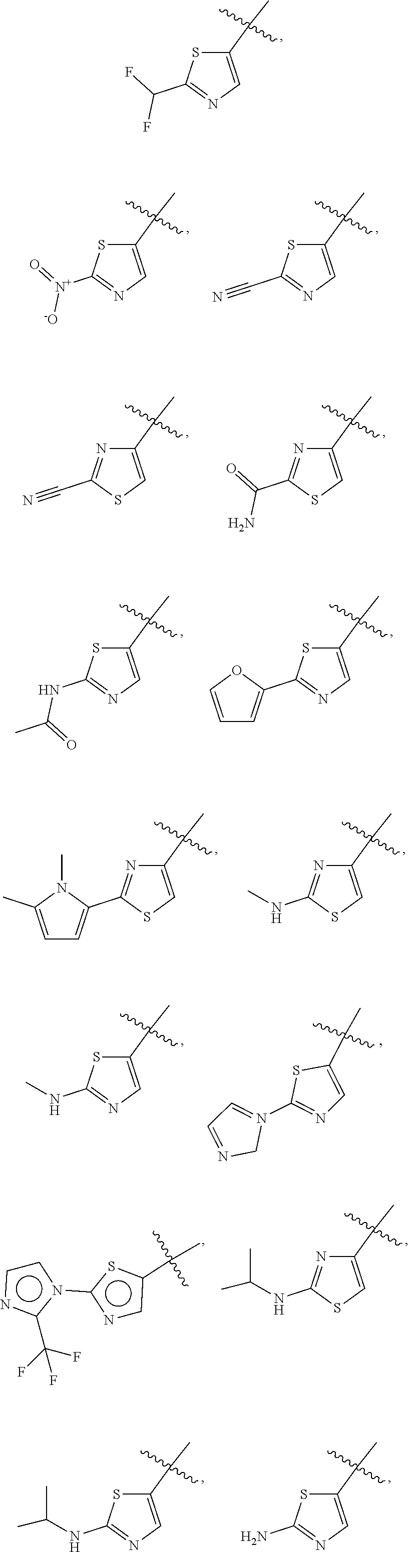

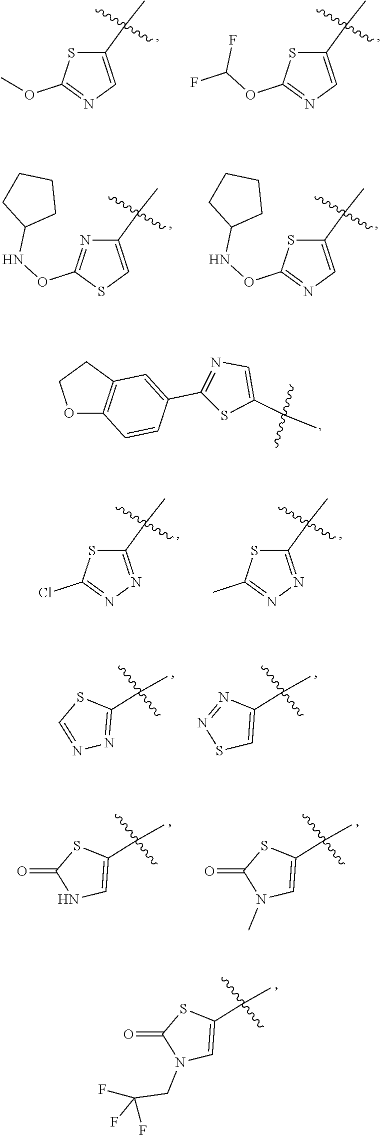

and tautomers thereof.

In some embodiments

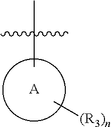

is selected from

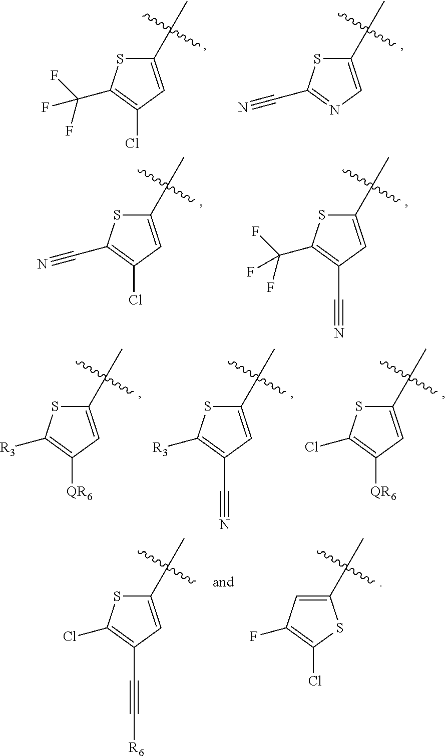

In some embodiments, the SMARCA2 inhibitor is a compound of Table 2 below:

TABLE 2
SMARCA2 inhibitors of the present disclosure.

| Compd. | | Mass |
|---|---|---|
| No. | Structure | (u) |
| 1 | 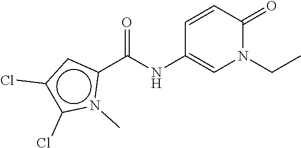 | 314 |
| 2 | 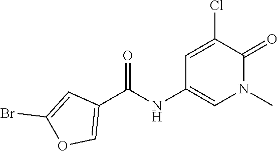 [SMILES: Cn1cc(NC(=O)c2coc(Br)c2)cc(Cl)c1=O] | 333 |
| 3 | 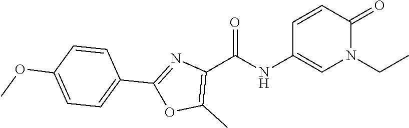 [SMILES: CCn1cc(NC(=O)c2nc(-c3ccc(OC)cc3)oc2C)ccc1=O] | 354 |
| 4 | 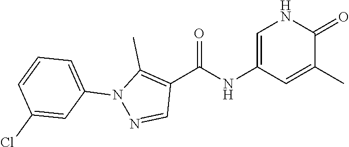 [SMILES: Cc1cc(NC(=O)c2cnn(-c3cccc(Cl)c3)c2C)c[nH]c1=O] | 343 |
| 5 | 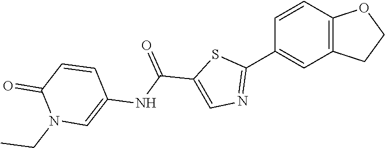 [SMILES: CCn1cc(NC(=O)c2cnc(-c3ccc4c(c3)CCO4)s2)ccc1=O] | 368 |
| 6 | 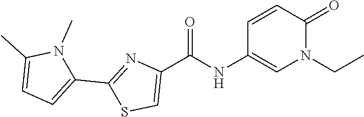 [SMILES: CCn1cc(NC(=O)c2csc(-c3ccc(C)n3C)n2)ccc1=O] | 343 |
| 7 | 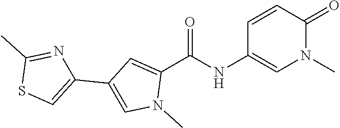 | 329 |
| 8 | 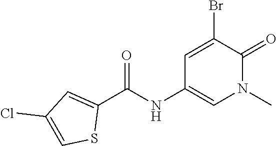 [SMILES: Cn1cc(NC(=O)c2cc(Cl)cs2)cc(Br)c1=O] | 349 |
| 9 | 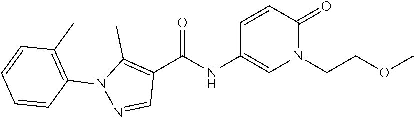 [SMILES: COCCn1cc(NC(=O)c2cnn(-c3ccccc3C)c2C)ccc1=O] | 367 |
| 10 | 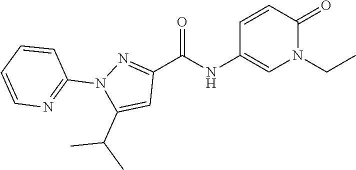 [SMILES: CCn1cc(NC(=O)c2cc(C(C)C)n(-c3ccccn3)n2)ccc1=O] | 352 |
| 11 | 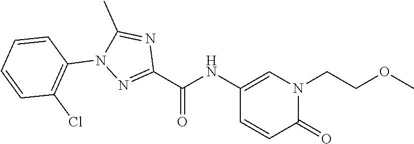 [SMILES: COCCn1cc(NC(=O)c2nc(C)n(-c3ccccc3Cl)n2)ccc1=O] | 388 |
| 12 | 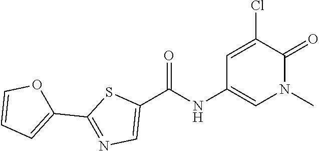 [SMILES: Cn1cc(NC(=O)c2cnc(-c3ccco3)s2)cc(Cl)c1=O] | 336 |
| 13 | 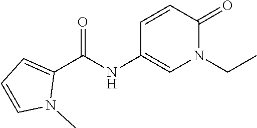 [SMILES: CCn1cc(NC(=O)c2cccn2C)ccc1=O] | 246 |
| 14 | 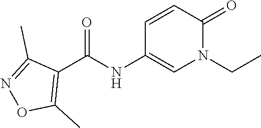 [SMILES: CCn1cc(NC(=O)c2c(C)noc2C)ccc1=O] | 262 |
| 15 | 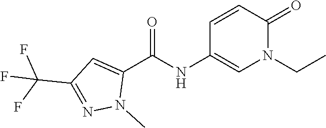 [SMILES: CCn1cc(NC(=O)c2cc(C(F)(F)F)nn2C)ccc1=O] | 315 |
| 16 | 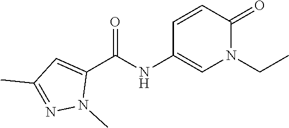 [SMILES: CCn1cc(NC(=O)c2cc(C)nn2C)ccc1=O] | 261 |
| 17 | 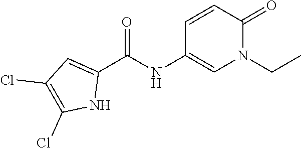 [SMILES: CCn1cc(NC(=O)c2cc(Cl)c(Cl)[nH]2)ccc1=O] | 300 |
| 18 | 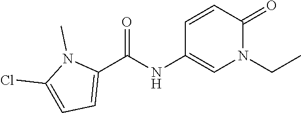 [SMILES: CCn1cc(NC(=O)c2ccc(Cl)n2C)ccc1=O] | 280 |
| 19 | 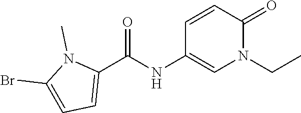 [SMILES: CCn1cc(NC(=O)c2ccc(Br)n2C)ccc1=O] | 326 |
| 20 | 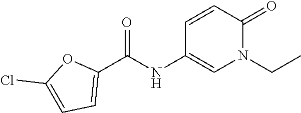 [SMILES: CCn1cc(NC(=O)c2ccc(Cl)o2)ccc1=O] | 267 |
| 21 | 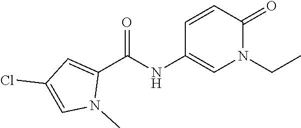 [SMILES: CCn1cc(NC(=O)c2cc(Cl)cn2C)ccc1=O] | 327 |
| 22 | 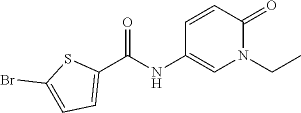 [SMILES: CCn1cc(NC(=O)c2ccc(Br)s2)ccc1=O] | 280 |
| 23 | 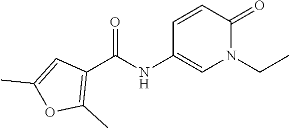 [SMILES: CCn1cc(NC(=O)c2cc(C)oc2C)ccc1=O] | 261 |
| 24 | 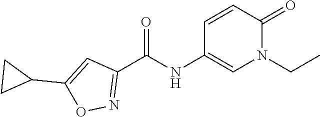 [SMILES: CCn1cc(NC(=O)c2cc(C3CC3)on2)ccc1=O] | 274 |
| 25 | 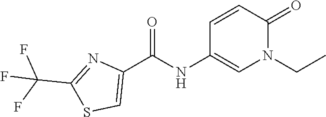 [SMILES: CCn1cc(NC(=O)c2csc(C(F)(F)F)n2)ccc1=O] | 318 |
| 26 | 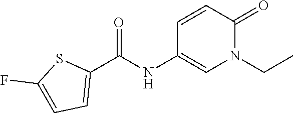 [SMILES: CCn1cc(NC(=O)c2ccc(F)s2)ccc1=O] | 267 |
| 27 | 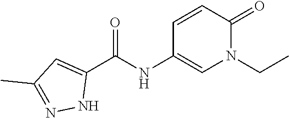 [SMILES: CCn1cc(NC(=O)c2cc(C)n[nH]2)ccc1=O] | 247 |
| 28 | 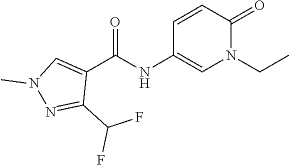 [SMILES: CCn1cc(NC(=O)c2cn(C)nc2C(F)F)ccc1=O] | 297 |
| 29 | 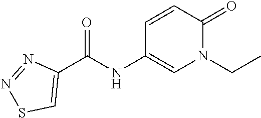 [SMILES: CCn1cc(NC(=O)c2csnn2)ccc1=O] | 251 |
| 30 | 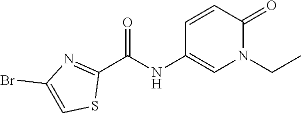 [SMILES: CCn1cc(NC(=O)c2nc(Br)cs2)ccc1=O] | 330 |
| 31 | 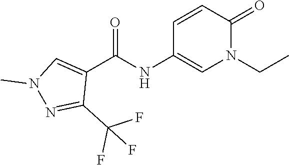 [SMILES: CCn1cc(NC(=O)c2cn(C)nc2C(F)(F)F)ccc1=O] | 315 |
| 32 | 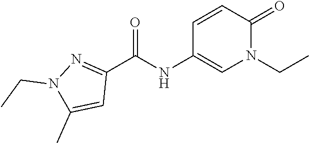 [SMILES: CCn1nc(C(=O)Nc2ccc(=O)n(CC)c2)cc1C] | 275 |
| 33 | 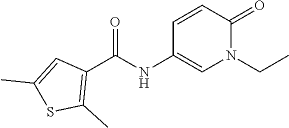 [SMILES: CCn1cc(NC(=O)c2cc(C)sc2C)ccc1=O] | 277 |
| 34 | 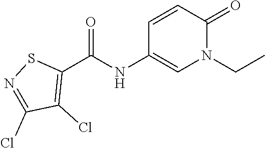 [SMILES: CCn1cc(NC(=O)c2snc(Cl)c2Cl)ccc1=O] | 317 |
| 35 | 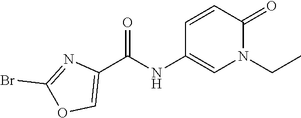 [SMILES: CCn1cc(NC(=O)c2coc(Br)n2)ccc1=O] | 311 |
| 36 | 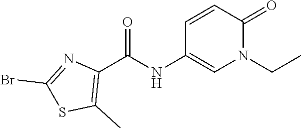 [SMILES: CCn1cc(NC(=O)c2nc(Br)sc2C)ccc1=O] | 344 |
| 37 | 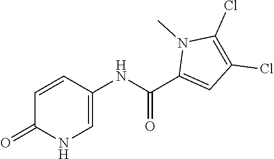 [SMILES: Cn1c(C(=O)Nc2ccc(=O)[nH]c2)cc(Cl)c1Cl] | 286 |
| 38 | 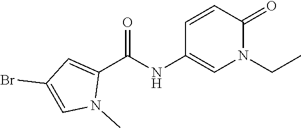 [SMILES: CCn1cc(NC(=O)c2cc(Br)cn2C)ccc1=O] | 324 |
| 39 | 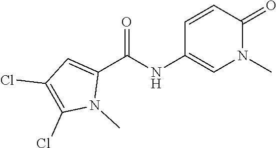 [SMILES: Cn1c(C(=O)Nc2ccc(=O)n(C)c2)cc(Cl)c1Cl] | 300 |
| 40 | 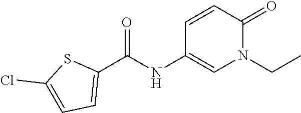 [SMILES: CCn1cc(NC(=O)c2ccc(Cl)s2)ccc1=O] | 283 |
| 41 | 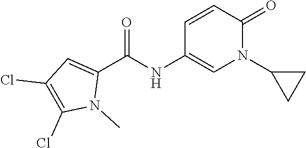 [SMILES: Cn1c(C(=O)Nc2ccc(=O)n(C3CC3)c2)cc(Cl)c1Cl] | 326 |
| 42 | 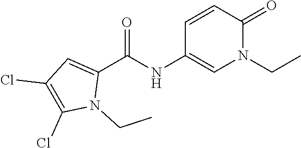 [SMILES: CCn1c(C(=O)Nc2ccc(=O)n(CC)c2)cc(Cl)c1Cl] | 328 |
| 43 | 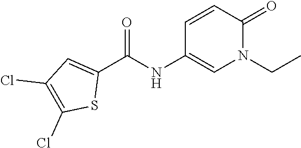 [SMILES: CCn1cc(NC(=O)c2cc(Cl)c(Cl)s2)ccc1=O] | 317 |
| 44 | 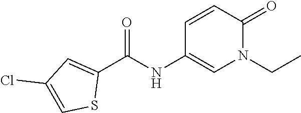 [SMILES: CCn1cc(NC(=O)c2cc(Cl)cs2)ccc1=O] | 283 |
| 45 | 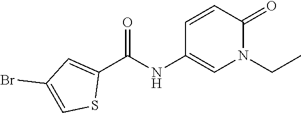 [SMILES: CCn1cc(NC(=O)c2cc(Br)cs2)ccc1=O] | 329 |
| 46 | 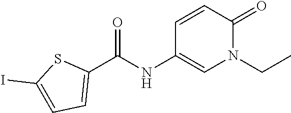 [SMILES: CCn1cc(NC(=O)c2ccc(I)s2)ccc1=O] | 375 |
| 47 | 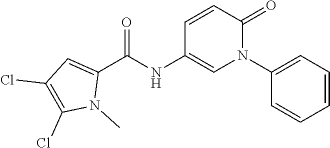 [SMILES: Cn1c(C(=O)Nc2ccc(=O)n(-c3ccccc3)c2)cc(Cl)c1Cl] | 362 |
| 48 | 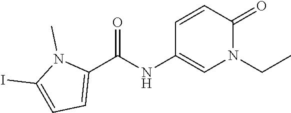 [SMILES: CCn1cc(NC(=O)c2ccc(I)n2C)ccc1=O] | 372 |
| 49 | 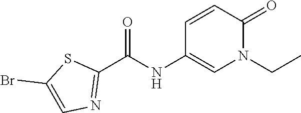 [SMILES: CCn1cc(NC(=O)c2ncc(Br)s2)ccc1=O] | 329 |
| 50 | 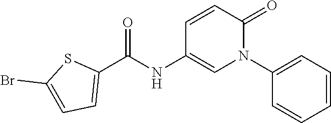 [SMILES: O=C(Nc1ccc(=O)n(-c2ccccc2)c1)c1ccc(Br)s1] | 377 |
| 51 | 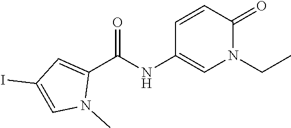 [SMILES: CCn1cc(NC(=O)c2cc(I)cn2C)ccc1=O] | 372 |
| 52 | 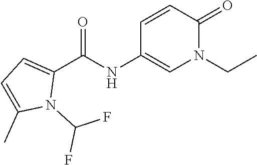 [SMILES: CCn1cc(NC(=O)c2ccc(C)n2C(F)F)ccc1=O] | 296 |
| 53 | 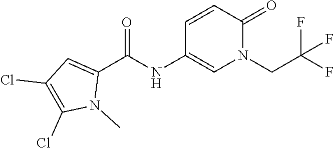 [SMILES: Cn1c(C(=O)Nc2ccc(=O)n(CC(F)(F)F)c2)cc(Cl)c1Cl] | 368 |
| 54 | 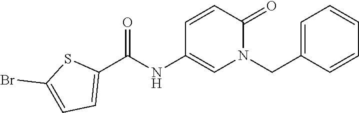 [SMILES: O=C(Nc1ccc(=O)n(Cc2ccccc2)c1)c1ccc(Br)s1] | 391 |
| 55 | 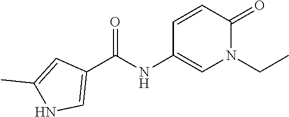 [SMILES: CCn1cc(NC(=O)c2c[nH]c(C)c2)ccc1=O] | 246 |
| 56 | 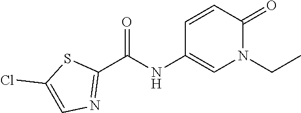 [SMILES: CCn1cc(NC(=O)c2ncc(Cl)s2)ccc1=O] | 284 |
| 57 | 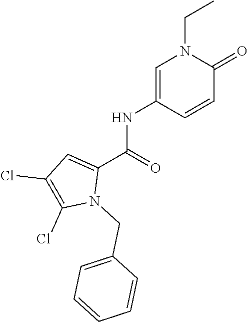 [SMILES: CCn1cc(NC(=O)c2cc(Cl)c(Cl)n2Cc2ccccc2)ccc1=O] | 390 |
| 58 | 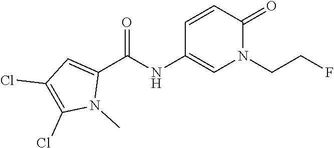 [SMILES: Cn1c(C(=O)Nc2ccc(=O)n(CCF)c2)cc(Cl)c1Cl] | 332 |
| 59 | 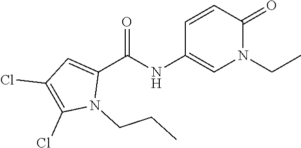 [SMILES: CCCn1c(C(=O)Nc2ccc(=O)n(CC)c2)cc(Cl)c1Cl] | 342 |
| 60 | 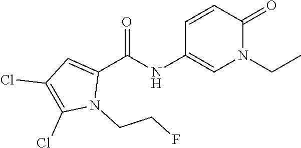 [SMILES: CCn1cc(NC(=O)c2cc(Cl)c(Cl)n2CCF)ccc1=O] | 346 |
| 61 | 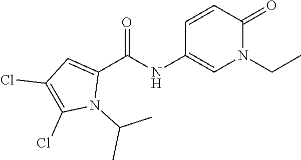 [SMILES: CCn1cc(NC(=O)c2cc(Cl)c(Cl)n2C(C)C)ccc1=O] | 342 |
| 62 | 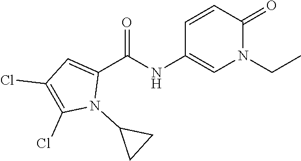 [SMILES: CCn1cc(NC(=O)c2cc(Cl)c(Cl)n2C2CC2)ccc1=O] | 340 |
| 63 | 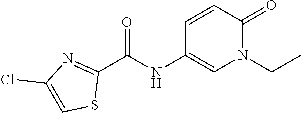 [SMILES: CCn1cc(NC(=O)c2nc(Cl)cs2)ccc1=O] | 284 |
| 64 | 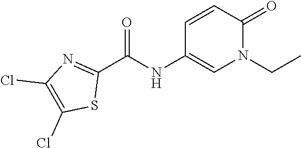 [SMILES: CCn1cc(NC(=O)c2nc(Cl)c(Cl)s2)ccc1=O] | 318 |
| 65 | 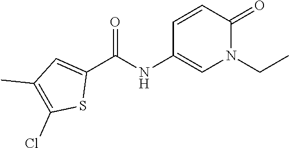 [SMILES: CCn1cc(NC(=O)c2cc(C)c(Cl)s2)ccc1=O] | 297 |
| 66 | 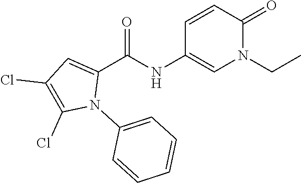 [SMILES: CCn1cc(NC(=O)c2cc(Cl)c(Cl)n2-c2ccccc2)ccc1=O] | 376 |
| 67 | 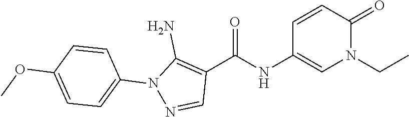 [SMILES: CCn1cc(NC(=O)c2cnn(-c3ccc(OC)cc3)c2N)ccc1=O] | 355 |
| 68 | 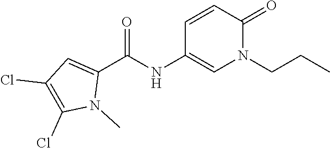 [SMILES: CCCn1cc(NC(=O)c2cc(Cl)c(Cl)n2C)ccc1=O] | 328 |
| 69 | 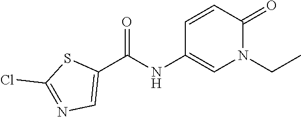 [SMILES: CCn1cc(NC(=O)c2cnc(Cl)s2)ccc1=O] | 284 |
| 70 | 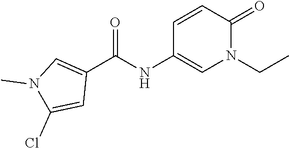 [SMILES: CCn1cc(NC(=O)c2cc(Cl)n(C)c2)ccc1=O] | 280 |
| 71 | 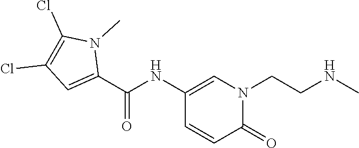 [SMILES: CNCCn1cc(NC(=O)c2cc(Cl)c(Cl)n2C)ccc1=O] | 343 |
| 72 | 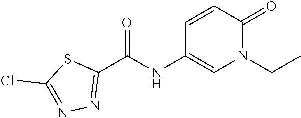 [SMILES: CCn1cc(NC(=O)c2nnc(Cl)s2)ccc1=O] | 285 |
| 73 | 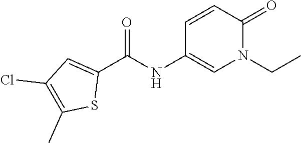 [SMILES: CCn1cc(NC(=O)c2cc(Cl)c(C)s2)ccc1=O] | 297 |
| 74 | 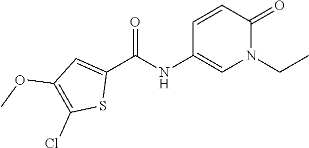 [SMILES: CCn1cc(NC(=O)c2cc(OC)c(Cl)s2)ccc1=O] | 313 |
| 75 | 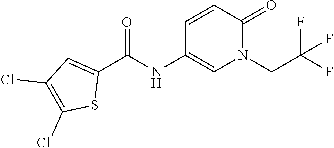 [SMILES: O=C(Nc1ccc(=O)n(CC(F)(F)F)c1)c1cc(Cl)c(Cl)s1] | 371 |
| 76 | 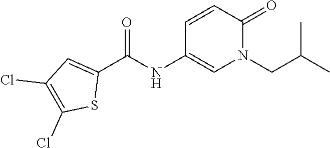 [SMILES: CC(C)Cn1cc(NC(=O)c2cc(Cl)c(Cl)s2)ccc1=O] | 345 |
| 77 | 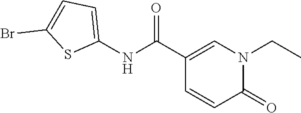 [SMILES: CCn1cc(C(=O)Nc2ccc(Br)s2)ccc1=O] | 327 |
| 78 | 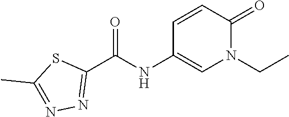 [SMILES: CCn1cc(NC(=O)c2nnc(C)s2)ccc1=O] | 265 |
| 79 | 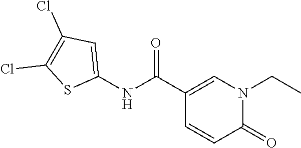 [SMILES: CCn1cc(C(=O)Nc2cc(Cl)c(Cl)s2)ccc1=O] | 317 |
| 80 | 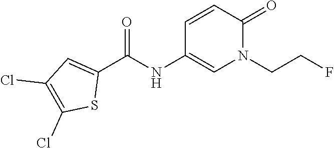 [SMILES: O=C(Nc1ccc(=O)n(CCF)c1)c1cc(Cl)c(Cl)s1] | 335 |
| 81 | 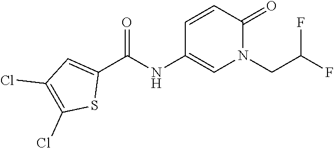 [SMILES: O=C(Nc1ccc(=O)n(CC(F)F)c1)c1cc(Cl)c(Cl)s1] | 353 |
| 82 | 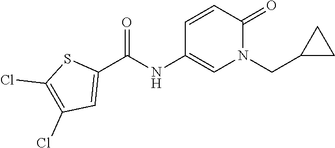 [SMILES: O=C(Nc1ccc(=O)n(CC2CC2)c1)c1cc(Cl)c(Cl)s1] | 343 |
| 83 | 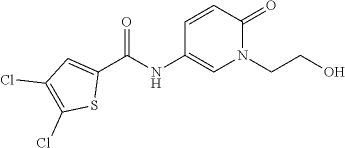 [SMILES: O=C(Nc1ccc(=O)n(CCO)c1)c1cc(Cl)c(Cl)s1] | 333 |
| 84 | 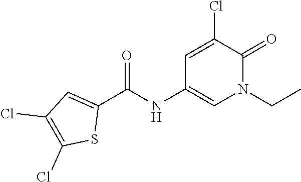 [SMILES: CCn1cc(NC(=O)c2cc(Cl)c(Cl)s2)cc(Cl)c1=O] | 353 |
| 85 | 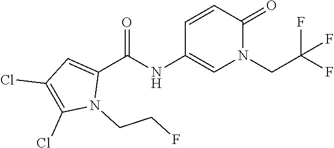 [SMILES: O=C(Nc1ccc(=O)n(CC(F)(F)F)c1)c1cc(Cl)c(Cl)n1CCF] | 400 |
| 86 | 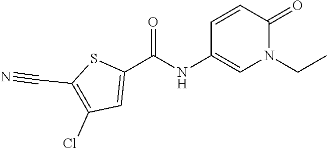 [SMILES: CCn1cc(NC(=O)c2cc(Cl)c(C#N)s2)ccc1=O] | 308 |
| 87 | 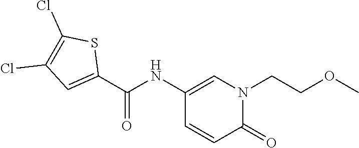 [SMILES: COCCn1cc(NC(=O)c2cc(Cl)c(Cl)s2)ccc1=O] | 347 |
| 88 | 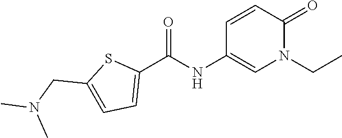 [SMILES: CCn1cc(NC(=O)c2ccc(CN(C)C)s2)ccc1=O] | 306 |
| 89 | 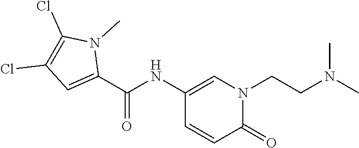 [SMILES: CN(C)CCn1cc(NC(=O)c2cc(Cl)c(Cl)n2C)ccc1=O] | 357 |
| 90 | 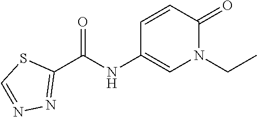 [SMILES: CCn1cc(NC(=O)c2nncs2)ccc1=O] | 251 |
| 91 | 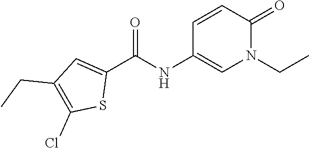 [SMILES: CCc1cc(C(=O)Nc2ccc(=O)n(CC)c2)sc1Cl] | 311 |
| 92 | 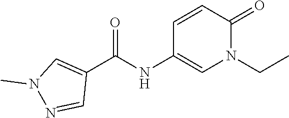 [SMILES: CCn1cc(NC(=O)c2cnn(C)c2)ccc1=O] | 247 |
| 93 | 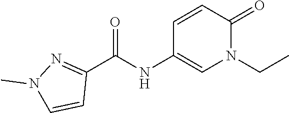 [SMILES: CCn1cc(NC(=O)c2ccn(C)n2)ccc1=O] | 247 |
| 94 | 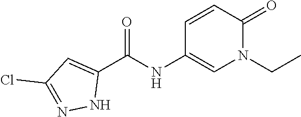 [SMILES: CCn1cc(NC(=O)c2cc(Cl)n[nH]2)ccc1=O] | 267 |
| 95 | 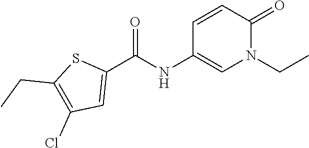 [SMILES: CCc1sc(C(=O)Nc2ccc(=O)n(CC)c2)cc1Cl] | 311 |
| 96 | 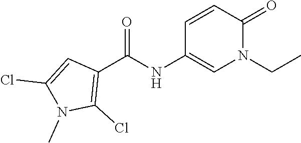 [SMILES: CCn1cc(NC(=O)c2cc(Cl)n(C)c2Cl)ccc1=O] | 314 |
| 97 | 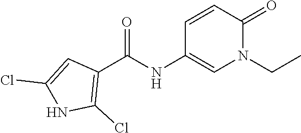 [SMILES: CCn1cc(NC(=O)c2cc(Cl)[nH]c2Cl)ccc1=O] | 300 |
| 98 | 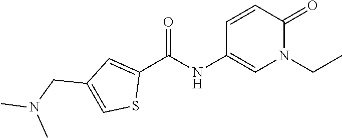 [SMILES: CCn1cc(NC(=O)c2cc(CN(C)C)cs2)ccc1=O] | 306 |
| 99 | 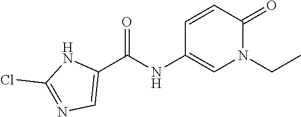 [SMILES: CCn1cc(NC(=O)c2cnc(Cl)[nH]2)ccc1=O] | 267 |
| 100 | 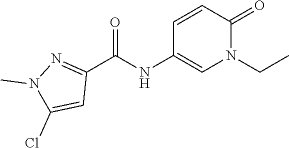 [SMILES: CCn1cc(NC(=O)c2cc(Cl)n(C)n2)ccc1=O] | 281 |
| 101 | 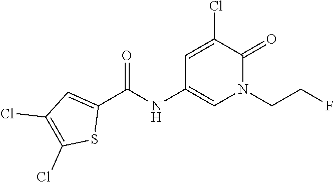 [SMILES: O=C(Nc1cc(Cl)c(=O)n(CCF)c1)c1cc(Cl)c(Cl)s1] | 371 |
| 102 | 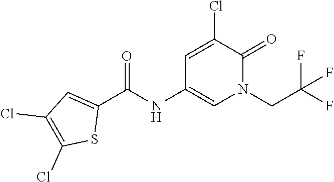 [SMILES: O=C(Nc1cc(Cl)c(=O)n(CC(F)(F)F)c1)c1cc(Cl)c(Cl)s1] | 405 |
| 103 | 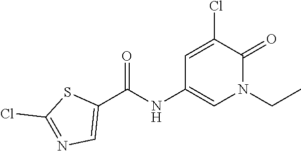 [SMILES: CCn1cc(NC(=O)c2cnc(Cl)s2)cc(Cl)c1=O] | 318 |
| 104 | 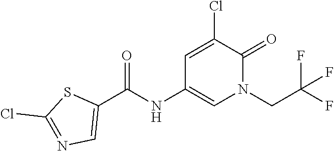 [SMILES: O=C(Nc1cc(Cl)c(=O)n(CC(F)(F)F)c1)c1cnc(Cl)s1] | 374 |
| 105 | 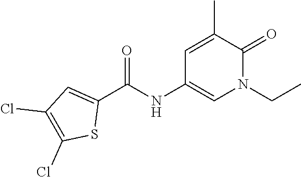 [SMILES: CCn1cc(NC(=O)c2cc(Cl)c(Cl)s2)cc(C)c1=O] | 331 |
| 106 | 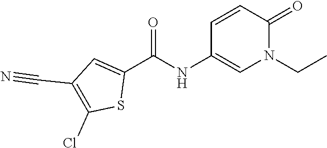 [SMILES: CCn1cc(NC(=O)c2cc(C#N)c(Cl)s2)ccc1=O] | 308 |
| 107 | 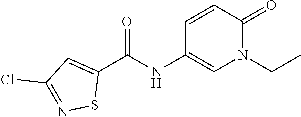 [SMILES: CCn1cc(NC(=O)c2cc(Cl)ns2)ccc1=O] | 284 |
| 108 | 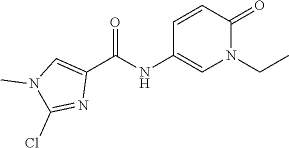 [SMILES: CCn1cc(NC(=O)c2cn(C)c(Cl)n2)ccc1=O] | 281 |
| 109 | 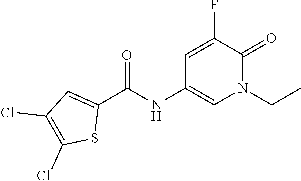 [SMILES: CCn1cc(NC(=O)c2cc(Cl)c(Cl)s2)cc(F)c1=O] | 335 |
| 110 | 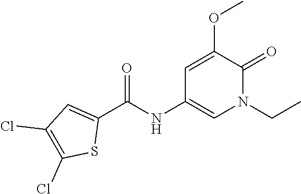 [SMILES: CCn1cc(NC(=O)c2cc(Cl)c(Cl)s2)cc(OC)c1=O] | 347 |
| 111 | 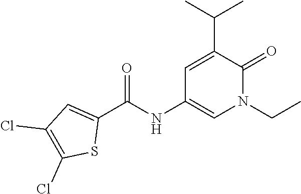 [SMILES: CCn1cc(NC(=O)c2cc(Cl)c(Cl)s2)cc(C(C)C)c1=O] | 359 |
| 112 | 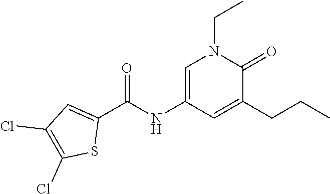 [SMILES: CCCc1cc(NC(=O)c2cc(Cl)c(Cl)s2)cn(CC)c1=O] | 359 |
| 113 | 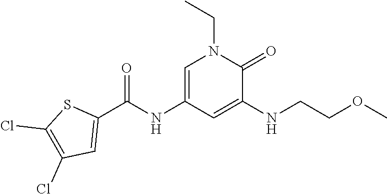 [SMILES: CCn1cc(NC(=O)c2cc(Cl)c(Cl)s2)cc(NCCOC)c1=O] | 390 |
| 114 | 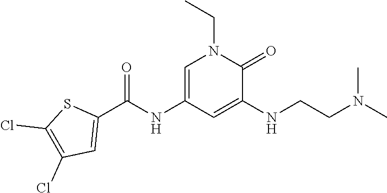 [SMILES: CCn1cc(NC(=O)c2cc(Cl)c(Cl)s2)cc(NCCN(C)C)c1=O] | 403 |
| 115 | 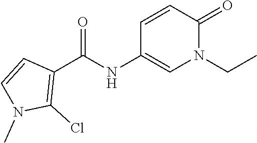 [SMILES: CCn1cc(NC(=O)c2ccn(C)c2Cl)ccc1=O] | 280 |
| 116 | 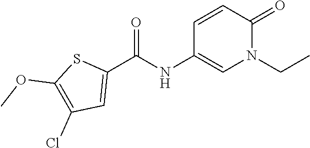 [SMILES: CCn1cc(NC(=O)c2cc(Cl)c(OC)s2)ccc1=O] | 313 |
| 117 | 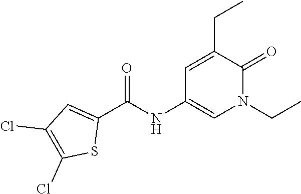 [SMILES: CCc1cc(NC(=O)c2cc(Cl)c(Cl)s2)cn(CC)c1=O] | 345 |
| 118 | 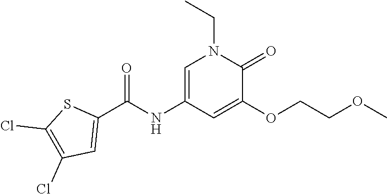 [SMILES: CCn1cc(NC(=O)c2cc(Cl)c(Cl)s2)cc(OCCOC)c1=O] | 391 |
| 119 | 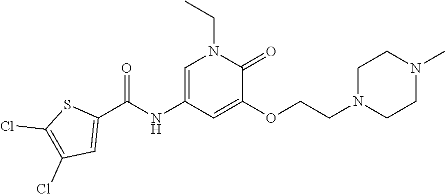 [SMILES: CCn1cc(NC(=O)c2cc(Cl)c(Cl)s2)cc(OCCN2CCN(C)CC2)c1=O] | 459 |
| 120 | 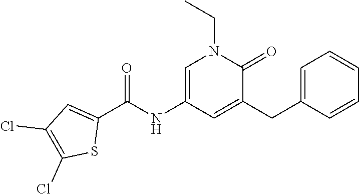 [SMILES: CCn1cc(NC(=O)c2cc(Cl)c(Cl)s2)cc(Cc2ccccc2)c1=O] | 407 |
| 121 | 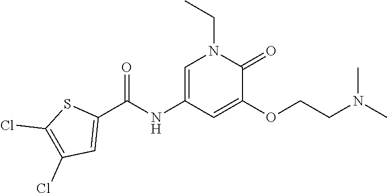 [SMILES: CCn1cc(NC(=O)c2cc(Cl)c(Cl)s2)cc(OCCN(C)C)c1=O] | 404 |
| 122 | 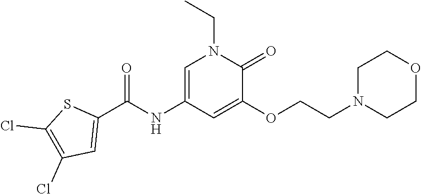 [SMILES: CCn1cc(NC(=O)c2cc(Cl)c(Cl)s2)cc(OCCN2CCOCC2)c1=O] | 446 |
| 123 | 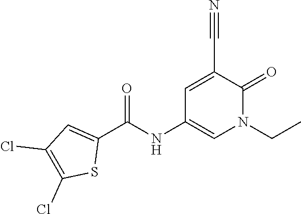 [SMILES: CCn1cc(NC(=O)c2cc(Cl)c(Cl)s2)cc(C#N)c1=O] | 342 |
| 124 | 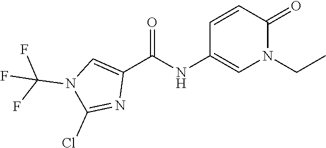 [SMILES: CCn1cc(NC(=O)c2cn(C(F)(F)F)c(Cl)n2)ccc1=O] | 335 |
| 125 | 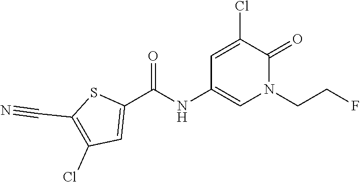 [SMILES: N#Cc1sc(C(=O)Nc2cc(Cl)c(=O)n(CCF)c2)cc1Cl] | 360 |
| 126 | 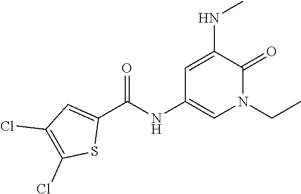 [SMILES: CCn1cc(NC(=O)c2cc(Cl)c(Cl)s2)cc(NC)c1=O] | 346 |
| 127 | 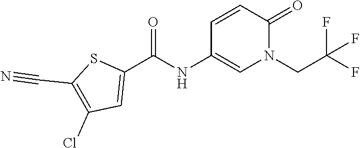 [SMILES: N#Cc1sc(C(=O)Nc2ccc(=O)n(CC(F)(F)F)c2)cc1Cl] | 362 |
| 128 | 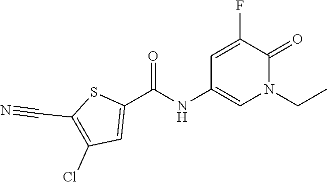 [SMILES: CCn1cc(NC(=O)c2cc(Cl)c(C#N)s2)cc(F)c1=O] | 326 |
| 129 | 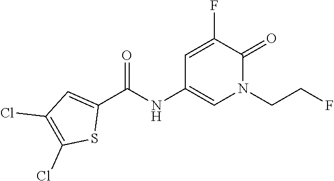 [SMILES: O=C(Nc1cc(F)c(=O)n(CCF)c1)c1cc(Cl)c(Cl)s1] | 353 |
| 130 | 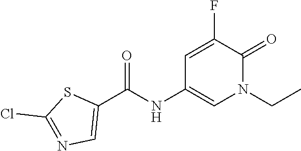 [SMILES: CCn1cc(NC(=O)c2cnc(Cl)s2)cc(F)c1=O] | 302 |
| 131 | 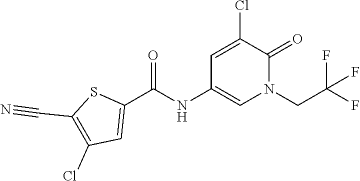 [SMILES: N#Cc1sc(C(=O)Nc2cc(Cl)c(=O)n(CC(F)(F)F)c2)cc1Cl] | 396 |
| 132 | 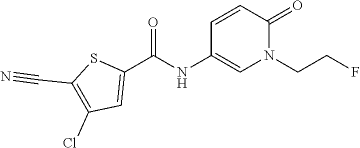 [SMILES: N#Cc1sc(C(=O)Nc2ccc(=O)n(CCF)c2)cc1Cl] | 326 |
| 133 | 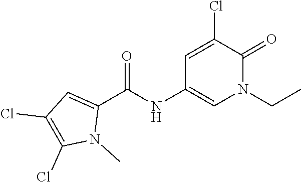 [SMILES: CCn1cc(NC(=O)c2cc(Cl)c(Cl)n2C)cc(Cl)c1=O] | 350 |
| 134 | 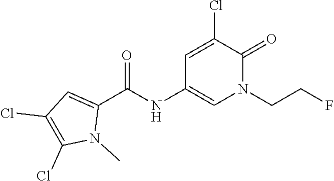 [SMILES: Cn1c(C(=O)Nc2cc(Cl)c(=O)n(CCF)c2)cc(Cl)c1Cl] | 368 |
| 135 | 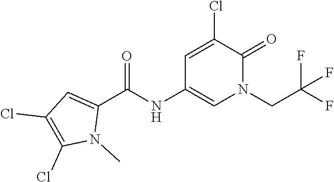 [SMILES: Cn1c(C(=O)Nc2cc(Cl)c(=O)n(CC(F)(F)F)c2)cc(Cl)c1Cl] | 404 |
| 136 | 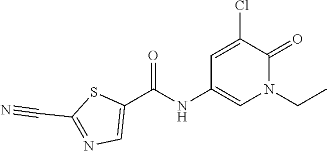 [SMILES: CCn1cc(NC(=O)c2cnc(C#N)s2)cc(Cl)c1=O] | 309 |
| 137 | 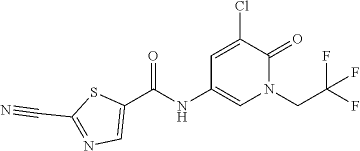 [SMILES: N#Cc1ncc(C(=O)Nc2cc(Cl)c(=O)n(CC(F)(F)F)c2)s1] | 363 |
| 138 | 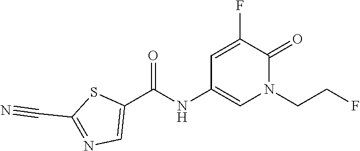 [SMILES: N#Cc1ncc(C(=O)Nc2cc(F)c(=O)n(CCF)c2)s1] | 311 |
| 139 | 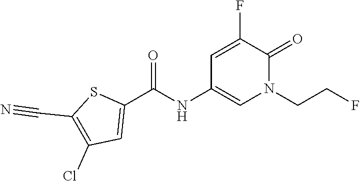 [SMILES: N#Cc1sc(C(=O)Nc2cc(F)c(=O)n(CCF)c2)cc1Cl] | 344 |
| 140 | 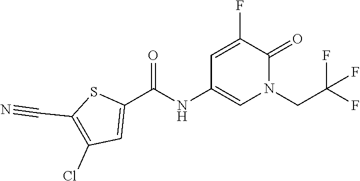 [SMILES: N#Cc1sc(C(=O)Nc2cc(F)c(=O)n(CC(F)(F)F)c2)cc1Cl] | 380 |
| 141 | 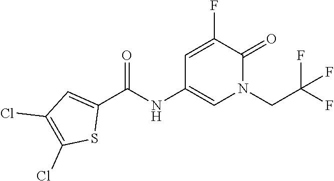 [SMILES: O=C(Nc1cc(F)c(=O)n(CC(F)(F)F)c1)c1cc(Cl)c(Cl)s1] | 389 |
| 142 | 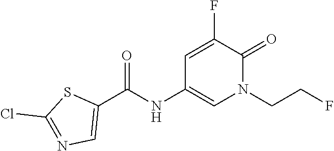 [SMILES: O=C(Nc1cc(F)c(=O)n(CCF)c1)c1cnc(Cl)s1] | 320 |
| 143 | 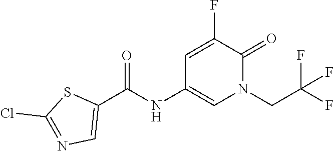 [SMILES: O=C(Nc1cc(F)c(=O)n(CC(F)(F)F)c1)c1cnc(Cl)s1] | 356 |
| 144 | 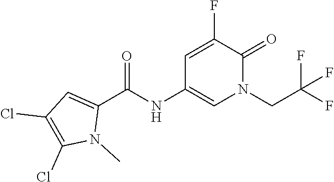 [SMILES: Cn1c(C(=O)Nc2cc(F)c(=O)n(CC(F)(F)F)c2)cc(Cl)c1Cl] | 386 |
| 145 | 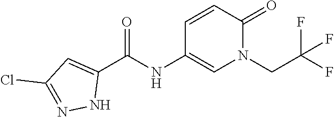 [SMILES: O=C(Nc1ccc(=O)n(CC(F)(F)F)c1)c1cc(Cl)n[nH]1] | 321 |
| 146 | 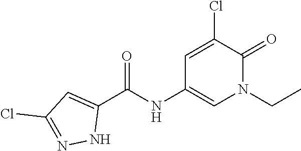 [SMILES: CCn1cc(NC(=O)c2cc(Cl)n[nH]2)cc(Cl)c1=O] | 301 |
| 147 | 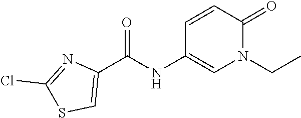 [SMILES: CCn1cc(NC(=O)c2csc(Cl)n2)ccc1=O] | 284 |
| 148 | 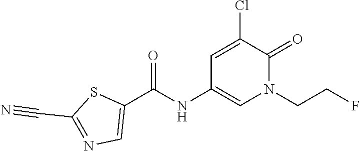 [SMILES: N#Cc1ncc(C(=O)Nc2cc(Cl)c(=O)n(CCF)c2)s1] | 327 |
| 149 | 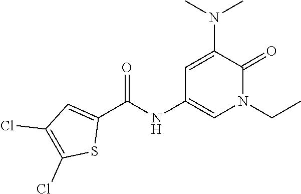 [SMILES: CCn1cc(NC(=O)c2cc(Cl)c(Cl)s2)cc(N(C)C)c1=O] | 360 |
| 150 | 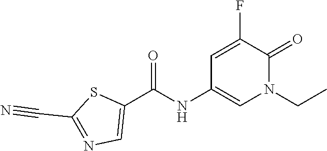 [SMILES: CCn1cc(NC(=O)c2cnc(C#N)s2)cc(F)c1=O] | 293 |
| 151 | 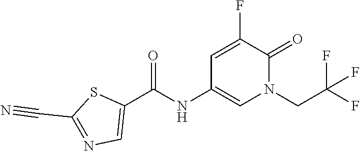 [SMILES: N#Cc1ncc(C(=O)Nc2cc(F)c(=O)n(CC(F)(F)F)c2)s1] | 347 |
| 152 | 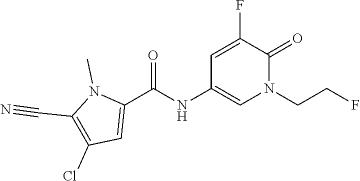 [SMILES: Cn1c(C(=O)Nc2cc(F)c(=O)n(CCF)c2)cc(Cl)c1C#N] | 340 |
| 153 | 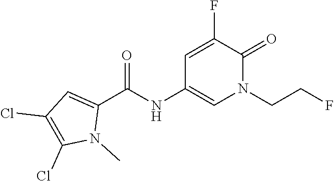 [SMILES: Cn1c(C(=O)Nc2cc(F)c(=O)n(CCF)c2)cc(Cl)c1Cl] | 350 |
| 154 | 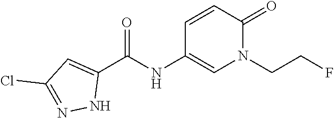 [SMILES: O=C(Nc1ccc(=O)n(CCF)c1)c1cc(Cl)n[nH]1] | 285 |
| 155 | 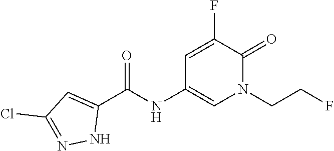 [SMILES: O=C(Nc1cc(F)c(=O)n(CCF)c1)c1cc(Cl)n[nH]1] | 303 |
| 156 | 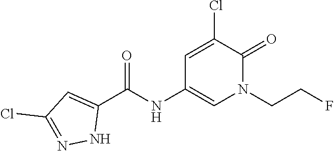 [SMILES: O=C(Nc1cc(Cl)c(=O)n(CCF)c1)c1cc(Cl)n[nH]1] | 319 |
| 157 | 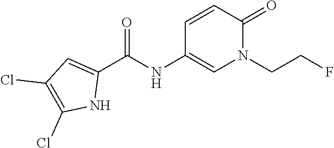 [SMILES: O=C(Nc1ccc(=O)n(CCF)c1)c1cc(Cl)c(Cl)[nH]1] | 318 |
| 158 | 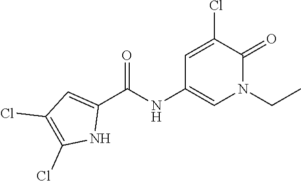 [SMILES: CCn1cc(NC(=O)c2cc(Cl)c(Cl)[nH]2)cc(Cl)c1=O] | 356 |
| 159 | 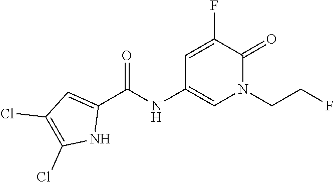 [SMILES: O=C(Nc1cc(F)c(=O)n(CCF)c1)c1cc(Cl)c(Cl)[nH]1] | 336 |
| 160 | 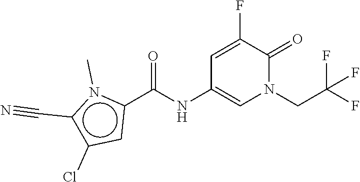 | 377 |
| 161 | 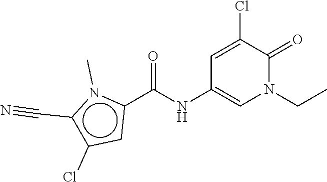 | 339 |
| 162 | 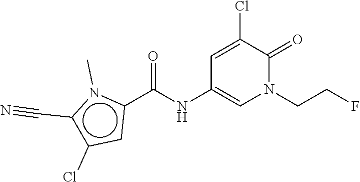 | 357 |
| 163 | 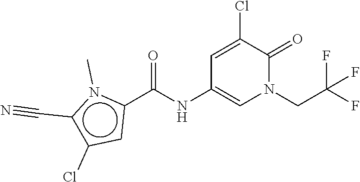 | 393 |
| 164 | 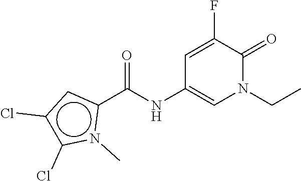 | 332 |
| 165 | 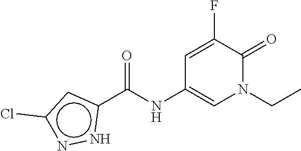 | 285 |
| 166 | 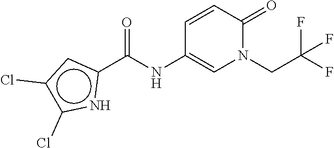 | 354 |
| 167 | 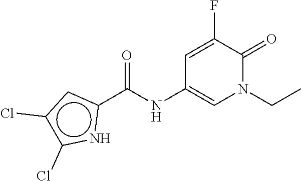 | 318 |
| 168 | 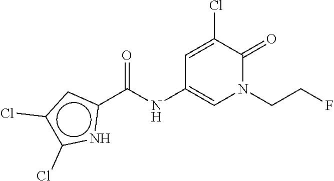 | 353 |
| 169 | 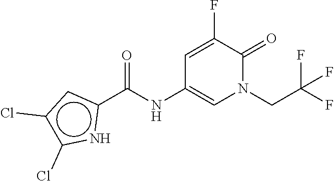 | 372 |
| 170 | 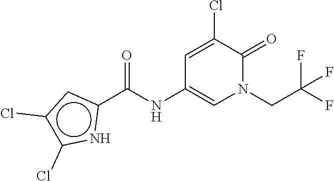 | 389 |
| 171 | 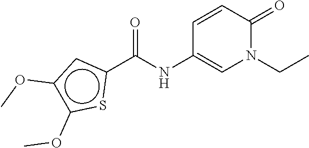 | 309 |
| 172 | 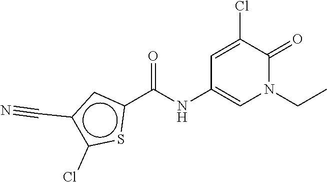 | 342 |
| 173 | 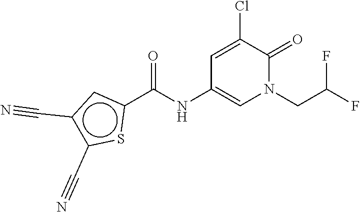 | 387 |
| 174 | 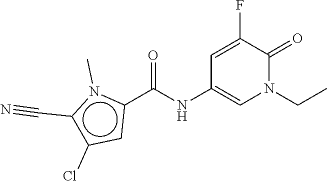 | 323 |
| 175 | 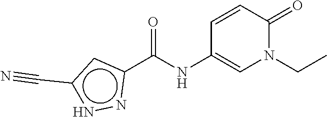 | 258 |
| 176 | 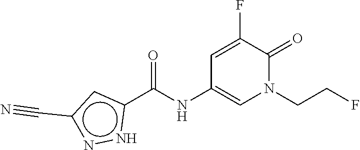 | 294 |
| 177 | 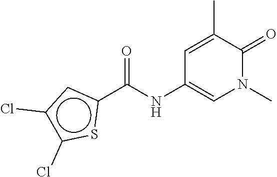 | 317 |
| 178 | 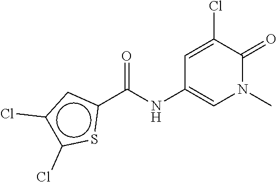 | 338 |
| 179 | 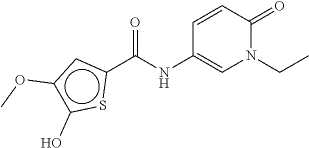 | 295 |
| 180 | 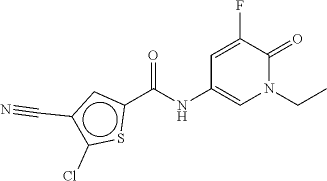 | 326 |
| 181 | 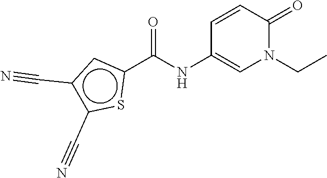 | 299 |
| 182 | 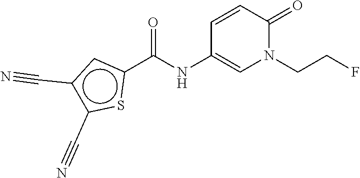 | 317 |
| 183 | 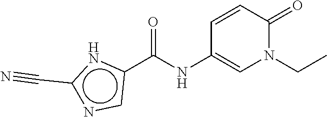 | 258 |
| 184 | 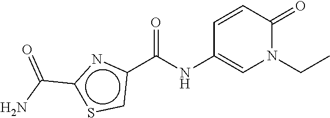 | 293 |
| 185 | 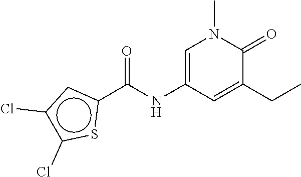 | 331 |
| 186 | 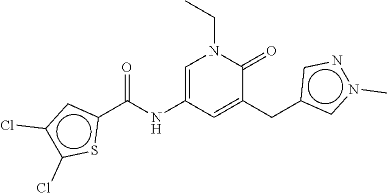 | 411 |
| 187 | 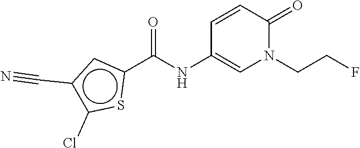 | 326 |
| 188 | 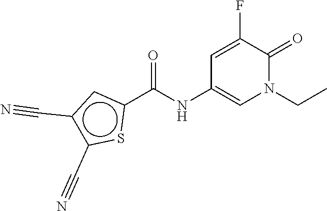 | 317 |
| 189 | 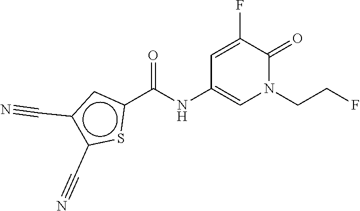 | 335 |
| 190 | 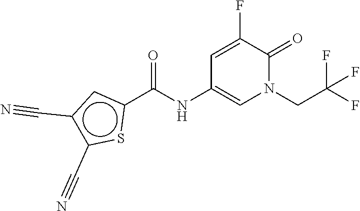 | 371 |
| 191 | 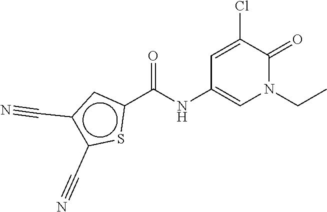 | 333 |
| 192 | 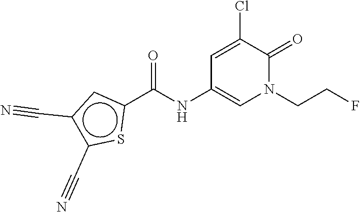 | 351 |
| 193 | 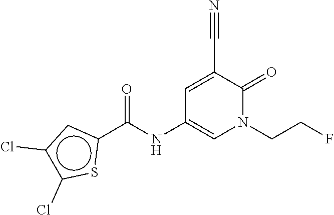 | 360 |
| 194 | 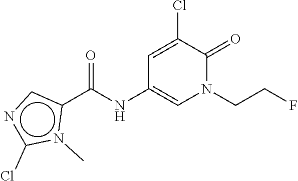 | 335 |
| 195 | 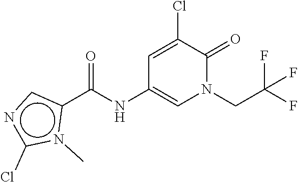 | 369 |
| 196 | 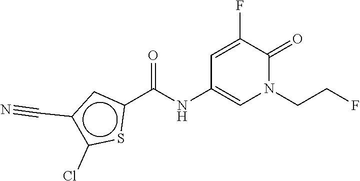 | 344 |
| 197 | 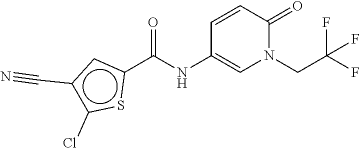 | 362 |
| 198 | 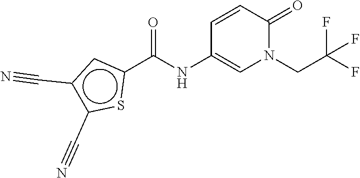 | 353 |
| 199 | 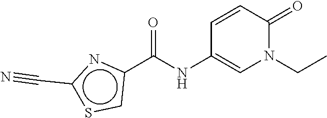 | 275 |
| 200 | 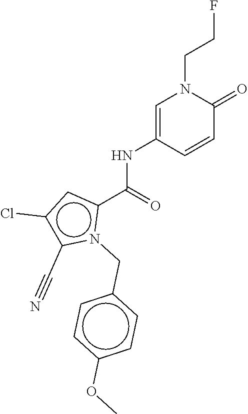 | 429 |
| 201 | 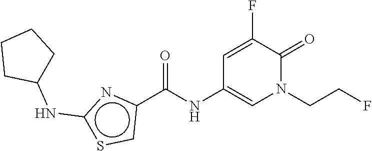 | 369 |
| 202 | 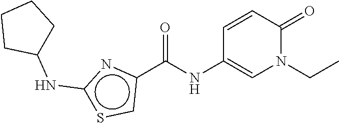 | 333 |
| 203 | 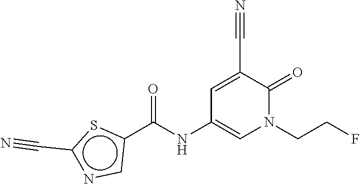 | 318 |
| 204 | 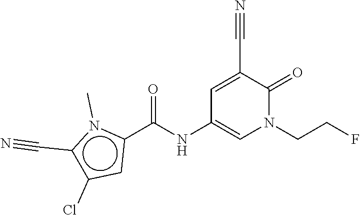 | 348 |
| 205 | 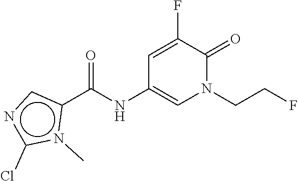 | 317 |
| 206 | 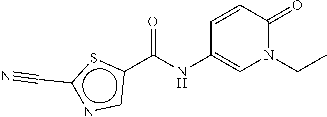 | 275 |
| 207 | 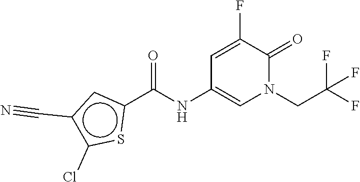 | 380 |
| 208 | 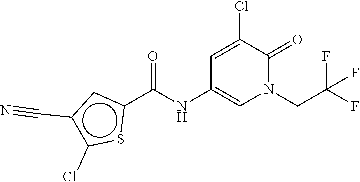 | 396 |
| 209 | 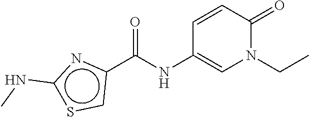 | 279 |
| 210 | 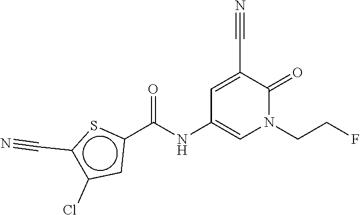 | 351 |
| 211 | 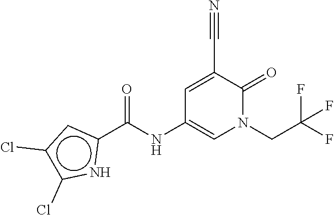 | 401 |
| 212 | 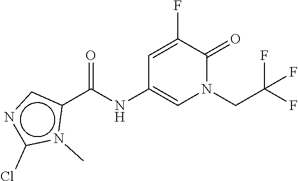 | 353 |
| 213 | 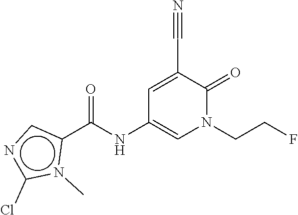 | 324 |
| 214 | 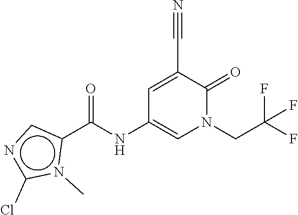 | 360 |
| 215 | 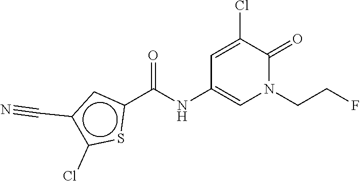 | 360 |
| 216 | 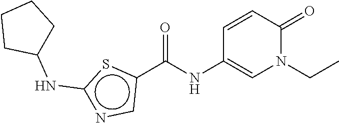 | 333 |
| 217 | 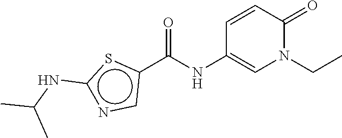 | 307 |
| 218 | 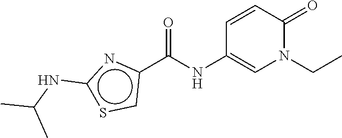 | 307 |
| 219 | 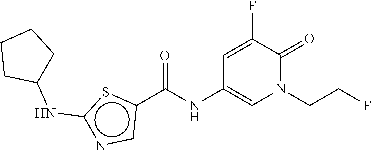 | 369 |
| 220 | 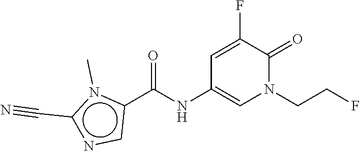 | 308 |
| 221 | 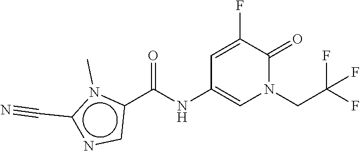 | 344 |
| 222 | 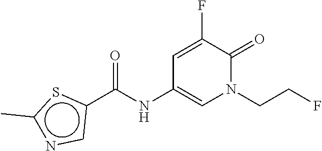 | 300 |
| 223 | 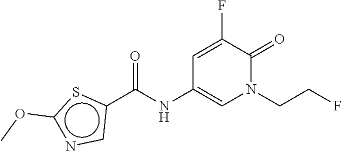 | 316 |
| 224 | 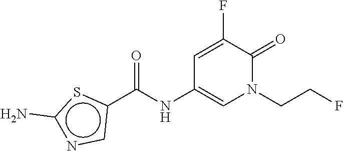 | 301 |
| 225 | 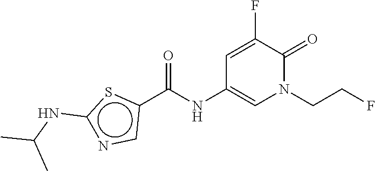 | 343 |
| 226 | 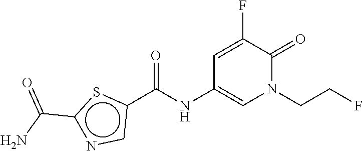 | 329 |
| 227 | 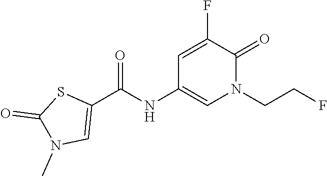 [SMILES: Cn1cc(C(=O)Nc2cc(F)c(=O)n(CCF)c2)sc1=O] | 316 |
| 228 | 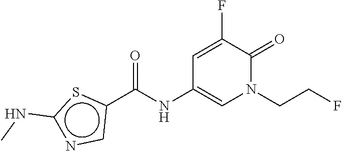 | 315 |
| 229 | 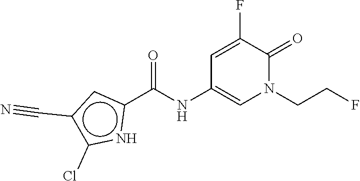 | 327 |
| 230 | 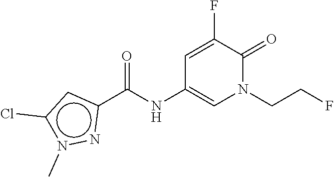 | 317 |
| 231 | 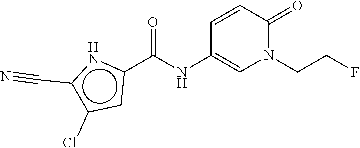 | 309 |
| 232 | 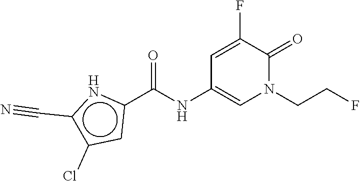 | 327 |
| 233 | 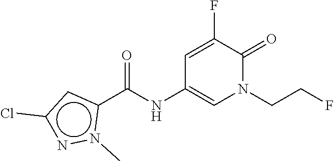 | 317 |
| 234 | 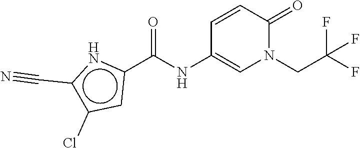 | 345 |
| 235 | 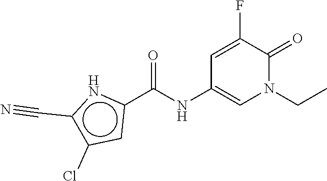 | 309 |
| 236 | 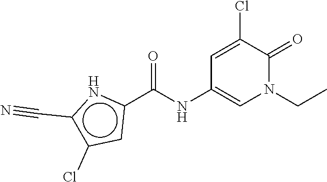 | 325 |
| 237 | 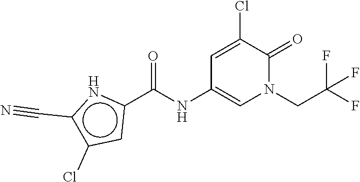 | 379 |
| 238 | 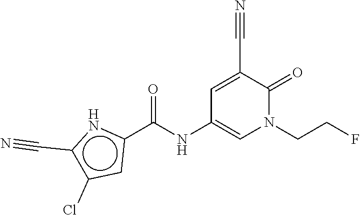 | 334 |
| 239 | 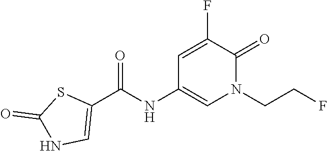 [SMILES: O=C(Nc1cc(F)c(=O)n(CCF)c1)c1c[nH]c(=O)s1] | 302 |
| 240 | 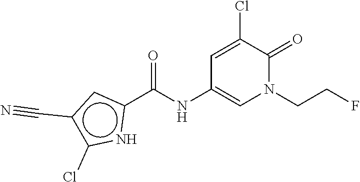 | 343 |
| 241 | 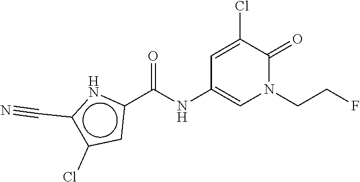 | 343 |
| 242 | 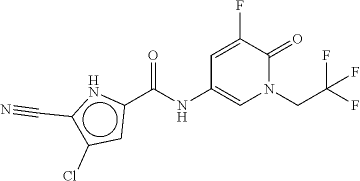 | 363 |
| 243 | 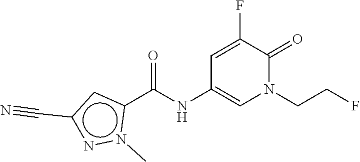 | 308 |
| 244 | 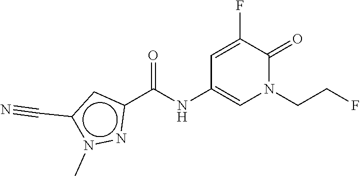 | 308 |
| 245 | 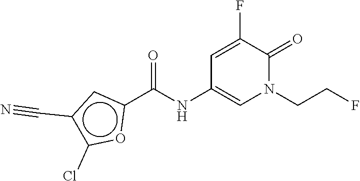 | 328 |
| 246 | 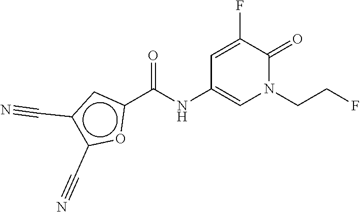 | 319 |
| 247 | 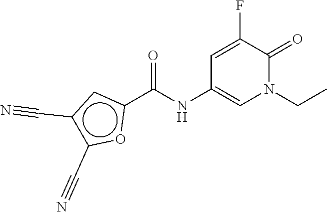 | 355 |
| 248 | 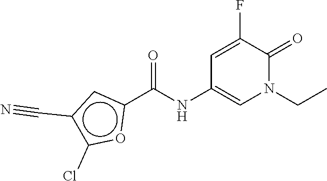 | 364 |
| 249 | 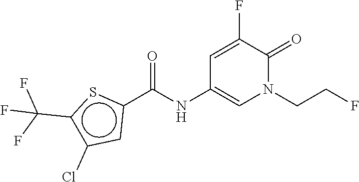 | 387 |
| 250 | 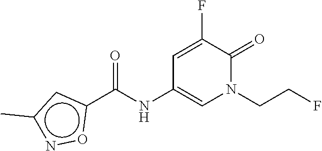 | 284 |
| 251 | 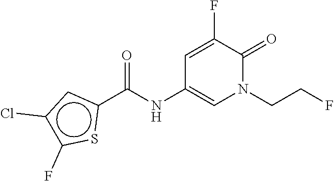 | 337 |
| 252 | 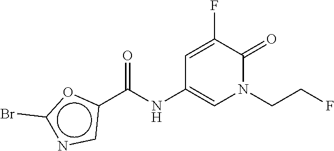 | 350 |
| 253 | 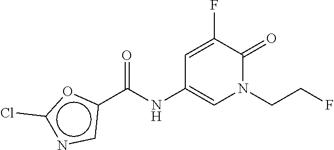 | 304 |
| 254 | 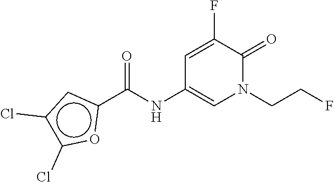 | 337 |
| 255 | 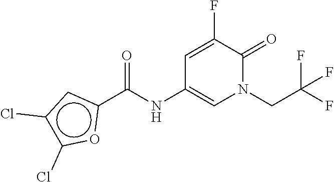 | 373 |
| 256 | 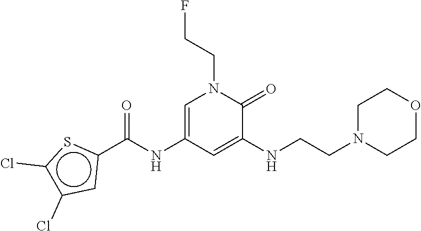 | 463 |
| 257 | 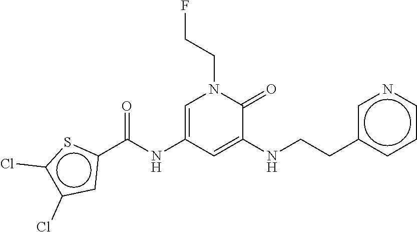 | 455 |
| 258 | 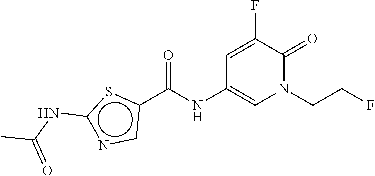 | 343 |
| 259 | 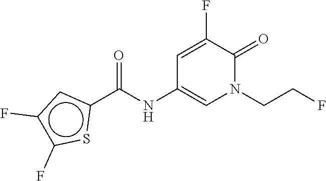 | 321 |
| 260 | 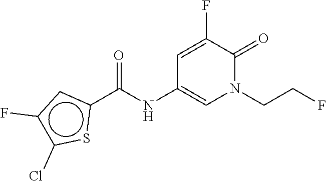 | 337 |
| 261 | 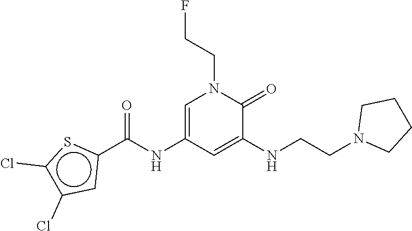 | 447 |
| 262 | 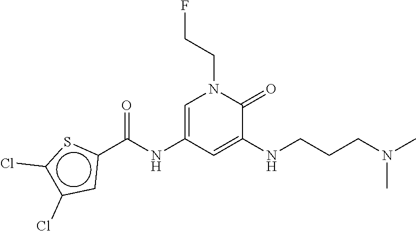 | 435 |
| 263 | 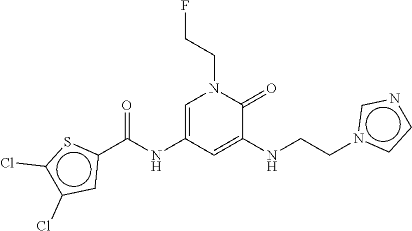 | 444 |
| 264 | 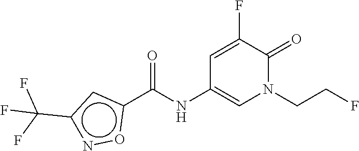 | 338 |
| 265 | 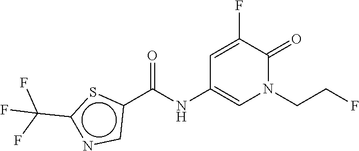 | 354 |
| 266 | 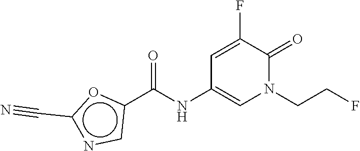 | 295 |
| 267 | 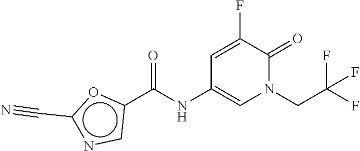 | 331 |
| 268 | 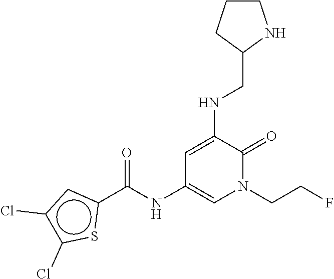 | 433 |
| 269 | 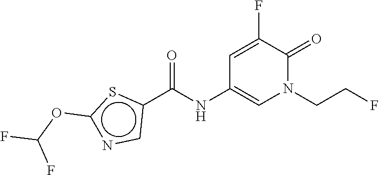 | 352 |
| 270 | 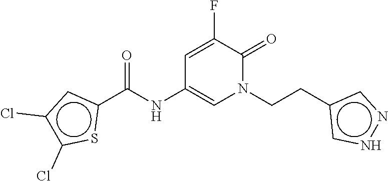 | 401 |
| 271 | 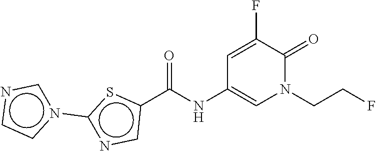 | 352 |
| 272 | 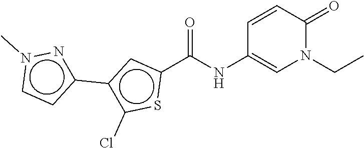 | 363 |
| 273 | 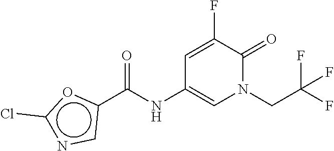 | 340 |
| 274 | 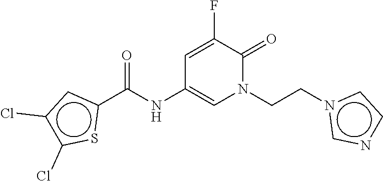 | 401 |
| 275 | 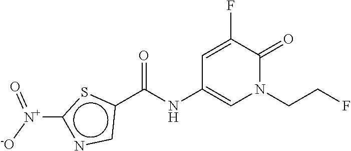 | 331 |
| 276 | 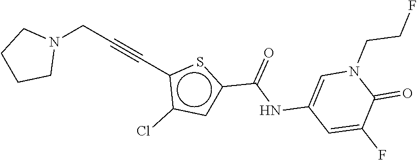 | 426 |
| 277 | 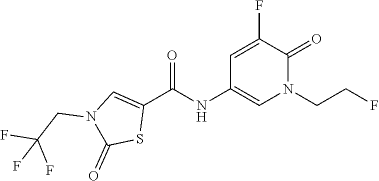 [SMILES: O=C(Nc1cc(F)c(=O)n(CCF)c1)c1cn(CC(F)(F)F)c(=O)s1] | 384 |
| 278 | 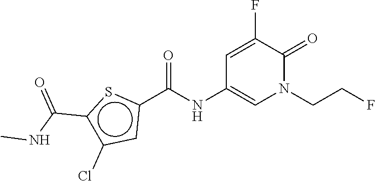 | 376 |
| 279 | 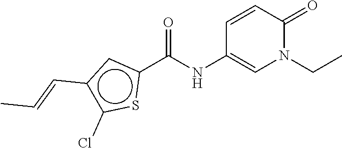 | 323 |
| 280 | 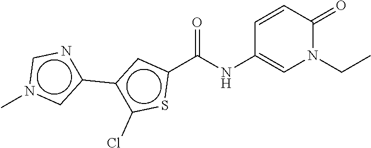 | 363 |
| 281 | 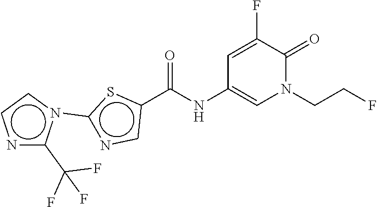 | 420 |
| 282 | 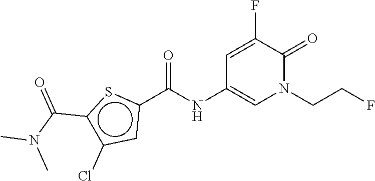 | 390 |
| 283 | 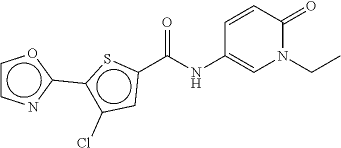 | 350 |
| 284 | 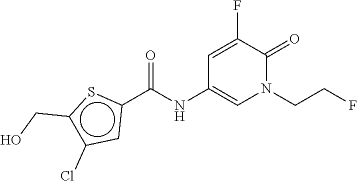 | 349 |
| 285 | 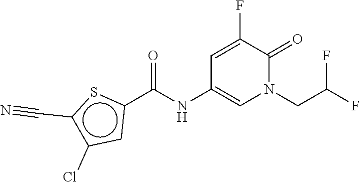 | 362 |
| 286 | 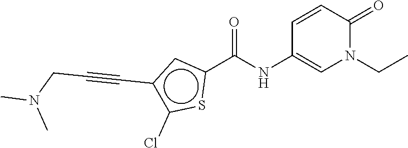 | 364 |
| 287 | 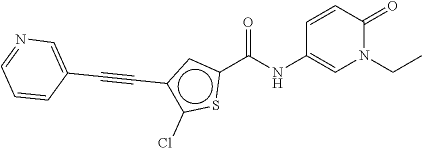 | 384 |
| 288 | 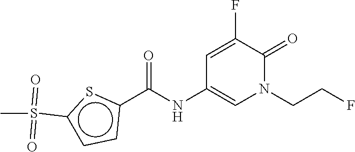 | 385 |
| 289 | 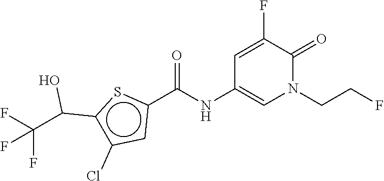 | 417 |
| 290 | 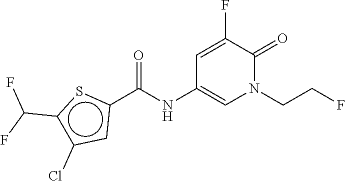 | 369 |
| 291 | 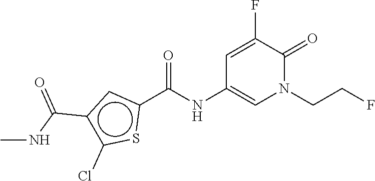 | 377 |
| 292 | 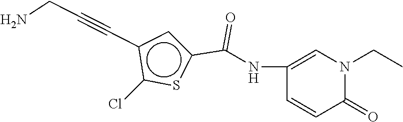 | 336 |
| 293 | 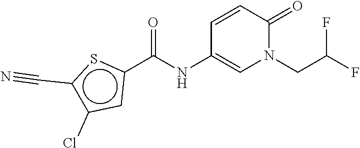 | 344 |
| 294 | 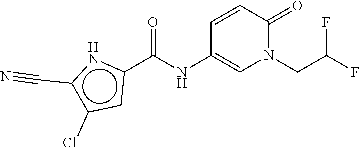 | 327 |
| 295 | 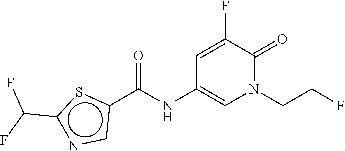 | 336 |
| 296 | 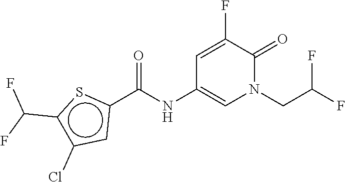 | 387 |
| 297 | 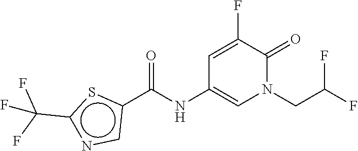 | 372 |
| 298 | 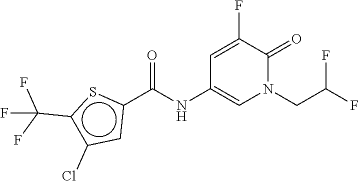 | 405 |
| 299 | 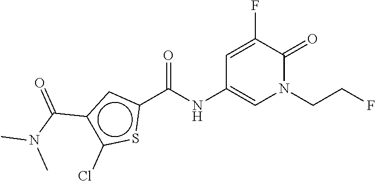 | 390 |
| 300 | 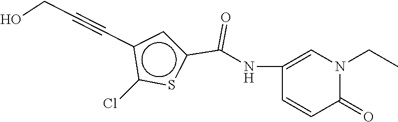 | 337 |
| 301 | 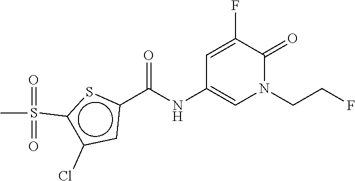 | 397 |
| 302 | 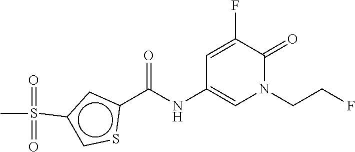 | 363 |
| 303 | 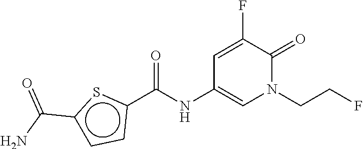 | 328 |
| 304 | 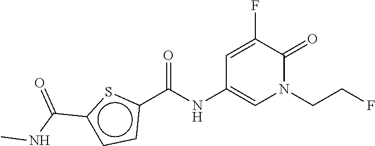 | 342 |
| 305 | 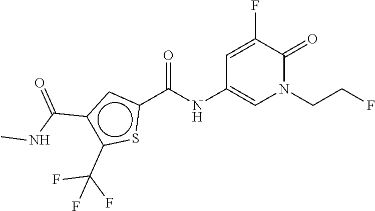 | 410 |
| 306 | 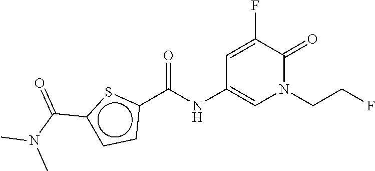 | 356 |
| 307 | 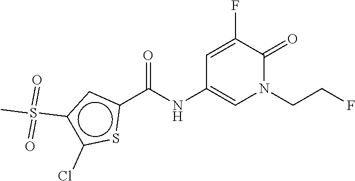 | 397 |
| 308 | 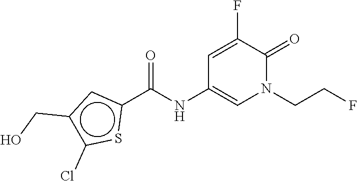 | 349 |
| 309 | 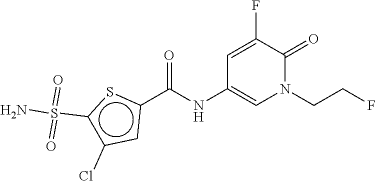 | 398 |
| 310 | 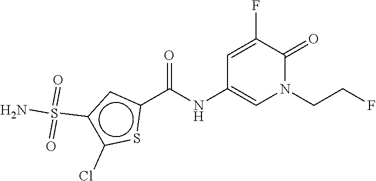 | 398 |
| 311 | 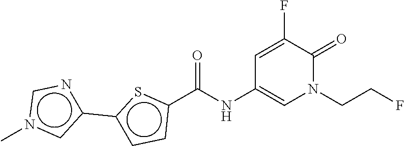 | 365 |
| 312 | 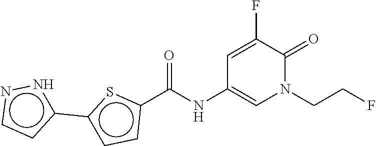 | 351 |
| 313 | 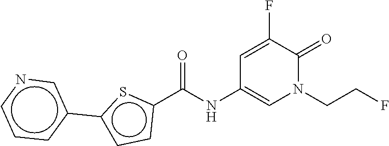 | 362 |
| 314 | 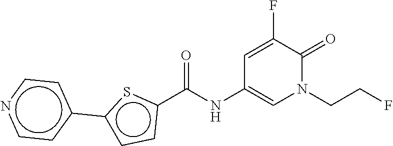 | 362 |
| 315 | 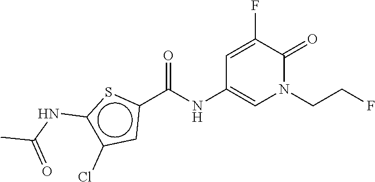 | 376 |
| 316 | 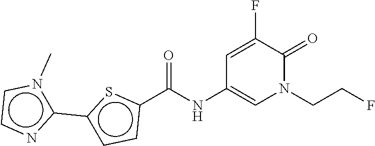 | 365 |
| 317 | 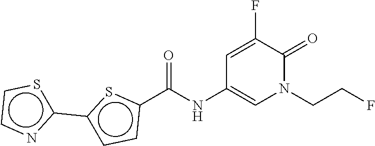 | 368 |
| 318 | 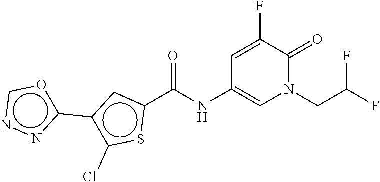 | 405 |
| 319 | 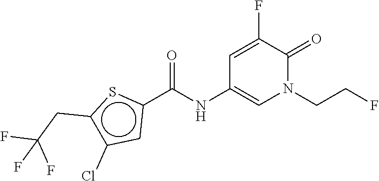 | 401 |
| 320 | 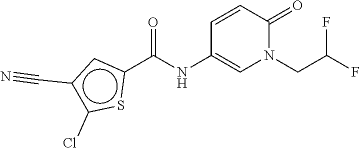 | 344 |
| 321 | 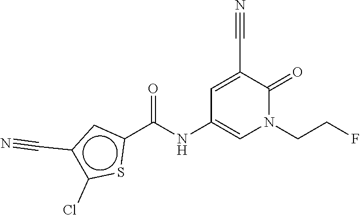 | 351 |
| 322 | 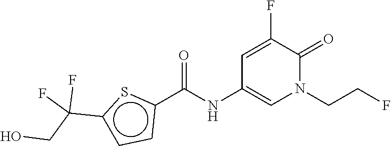 | 365 |
| 323 | 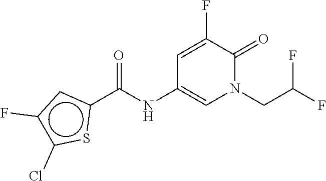 | 355 |
| 324 | 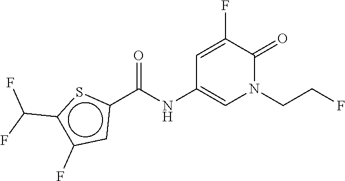 | 353 |
| 325 | 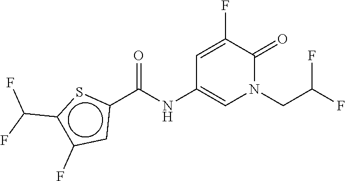 | 371 |
| 326 | 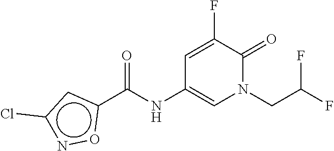 | 322 |
| 327 | 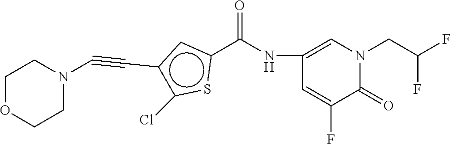 | 460 |
| 328 | 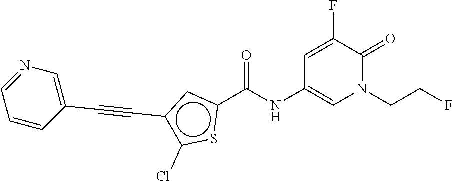 | 420 |
| 329 | 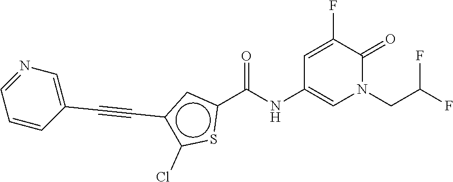 | 438 |
| 330 | 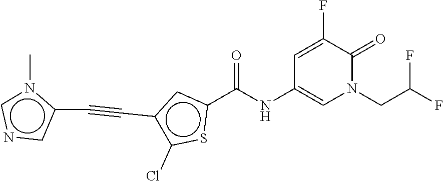 | 441 |
| 331 | 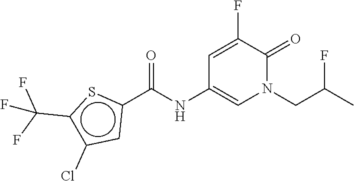 | 401 |
| 332 | 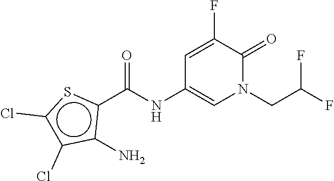 | 386 |
| 333 | 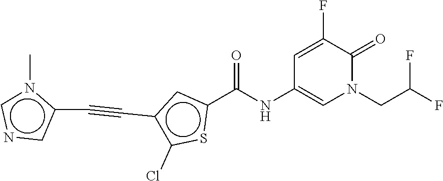 | 441 |
| 334 | 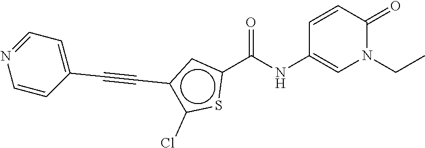 | 384 |
| 335 | 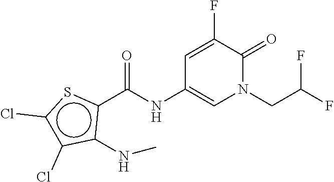 | 400 |
| 336 | 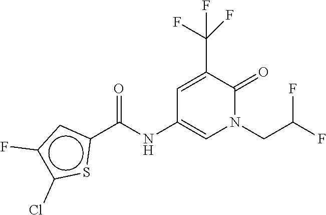 | 405 |
| 337 | 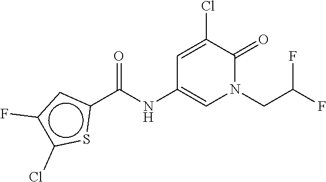 | 371 |
| 338 | 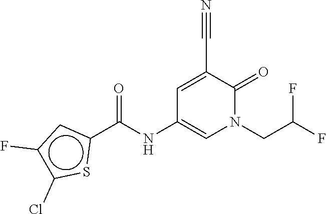 | 362 |
| 339 | 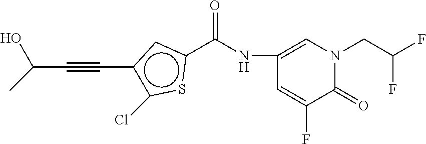 | 405 |
| 340 | 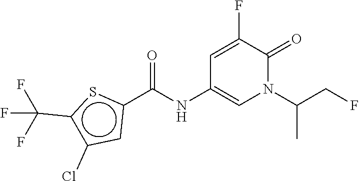 | 401 |
| 341 | 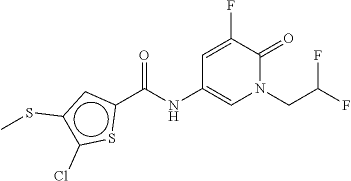 | 383 |
| 342 | 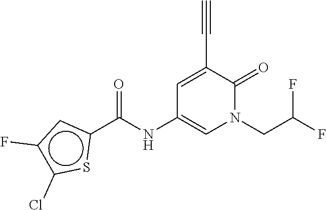 | 361 |
| 343 | 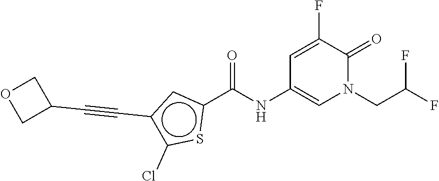 | 417 |
| 345 | 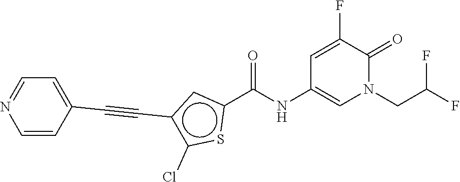 | 438 |
| 346 | 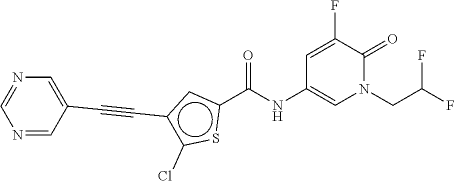 | 439 |

TABLE 2a
SMARCA2 inhibitors of the present disclosure.

| Compd. | | |
|---|---|---|
| No. | Structure | Mass (u) |
| 1a | 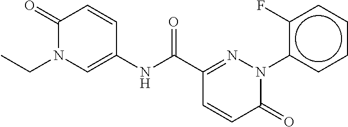 | 355 |
| 2a | 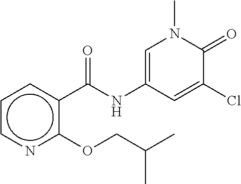 | 336 |
| 3a | 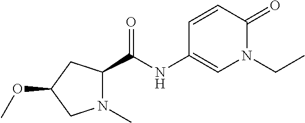 [SMILES: CCn1cc(NC(=O)[C@@H]2C[C@H](OC)CN2C)ccc1=O] | 280 |
| 4a | 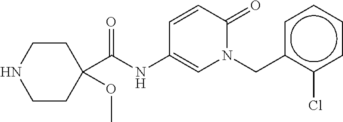 | 376 |
| 5a | 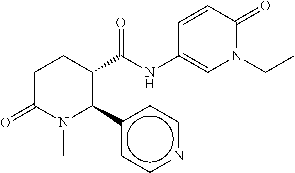 | ND |
| 6a | 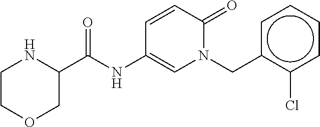 | 348 |
| 7a | 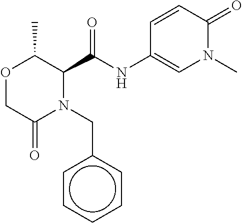 | 356 |
| 8a | 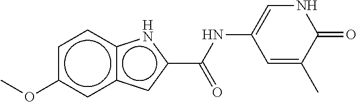 | 298 |
| 9a | 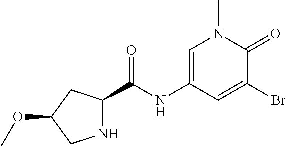 [SMILES: CO[C@@H]1CN[C@H](C(=O)Nc2cc(Br)c(=O)n(C)c2)C1] | 332 |
| 10a | 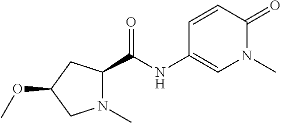 | 266 |
| 11a | 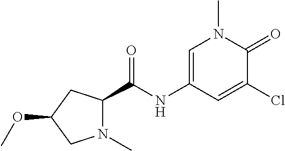 [SMILES: CO[C@H]1C[C@@H](C(=O)Nc2cc(Cl)c(=O)n(C)c2)N(C)C1] | 300 |
| 12a | 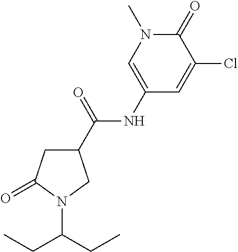 [SMILES: CCC(CC)N1CC(C(=O)Nc2cc(Cl)c(=O)n(C)c2)CC1=O] | 340 |
| 13a | 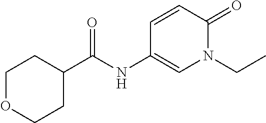 [SMILES: CCn1cc(NC(=O)C2CCOCC2)ccc1=O] | 251 |
| 14a | 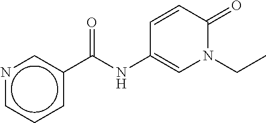 | 244 |
| 15a | 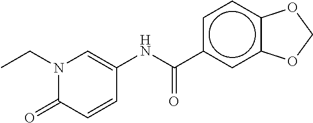 | 287 |
| 16a | 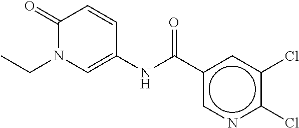 | 312 |
| 17a | 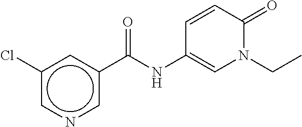 | 278 |
| 18a | 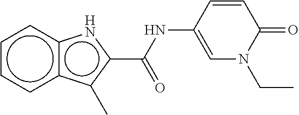 | 296 |
| 19a | 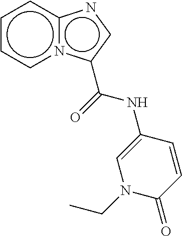 | 283 |
| 20a | 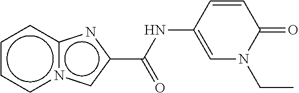 | 283 |
| 21a | 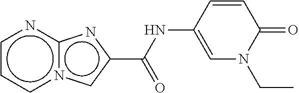 | 284 |
| 22a | 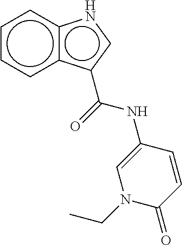 | 282 |
| 23a | 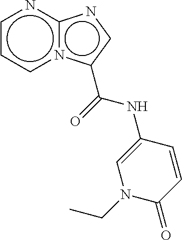 | 284 |
| 24a | 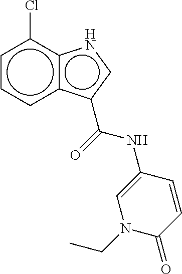 | 316 |
| 25a | 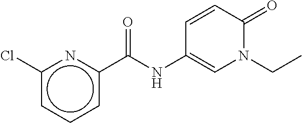 | 278 |
| 26a | 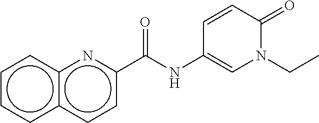 | 294 |
| 27a | 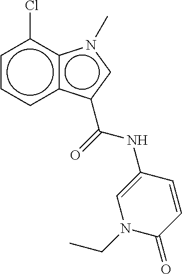 | 330 |
| 28a | 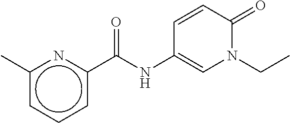 | 258 |
| 29a | 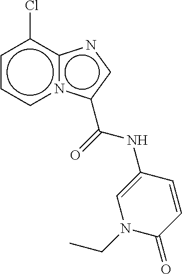 | 317 |
| 30a | 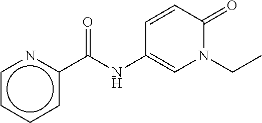 | 244 |
| 31a | 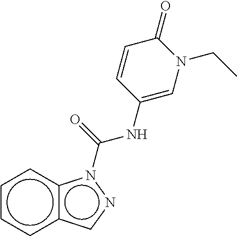 | 283 |
| 32a | 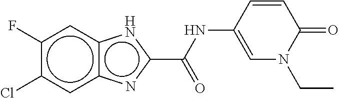 | 335 |
| 33a | 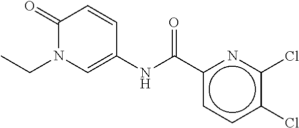 | 312 |
| 34a | 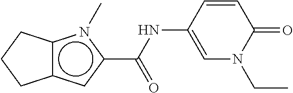 | 286 |
| 35a | 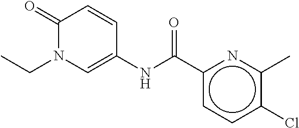 | 292 |
| 36a | 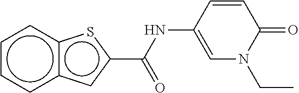 | 299 |
| 37a | 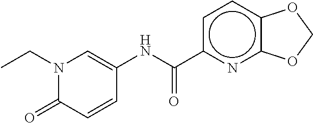 | 288 |
| 38a | 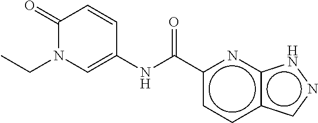 | 284 |
| 39a | 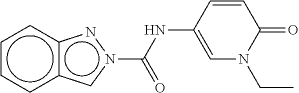 | 283 |
| 40a | 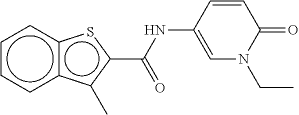 | 313 |
| 41a | 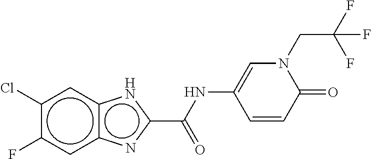 | 389 |
| 42a | 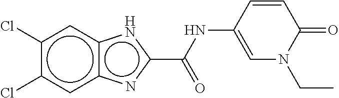 | 351 |
| 43a | 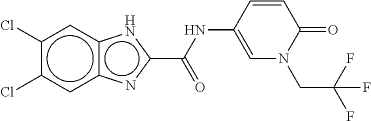 | 405 |
| 44a | 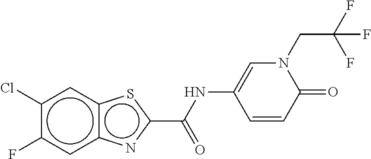 | 406 |
| 45a | 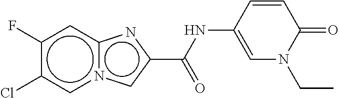 | 335 |
| 46a | 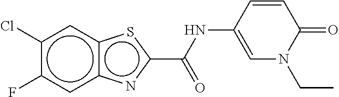 | 352 |
| 47a | 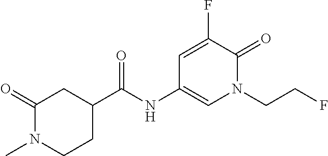 [SMILES: CN1CCC(C(=O)Nc2cc(F)c(=O)n(CCF)c2)CC1=O] | 314 |
| 48a | 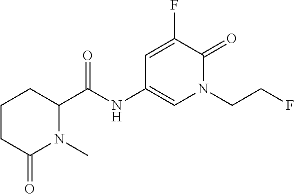 [SMILES: CN1C(=O)CCCC1C(=O)Nc1cc(F)c(=O)n(CCF)c1] | 314 |
| 49a | 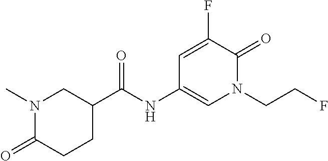 [SMILES: CN1CC(C(=O)Nc2cc(F)c(=O)n(CCF)c2)CCC1=O] | 314 |
| 50a | 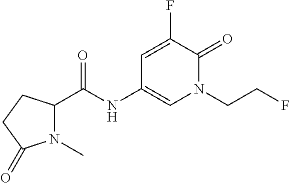 [SMILES: CN1C(=O)CCC1C(=O)Nc1cc(F)c(=O)n(CCF)c1] | 300 |
| 51a | 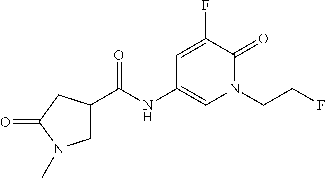 [SMILES: CN1CC(C(=O)Nc2cc(F)c(=O)n(CCF)c2)CC1=O] | 300 |
| 52a | 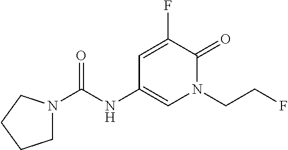 [SMILES: O=C(Nc1cc(F)c(=O)n(CCF)c1)N1CCCC1] | 272 |
| 53a | 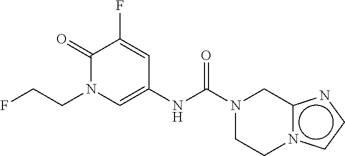 | 324 |
| 54a | 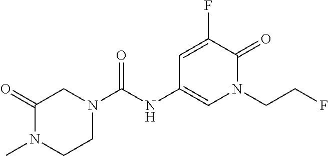 [SMILES: CN1CCN(C(=O)Nc2cc(F)c(=O)n(CCF)c2)CC1=O] | 315 |
| 55a | 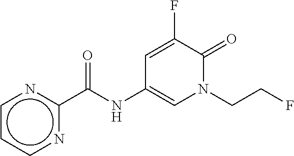 | 281 |
| 56a | 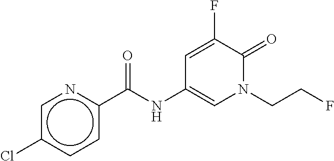 | 314 |
| 57a | 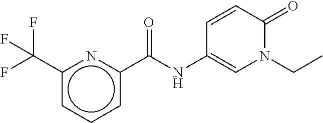 | 312 |
| 58a | 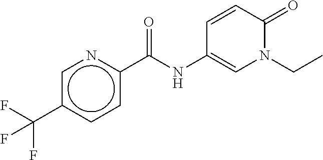 | 312 |
| 59a | 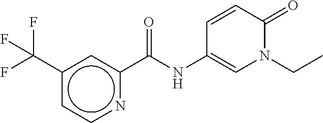 | 312 |
| 60a | 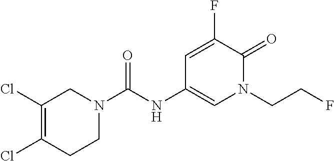 [SMILES: O=C(Nc1cc(F)c(=O)n(CCF)c1)N1CCC(Cl)=C(Cl)C1] | 352 |
| 61a | 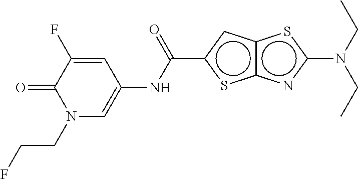 | 413 |
| 62a | 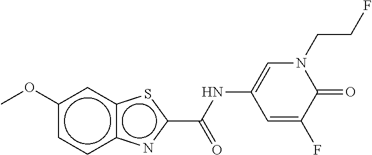 | 366 |
| 63a | 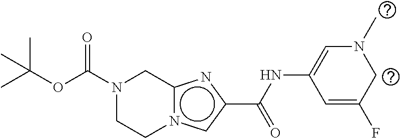 | 424 |
| 64a | 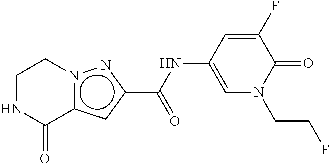 | 338 |
| 65a | 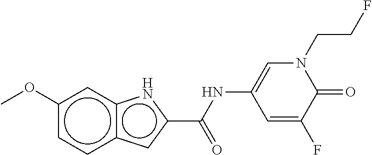 | 348 |
| 66a | 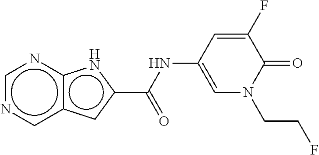 | 320 |
| 67a | 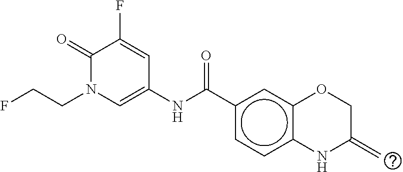 | 350 |
| 68a | 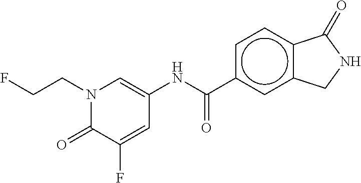 | 334 |
| 69a | 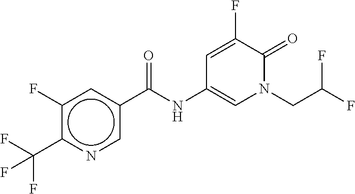 | 384 |
| 70a | 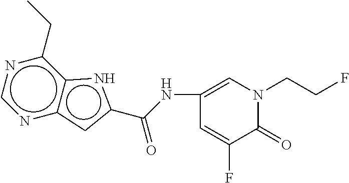 | 350 |
| 71a | 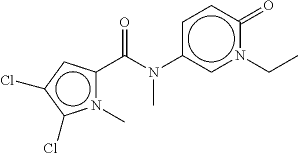 | ND |

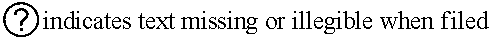

TABLE 2b
SMARCA2 inhibitors of the present disclosure.

| Compd. No. | Structure | Mass (u) |
|---|---|---|
| 1b | 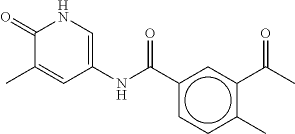 | 285 |
| 2b | 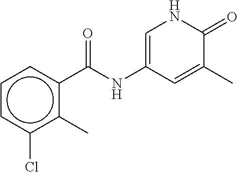 | 277 |
| 3b | 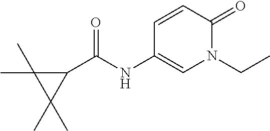 [SMILES: CCn1cc(NC(=O)C2C(C)(C)C2(C)C)ccc1=O] | 263 |
| 4b | 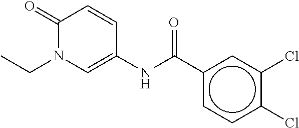 | 311 |
| 5b | 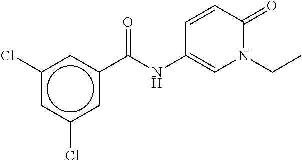 | 311 |
| 6b | 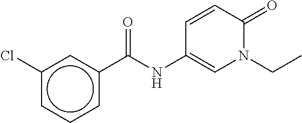 | 277 |
| 7b | 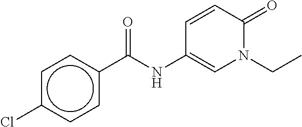 | 277 |
| 8b | 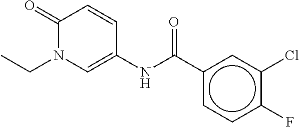 | 295 |
| 9b | 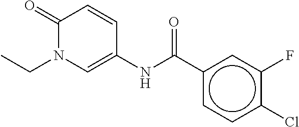 | 295 |
| 10b | 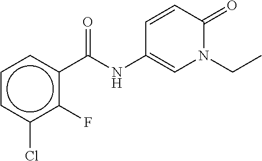 | 295 |
| 11b | 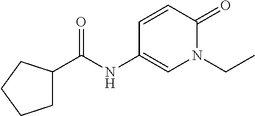 [SMILES: CCn1cc(NC(=O)C2CCCC2)ccc1=O] | 235 |
| 12b | 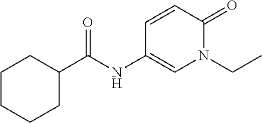 [SMILES: CCn1cc(NC(=O)C2CCCCC2)ccc1=O] | 249 |
| 13b | 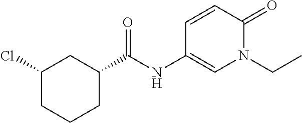 [SMILES: CCn1cc(NC(=O)[C@@H]2CCC[C@H](Cl)C2)ccc1=O] | 283 |
| 14b | 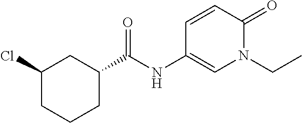 [SMILES: CCn1cc(NC(=O)[C@@H]2CCC[C@@H](Cl)C2)ccc1=O] | 283 |
| 15b | 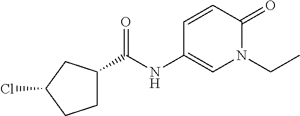 [SMILES: CCn1cc(NC(=O)[C@@H]2CC[C@H](Cl)C2)ccc1=O] | 269 |
| 16b | 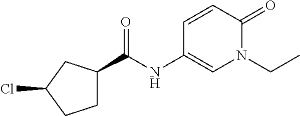 [SMILES: CCn1cc(NC(=O)[C@H]2CC[C@@H](Cl)C2)ccc1=O] | 269 |
| 17b | 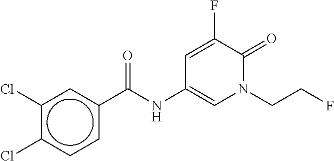 | 347 |
| 18b | 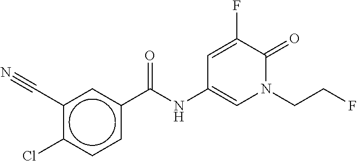 | 338 |
| 19b | 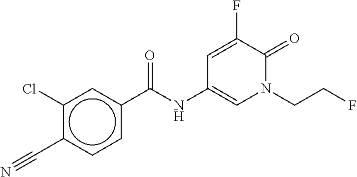 | 338 |
| 20b | 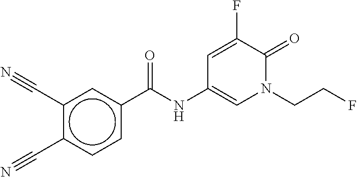 | 329 |
| 21b | 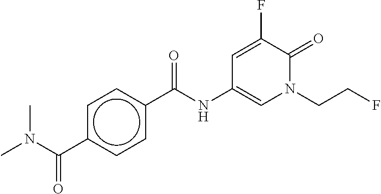 | 350 |
| 22b | 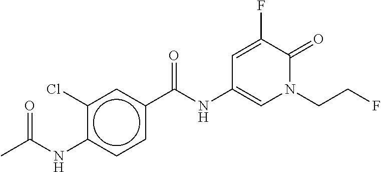 | 370 |
| 23b | 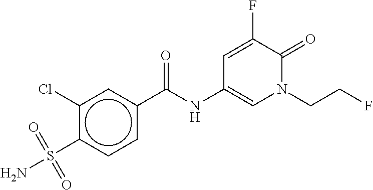 | 392 |

TABLE 2c
SMARCA2 inhibitors of the present disclosure.

| Compd. | | Mass |
|---|---|---|
| No. | Structure | (u) |
| 1c | 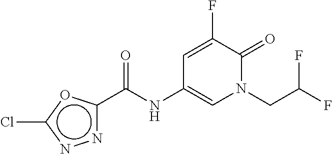 | 323 |
| 2c | 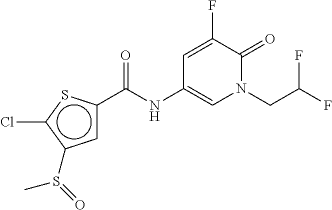 | 399 |
| 3c | 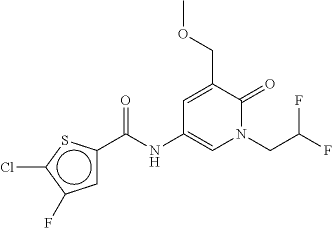 | 381 |
| 4c | 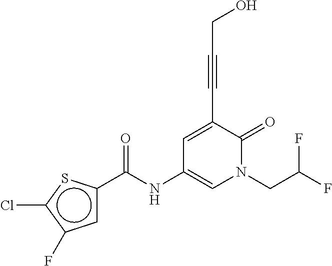 | 391 |
| 5c | 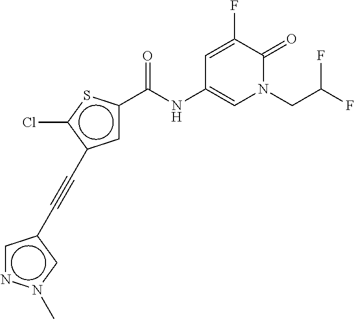 | 441 |
| 6c | 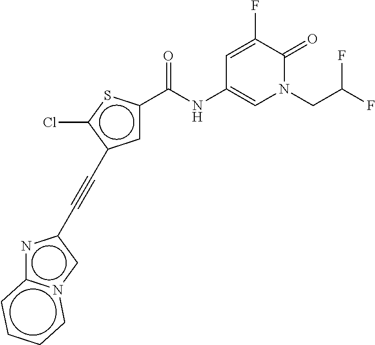 | 477 |
| 7c | 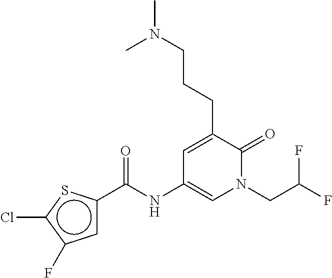 | 422 |
| 8c | 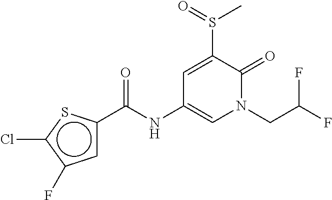 | 399 |
| 9c | 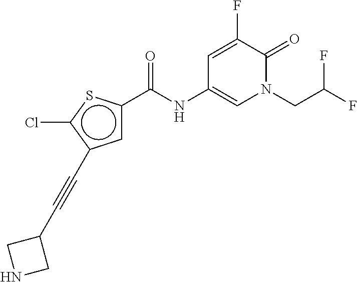 | 416 |
| 10c | 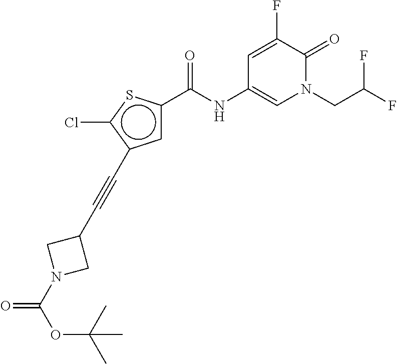 | 416 |
| 11c | 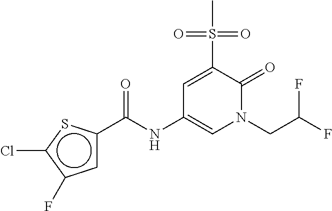 | 415 |
| 12c | 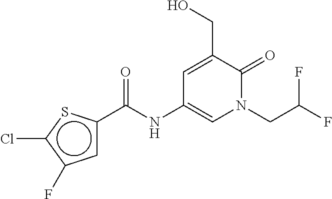 | 367 |
| 13c | 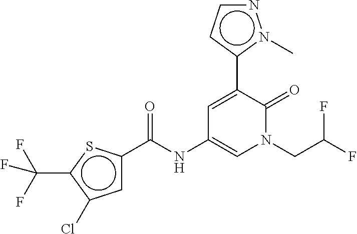 | 467 |
| 14c | 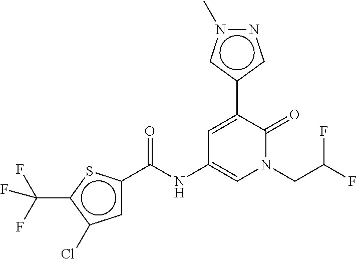 | 467 |
| 15c | 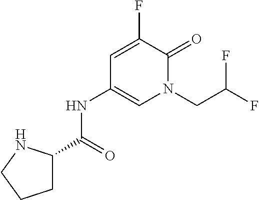 [SMILES: O=C(Nc1cc(F)c(=O)n(CC(F)F)c1)[C@@H]1CCCN1] | 290 |
| 16c | 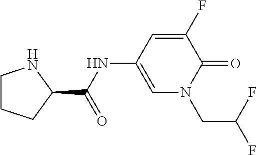 [SMILES: O=C(Nc1cc(F)c(=O)n(CC(F)F)c1)[C@H]1CCCN1] | 290 |
| 17c | 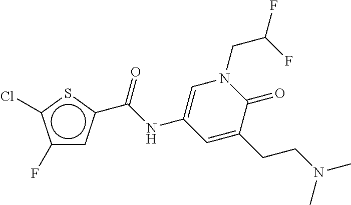 | 408 |
| 18c | 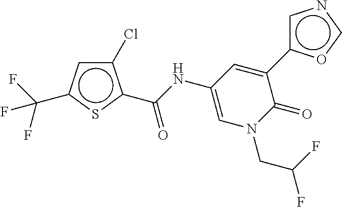 | 454 |
| 19c | 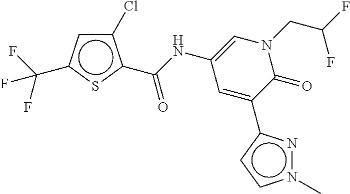 | 467 |
| 20c | 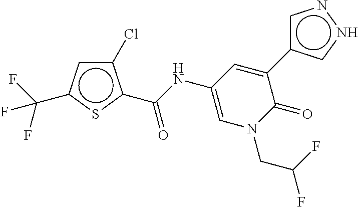 | 453 |
| 21c | 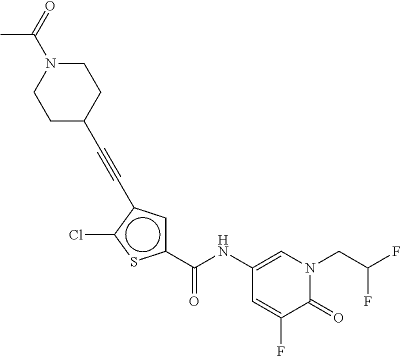 | 408 |
| 22c | 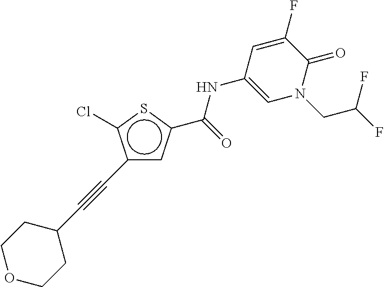 | 445 |
| 23c | 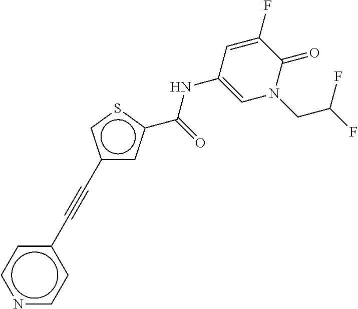 | 404 |
| 24c | 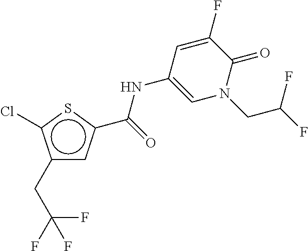 | 419 |
| 25c | 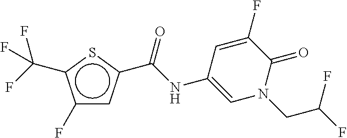 | 389 |
| 26c | 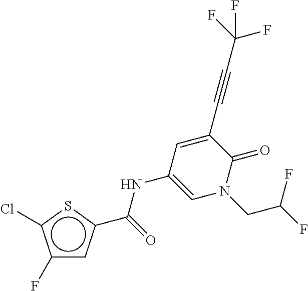 | 429 |
| 27c | 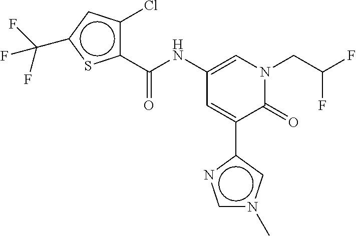 | 467 |
| 28c | 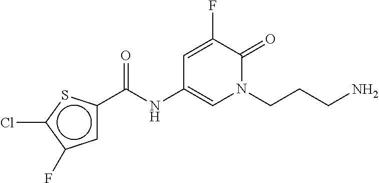 | 348 |
| 29c | 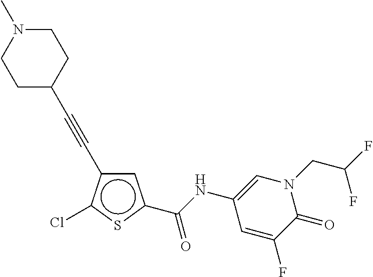 | 458 |
| 30c | 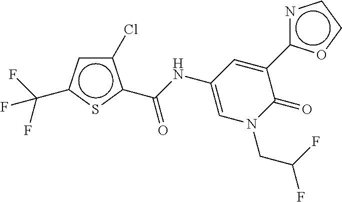 | 454 |
| 31c | 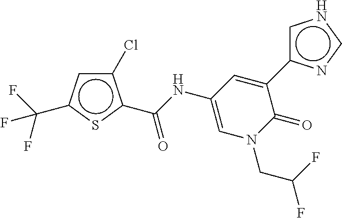 | 453 |
| 32c | 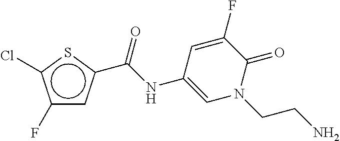 | 334 |
| 33c | 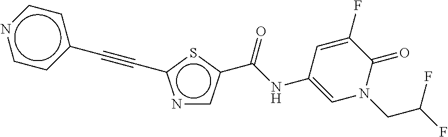 | 405 |
| 34c | 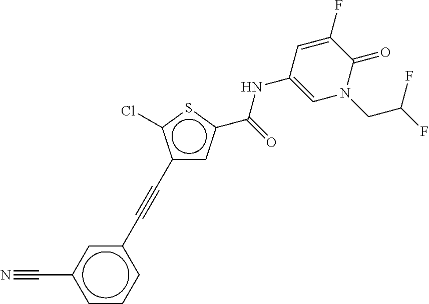 | 462 |
| 35c | 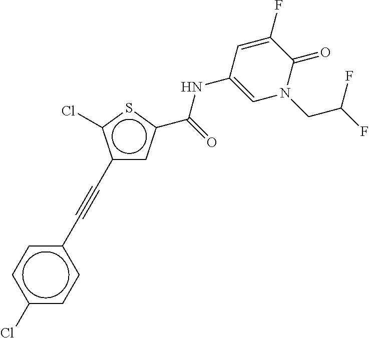 | 471 |
| 36c | 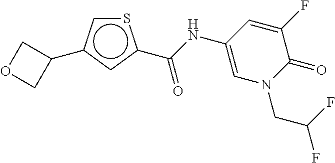 | 359 |
| 37c | 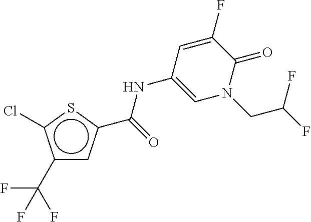 | 405 |
| 38c | 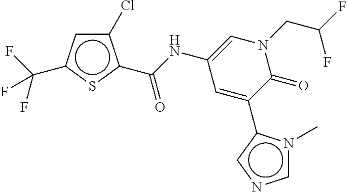 | 467 |
| 39c | 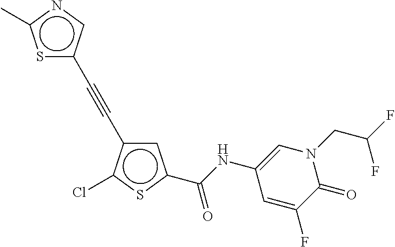 | 458 |
| 40c | 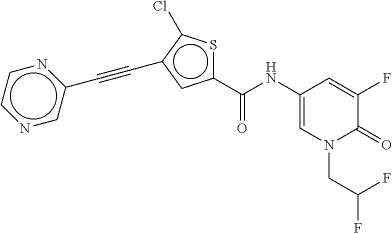 | 439 |
| 41c | 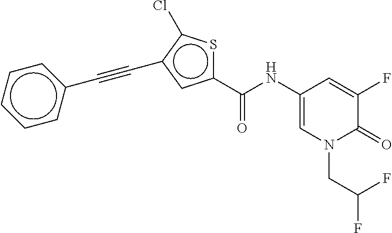 | 437 |
| 42c | 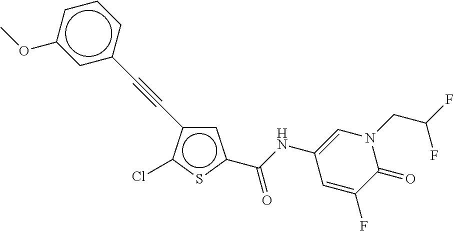 | 467 |
| 43c | 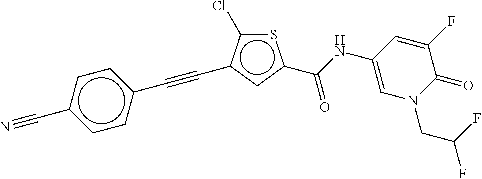 | 462 |
| 44c | 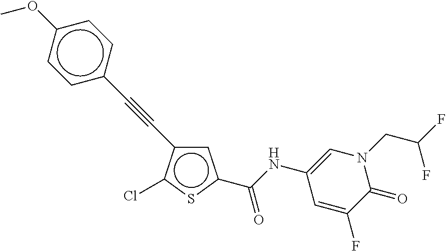 | 467 |
| 45c | 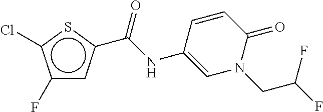 | 337 |
| 46c | 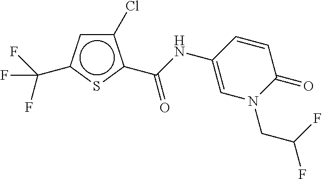 | 387 |
| 47c | 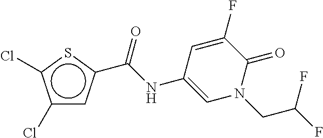 | 371 |
| 48c | 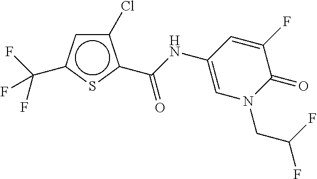 | 405 |
| 49c | 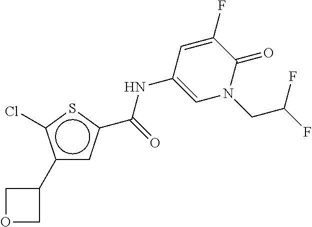 | 393 |
| 50c | 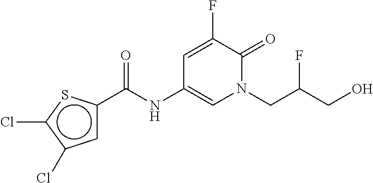 | 383 |
| 51c | 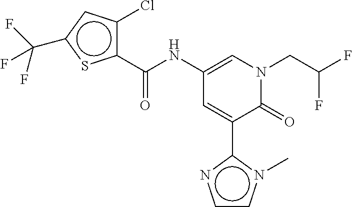 | 467 |
| 52c | 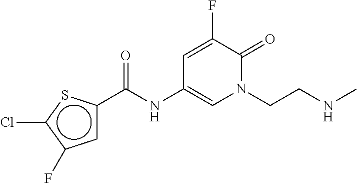 | 348 |
| 53c | 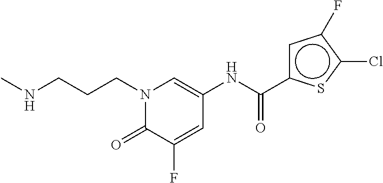 | 362 |
| 54c | 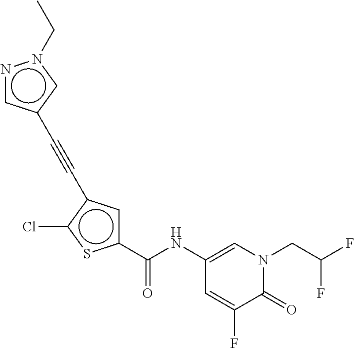 | 455 |
| 55c | 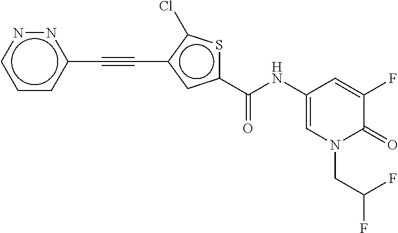 | 439 |
| 56c | 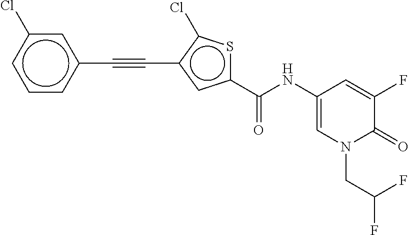 | 471 |
| 57c | 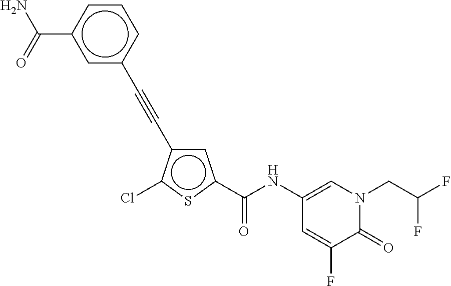 | 480 |
| 58c | 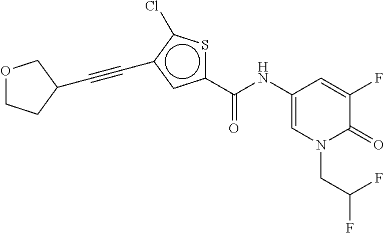 | 431 |
| 59c | 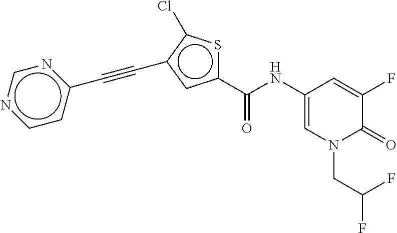 | 439 |
| 60c | 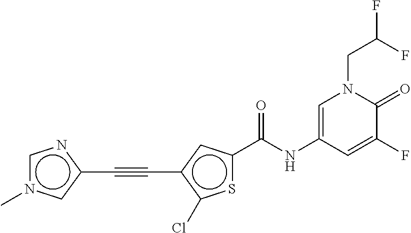 | 441 |
| 61c | 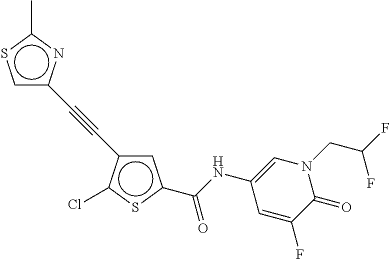 | 458 |
| 62c | 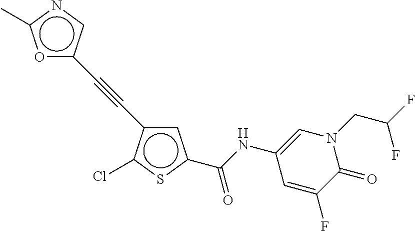 | 442 |
| 63c | 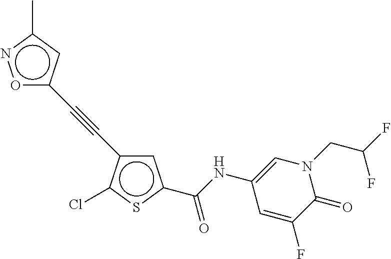 | 442 |
| 64c | 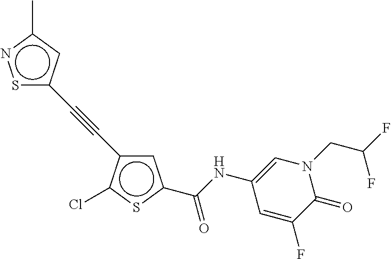 | 458 |
| 65c | 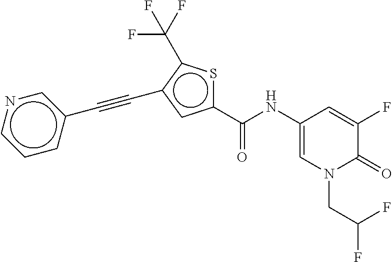 | 472 |
| 66c | 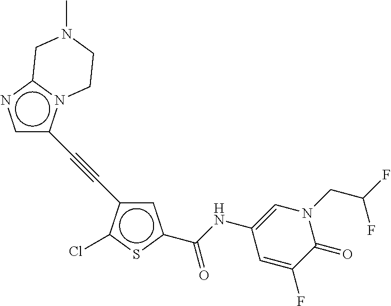 | 496 |
| 67c | 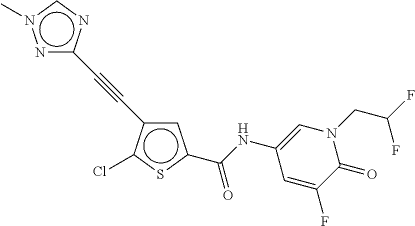 | 442 |
| 68c | 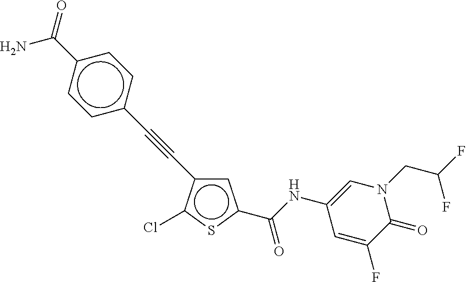 | 480 |
| 69c | 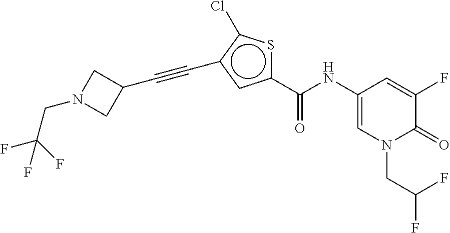 | 498 |
| 70c | 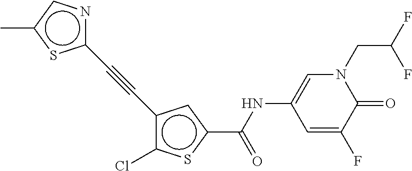 | 458 |
| 71c | 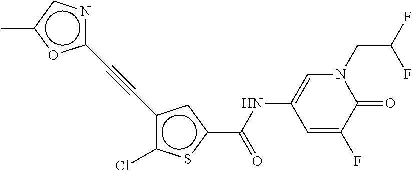 | 442 |
| 72c | 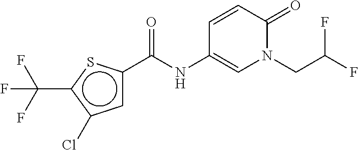 | 387 |
| 73c | 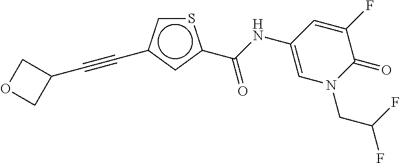 | 383 |
| 74c | 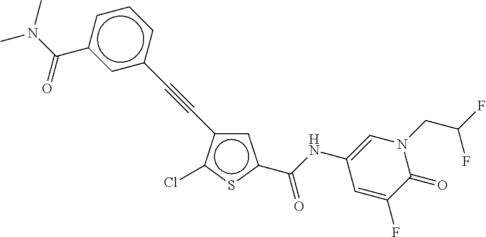 | 508 |
| 75c | 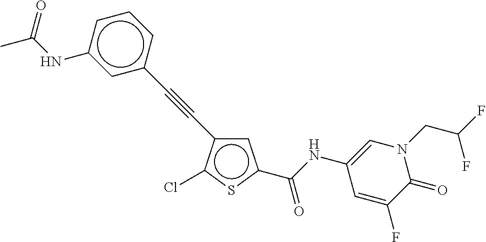 | 494 |
| 76c | 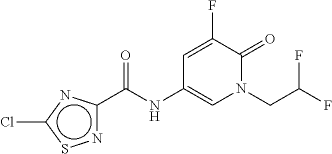 | 361 |
| 77c | 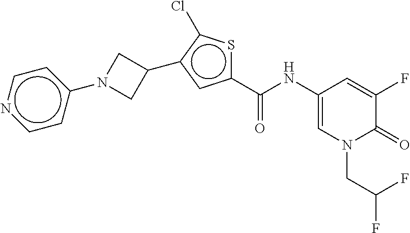 | 469 |
| 78c | 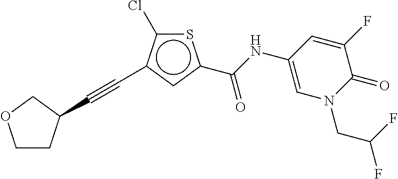 | 431 |
| 79c | 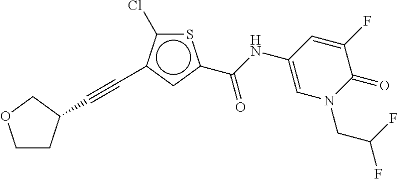 | 431 |
| 80c | 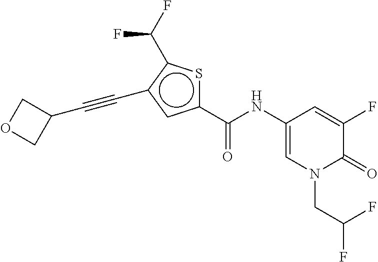 | 433 |
| 81c | 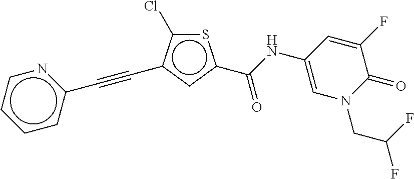 | 438 |
| 82c | 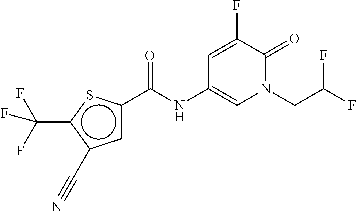 | 395 |
| 165c | 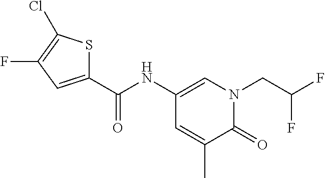 [SMILES: Cc1cc(NC(=O)c2cc(F)c(Cl)s2)cn(CC(F)F)c1=O] | ND |
| 167c | 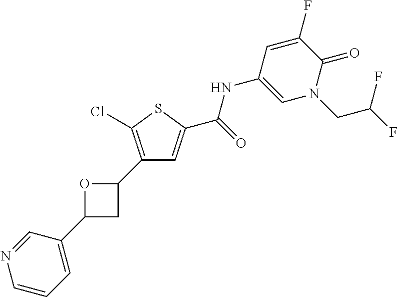 [SMILES: O=C(Nc1cc(F)c(=O)n(CC(F)F)c1)c1cc(C2CC(c3cccnc3)O2)c(Cl)s1] | ND |
| 169c | 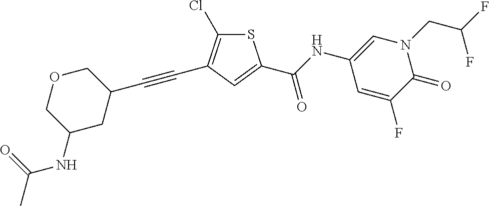 [SMILES: CC(=O)NC1COCC(C#Cc2cc(C(=O)Nc3cc(F)c(=O)n(CC(F)F)c3)sc2Cl)C1] | 502 |
| 171c | 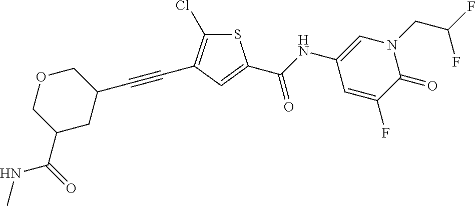 [SMILES: CNC(=O)C1COCC(C#Cc2cc(C(=O)Nc3cc(F)c(=O)n(CC(F)F)c3)sc2Cl)C1] | ND |
| 173c | 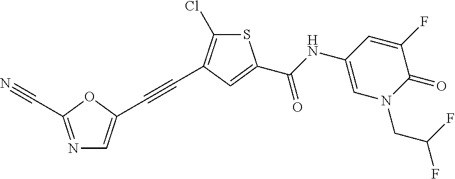 [SMILES: N#Cc1ncc(C#Cc2cc(C(=O)Nc3cc(F)c(=O)n(CC(F)F)c3)sc2Cl)o1] | 451 |
| 175c | 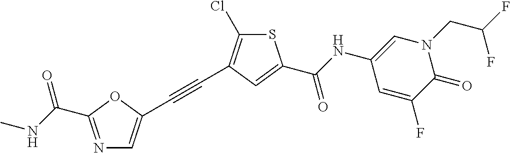 [SMILES: CNC(=O)c1ncc(C#Cc2cc(C(=O)Nc3cc(F)c(=O)n(CC(F)F)c3)sc2Cl)o1] | 485 |
| 177c | 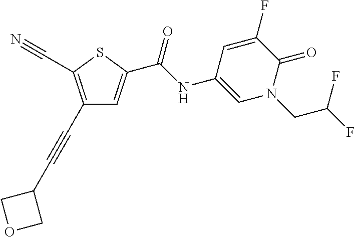 [SMILES: N#Cc1sc(C(=O)Nc2cc(F)c(=O)n(CC(F)F)c2)cc1C#CC1COC1] | 408 |
| 179c | 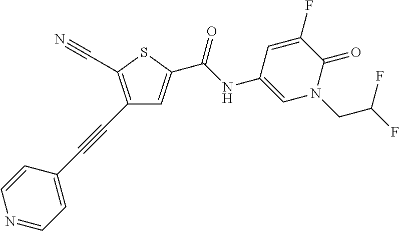 [SMILES: N#Cc1sc(C(=O)Nc2cc(F)c(=O)n(CC(F)F)c2)cc1C#Cc1ccncc1] | 429 |
| 83c | 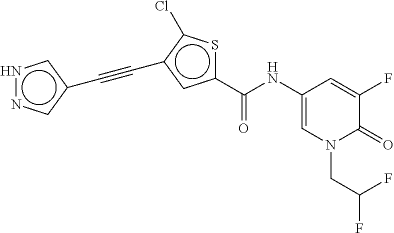 | 427 |
| 84c | 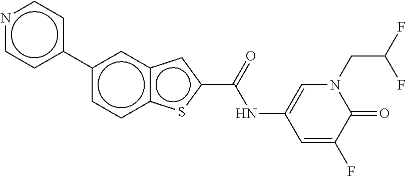 | 430 |
| 85c | 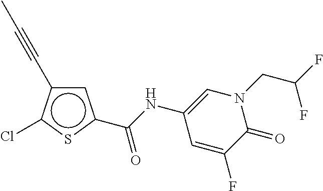 | 375 |
| 86c | 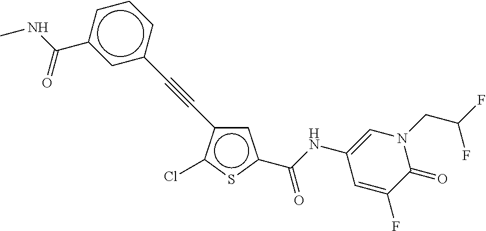 | 494 |
| 87c | 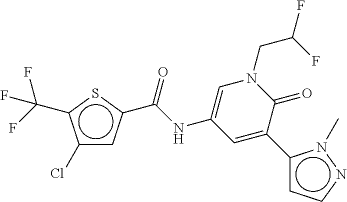 | 467 |
| 88c | 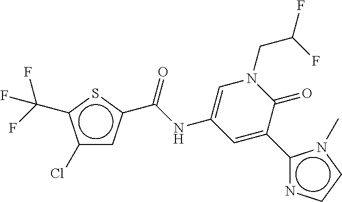 | 467 |
| 89c | 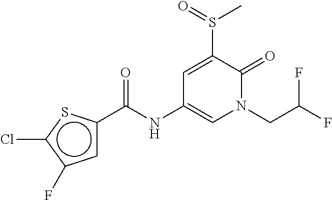 | 467 |
| 90c | 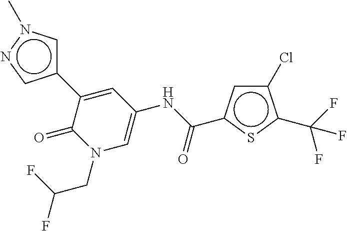 | 467 |
| 91c | 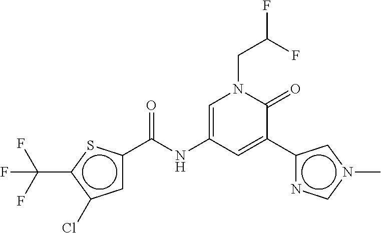 | 467 |
| 92c | 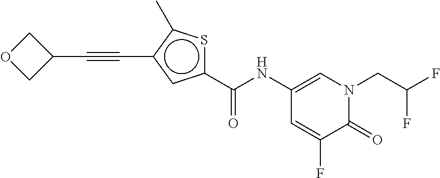 | 397 |
| 93c | 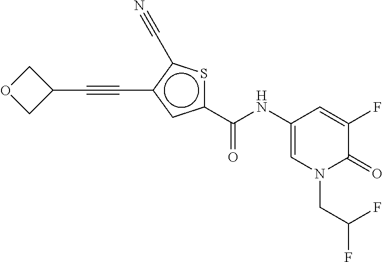 | 408 |
| 94c | 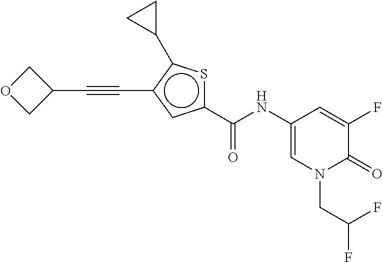 | 423 |
| 95c | 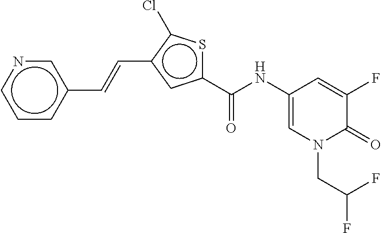 | 440 |
| 96c | 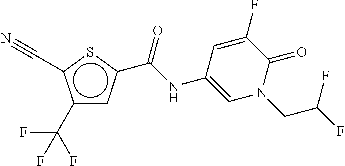 | 396 |
| 97c | 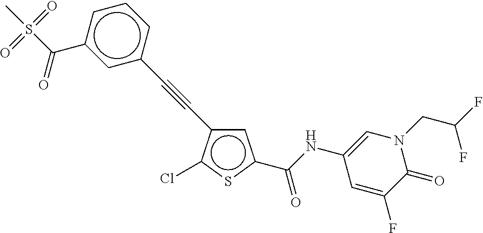 | 530 |
| 98c | 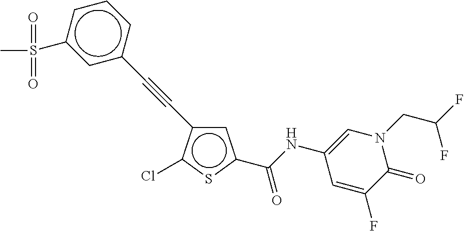 | 515 |
| 99c | 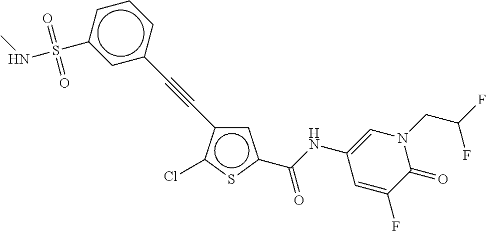 | 530 |
| 100c | 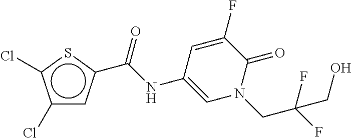 | 401 |
| 101c | 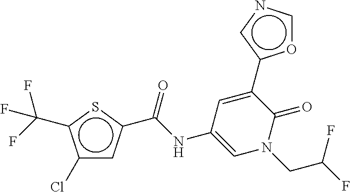 | 454 |
| 102c | 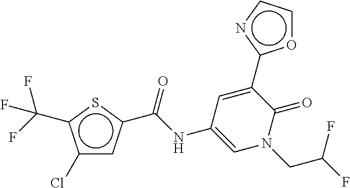 | 454 |
| 103c | 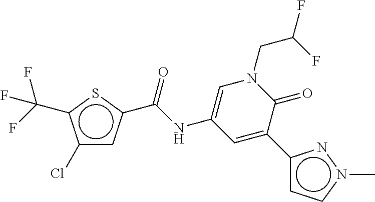 | 467 |
| 104c | 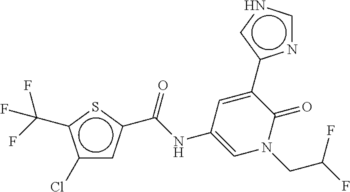 | 453 |
| 105c | 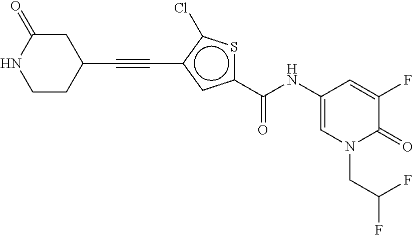 | 458 |
| 106c | 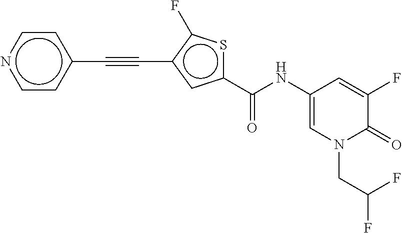 | 422 |
| 107c | 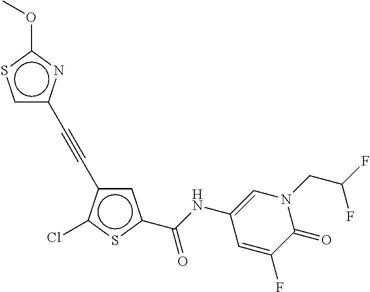 | 473 |
| 108c | 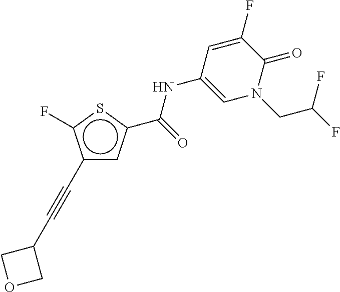 | 401 |
| 109c | 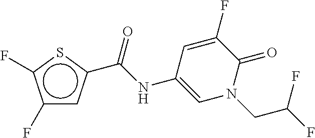 | 338 |
| 110c | 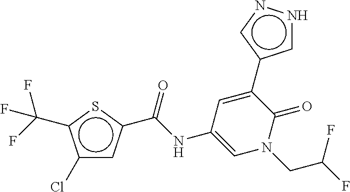 | 453 |
| 111c | 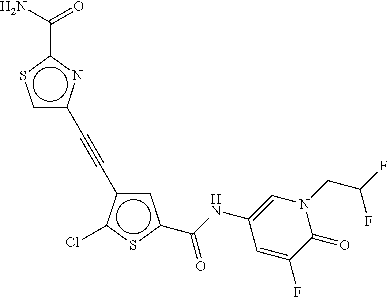 | 487 |
| 112c | 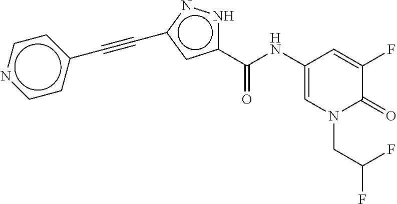 | 388 |
| 113c | 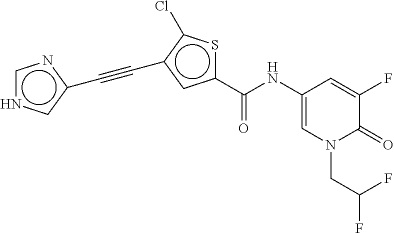 | 427 |
| 114c | 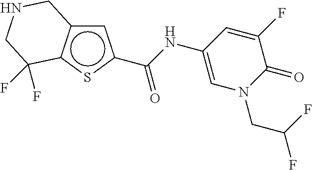 | 394 |
| 115c | 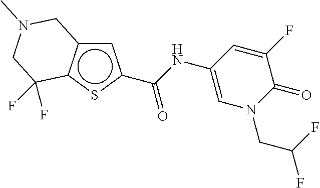 | 408 |
| 116c | 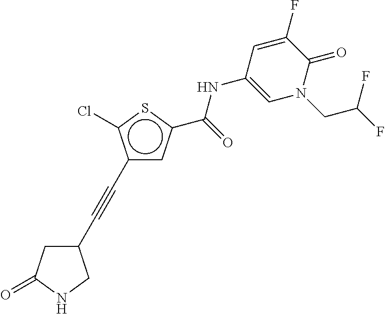 | 444 |
| 117c | 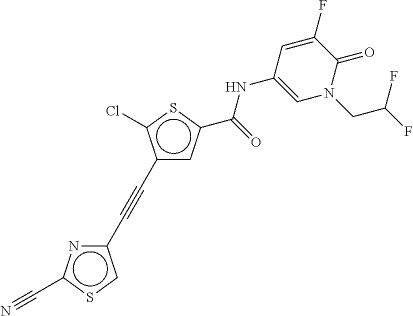 | 469 |
| 118c | 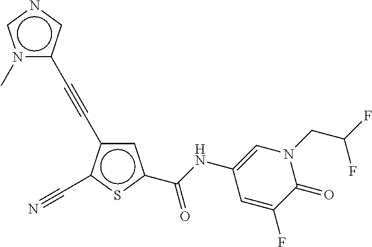 | 432 |
| 119c | 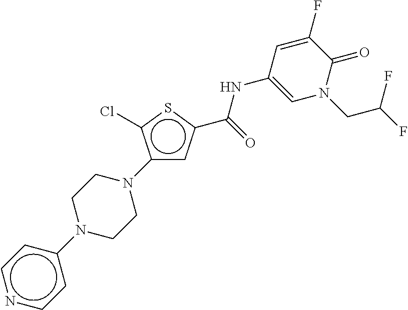 | 498 |
| 120c | 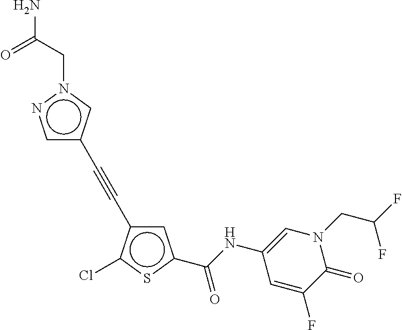 | 484 |
| 121c | 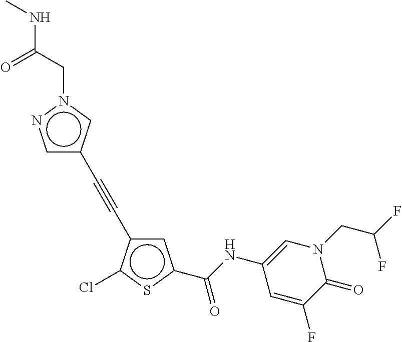 | 498 |
| 122c | 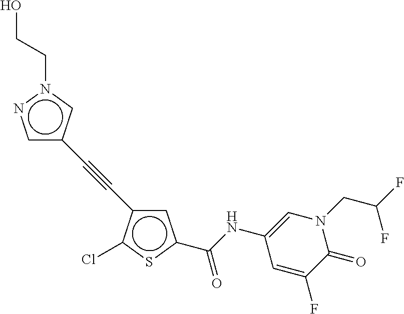 | 471 |
| 123c | 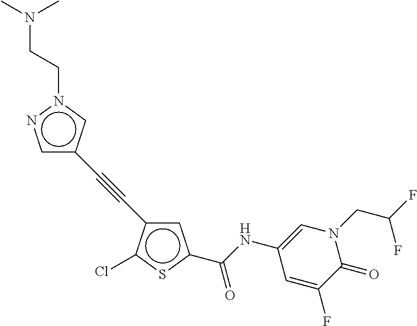 | 498 |
| 124c | 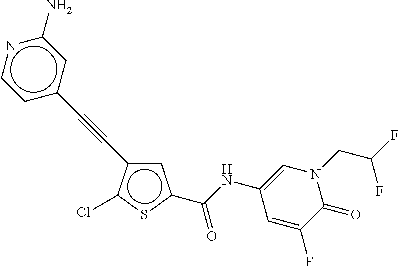 | 453 |
| 125c | 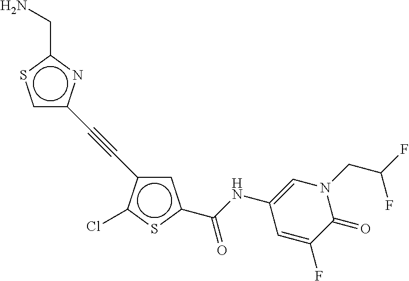 | 473 |
| 126c | 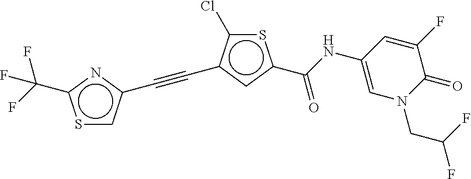 | 512 |
| 127c | 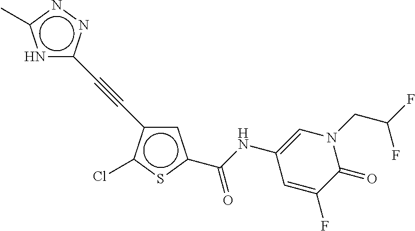 | 442 |
| 128c | 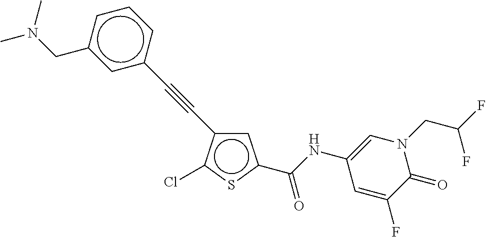 | 494 |
| 129c | 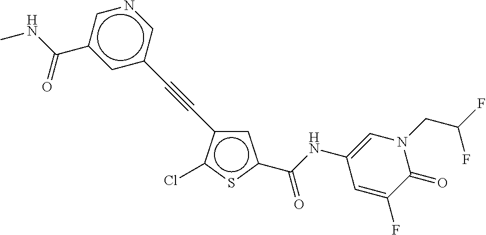 | 495 |
| 130c | 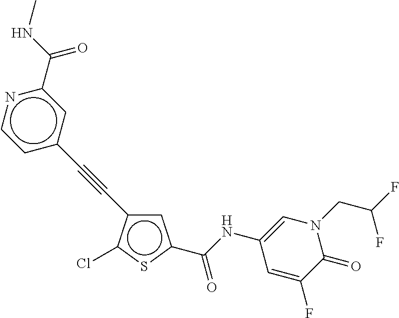 | 495 |
| 131c | 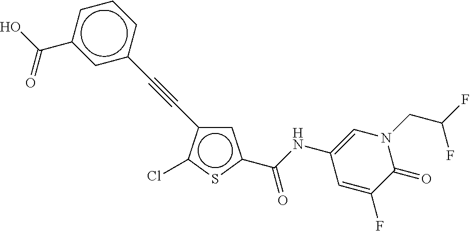 | 481 |
| 132c | 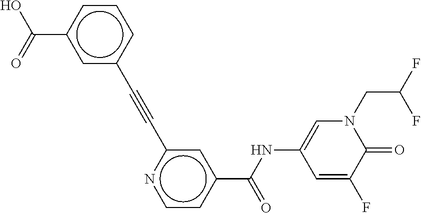 | 442 |
| 133c | 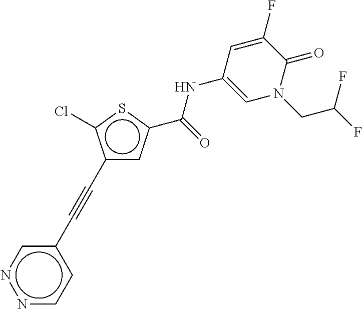 | 439 |
| 134c | 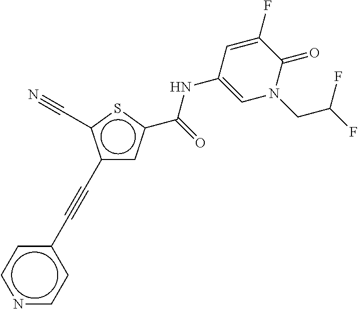 | 429 |
| 135c | 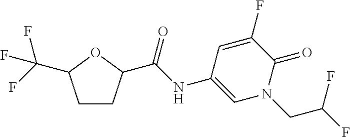 [SMILES: O=C(Nc1cc(F)c(=O)n(CC(F)F)c1)C1CCC(C(F)(F)F)O1] | 359 |
| 136c | 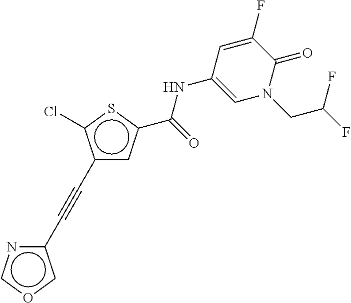 | 428 |
| 137c | 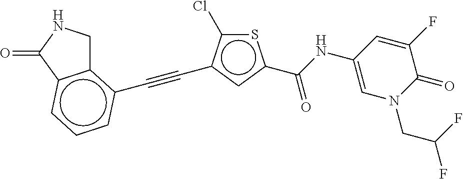 | 492 |
| 138c | 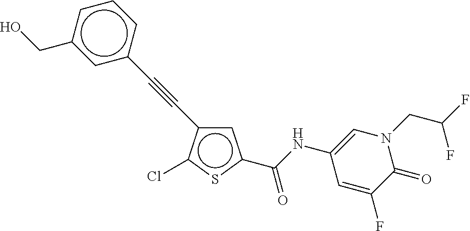 | 467 |
| 139c | 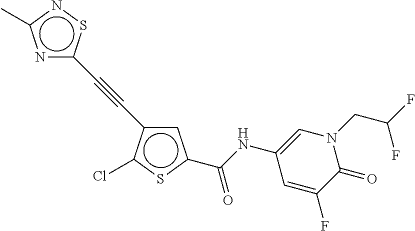 | 459 |
| 140c | 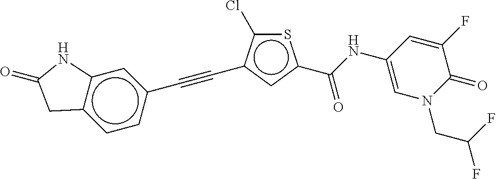 | 492 |
| 141c | 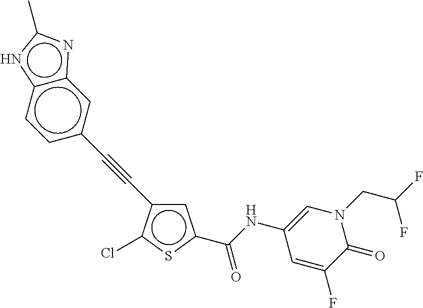 | 491 |
| 142c | 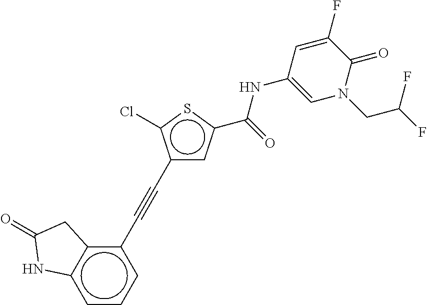 | 490 |
| 143c | 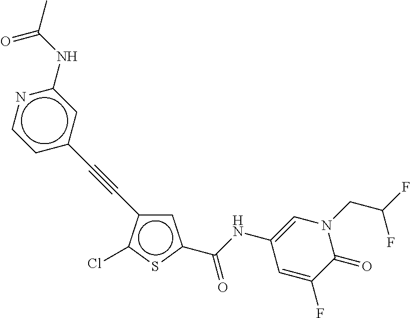 | 495 |
| 144c | 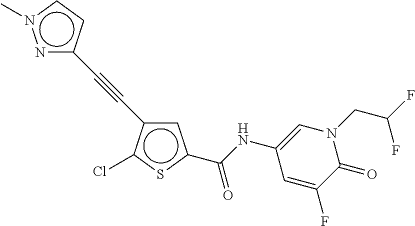 | 441 |
| 145c | 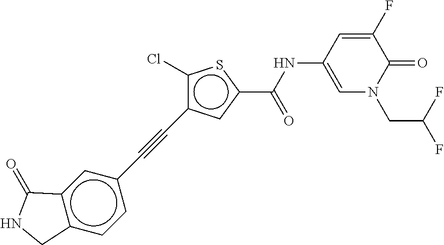 | 492 |
| 146c | 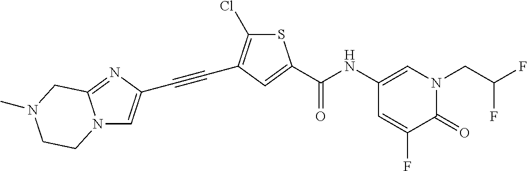 [SMILES: CN1CCn2cc(C#Cc3cc(C(=O)Nc4cc(F)c(=O)n(CC(F)F)c4)sc3Cl)nc2C1] | 496 |
| 147c | 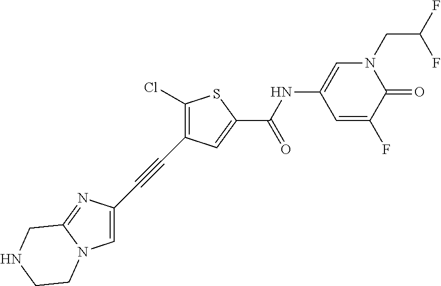 [SMILES: O=C(Nc1cc(F)c(=O)n(CC(F)F)c1)c1cc(C#Cc2cn3c(n2)CNCC3)c(Cl)s1] | 482 |
| 148c | 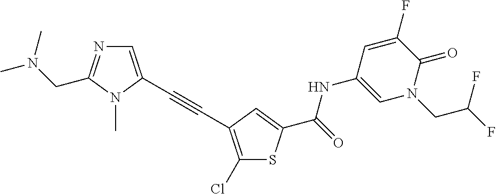 [SMILES: CN(C)Cc1ncc(C#Cc2cc(C(=O)Nc3cc(F)c(=O)n(CC(F)F)c3)sc2Cl)n1C] | 498 |
| 149c | 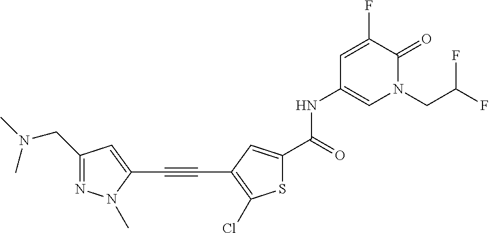 [SMILES: CN(C)Cc1cc(C#Cc2cc(C(=O)Nc3cc(F)c(=O)n(CC(F)F)c3)sc2Cl)n(C)n1] | 498 |
| 150c | 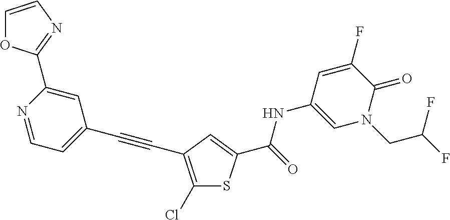 [SMILES: O=C(Nc1cc(F)c(=O)n(CC(F)F)c1)c1cc(C#Cc2ccnc(-c3ncco3)c2)c(Cl)s1] | 505 |
| 151c | 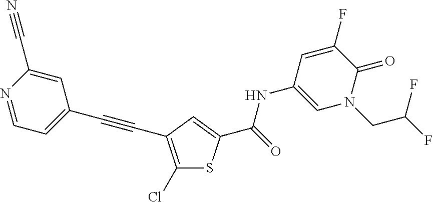 [SMILES: N#Cc1cc(C#Cc2cc(C(=O)Nc3cc(F)c(=O)n(CC(F)F)c3)sc2Cl)ccn1] | 463 |
| 152c | 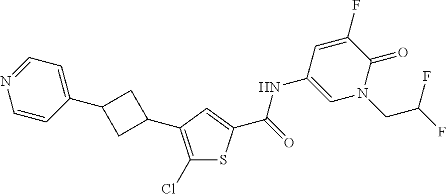 [SMILES: O=C(Nc1cc(F)c(=O)n(CC(F)F)c1)c1cc(C2CC(c3ccncc3)C2)c(Cl)s1] | 468 |
| 153c | 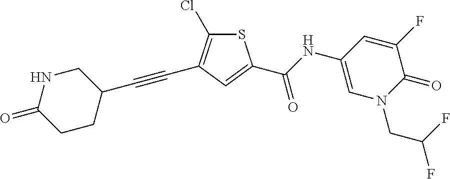 [SMILES: O=C1CCC(C#Cc2cc(C(=O)Nc3cc(F)c(=O)n(CC(F)F)c3)sc2Cl)CN1] | 458 |
| 154c | 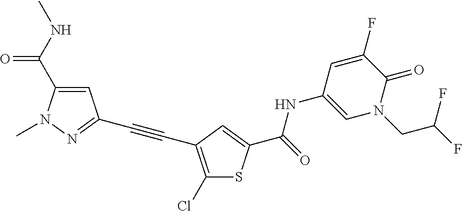 [SMILES: CNC(=O)c1cc(C#Cc2cc(C(=O)Nc3cc(F)c(=O)n(CC(F)F)c3)sc2Cl)nn1C] | 498 |
| 155c | 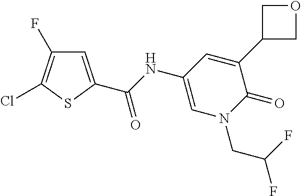 [SMILES: O=C(Nc1cc(C2COC2)c(=O)n(CC(F)F)c1)c1cc(F)c(Cl)s1] | 393 |
| 156c | 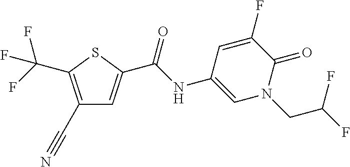 [SMILES: N#Cc1cc(C(=O)Nc2cc(F)c(=O)n(CC(F)F)c2)sc1C(F)(F)F] | 405 |
| 157c | 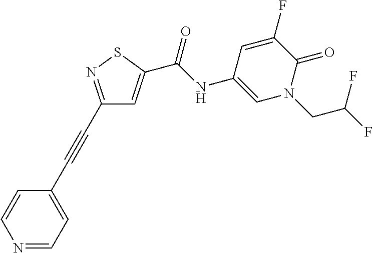 [SMILES: O=C(Nc1cc(F)c(=O)n(CC(F)F)c1)c1cc(C#Cc2ccncc2)ns1] | 389 |
| 158c | 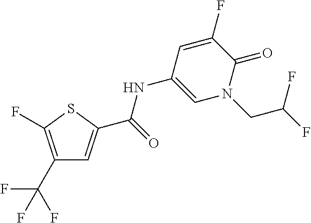 [SMILES: O=C(Nc1cc(F)c(=O)n(CC(F)F)c1)c1cc(C(F)(F)F)c(F)s1] | 423 |
| 159c | 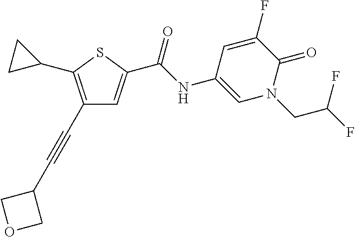 [SMILES: O=C(Nc1cc(F)c(=O)n(CC(F)F)c1)c1cc(C#CC2COC2)c(C2CC2)s1] | 472 |
| 160c | 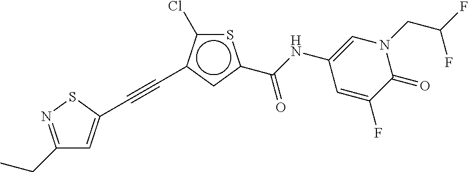 | 427 |
| 161c | 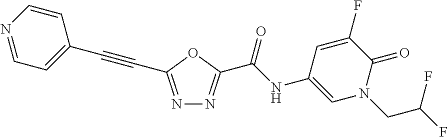 [SMILES: O=C(Nc1cc(F)c(=O)n(CC(F)F)c1)c1nnc(C#Cc2ccncc2)o1] | ND |
| 162c | 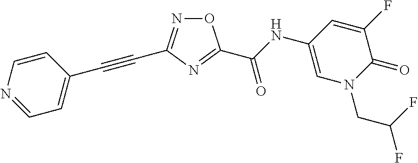 [SMILES: O=C(Nc1cc(F)c(=O)n(CC(F)F)c1)c1nc(C#Cc2ccncc2)no1] | ND |
| 163c | 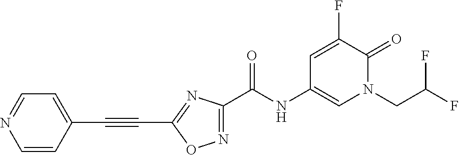 [SMILES: O=C(Nc1cc(F)c(=O)n(CC(F)F)c1)c1noc(C#Cc2ccncc2)n1] | ND |
| 164c | 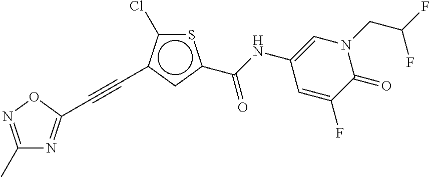 | ND |
| 166c | 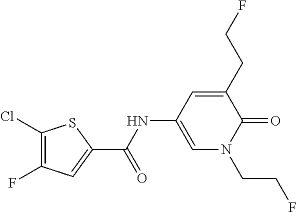 [SMILES: O=C(Nc1cc(CCF)c(=O)n(CCF)c1)c1cc(F)c(Cl)s1] | ND |
| 168c | 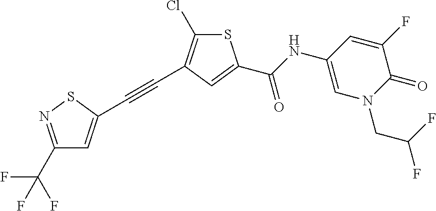 [SMILES: O=C(Nc1cc(F)c(=O)n(CC(F)F)c1)c1cc(C#Cc2cc(C(F)(F)F)ns2)c(Cl)s1] | ND |
| 170c | 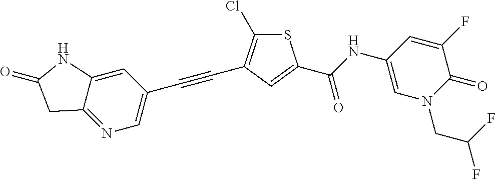 [SMILES: O=C1Cc2ncc(C#Cc3cc(C(=O)Nc4cc(F)c(=O)n(CC(F)F)c4)sc3Cl)cc2N1] | 493 |
| 172c | 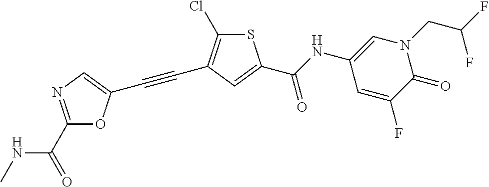 [SMILES: CNC(=O)c1ncc(C#Cc2cc(C(=O)Nc3cc(F)c(=O)n(CC(F)F)c3)sc2Cl)o1] | 485 |
| 174c | 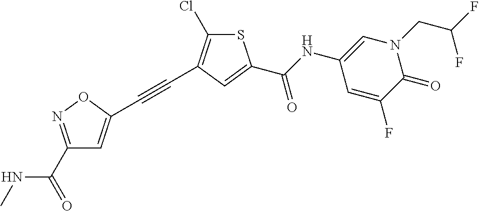 [SMILES: CNC(=O)c1cc(C#Cc2cc(C(=O)Nc3cc(F)c(=O)n(CC(F)F)c3)sc2Cl)on1] | 485 |
| 176c | 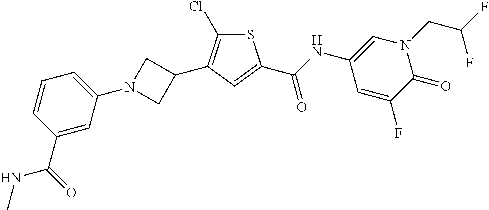 [SMILES: CNC(=O)c1cccc(N2CC(c3cc(C(=O)Nc4cc(F)c(=O)n(CC(F)F)c4)sc3Cl)C2)c1] | 525 |
| 178c | 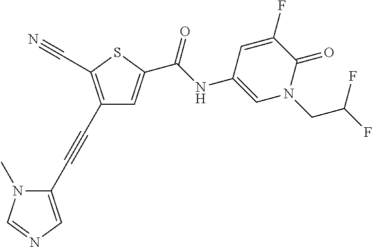 [SMILES: Cn1cncc1C#Cc1cc(C(=O)Nc2cc(F)c(=O)n(CC(F)F)c2)sc1C#N] | 432 |

TABLE 2d
SMARCA2 inhibitors of the present disclosure.

| Compd. | | |
|---|---|---|
| No. | Structure | Mass (u) |
| 1d | 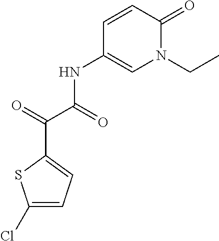 [SMILES: CCn1cc(NC(=O)C(=O)c2ccc(Cl)s2)ccc1=O] | 311 |
| 2d | 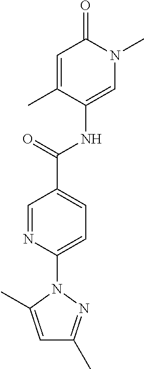 | 338 |
| 3d | 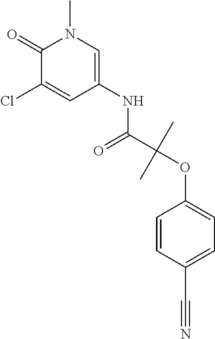 [SMILES: Cn1cc(NC(=O)C(C)(C)Oc2ccc(C#N)cc2)cc(Cl)c1=O] | 346 |
| 4d | 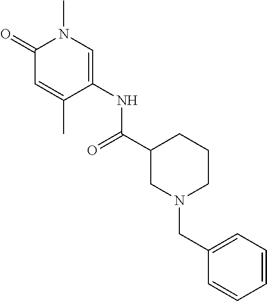 [SMILES: Cc1cc(=O)n(C)cc1NC(=O)C1CCCN(Cc2ccccc2)C1] | 340 |
| 5d | 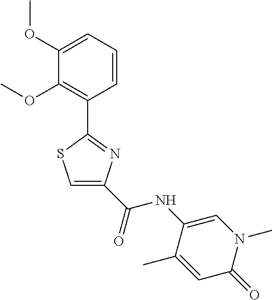 | 386 |
| 6d | 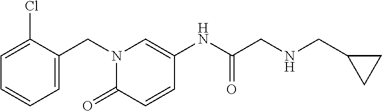 [SMILES: O=C(CNCC1CC1)Nc1ccc(=O)n(Cc2ccccc2Cl)c1] | 346 |
| 7d | 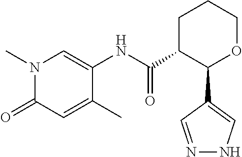 [SMILES: Cc1cc(=O)n(C)cc1NC(=O)[C@@H]1CCCO[C@H]1c1cn[nH]c1] | 317 |
| 8d | 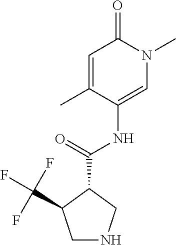 [SMILES: Cc1cc(=O)n(C)cc1NC(=O)[C@@H]1CNC[C@H]1C(F)(F)F] | 304 |
| 9d | 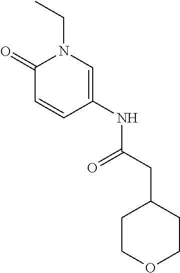 [SMILES: CCn1cc(NC(=O)CC2CCOCC2)ccc1=O] | 265 |
| 10d | 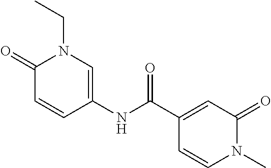 [SMILES: CCn1cc(NC(=O)c2ccn(C)c(=O)c2)ccc1=O] | 274 |
| 11d | 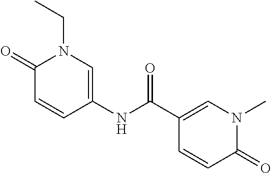 [SMILES: CCn1cc(NC(=O)c2ccc(=O)n(C)c2)ccc1=O] | 274 |
| 12d | 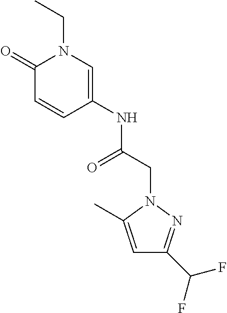 [SMILES: CCn1cc(NC(=O)Cn2nc(C(F)F)cc2C)ccc1=O] | 311 |
| 13d | 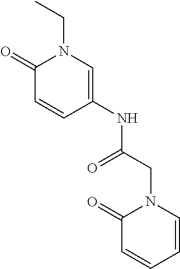 [SMILES: CCn1cc(NC(=O)Cn2ccccc2=O)ccc1=O] | 274 |
| 14d | 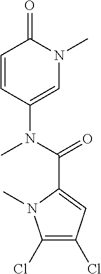 [SMILES: CN(C(=O)c1cc(Cl)c(Cl)n1C)c1ccc(=O)n(C)c1] | 328 |
| 15d | 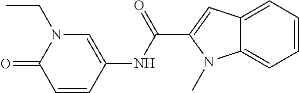 [SMILES: CCn1cc(NC(=O)c2cc3ccccc3n2C)ccc1=O] | 296 |
| 16d | 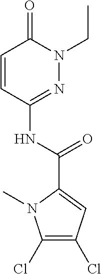 [SMILES: CCn1nc(NC(=O)c2cc(Cl)c(Cl)n2C)ccc1=O] | 315 |
| 17d | 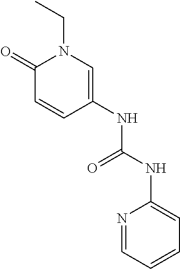 [SMILES: CCn1cc(NC(=O)Nc2ccccn2)ccc1=O] | 259 |
| 18d | 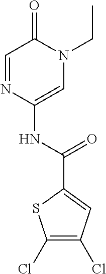 [SMILES: CCn1cc(NC(=O)c2cc(Cl)c(Cl)s2)ncc1=O] | 318 |
| 19d | 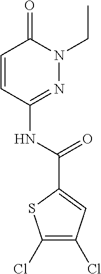 [SMILES: CCn1nc(NC(=O)c2cc(Cl)c(Cl)s2)ccc1=O] | 318 |
| 20d | 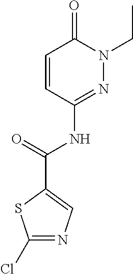 [SMILES: CCn1nc(NC(=O)c2cnc(Cl)s2)ccc1=O] | 285 |
| 21d | 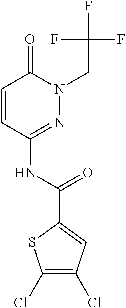 [SMILES: O=C(Nc1ccc(=O)n(CC(F)(F)F)n1)c1cc(Cl)c(Cl)s1] | 372 |
| 22d | 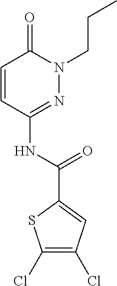 [SMILES: CCCn1nc(NC(=O)c2cc(Cl)c(Cl)s2)ccc1=O] | 332 |
| 23d | 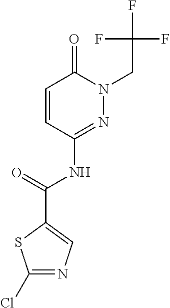 [SMILES: O=C(Nc1ccc(=O)n(CC(F)(F)F)n1)c1cnc(Cl)s1] | 339 |
| 24d | 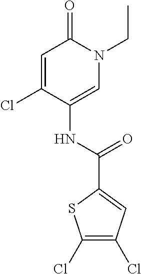 [SMILES: CCn1cc(NC(=O)c2cc(Cl)c(Cl)s2)c(Cl)cc1=O] | 351 |
| 25d | 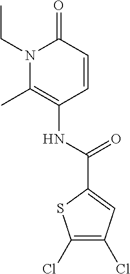 [SMILES: CCn1c(C)c(NC(=O)c2cc(Cl)c(Cl)s2)ccc1=O] | 331 |
| 26d | 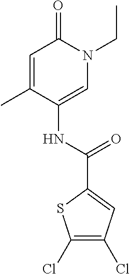 [SMILES: CCn1cc(NC(=O)c2cc(Cl)c(Cl)s2)c(C)cc1=O] | 331 |
| 27d | 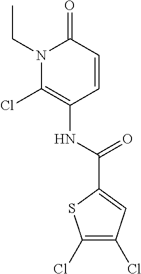 [SMILES: CCn1c(Cl)c(NC(=O)c2cc(Cl)c(Cl)s2)ccc1=O] | 353 |
| 28d | 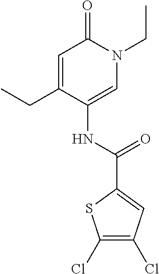 [SMILES: CCc1cc(=O)n(CC)cc1NC(=O)c1cc(Cl)c(Cl)s1] | 345 |
| 29d | 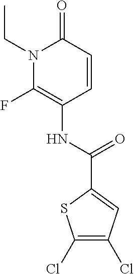 [SMILES: CCn1c(F)c(NC(=O)c2cc(Cl)c(Cl)s2)ccc1=O] | 335 |
| 30d | 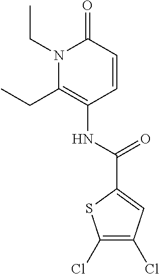 [SMILES: CCc1c(NC(=O)c2cc(Cl)c(Cl)s2)ccc(=O)n1CC] | 345 |
| 31d | 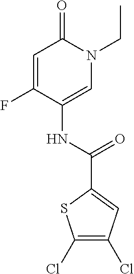 [SMILES: CCn1cc(NC(=O)c2cc(Cl)c(Cl)s2)c(F)cc1=O] | 335 |
| 32d | 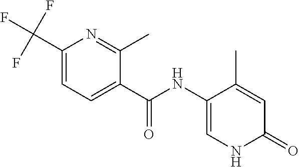 [SMILES: Cc1cc(=O)[nH]cc1NC(=O)c1ccc(C(F)(F)F)nc1C] | 312 |
| 33d | 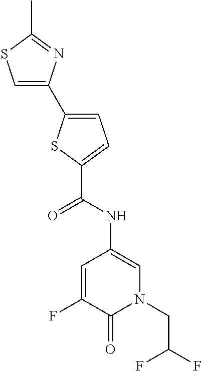 [SMILES: Cc1nc(-c2ccc(C(=O)Nc3cc(F)c(=O)n(CC(F)F)c3)s2)cs1] | 400 |
| 34d | 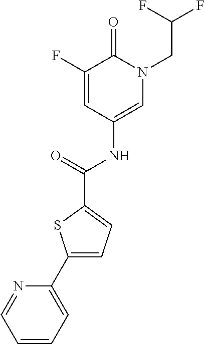 [SMILES: O=C(Nc1cc(F)c(=O)n(CC(F)F)c1)c1ccc(-c2ccccn2)s1] | 380 |
| 35d | 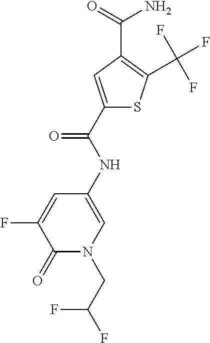 [SMILES: NC(=O)c1cc(C(=O)Nc2cc(F)c(=O)n(CC(F)F)c2)sc1C(F)(F)F] | 414 |
| 36d | 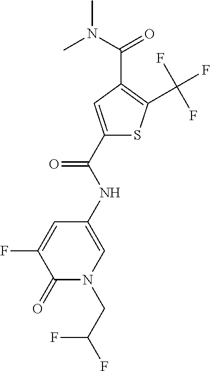 [SMILES: CN(C)C(=O)c1cc(C(=O)Nc2cc(F)c(=O)n(CC(F)F)c2)sc1C(F)(F)F] | 442 |
| 37d | 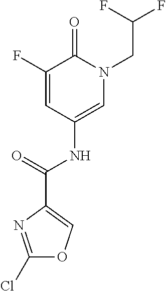 [SMILES: O=C(Nc1cc(F)c(=O)n(CC(F)F)c1)c1coc(Cl)n1] | 322 |
| 38d | 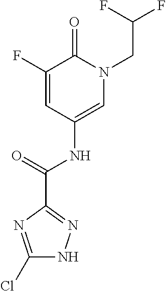 [SMILES: O=C(Nc1cc(F)c(=O)n(CC(F)F)c1)c1n[nH]c(Cl)n1] | 322 |
| 39d | 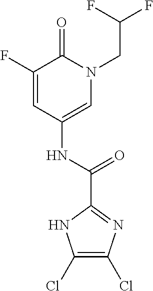 [SMILES: O=C(Nc1cc(F)c(=O)n(CC(F)F)c1)c1nc(Cl)c(Cl)[nH]1] | 355 |
| 40d | 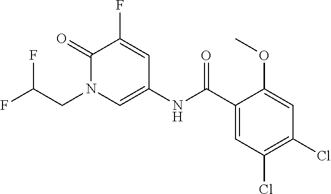 [SMILES: COc1cc(Cl)c(Cl)cc1C(=O)Nc1cc(F)c(=O)n(CC(F)F)c1] | 395 |
| 41d | 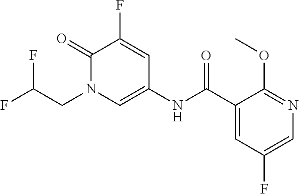 [SMILES: COc1ncc(F)cc1C(=O)Nc1cc(F)c(=O)n(CC(F)F)c1] | 346 |
| 42d | 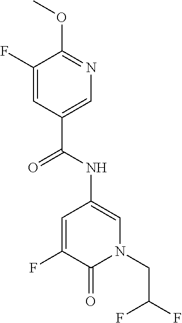 [SMILES: COc1ncc(C(=O)Nc2cc(F)c(=O)n(CC(F)F)c2)cc1F] | 346 |
| 43d | 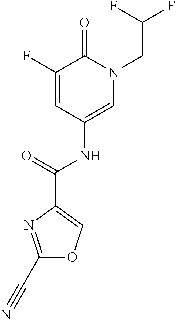 [SMILES: N#Cc1nc(C(=O)Nc2cc(F)c(=O)n(CC(F)F)c2)co1] | 313 |
| 44d | 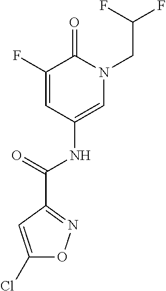 [SMILES: O=C(Nc1cc(F)c(=O)n(CC(F)F)c1)c1cc(Cl)on1] | 322 |
| 45d | 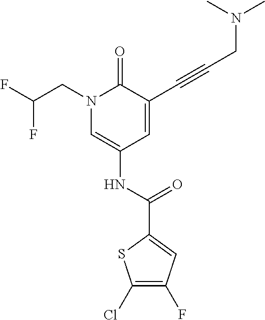 [SMILES: CN(C)CC#Cc1cc(NC(=O)c2cc(F)c(Cl)s2)cn(CC(F)F)c1=O] | 440 |
| 46d | 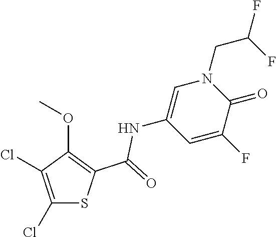 [SMILES: COc1c(C(=O)Nc2cc(F)c(=O)n(CC(F)F)c2)sc(Cl)c1Cl] | 401 |
| 47d | 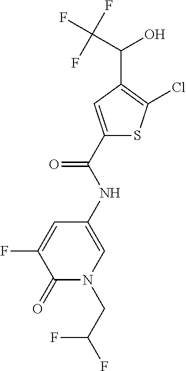 [SMILES: O=C(Nc1cc(F)c(=O)n(CC(F)F)c1)c1cc(C(O)C(F)(F)F)c(Cl)s1] | 435 |
| 48d | 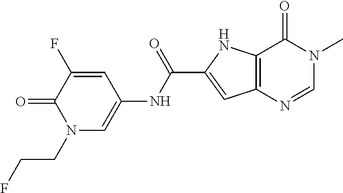 [SMILES: Cn1cnc2cc(C(=O)Nc3cc(F)c(=O)n(CCF)c3)[nH]c2c1=O] | 350 |
| 49d | 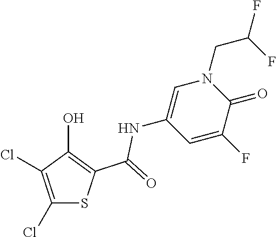 [SMILES: O=C(Nc1cc(F)c(=O)n(CC(F)F)c1)c1sc(Cl)c(Cl)c1O] | 385 |
| 50d | 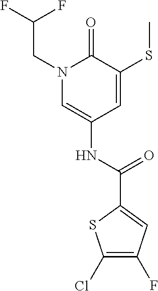 [SMILES: CSc1cc(NC(=O)c2cc(F)c(Cl)s2)cn(CC(F)F)c1=O] | 383 |
| 51d | 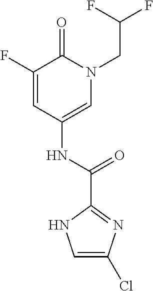 [SMILES: O=C(Nc1cc(F)c(=O)n(CC(F)F)c1)c1nc(Cl)c[nH]1] | 321 |
| 52d | 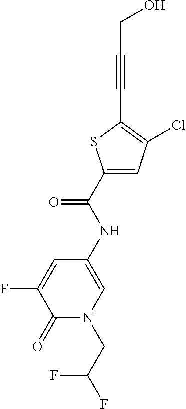 [SMILES: O=C(Nc1cc(F)c(=O)n(CC(F)F)c1)c1cc(Cl)c(C#CCO)s1] | 391 |
| 53d | 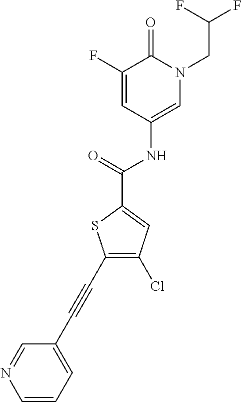 [SMILES: O=C(Nc1cc(F)c(=O)n(CC(F)F)c1)c1cc(Cl)c(C#Cc2cccnc2)s1] | 438 |
| 54d | 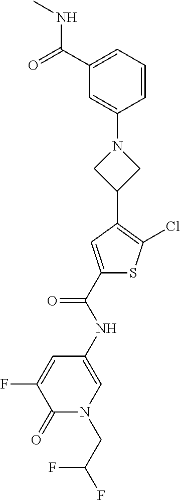 [SMILES: CNC(=O)c1cccc(N2CC(c3cc(C(=O)Nc4cc(F)c(=O)n(CC(F)F)c4)sc3Cl)C2)c1] | 638 |
| 55d | 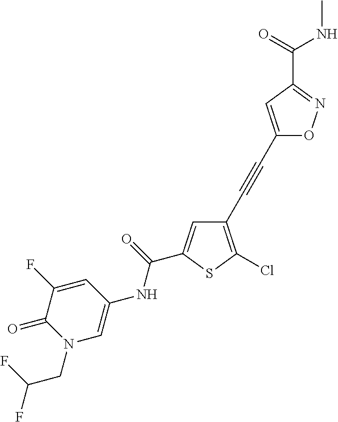 [SMILES: CNC(=O)c1cc(C#Cc2cc(C(=O)Nc3cc(F)c(=O)n(CC(F)F)c3)sc2Cl)on1] | 485 |
| 56d | 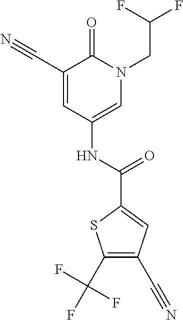 [SMILES: N#Cc1cc(C(=O)Nc2cc(C#N)c(=O)n(CC(F)F)c2)sc1C(F)(F)F] | 403 |
| 57d | 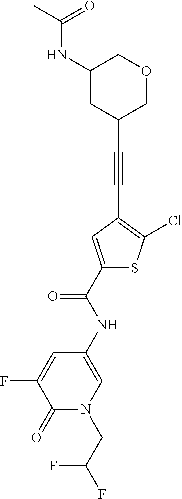 [SMILES: CC(=O)NC1COCC(C#Cc2cc(C(=O)Nc3cc(F)c(=O)n(CC(F)F)c3)sc2Cl)C1] | 502 |
| 58d | 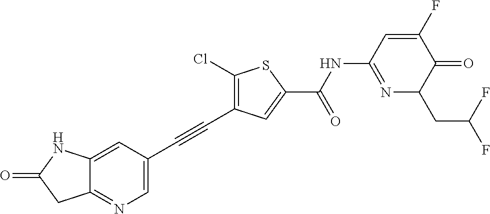 [SMILES: O=C1Cc2ncc(C#Cc3cc(C(=O)NC4=NC(CC(F)F)C(=O)C(F)=C4)sc3Cl)cc2N1] | 493 |
| 59d | 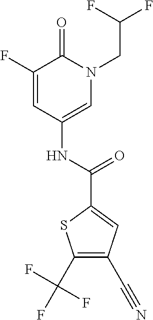 [SMILES: N#Cc1cc(C(=O)Nc2cc(F)c(=O)n(CC(F)F)c2)sc1C(F)(F)F] | 412 |
| 60d | 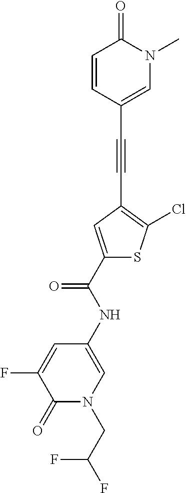 [SMILES: Cn1cc(C#Cc2cc(C(=O)Nc3cc(F)c(=O)n(CC(F)F)c3)sc2Cl)ccc1=O] | 468 |
| 61d | 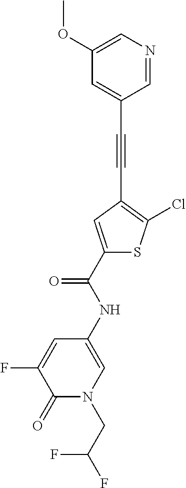 [SMILES: COc1cncc(C#Cc2cc(C(=O)Nc3cc(F)c(=O)n(CC(F)F)c3)sc2Cl)c1] | 468 |
| 62d | 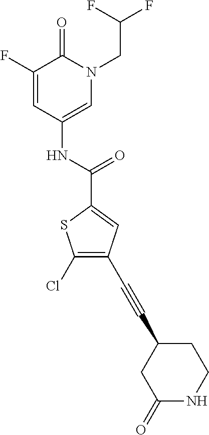 [SMILES: O=C1C[C@@H](C#Cc2cc(C(=O)Nc3cc(F)c(=O)n(CC(F)F)c3)sc2Cl)CCN1] | 458 |
| 63d | 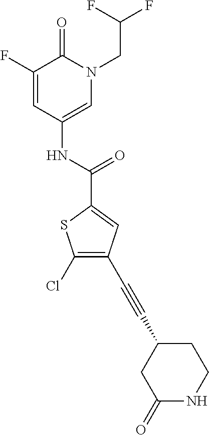 [SMILES: O=C1C[C@H](C#Cc2cc(C(=O)Nc3cc(F)c(=O)n(CC(F)F)c3)sc2Cl)CCN1] | 458 |
| 64d | 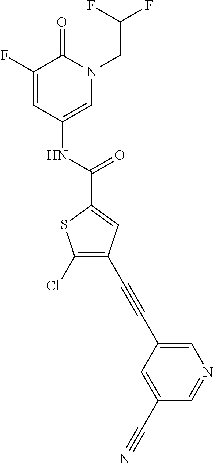 [SMILES: N#Cc1cncc(C#Cc2cc(C(=O)Nc3cc(F)c(=O)n(CC(F)F)c3)sc2Cl)c1] | 463 |
| 65d | 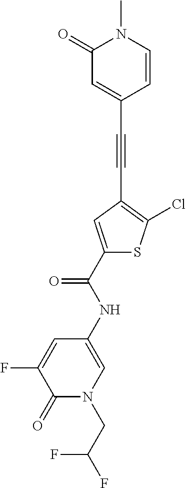 [SMILES: Cn1ccc(C#Cc2cc(C(=O)Nc3cc(F)c(=O)n(CC(F)F)c3)sc2Cl)cc1=O] | 468 |
| 66d | 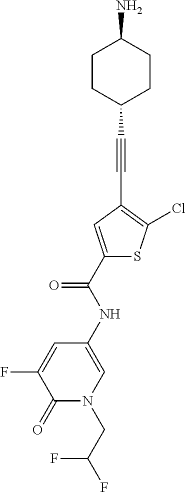 [SMILES: N[C@H]1CC[C@H](C#Cc2cc(C(=O)Nc3cc(F)c(=O)n(CC(F)F)c3)sc2Cl)CC1] | 458 |
| 67d | 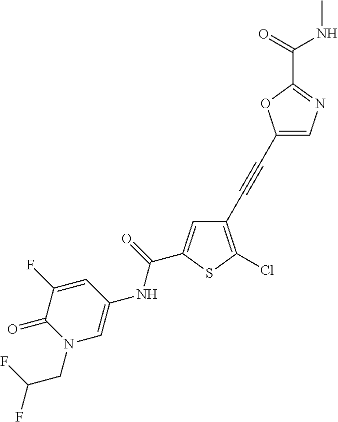 [SMILES: CNC(=O)c1ncc(C#Cc2cc(C(=O)Nc3cc(F)c(=O)n(CC(F)F)c3)sc2Cl)o1] | 485 |
| 68d | 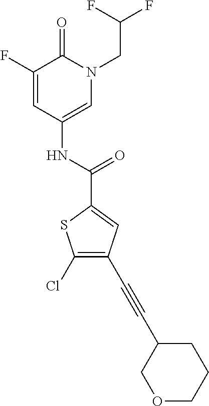 [SMILES: O=C(Nc1cc(F)c(=O)n(CC(F)F)c1)c1cc(C#CC2CCCOC2)c(Cl)s1] | 445 |
| 69d | 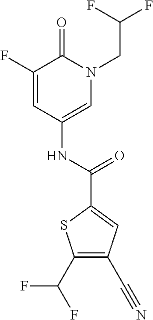 [SMILES: N#Cc1cc(C(=O)Nc2cc(F)c(=O)n(CC(F)F)c2)sc1C(F)F] | 378 |
| 70d | 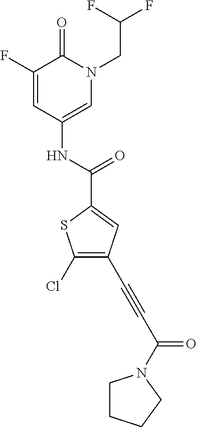 [SMILES: O=C(Nc1cc(F)c(=O)n(CC(F)F)c1)c1cc(C#CC(=O)N2CCCC2)c(Cl)s1] | 458 |
| 71d | 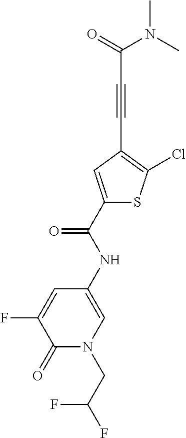 [SMILES: CN(C)C(=O)C#Cc1cc(C(=O)Nc2cc(F)c(=O)n(CC(F)F)c2)sc1Cl] | 432 |
| 72d | 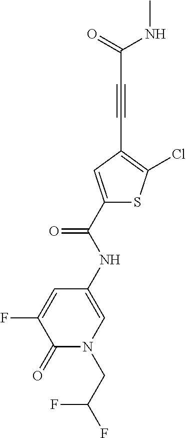 [SMILES: CNC(=O)C#Cc1cc(C(=O)Nc2cc(F)c(=O)n(CC(F)F)c2)sc1Cl] | 418 |
| 73d | 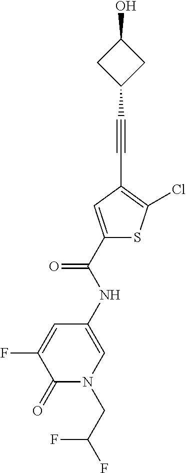 [SMILES: O=C(Nc1cc(F)c(=O)n(CC(F)F)c1)c1cc(C#C[C@H]2C[C@H](O)C2)c(Cl)s1] | 431 |
| 74d | 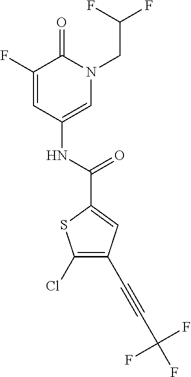 [SMILES: O=C(Nc1cc(F)c(=O)n(CC(F)F)c1)c1cc(C#CC(F)(F)F)c(Cl)s1] | 429 |
| 75d | 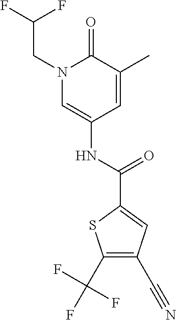 [SMILES: Cc1cc(NC(=O)c2cc(C#N)c(C(F)(F)F)s2)cn(CC(F)F)c1=O] | 392 |
| 76d | 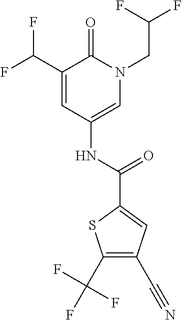 [SMILES: N#Cc1cc(C(=O)Nc2cc(C(F)F)c(=O)n(CC(F)F)c2)sc1C(F)(F)F] | 428 |
| 77d | 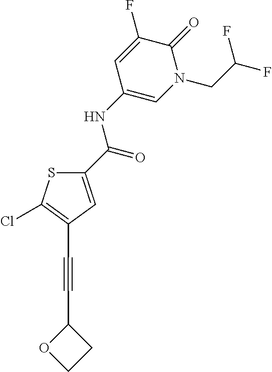 [SMILES: O=C(Nc1cc(F)c(=O)n(CC(F)F)c1)c1cc(C#CC2CCO2)c(Cl)s1] | 415 |
| 78d | 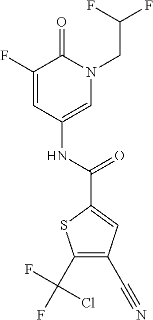 [SMILES: N#Cc1cc(C(=O)Nc2cc(F)c(=O)n(CC(F)F)c2)sc1C(F)(F)Cl] | 412 |
| 79d | 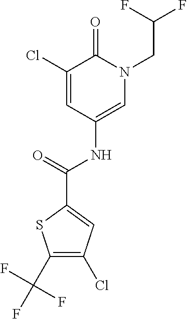 [SMILES: O=C(Nc1cc(Cl)c(=O)n(CC(F)F)c1)c1cc(Cl)c(C(F)(F)F)s1] | 421 |
| 80d | 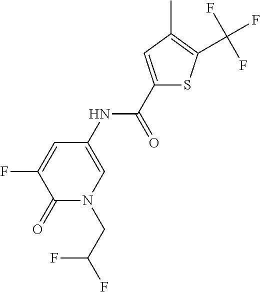 [SMILES: Cc1cc(C(=O)Nc2cc(F)c(=O)n(CC(F)F)c2)sc1C(F)(F)F] | 385 |
| 81d | 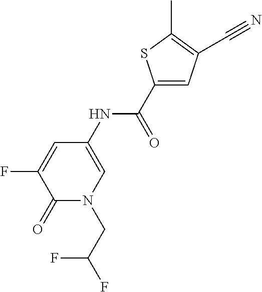 [SMILES: Cc1sc(C(=O)Nc2cc(F)c(=O)n(CC(F)F)c2)cc1C#N] | 342 |
| 82d | 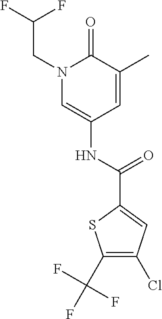 [SMILES: Cc1cc(NC(=O)c2cc(Cl)c(C(F)(F)F)s2)cn(CC(F)F)c1=O] | 401 |
| 83d | 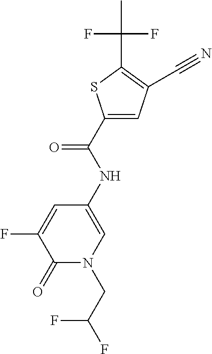 [SMILES: CC(F)(F)c1sc(C(=O)Nc2cc(F)c(=O)n(CC(F)F)c2)cc1C#N] | 392 |
| 84d | 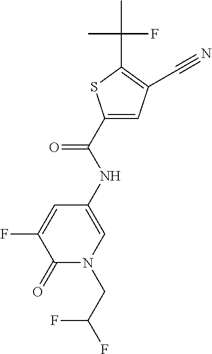 [SMILES: CC(C)(F)c1sc(C(=O)Nc2cc(F)c(=O)n(CC(F)F)c2)cc1C#N] | 388 |
| 85d | 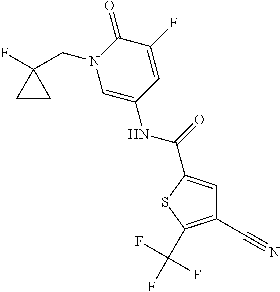 [SMILES: N#Cc1cc(C(=O)Nc2cc(F)c(=O)n(CC3(F)CC3)c2)sc1C(F)(F)F] | 404 |
| 86d | 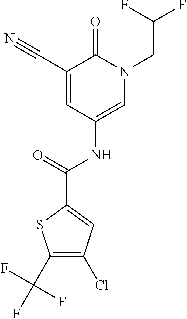 [SMILES: N#Cc1cc(NC(=O)c2cc(Cl)c(C(F)(F)F)s2)cn(CC(F)F)c1=O] | 412 |
| 87d | 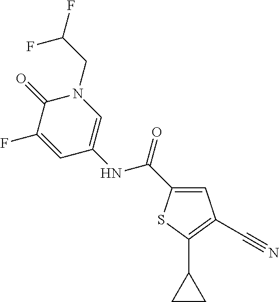 [SMILES: N#Cc1cc(C(=O)Nc2cc(F)c(=O)n(CC(F)F)c2)sc1C1CC1] | 368 |
| 88d | 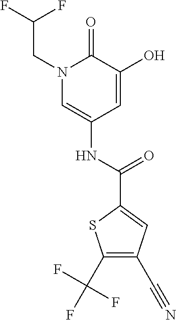 [SMILES: N#Cc1cc(C(=O)Nc2cc(O)c(=O)n(CC(F)F)c2)sc1C(F)(F)F] | 394 |
| 89d | 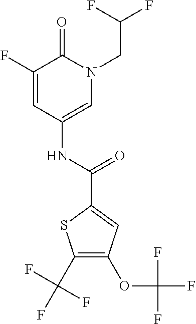 [SMILES: O=C(Nc1cc(F)c(=O)n(CC(F)F)c1)c1cc(OC(F)(F)F)c(C(F)(F)F)s1] | 455 |
| 90d | 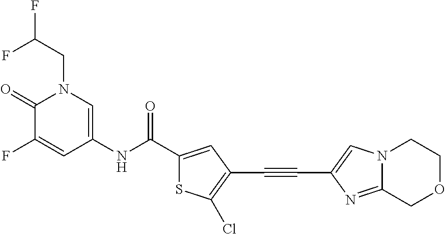 [SMILES: O=C(Nc1cc(F)c(=O)n(CC(F)F)c1)c1cc(C#Cc2cn3c(n2)COCC3)c(Cl)s1] | 483 |
| 91d | 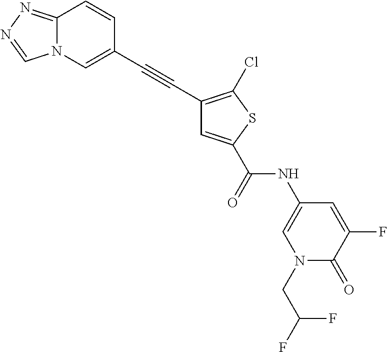 [SMILES: O=C(Nc1cc(F)c(=O)n(CC(F)F)c1)c1cc(C#Cc2ccc3nncn3c2)c(Cl)s1] | 478 |
| 92d | 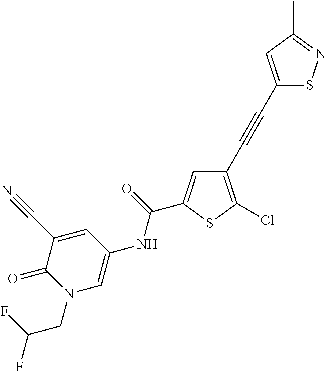 [SMILES: Cc1cc(C#Cc2cc(C(=O)Nc3cc(C#N)c(=O)n(CC(F)F)c3)sc2Cl)sn1] | 465 |
| 93d | 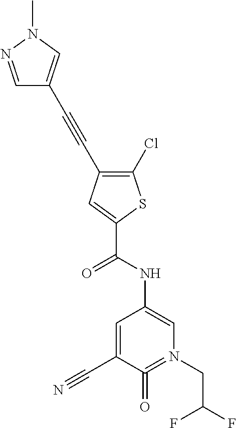 | 448 |
| 94d | 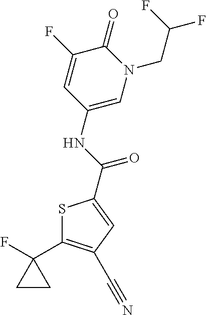 [SMILES: N#Cc1cc(C(=O)Nc2cc(F)c(=O)n(CC(F)F)c2)sc1C1(F)CC1] | 386 |
| 95d | 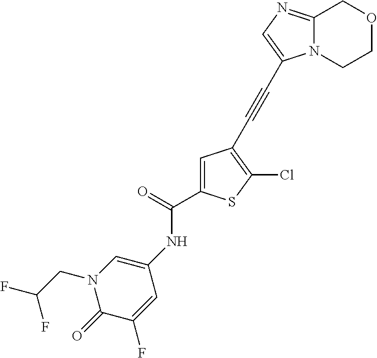 [SMILES: O=C(Nc1cc(F)c(=O)n(CC(F)F)c1)c1cc(C#Cc2cnc3n2CCOC3)c(Cl)s1] | 483 |
| 96d | 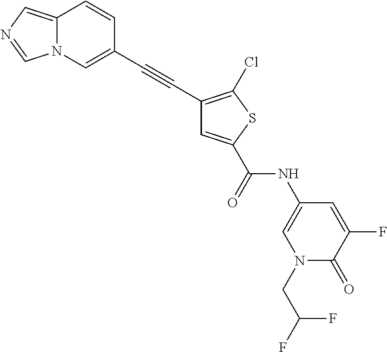 [SMILES: O=C(Nc1cc(F)c(=O)n(CC(F)F)c1)c1cc(C#Cc2ccc3cncn3c2)c(Cl)s1] | 477 |
| 97d | 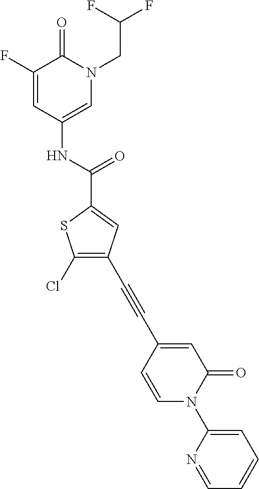 [SMILES: O=C(Nc1cc(F)c(=O)n(CC(F)F)c1)c1cc(C#Cc2ccn(-c3ccccn3)c(=O)c2)c(Cl)s1] | 531 |
| 98d | 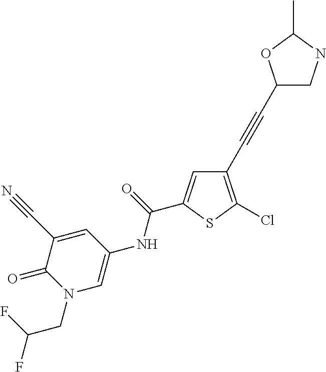 [SMILES: CC1NCC(C#Cc2cc(C(=O)Nc3cc(C#N)c(=O)n(CC(F)F)c3)sc2Cl)O1] | 449 |
| 99d | 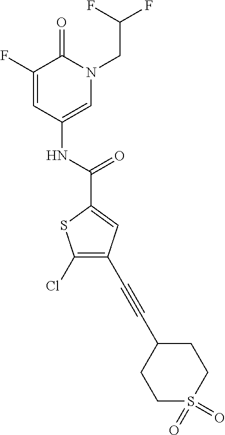 [SMILES: O=C(Nc1cc(F)c(=O)n(CC(F)F)c1)c1cc(C#CC2CCS(=O)(=O)CC2)c(Cl)s1] | 493 |
| 100d | 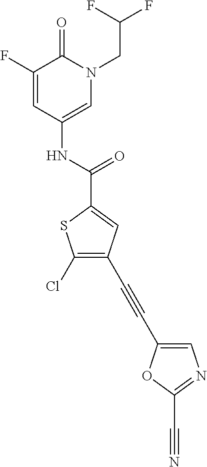 [SMILES: N#Cc1ncc(C#Cc2cc(C(=O)Nc3cc(F)c(=O)n(CC(F)F)c3)sc2Cl)o1] | 451 |
| 101d | 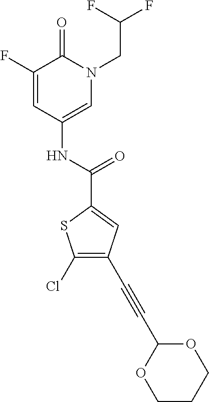 [SMILES: O=C(Nc1cc(F)c(=O)n(CC(F)F)c1)c1cc(C#CC2OCCCO2)c(Cl)s1] | 447 |
| 102d | 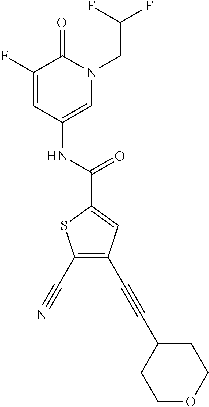 [SMILES: N#Cc1sc(C(=O)Nc2cc(F)c(=O)n(CC(F)F)c2)cc1C#CC1CCOCC1] | 436 |
| 103d | 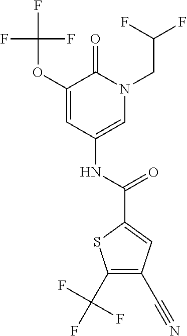 [SMILES: N#Cc1cc(C(=O)Nc2cc(OC(F)(F)F)c(=O)n(CC(F)F)c2)sc1C(F)(F)F] | 462 |
| 104d | 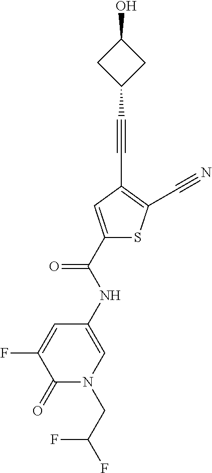 [SMILES: N#Cc1sc(C(=O)Nc2cc(F)c(=O)n(CC(F)F)c2)cc1C#C[C@H]1C[C@H](O)C1] | 422 |
| 105d | 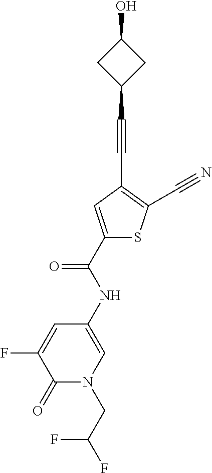 [SMILES: N#Cc1sc(C(=O)Nc2cc(F)c(=O)n(CC(F)F)c2)cc1C#C[C@H]1C[C@@H](O)C1] | 422 |
| 106d | 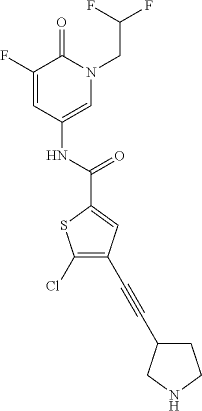 [SMILES: O=C(Nc1cc(F)c(=O)n(CC(F)F)c1)c1cc(C#CC2CCNC2)c(Cl)s1] | 430 |
| 107d | 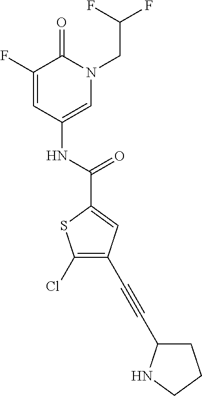 [SMILES: O=C(Nc1cc(F)c(=O)n(CC(F)F)c1)c1cc(C#CC2CCCN2)c(Cl)s1] | 430 |
| 108d | 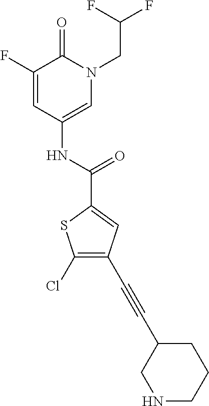 [SMILES: O=C(Nc1cc(F)c(=O)n(CC(F)F)c1)c1cc(C#CC2CCCNC2)c(Cl)s1] | 444 |
| 109d | 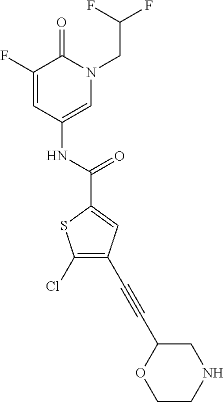 [SMILES: O=C(Nc1cc(F)c(=O)n(CC(F)F)c1)c1cc(C#CC2CNCCO2)c(Cl)s1] | 446 |
| 110d | 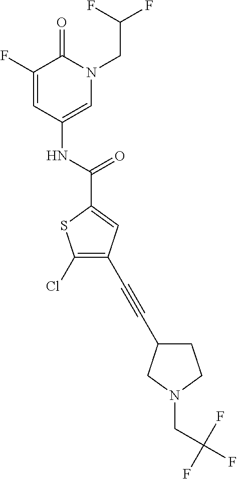 [SMILES: O=C(Nc1cc(F)c(=O)n(CC(F)F)c1)c1cc(C#CC2CCN(CC(F)(F)F)C2)c(Cl)s1] | 512 |
| 111d | 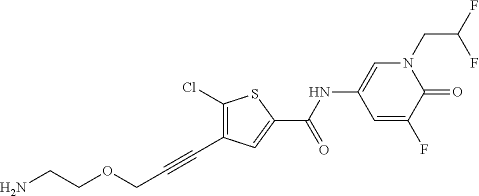 [SMILES: NCCOCC#Cc1cc(C(=O)Nc2cc(F)c(=O)n(CC(F)F)c2)sc1Cl] | 456 |
| 112d | 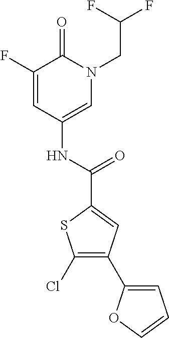 [SMILES: O=C(Nc1cc(F)c(=O)n(CC(F)F)c1)c1cc(-c2ccco2)c(Cl)s1] | 403 |
| 113d | 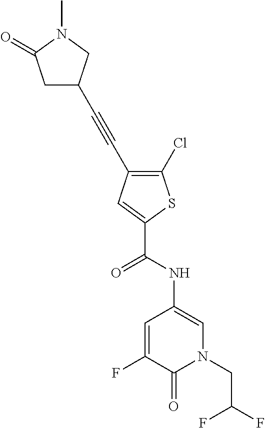 [SMILES: CN1CC(C#Cc2cc(C(=O)Nc3cc(F)c(=O)n(CC(F)F)c3)sc2Cl)CC1=O] | 458 |
| 114d | 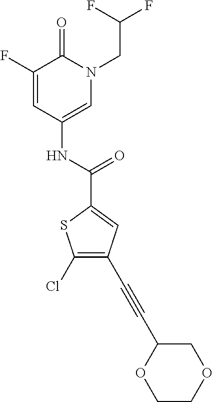 [SMILES: O=C(Nc1cc(F)c(=O)n(CC(F)F)c1)c1cc(C#CC2COCCO2)c(Cl)s1] | 447 |
| 115d | 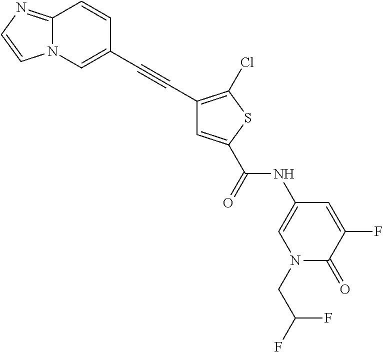 [SMILES: O=C(Nc1cc(F)c(=O)n(CC(F)F)c1)c1cc(C#Cc2ccc3nccn3c2)c(Cl)s1] | 477 |
| 116d | 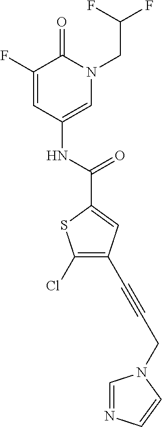 [SMILES: O=C(Nc1cc(F)c(=O)n(CC(F)F)c1)c1cc(C#CCn2ccnc2)c(Cl)s1] | 441 |
| 117d | 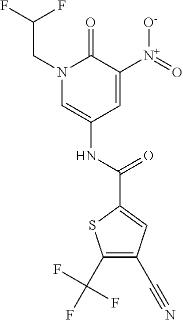 [SMILES: N#Cc1cc(C(=O)Nc2cc([N+](=O)[O-])c(=O)n(CC(F)F)c2)sc1C(F)(F)F] | 421 |
| 118d | 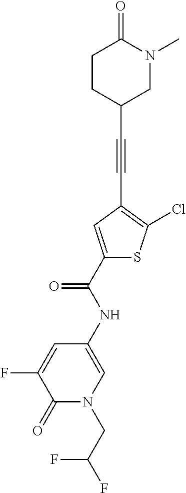 [SMILES: CN1CC(C#Cc2cc(C(=O)Nc3cc(F)c(=O)n(CC(F)F)c3)sc2Cl)CCC1=O] | 472 |
| 119d | 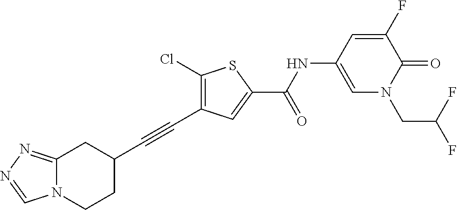 [SMILES: O=C(Nc1cc(F)c(=O)n(CC(F)F)c1)c1cc(C#CC2CCn3cnnc3C2)c(Cl)s1] | 482 |
| 120d | 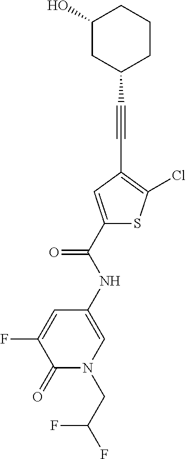 [SMILES: O=C(Nc1cc(F)c(=O)n(CC(F)F)c1)c1cc(C#C[C@H]2CCC[C@@H](O)C2)c(Cl)s1] | 459 |
| 121d | 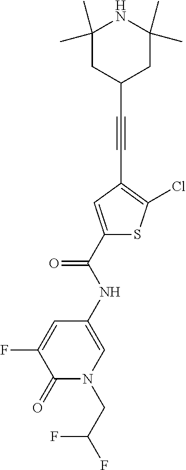 [SMILES: CC1(C)CC(C#Cc2cc(C(=O)Nc3cc(F)c(=O)n(CC(F)F)c3)sc2Cl)CC(C)(C)N1] | 500 |
| 122d | 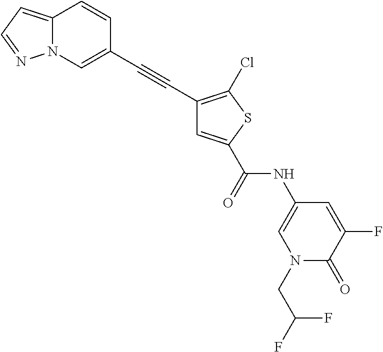 [SMILES: O=C(Nc1cc(F)c(=O)n(CC(F)F)c1)c1cc(C#Cc2ccc3ccnn3c2)c(Cl)s1] | 476 |
| 123d | 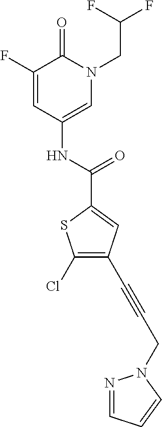 [SMILES: O=C(Nc1cc(F)c(=O)n(CC(F)F)c1)c1cc(C#CCn2cccn2)c(Cl)s1] | 441 |
| 124d | 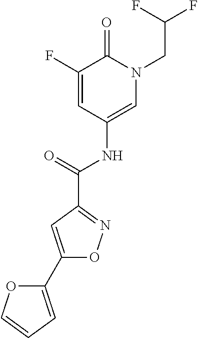 [SMILES: O=C(Nc1cc(F)c(=O)n(CC(F)F)c1)c1cc(-c2ccco2)on1] | 354 |
| 125d | 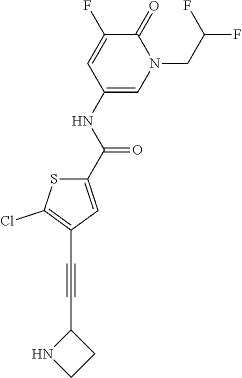 [SMILES: O=C(Nc1cc(F)c(=O)n(CC(F)F)c1)c1cc(C#CC2CCN2)c(Cl)s1] | 460 |
| 126d | 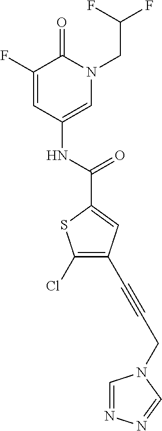 [SMILES: O=C(Nc1cc(F)c(=O)n(CC(F)F)c1)c1cc(C#CCn2cnnc2)c(Cl)s1] | 442 |
| 127d | 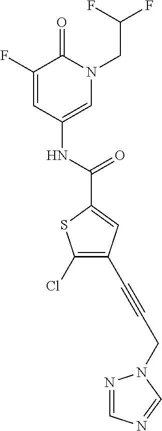 [SMILES: O=C(Nc1cc(F)c(=O)n(CC(F)F)c1)c1cc(C#CCn2cncn2)c(Cl)s1] | 442 |
| 128d | 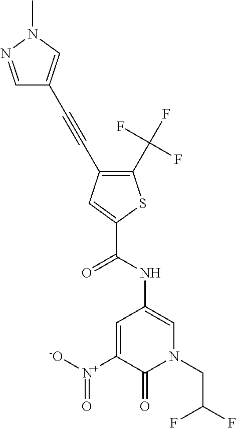 | 500 |
| 129d | 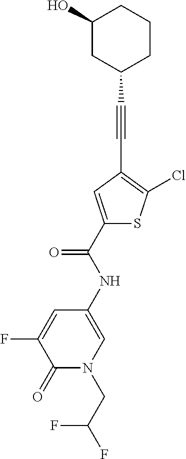 [SMILES: O=C(Nc1cc(F)c(=O)n(CC(F)F)c1)c1cc(C#C[C@H]2CCC[C@H](O)C2)c(Cl)s1] | 459 |
| 130d | 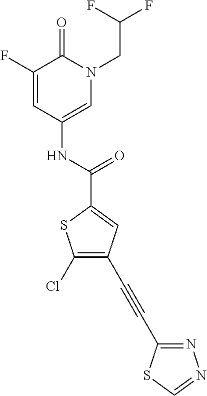 [SMILES: O=C(Nc1cc(F)c(=O)n(CC(F)F)c1)c1cc(C#Cc2nncs2)c(Cl)s1] | 443 |
| 131d | 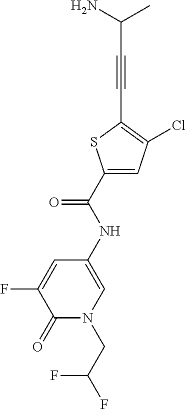 [SMILES: CC(N)C#Cc1sc(C(=O)Nc2cc(F)c(=O)n(CC(F)F)c2)cc1Cl] | 426 |
| 132d | 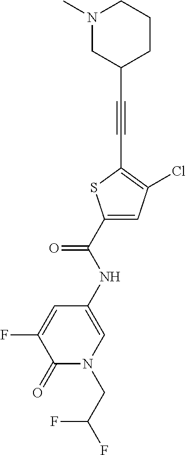 [SMILES: CN1CCCC(C#Cc2sc(C(=O)Nc3cc(F)c(=O)n(CC(F)F)c3)cc2Cl)C1] | 458 |
| 133d | 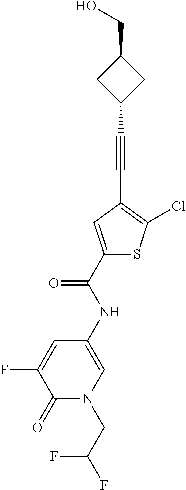 [SMILES: O=C(Nc1cc(F)c(=O)n(CC(F)F)c1)c1cc(C#C[C@H]2C[C@H](CO)C2)c(Cl)s1] | 445 |
| 134d | 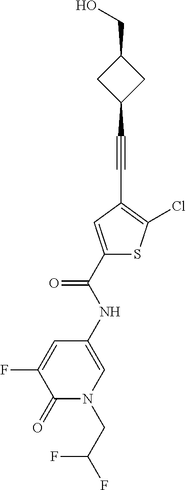 [SMILES: O=C(Nc1cc(F)c(=O)n(CC(F)F)c1)c1cc(C#C[C@H]2C[C@@H](CO)C2)c(Cl)s1] | 445 |
| 135d | 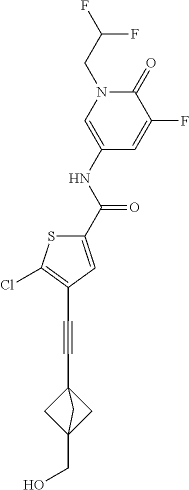 [SMILES: O=C(Nc1cc(F)c(=O)n(CC(F)F)c1)c1cc(C#CC23CC(CO)(C2)C3)c(Cl)s1] | 457 |
| 136d | 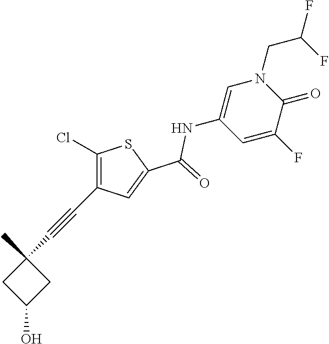 [SMILES: C[C@]1(C#Cc2cc(C(=O)Nc3cc(F)c(=O)n(CC(F)F)c3)sc2Cl)C[C@@H](O)C1] | 445 |
| 137d | 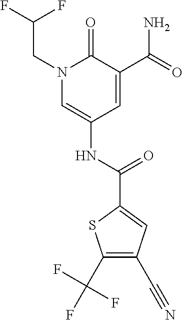 [SMILES: N#Cc1cc(C(=O)Nc2cc(C(N)=O)c(=O)n(CC(F)F)c2)sc1C(F)(F)F] | 421 |
| 138d | 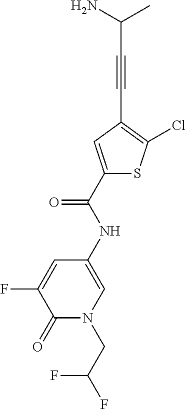 [SMILES: CC(N)C#Cc1cc(C(=O)Nc2cc(F)c(=O)n(CC(F)F)c2)sc1Cl] | 426 |
| 139d | 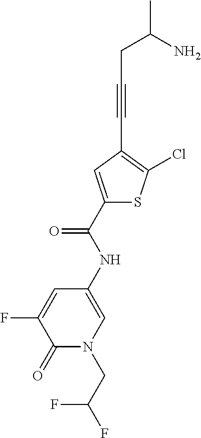 [SMILES: CC(N)CC#Cc1cc(C(=O)Nc2cc(F)c(=O)n(CC(F)F)c2)sc1Cl] | 418 |
| 140d | 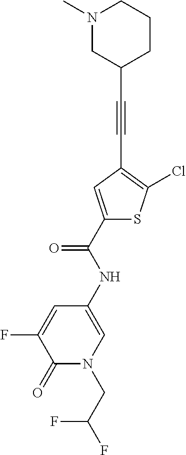 [SMILES: CN1CCCC(C#Cc2cc(C(=O)Nc3cc(F)c(=O)n(CC(F)F)c3)sc2Cl)C1] | 458 |
| 141d | 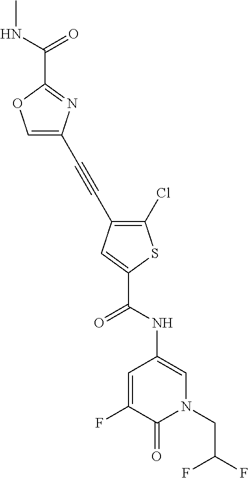 [SMILES: CNC(=O)c1nc(C#Cc2cc(C(=O)Nc3cc(F)c(=O)n(CC(F)F)c3)sc2Cl)co1] | 485 |
| 142d | 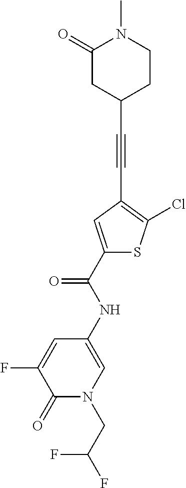 [SMILES: CN1CCC(C#Cc2cc(C(=O)Nc3cc(F)c(=O)n(CC(F)F)c3)sc2Cl)CC1=O] | 472 |
| 143d | 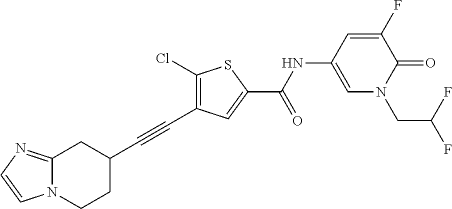 [SMILES: O=C(Nc1cc(F)c(=O)n(CC(F)F)c1)c1cc(C#CC2CCn3ccnc3C2)c(Cl)s1] | 481 |
| 144d | 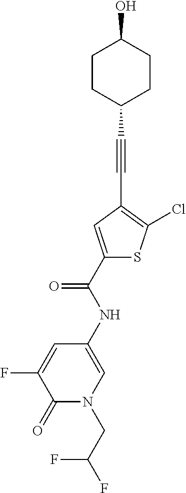 [SMILES: O=C(Nc1cc(F)c(=O)n(CC(F)F)c1)c1cc(C#C[C@H]2CC[C@H](O)CC2)c(Cl)s1] | 459 |
| 145d | 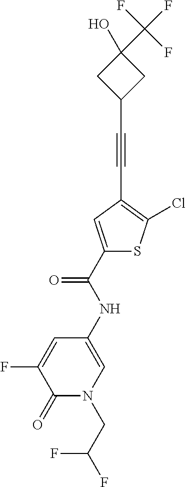 [SMILES: O=C(Nc1cc(F)c(=O)n(CC(F)F)c1)c1cc(C#CC2CC(O)(C(F)(F)F)C2)c(Cl)s1] | 499 |
| 146d | 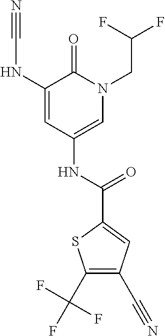 [SMILES: N#CNc1cc(NC(=O)c2cc(C#N)c(C(F)(F)F)s2)cn(CC(F)F)c1=O] | 416 |

In some embodiments, the compound is not:

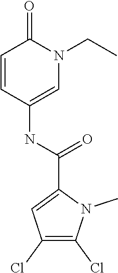

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. In some embodiments, C₁-C₆ alkyl is intended to include C₁, C₂, C₃, C₄, C₅ or C₆ alkyl groups. Examples of alkyl include moieties having from one to six carbon atoms, such as, but not limited to, methyl, ethyl, n-propyl, i-propyl, n-butyl, s-butyl, t-butyl, n-pentyl, s-pentyl or n-hexyl.

In certain embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “cycloalkyl” refers to a saturated or unsaturated nonaromatic hydrocarbon mono- or multi-ring (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. Bridged rings are also included in the definition of cycloalkyl, including, for example, [3.3.0]bicyclooctane, [4.3.0]bicyclononane, and [4.4.0] bicyclodecane and [2.2.2]bicyclooctane. A bridged ring occurs when one or more carbon atoms link two non-adjacent carbon atoms. In one embodiment, bridge rings are one or two carbon atoms. It is noted that a bridge always converts a monocyclic ring into a tricyclic ring. When a ring is bridged, the substituents recited for the ring may also be present on the bridge. Fused (e.g., tetrahydronaphthyl) and spiro rings are also included.

As used herein, the term “heterocycloalkyl” refers to a saturated or unsaturated nonaromatic 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic non-aromatic rings, only one of the rings needs to be non-aromatic (e.g., 1,2,3,4-tetrahydronaphthalenyl, 2,3-dihydroindolyl, benzo[d][1,3]dioxolyl, [1,3]dioxolo[4,5-b]pyridinyl, 5,6,7,8-tetrahydroimidazo[1,2-a]pyrazinyl, and 4,5,6,6a-tetrahydrocyclopenta[b]pyrrolyl).

The term “unsubstituted or substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, aminosulfonyl, alkylsulfonyl, sulfonamido, nitro, trifluoromethyl, cyano, azido, cycloalkyl, heterocyclyl, alkylaryl, or an aromatic or heteroaromatic moiety (i.e., aryl or heteroaryl).

“Alkenyl” includes unsaturated aliphatic groups analogous in length and possible substitution to the alkyls described above, but that contain at least one double bond. In some embodiments, the term “alkenyl” includes straight chain alkenyl groups (e.g., ethenyl, propenyl, butenyl, pentenyl, hexenyl, heptenyl, octenyl, nonenyl, decenyl), and branched alkenyl groups.

In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

The term “unsubstituted or substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, aminosulfonyl, alkylsulfonyl, sulfonamido, nitro, trifluoromethyl, cyano, cycloalkyl, heterocyclyl, alkylaryl, or an aromatic or heteroaromatic moiety (i.e., aryl or heteroaryl).

“Alkynyl” includes unsaturated aliphatic groups analogous in length and possible substitution to the alkyls described above, but which contain at least one triple bond. In some embodiments, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. In some embodiments, C₂-C₆ alkenylene linker is intended to include C₂, C₃, C₄, C₅ and C₆ alkenylene linker groups.

The term “unsubstituted or substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, aminosulfonyl, alkylsulfonyl, sulfonamido, nitro, trifluoromethyl, cyano, azido, cycloalkyl, heterocyclyl, alkylaryl, or an aromatic or heteroaromatic moiety (i.e., aryl or heteroaryl).

Other unsubstituted or substituted moieties (such as unsubstituted or substituted cycloalkyl, heterocycloalkyl, aryl, or heteroaryl) include both the unsubstituted moieties and the moieties having one or more of the designated substituents. In some embodiments, substituted heterocycloalkyl, cycloalkyl, aryl, or heteroaryl includes those substituted with one or more alkyl groups, such as 2,2,6,6-tetramethyl-piperidinyl and 2,2,6,6-tetramethyl-1,2,3,6-tetrahydropyridinyl. In some embodiments, substituted heterocycloalkyl, cycloalkyl, aryl, or heteroaryl includes those substituted with one or more oxo groups, such as thiazol-2-onyl, pyrrolidin-3-onyl, piperidin-2-onyl, morpholin-3-onyl, pyridin-2(3H)-onyl, pyridin-3(4H)-onyl, pyridin-4(3H)-only, pyridazin-3(4H)-only, dihydro-2H-pyran-3(4H)-onyl, isoindolin-1-onyl 6,7-dihydropyrazolo[1,5-a]pyrazin-4(5H)-onyl, and 2H-benzo[b][1,4]oxazin-3(4H)-only.

“Aryl” includes groups with aromaticity, including “conjugated,” or multicyclic systems with one or more aromatic rings and do not contain any heteroatom in the ring structure. Examples include phenyl, naphthalenyl, etc.

“Heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1.

Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine, indazole, 1H-pyrazolo[3,4-b]pyridine. 1H-benzo[d]imidazole.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, aminosulfonyl, alkylsulfonyl, sulfonamido, nitro, trifluoromethyl, cyano, azido, heterocyclyl, alkylaryl, or an aromatic or heteroaromatic moiety (i.e., aryl or heteroaryl). Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As described herein, compounds of the application may optionally be substituted with one or more substituents, such as are illustrated generally above, or as exemplified by particular classes, subclasses, and species of the application. It will be appreciated that the phrase “unsubstituted or substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an unsubstituted or substituted group may have a substituent at each substitutable position of the group, and when more than one position in any given structure may be substituted with more than one substituent selected from a specified group, the substituent may be either the same or different at every position. The terms “unsubstituted or substituted”, “unsubstituted or substituted alkyl,” “unsubstituted or substituted “unsubstituted or substituted alkenyl,” “unsubstituted or substituted alkynyl”, “unsubstituted or substituted cycloalkyl,” “unsubstituted or substituted cycloalkenyl,” “unsubstituted or substituted aryl”, “unsubstituted or substituted heteroaryl,” “unsubstituted or substituted aralkyl”, “unsubstituted or substituted heteroaralkyl,” “unsubstituted or substituted heterocycloalkyl,” “optionally substituted”, “optionally substituted alkyl,” “optionally substituted “optionally substituted alkenyl,” “optionally substituted alkynyl”, “optionally substituted cycloalkyl,” “optionally substituted cycloalkenyl,” “optionally substituted aryl”, “optionally substituted heteroaryl,” “optionally substituted aralkyl”, “optionally substituted heteroaralkyl,” “optionally substituted heterocycloalkyl,” and any other optionally substituted and/or any other unsubstituted or substituted group as used herein, refer to groups that are optionally substituted and/or substituted or unsubstituted by independent replacement of one, two, or three or more of the hydrogen atoms thereon with substituents including, but not limited to:

—F, —Cl, —Br, —I, —OH, protected hydroxy, —NO₂, —CN, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkenyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkenyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkenyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₁₂-alkenyl, —OCO₂—C₂-C₁₂-alkenyl, —OCO₂—C₃-C₁₂-cycloalkyl, —OCO₂-aryl, —OCO-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkenyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₁₂-alkenyl, —NHC(O)—C₂-C₁₂-alkenyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkenyl, —NHCO₂—C₃-C₁₂-cycloalkyl, —NHCO₂-aryl, —NHCO₂-heteroaryl, —NHCO₂— heterocycloalkyl, NHC(O)NH₂, —NHC(O)NH—C₁-C₁₂-alkyl, —NHC(O)NH—C₂-C₁₂-alkenyl, —NHC(O)NH—C₂-C₁₂-alkenyl, —NHC(O)NH—C₃-C₁₂-cycloalkyl, —NHC(O)NH-aryl, —NHC(O)NH-heteroaryl, NHC(O)NH-heterocycloalkyl, —NHC(S)NH₂, —NHC(S)NH—C₁-C₁₂-alkyl, —NHC(S)NH—C₂-C₁₂-alkenyl, —NHC(S)NH—C₂-C₁₂-alkenyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₁₂-alkenyl, —NHC(NH)NH—C₂-C₁₂-alkenyl, —NHC(NH)NH—C₃-C₁₂-cycloalkyl, —NHC(NH)NH-aryl, —NHC(NH)NH-heteroaryl, —NHC(NH)NHheterocycloalkyl, —NHC(NH)—C₁-C₁₂-alkyl, —NHC(NH)—C₂-C₁₂-alkenyl, —NHC(NH)—C₂-C₁₂-alkenyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkenyl, C(NH)NH—C₃-C₁₂-cycloalkyl, —C(NH)NH-aryl, —C(NH)NH-heteroaryl, —C(NH)NHheterocycloalkyl, —S(O)—C₁-C₁₂-alkyl, —S(O)—C₂-C₁₂-alkenyl, —S(O)—C₂-C₁₂-alkenyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₁₂-alkenyl, —SO₂NH—C₂-C₁₂-alkenyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkenyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkenyl, —S—C₃-C₁₂-cycloalkyl, —S-aryl, —S-heteroaryl, —S-heterocycloalkyl, or methylthiomethyl.

The term “substituted,” as used herein, means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

The term “optionally substituted,” as used herein, means not being substituted (e.g., none of the one or more hydrogen atoms on the designated variable is replaced with any other group) or being substituted (e.g., any one or more hydrogen atoms on the designated variable is replaced with a suitable group, provided that the designated atom's normal valency is not exceeded, and that the substitution results in a stable compound).

Any of the substituents on compounds or moieties defined herein may be further substituted as described herein for the compounds or moieties constituting those substituents. For example, an alkyl substituent on any group can be “substituted alkyl” as described herein.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, in some embodiments, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

The term “hydroxy” or “hydroxyl” includes groups with an —OH or —O⁻.

As used herein, “halo” or “halogen” refers to fluoro, chloro, bromo and iodo. The term “perhalogenated” generally refers to a moiety wherein all hydrogen atoms are replaced by halogen atoms. The term “haloalkyl” or “haloalkoxyl” refers to an alkyl or alkoxyl substituted with one or more halogen atoms.

As used herein, “nitro” means a group of the formula —NO₂.

The term “carbonyl” includes compounds and moieties which contain a carbon connected with a double bond to an oxygen atom. Examples of moieties containing a carbonyl include, but are not limited to, aldehydes, ketones, carboxylic acids, amides, esters, anhydrides, etc. Carbonyl groups may be further substituted so as to include, e.g. alkylcarbonyl, arylcarbonyl or aminocarbonyl.

The term “alkylcarbonyl” refers to compounds and moieties which contain an alkyl group connected to a carbonyl (i.e., carbon connected with a double bond to an oxygen atom). The term includes compounds wherein the alkyl group connected to the carbonyl may be further substituted.

The term “aminocarbonyl” includes compounds or moieties that contain a nitrogen atom that is bound to the carbon of a carbonyl group. The term includes “alkylaminocarbonyl” and “dialkylaminocarbonyl” groups that include alkyl, alkenyl or alkynyl groups bound to a nitrogen atom which is bound to the carbon of a carbonyl group. It also includes “arylaminocarbonyl” groups that include aryl or heteroaryl moieties bound to a nitrogen atom that is bound to the carbon of a carbonyl group. The terms “alkylaminocarbonyl”, “alkenylaminocarbonyl”, “alkynylaminocarbonyl” and “arylaminocarbonyl” include moieties wherein alkyl, alkenyl, alkynyl and aryl moieties, respectively, are bound to a nitrogen atom which is in turn bound to the carbon of a carbonyl group. Substituents on aminocarbonyl groups may be further substituted.

The term “carboxyl” refers to —COOH or its C₁-C₆ alkyl ester.

The term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, aminosulfonyl, alkylsulfonyl, sulfonamido, nitro, trifluoromethyl, cyano, azido, heterocyclyl, alkylaryl, or an aromatic or heteroaromatic moieties (i.e., aryl or heteroaryl). Examples of halogen substituted alkoxy groups include, but are not limited to, fluoromethoxy, difluoromethoxy, trifluoromethoxy, chloromethoxy, dichloromethoxy and trichloromethoxy.

The term “aryloxy” or “aryloxyl” includes substituted and unsubstituted aryl groups covalently linked to an oxygen atom, where aryl is as defined herein. Examples of aryloxy groups include, but are not limited to, phenoxy and naphthoxy.

The term “alkylsulfonyl” includes compounds and moieties which contain an alkyl group connected with a single bond to a sulfonyl group (i.e., a sulfur atom connected with double bonds to two oxygen atoms. Examples of alkylsulfonyl groups include, but are not limited to methylsulfonyl, ethylsulfonyl, n-propylsulfonyl, i-propylsulfonyl, n-butylsulfonyl, s-butylsulfonyl, t-butylsulfonyl, n-pentylsulfonyl, s-pentylsulfonyl and n-hexylsulfonyl. The alkylsulfonyl groups can be substituted with groups such as alkyl, alkenyl, alkynyl, halogen, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, aminosulfonyl, alkylsulfonyl, sulfonamido, nitro, trifluoromethyl, cyano, azido, heterocyclyl, alkylaryl, or an aromatic or heteroaromatic moieties.

As used herein, “amine” or “amino” refers to —NH₂. Amino groups may be further substituted so as to include, e.g. alkylamino, dialkylamino, arylamino, diarylamino and alkylarylamino. “Alkylamino” includes groups of compounds wherein the nitrogen of —NH₂ is bound to at least one alkyl group. Examples of alkylamino groups include benzylamino, methylamino, ethylamino, phenethylamino, etc. “Dialkylamino” includes groups wherein the nitrogen of —NH₂ is bound to two alkyl groups. Examples of dialkylamino groups include, but are not limited to, dimethylamino and diethylamino. “Arylamino” and “diarylamino” include groups wherein the nitrogen is bound to at least one or two aryl groups, respectively. “Aminoaryl” and “aminoaryloxy” refer to aryl and aryloxyl substituted with amino. “Alkylarylamino,” “alkylaminoaryl” or “arylaminoalkyl” refers to an amino group which is bound to at least one alkyl group and at least one aryl group. “Alkaminoalkyl” refers to an alkyl, alkenyl, or alkynyl group bound to a nitrogen atom which is also bound to an alkyl group. “Acylamino” includes groups wherein nitrogen is bound to an acyl group. Examples of acylamino include, but are not limited to, alkylcarbonylamino, arylcarbonylamino, carbamoyl and ureido groups.

The term “aminosulfonyl” includes compounds and moieties which contain an amino group connected with a single bond to a sulfonyl group (i.e., a sulfur atom connected with double bonds to two oxygen atoms. The term includes “alkylaminosulfonyl” or “dialkylaminosulfonyl” groups that include alkyl, alkenyl or alkynyl groups bound to a nitrogen atom which is bound to the sulfur of a sulfonyl group. It also includes “arylaminosulfonyl” groups that include aryl or heteroaryl moieties bound to a nitrogen atom that is bound to the sulfur of a sulfonyl group.

“Cyano” or “nitrile” refers to the group —CN.

Compounds of the present disclosure that contain nitrogens can be converted to N-oxides by treatment with an oxidizing agent (e.g., 3-chloroperoxybenzoic acid (mCPBA) and/or hydrogen peroxides) to afford other compounds of the present disclosure. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds of the present disclosure can be converted to N-hydroxy or N-alkoxy compounds. In some embodiments, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. In addition, a crystal polymorphism may be present for the compounds represented by the formula. It is noted that any crystal form, crystal form mixture, or anhydride or hydrate thereof is included in the scope of the present disclosure.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds or a cycloalkyl linker (e.g., 1,3-cylcobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It should also be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

Furthermore, the structures and other compounds discussed in this disclosure include all atropic isomers thereof, it being understood that not all atropic isomers may have the same level of activity. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerization is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. Examples of lactam-lactim tautomerism are as shown below.

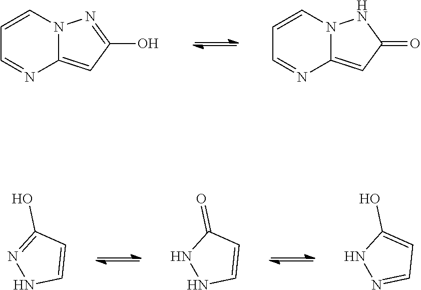 [SMILES: Oc1cc2ncccn2n1 ; O=c1cc2ncccn2[nH]1 ; Oc1cc[nH]n1 ; O=c1cc[nH][nH]1 ; Oc1ccn[nH]1]

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

The compounds of any Formula described herein include the compounds themselves, as well as their salts, and their solvates, if applicable. A salt, for example, can be formed between an anion and a positively charged group (e.g., amino) on a substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on a substituted benzene compound. Suitable cations include sodium ion, potassium ion, magnesium ion, calcium ion, and an ammonium cation such as tetramethylammonium ion. The substituted benzene compounds also include those salts containing quaternary nitrogen atoms.

Additionally, the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

The present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

### Pharmaceutical Formulations

The disclosure also provides pharmaceutical compositions comprising a compound of the disclosure or pharmaceutically acceptable salts thereof, and one or more other therapeutic agents disclosed herein, mixed with pharmaceutically suitable carriers or excipient(s) at doses to treat or prevent a disease or condition as described herein. The pharmaceutical compositions of the disclosure can also be administered in combination with other therapeutic agents or therapeutic modalities simultaneously, sequentially, or in alternation.

Mixtures of compositions of the disclosure can also be administered to the patient as a simple mixture or in suitable formulated pharmaceutical compositions. For example, some aspects of the disclosure relate to a pharmaceutical composition comprising a therapeutically effective dose of a compound of the disclosure, or a pharmaceutically acceptable salt, hydrate, enantiomer or stereoisomer thereof, one or more other therapeutic agents, and a pharmaceutically acceptable diluent or carrier.

A “pharmaceutical composition” is a formulation containing the compounds of the disclosure in a form suitable for administration to a subject. A compound of the disclosure and one or more other therapeutic agents described herein each can be formulated individually or in multiple pharmaceutical compositions in any combinations of the active ingredients. Accordingly, one or more administration routes can be properly elected based on the dosage form of each pharmaceutical composition. Alternatively, a compound of the disclosure and one or more other therapeutic agents described herein can be formulated as one pharmaceutical composition.

In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In some embodiments, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A composition of the disclosure can be administered to a subject in many of the well-known methods currently used for chemotherapeutic treatment. For example, for treatment of cancers, a compound of the disclosure may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In some aspects, the disease or condition to be treated is cancer. In some aspects, the disease or condition to be treated is a cell proliferative disorder.

In certain embodiments the therapeutically effective amount of each pharmaceutical agent used in combination will be lower when used in combination in comparison to monotherapy with each agent alone. Such lower therapeutically effective amount could afford for lower toxicity of the therapeutic regimen.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL Q (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the SMARCA2 antagonists (e.g., inhibitors) described herein, other therapeutic agents described herein, compositions comprising a compound of the disclosure and one or more other therapeutic agents, or the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.01 mg/kg per day to about 5000 mg/kg per day. In some aspects, dosages can range from about 1 mg/kg per day to about 1000 mg/kg per day. In some aspects, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

The composition of the disclosure is capable of further forming salts. The composition of the disclosure is capable of forming more than one salt per molecule, e.g., mono-, di-, tri-. All of these forms are also contemplated within the scope of the claimed invention.

As used herein, “pharmaceutically acceptable salts” refer to derivatives of the compounds of the disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates), of the same salt.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, /toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. For example, inorganic salts include, but are not limited to, ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Example organic bases used in certain embodiments include isopropylamine, diethylamine, ethanolamine, trimethylamine, dicyclohexylamine, choline and caffeine.

The composition of the disclosure may also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The composition, or pharmaceutically acceptable salts or solvates thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, Pa. (1995). In some embodiments, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

A composition of the disclosure may comprise a compound of Formula (I), (IA), (IB), (IC), (ID), or (IE) or a pharmaceutically acceptable salt thereof, or a pharmaceutically acceptable salt thereof, and one or more other therapeutic agents, or a pharmaceutically acceptable salt thereof. The disclosure also provides for the administration of a compound of Formula (I), (IA), (IB), (IC), (ID), or (IE) or a pharmaceutically acceptable salt thereof, or a pharmaceutically acceptable salt thereof, and one or more therapeutic agents or a pharmaceutically acceptable salt thereof, as a co-formulation or in separate formulations, wherein the administration of formulations is simultaneous, sequential, or in alternation. In certain embodiments, the other therapeutic agents can be an agent that is recognized in the art as being useful to treat the disease or condition being treated by the composition of the disclosure. In some embodiments, the other therapeutic agent can be an agent that is not recognized in the art as being useful to treat the disease or condition being treated by the composition of the disclosure. In some aspects, the other therapeutic agent can be an agent that imparts a beneficial attribute to the composition of the disclosure (e.g., an agent that affects the viscosity of the composition). The beneficial attribute to the composition of the disclosure includes, but is not limited to, pharmacokinetic or pharmacodynamic co-action resulting from the combination of a compound of Formula (I), (IA), (IB), (IC), (ID), or (IE) or a pharmaceutically acceptable salt thereof, and one or more other therapeutic agents.

The therapeutic agents set forth below are for illustrative purposes and not intended to be limiting. The disclosure includes at least one other therapeutic agent selected from the lists below. The disclosure can include more than one other therapeutic agent, e.g., two, three, four, or five other therapeutic agents such that the composition of the disclosure can perform its intended function.

In some embodiments, the other therapeutic agent is an anticancer agent. In some embodiments, the anticancer agent is a compound that affects histone modifications, such as an HDAC inhibitor (such as Zolinza® or Farydak®). In certain embodiments, an anticancer agent is selected from the group consisting of chemotherapeutics (such as 2CdA, 5-FU, 6-Mercaptopurine, 6-TG, Abraxane™, Accutane®, Actinomycin-D, Adriamycin®, Alimta®, Alkeran® all-trans retinoic acid, amethopterin, Ara-C, Azacitadine, BCNU, Blenoxane®, Camptosar®, CeeNU®, Clofarabine, Clolar™, Cytoxan®, daunorubicin hydrochloride, DaunoXome®, Dacogen®, DIC, Doxil®, Ellence®, Eloxatin®, Emcyt®, etoposide phosphate, Etopophos®, Fludara®, FUDR®, Gemzar®, Gleevec®, hexamethylmelamine, Hycamtin®, Hydrea®, IDamycin®, Ifex®, Imbruvica®, ixabepilone, Ixempra®, L-asparaginase, Leukeran®, liposomal Ara-C, L-PAM, Lysodren, mafosfamide, Marqibo®, Matulane®, mithracin, Mitomycin-C, Myleran®, Navelbine®, Neutrexin®, nilotinib, Nipent®, Nitrogen Mustard, Novantrone®, Oncaspar®, Panretin®, Paraplatin®, Platinol®, prolifeprospan 20 with carmustine implant, Sandostatin®, Targretin®, Tasigna®, Taxotere®, Temodar®, TESPA, Toposar®, Treanda®, Trisenox®, Valstar®, Velban®, Vidaza™, vincristine sulfate, VM 26, Xeloda® and Zanosar®); biologics (such as Alpha Interferon, Bacillus Calmette-Guerin, Bexxar®, Campath®, Ergamisol®, Erlotinib, Herceptin®, Interleukin-2, Iressa®, lenalidomide, Mylotarg®, Ontak®, Pegasys®, Revlimid®, Rituxan®, Tarceva™, Thalomid®, Tykerb®, Velcade® and Zevalin™); corticosteroids, (such as dexamethasone sodium phosphate, DeltaSone® and Delta-Cortef®); glucocorticoid receptor agonists (such as Baycadron®, Maxidex®, Ozurdex®, Econopred®, Omnipred®, or Millipred®); hormonal therapies (such as Arimidex®, Aromasin®, Casodex®, Cytadren®, Eligard®, Eulexin®, Evista®, Faslodex®, Femara®, Halotestin®, Megace®, Nilandron®, Nolvadex®, Plenaxis™ and Zoladex®); and radiopharmaceuticals (such as Iodotope®, Metastron®, Phosphocol® and Samarium SM-153); immunomodulatory drugs (such as Pomalyst®, Revlimid® and Thalidomid®); proteasome inhibitors (such as Kyprolis®, Ninlaro® and Velcade®); bcl-2 inhibitors (such as Venclexta®).

Exemplary glucocorticoid receptor agonists include but are not limited to, dexamethasone (Baycadron®, Maxidex®, Ozurdex®), methylprednisolone (Depo-Medrol®, Solu-Medrol®), or prednisolone (Econopred®, Omnipred®, Millipred®).

Exemplary immunomodulatory drugs include, but are not limited to, lenalidomide (Revlimid®), pomalidomide (Pomalyst®) and thalidomide (Thalidomid®);

Exemplary proteasome inhibitors, include but are not limited to, bortezomib (Velcade®), carfilzomib (Kyprolis®) and ixazomib (Ninlaro®),

Exemplary Bcl-2 inhibitors include, but are not limited to, venetoclax (Venclexta®).

In some embodiments, the other therapeutic agent is a chemotherapeutic agent (also referred to as an anti-neoplastic agent or anti-proliferative agent), selected from the group including an alkylating agent; an antibiotic; an anti-metabolite; a detoxifying agent; an interferon; a polyclonal or monoclonal antibody; an EGFR inhibitor; a HER2 inhibitor; a histone deacetylase inhibitor; a hormone; a mitotic inhibitor; an mTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase or a protein methyltransferase), a cytidine analogue drug or any chemotherapeutic, anti-neoplastic or anti-proliferative agent listed in www.cancer.org/docroot/cdg/cdg_0.asp.

Exemplary alkylating agents include, but are not limited to, cyclophosphamide (Cytoxan®; Neosar®); chlorambucil (Leukeran®); melphalan (Alkeran®); carmustine (BiCNU@); busulfan (Busulfex®); lomustine (CeeNU@); dacarbazine (DTIC-Dome®); oxaliplatin (Eloxatin®); carmustine (Gliadel®); ifosfamide (Ifex®); mechlorethamine (Mustargen); busulfan (Myleran®); carboplatin (Paraplatin®); cisplatin (CDDP®; Platinol®); temozolomide (Temodar®); thiotepa (Thioplex®); bendamustine (Treanda®); or streptozocin (Zanosar®).

Exemplary antibiotics include, but are not limited to, doxorubicin (Adriamycin®); doxorubicin liposomal (Doxil®); mitoxantrone (Novantrone®); bleomycin (Blenoxane®); daunorubicin (Cerubidine®); daunorubicin liposomal (DaunoXome®); dactinomycin (Cosmegen®); epirubicin (Ellence®); idarubicin (IDamycin®); plicamycin (Mithracin®); mitomycin (Mutamycin®); pentostatin (Nipent®); or valrubicin (Valstar®).

Exemplary anti-metabolites include, but are not limited to, fluorouracil (Adrucil®); capecitabine (Xeloda®); hydroxyurea (Hydrea®); mercaptopurine (Purinethol®); pemetrexed (Alimta); fludarabine (Fludara®); nelarabine (Arranon®); cladribine (Cladribine Novaplus®); clofarabine (Clolar®); cytarabine (Cytosar-U®); decitabine (Dacogen®); cytarabine liposomal (DepoCyt®); hydroxyurea (Droxia®); pralatrexate (Folotyn®); floxuridine (FUDR®); gemcitabine (Gemzar®); cladribine (Leustatin®); fludarabine (Oforta®); methotrexate (MTX®; Rheumatrex®); methotrexate (Trexall®); thioguanine (Tabloid®); TS-1 or cytarabine (Tarabine PFS®).

Exemplary detoxifying agents include, but are not limited to, amifostine (Ethyol®) or mesna (Mesnex®).

Exemplary interferons include, but are not limited to, interferon alfa-2b (Intron A®) or interferon alfa-2a (Roferon-A®).

Exemplary polyclonal or monoclonal antibodies include, but are not limited to, trastuzumab (Herceptin®); ofatumumab (Arzerra®); bevacizumab (Avastin®); rituximab (Rituxan®); cetuximab (Erbitux®); panitumumab (Vectibix®); tositumomab/iodinel31 tositumomab (Bexxar®); alemtuzumab (Campath®); ibritumomab (Zevalin®; In-111@; Y-90 Zevalin®); gemtuzumab (Mylotarg®); eculizumab (Soliris®) ordenosumab.

Exemplary EGFR inhibitors include, but are not limited to, gefitinib (Iressa); lapatinib (Tykerb®); cetuximab (Erbitux®); erlotinib (Tarceva®); panitumumab (Vectibix®); PKI-166; canertinib (CI-1033); matuzumab (Emd7200) or EKB-569.

Exemplary HER2 inhibitors include, but are not limited to, trastuzumab (Herceptin®); lapatinib (Tykerb®) or AC-480.

Histone Deacetylase Inhibitors include, but are not limited to, vorinostat (Zolinza®) and panobinostat (Farydak®).

Exemplary hormones include, but are not limited to, tamoxifen (Soltamox; Nolvadex®); raloxifene (Evista®); megestrol (Megace®); leuprolide (Lupron®; Lupron Depot®; Eligard®; Viadur®); fulvestrant (Faslodex®); letrozole (Femara®); triptorelin (Trelstar LA®; Trelstar Depot®); exemestane (Aromasin®); goserelin (Zoladex®); bicalutamide (Casodex®); anastrozole (Arimidex®); fluoxymesterone (Androxy®; Halotestin®); medroxyprogesterone (Provera®; Depo-Provera®); estramustine (Emcyt®); flutamide (Eulexin®); toremifene (Fareston®); degarelix (Firmagon®); nilutamide (Nilandron®); abarelix (Plenaxis®); or testolactone (Teslac®).

Exemplary mitotic inhibitors include, but are not limited to, paclitaxel (Taxol®; Onxol®; Abraxane®); docetaxel (Taxotere®); vincristine (Oncovin®; Vincasar PFS®); vinblastine (Velban®); etoposide (Toposar®; Etopophos®; VePesid®); teniposide (Vumon®); ixabepilone (Ixempra®); nocodazole; epothilone; vinorelbine (Navelbine®); camptothecin (CPT); irinotecan (Camptosar®); topotecan (Hycamtin®); amsacrine or lamellarin D (LAM-D).

Exemplary mTOR inhibitors include, but are not limited to, everolimus (Afinitor®) or temsirolimus (Torisel®); rapamune, ridaforolimus; or AP23573.

Exemplary VEGF/VEGFR inhibitors include, but are not limited to, bevacizumab (Avastin®); sorafenib (Nexavar®); sunitinib (Sutent®); ranibizumab; pegaptanib; or vandetinib.

Exemplary microtubule targeting drugs include, but are not limited to, paclitaxel, docetaxel, vincristine, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, paclitaxel and docetaxol.

Exemplary general chemotherapeutic, anti-neoplastic, anti-proliferative agents include, but are not limited to, altretamine (Hexalen); isotretinoin (Accutane; Amnesteem; Claravis; Sotret); tretinoin (Vesanoid®); azacitidine (Vidaza®); bortezomib (Velcade®) asparaginase (Elspar®); ibrutinib (Imbruvica®); levamisole (Ergamisol®); mitotane (Lysodren®); procarbazine (Matulane); pegaspargase (Oncaspar®); denileukin diftitox (Ontak®); porfimer (Photofrin®); aldesleukin (Proleukin®); lenalidomide (Revlimid®); bexarotene (Targretin®); thalidomide (Thalomid®); temsirolimus (Torisel®); arsenic trioxide (Trisenox®); verteporfin (Visudyn®); mimosine (Leucenol®); (1M tegafur-0.4 M 5-chloro-2,4-dihydroxypyrimidine-1 M potassium oxonate), or lovastatin.

In further aspects, the other therapeutic agent is a chemotherapeutic agent or a cytokine such as G-CSF (granulocyte colony stimulating factor).

In yet further aspects, the other therapeutic agents can be standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0.4:1), Camptothecin-11 (CPT-11, Irinotecan or Camptosar™), CHOP (cyclophosphamide, hydroxydaunorubicin, oncovin, and prednisone or prednisolone), R-CHOP (rituximab, cyclophosphamide, hydroxydaunorubicin, oncovin, prednisone or prednisolone), CVP (cyclophosphamide, vincristine, and prednisone), hyper-CVAD (hyperfractionated cyclophosphamide, vincristine, doxorubicin, and prednisone), or CMFP (cyclophosphamide, methotrexate, 5-fluorouracil and prednisone).

In other aspects, the other therapeutic agents can be an inhibitor of an enzyme, such as a receptor or non-receptor kinase. Receptor and non-receptor kinases are, for example, tyrosine kinases or serine/threonine kinases. Kinase inhibitors described herein are small molecules, polynucleic acids, polypeptides, or antibodies.

Exemplary kinase inhibitors include, but are not limited to, Bevacizumab (targets VEGF), BIBW 2992 (targets EGFR and Erb2), Cetuximab/Erbitux (targets Erb1), Imatinib/Gleevic (targets Bcr-Abl), Trastuzumab (targets Erb2), Gefitinib/Iressa (targets EGFR), Ranibizumab (targets VEGF), Pegaptanib (targets VEGF), Erlotinib/Tarceva (targets Erb1), Nilotinib (targets Bcr-Abl), Lapatinib (targets Erb1 and Erb2/Her2), GW-572016/lapatinib ditosylate (targets HER2/Erb2), Panitumumab/Vectibix (targets EGFR), Vandetinib (targets RET/VEGFR), E7080 (multiple targets including RET and VEGFR), Herceptin (targets HER2/Erb2), PKI-166 (targets EGFR), Canertinib/CI-1033 (targets EGFR), Sunitinib/SU-11464/Sutent (targets EGFR and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P154 (targets JAK), WHI-P131 (targets JAK), sorafenib/Nexavar (targets RAF kinase, VEGFR-1, VEGFR-2, VEGFR-3, PDGFR-β, KIT, FLT-3, and RET), Dasatinib/Sprycel (BCR/ABL and Src), AC-220 (targets Flt3), AC-480 (targets all HER proteins, “panHER”), Motesanib diphosphate (targets VEGF1-3, PDGFR, and c-kit), Denosumab (targets RANKL, inhibits SRC), AMG888 (targets HER3), and AP24534 (multiple targets including Flt3).

Exemplary serine/threonine kinase inhibitors include, but are not limited to, Rapamune (targets mTOR/FRAP1), Deforolimus (targets mTOR), Certican/Everolimus (targets mTOR/FRAP1), AP23573 (targets mTOR/FRAP1), Eril/Fasudil hydrochloride (targets RHO), Flavopiridol (targets CDK), Seliciclib/CYC202/Roscovitrine (targets CDK), SNS-032/BMS-387032 (targets CDK), Ruboxistaurin (targets PKC), Pkc412 (targets PKC), Bryostatin (targets PKC), KAI-9803 (targets PKC), SF1126 (targets PI3K), VX-680 (targets Aurora kinase), Azd1152 (targets Aurora kinase), Arry-142886/AZD-6244 (targets MAP/MEK), SCIO-469 (targets MAP/MEK), GW681323 (targets MAP/MEK), CC-401 (targets INK), CEP-1347 (targets JNK), and PD 332991 (targets CDK).

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WHI-P154; WHI-P131; AC-220; or AMG888.

In some embodiments, the other therapeutic agent is a SMARCA2 antagonist or inhibitor. Exemplary SMARCA2 inhibitors include BMCL 2968, I-BET151, JQ1, and PFI-3. Exemplary SMARCA2 antagonists include antisense RNA, shRNA, siRNA, CRISPR/Cas9, transcription activator-like effector nucleases (TALEN), Zinc Finger nucleases (ZFN), antibodies, antibody fragments and antibody mimetics.

All percentages and ratios used herein, unless otherwise indicated, are by weight. Other features and advantages of the disclosure are apparent from the different examples. The provided examples illustrate different components and methodology useful in practicing the disclosure. The examples do not limit the claimed invention. Based on the present disclosure the skilled artisan can identify and employ other components and methodology useful for practicing the disclosure.

A “subject” includes a mammal. The mammal can be e.g., any mammal, e.g., a human, primate, mouse, rat, dog, cat, cow, horse, goat, camel, sheep or a pig. Preferably, the mammal is a human.

As used herein, a “subject in need thereof” is a subject that has cancer or a precancerous condition. In some embodiments, a subject in need thereof has cancer.

In some embodiments, a subject in need thereof is a subject having a disorder associated with a SMARCA4 mutation, a change in level of activity or function of SMARCA4, a change in level of SMARCA4 protein expression as compared to a control level, a change in level of SMARCA4 mRNA expression as compared to a control level, and/or an increased risk of developing such disorder relative to the population at large. In some embodiments, the disorder associated with a SMARCA4 mutation is a cancer. In some embodiments, the change in level of activity or function of SMARCA4 as compared to a control level is a decrease. In some embodiments, the change in level of SMARCA4 protein expression as compared to a control level is a decrease. In some embodiments, the change in level of SMARCA4 mRNA expression as compared to a control level is a decrease. In some embodiments, a subject in need thereof is a subject having a disorder associated with a SMARCA4 mutation, a decrease in level of activity or function of SMARCA4, a decrease in level of SMARCA4 protein expression as compared to a control level, a decrease in level of SMARCA4 mRNA expression as compared to a control level, and/or an increased risk of developing such disorder relative to the population at large

In some embodiments, a subject in need thereof is a subject having a disorder associated with a SMARCA4 mutation, decreased level of activity or function of SMARCA4, a decreased level of SMARCA4 protein expression, a decreased level of SMARCA4 mRNA expression compared to a control level, and/or a subject having an increased risk of developing such disorder relative to the population at large. In some embodiments, the subject or a cell of the subject exhibits a SMARCA4 mutation as compared to wild-type SMARCA4. In some embodiments, the SMARCA4 mutation is a change in at least one nucleotide as compared to the wild-type SMARCA4.

In some embodiments, the subject or a cell of the subject exhibits a decrease of SMARCA4 protein expression as compared to a control level. In some embodiments, the subject or a cell of the subject exhibits a loss of SMARCA4 protein expression as compared to a control level. In some embodiments, the subject or a cell of the subject exhibits a loss of SMARCA4 mRNA expression as compared to a control level. In some embodiments, the subject or a cell of the subject exhibits a decreased SMARCA4 activity as compared to a control level. In some embodiments, the subject or a cell of the subject exhibits a decreased SMARCA4 function as compared to a control level.

In some embodiments, the control level is a level of SMARCA4 protein expression, a level of SMARCA4 mRNA expression, a level of SMARCA4 activity or a level of SMARCA4 function in a subject or cell from a subject that does not have cancer. In some embodiments, the control level may be a level of SMARCA4 protein expression, a level of SMARCA4 mRNA expression, a level of SMARCA4 activity or a level of SMARCA4 function in a subject or cell from a subject belonging to a certain population, wherein the level is equal or about equal to the average level of protein expression, mRNA expression, activity or function of SMARCA4 observed in said population. In some embodiments, the control level may be a level of protein expression, mRNA expression, activity or function of SMARCA4 that is equal or about equal to the average level of protein expression, mRNA expression, activity or function of SMARCA4 in the population at large. In some embodiments, the control level is a level of SMARCA4 protein expression in a subject or cell from a subject that does not have cancer. In some embodiments, the control level is a level of SMARCA4 mRNA expression in a subject or cell from a subject that does not have cancer. In some embodiments, the control level is a level of SMARCA4 activity in a subject or cell from a subject that does not have cancer. In some embodiments, the control level is a level of SMARCA4 function in a subject or cell from a subject that does not have cancer.

The subject of the disclosure includes any human subject who has been diagnosed with, has symptoms of, or is at risk of developing a cancer or a precancerous condition. The subject of the disclosure includes any human subject expressing a mutant SMARCA4 gene. For example, a mutant SMARCA4 comprises one or more mutations, wherein the mutation is a substitution, a point mutation, a nonsense mutation, a missense mutation, a deletion, an insertion, or a translocation or any other SMARCA4 mutation described herein or otherwise known in the art to be associated with a loss of function of SMARCA4.

A subject in need thereof may have refractory or resistant cancer. “Refractory or resistant cancer” means cancer that does not respond to an established line of treatment. The cancer may be resistant at the beginning of treatment or it may become resistant during treatment. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy. In certain embodiments, the prior therapy is monotherapy. In certain embodiments, the prior therapy is combination therapy.

In some embodiments, a subject in need thereof may have a secondary cancer as a result of a previous therapy. “Secondary cancer” means cancer that arises due to or as a result from previous carcinogenic therapies, such as chemotherapy.

The subject may also exhibit decreased function or expression of SMARCA4, or loss of function of SMARCA4.

In some embodiments, the subject is a participant in a clinical trial. In some embodiments, a criterion for participation of a subject in the clinical trial is a decreased activity or function of SMARCA4, or loss of function of SMARCA4, in said subject or a cell of said subject.

As used herein, the term “responsiveness” is interchangeable with terms “responsive”, “sensitive”, and “sensitivity”, and it is meant that a subject is showing therapeutic responses when administered a composition of the disclosure, e.g., tumor cells or tumor tissues of the subject undergo apoptosis and/or necrosis, and/or display reduced growing, dividing, or proliferation. This term is also meant that a subject will or has a higher probability, relative to the population at large, of showing therapeutic responses when administered a composition of the disclosure, e.g., tumor cells or tumor tissues of the subject undergo apoptosis and/or necrosis, and/or display reduced growing, dividing, or proliferation.

As used herein, “sample” means any biological sample derived from the subject, includes but is not limited to, cells, tissues samples, body fluids (including, but not limited to, mucus, blood, plasma, serum, urine, saliva, and semen), tumor cells, and tumor tissues. Preferably, the sample is selected from bone marrow, peripheral blood cells, blood, plasma and serum. Samples can be provided by the subject under treatment or testing. Alternatively samples can be obtained by the physician according to routine practice in the art.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “candidate compound” refers to a compound of the disclosure, or a pharmaceutically acceptable salt or solvate thereof, that has been or will be tested in one or more in vitro or in vivo biological assays, in order to determine if that compound is likely to elicit a desired biological or medical response in a cell, tissue, system, animal or human that is being sought by a researcher or clinician. A candidate compound is a compound of the disclosure, or a pharmaceutically acceptable salt or solvate thereof. The biological or medical response can be the treatment of cancer. The biological or medical response can be treatment or prevention of a cell proliferative disorder. In vitro or in vivo biological assays can include, but are not limited to, enzymatic activity assays, electrophoretic mobility shift assays, reporter gene assays, in vitro cell viability assays, and the assays described herein.

As used herein, “treating” or “treat” describes the management and care of a patient for the purpose of combating a disease, condition, or disorder and includes the administration of a compound of the disclosure, or a pharmaceutically acceptable salt or solvate thereof, to alleviate the symptoms or complications of a disease, condition or disorder, or to eliminate the disease, condition or disorder.

A composition of the disclosure, or a pharmaceutically acceptable salt or solvate thereof, can also be used to prevent a disease, condition, or disorder. As used herein, “preventing” or “prevent” describes reducing or eliminating the onset of the symptoms or complications of the disease, condition, or disorder.

As used herein, the term “alleviate” is meant to describe a process by which the severity of a sign or symptom of a disorder is decreased. Importantly, a sign or symptom can be alleviated without being eliminated. In some embodiments, the administration of pharmaceutical compositions of the disclosure leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In some aspects of the disclosure, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

### Cancer

A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

A “cell proliferative disorder of the hematologic system” is a cell proliferative disorder involving cells of the hematologic system. A cell proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system can include hyperplasia, dysplasia, and metaplasia of cells of the hematologic system. Preferably, compositions of the disclosure may be used to treat a cancer selected from the group consisting of a hematologic cancer of the disclosure or a hematologic cell proliferative disorder of the disclosure. A hematologic cancer of the disclosure can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

A “cell proliferative disorder of the lung” is a cell proliferative disorder involving cells of the lung. Cell proliferative disorders of the lung can include all forms of cell proliferative disorders affecting lung cells. Cell proliferative disorders of the lung can include lung cancer, a precancer or precancerous condition of the lung, benign growths or lesions of the lung, and malignant growths or lesions of the lung, and metastatic lesions in tissue and organs in the body other than the lung. Preferably, compositions of the disclosure may be used to treat lung cancer or cell proliferative disorders of the lung. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), squamous cell carcinoma, adenocarcinoma, small cell carcinoma, large cell carcinoma, adenosquamous cell carcinoma, and mesothelioma. Lung cancer can include “scar carcinoma,” bronchioalveolar carcinoma, giant cell carcinoma, spindle cell carcinoma, and large cell neuroendocrine carcinoma. Lung cancer can include lung neoplasms having histologic and ultrastructural heterogeneity (e.g., mixed cell types).

Cell proliferative disorders of the lung can include all forms of cell proliferative disorders affecting lung cells. Cell proliferative disorders of the lung can include lung cancer, precancerous conditions of the lung. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

A “cell proliferative disorder of the colon” is a cell proliferative disorder involving cells of the colon. Preferably, the cell proliferative disorder of the colon is colon cancer. Preferably, compositions of the disclosure may be used to treat colon cancer or cell proliferative disorders of the colon. Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. Colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

Cell proliferative disorders of the colon can include all forms of cell proliferative disorders affecting colon cells. Cell proliferative disorders of the colon can include colon cancer, precancerous conditions of the colon, adenomatous polyps of the colon, and metachronous lesions of the colon. A cell proliferative disorder of the colon can include adenoma. Cell proliferative disorders of the colon can be characterized by hyperplasia, metaplasia, and dysplasia of the colon. Prior colon diseases that may predispose individuals to development of cell proliferative disorders of the colon can include prior colon cancer. Current disease that may predispose individuals to development of cell proliferative disorders of the colon can include Crohn's disease and ulcerative colitis. A cell proliferative disorder of the colon can be associated with a mutation in a gene selected from the group consisting of p53, ras, FAP and DCC. An individual can have an elevated risk of developing a cell proliferative disorder of the colon due to the presence of a mutation in a gene selected from the group consisting of p53, ras, FAP and DCC.

A “cell proliferative disorder of the pancreas” is a cell proliferative disorder involving cells of the pancreas. Cell proliferative disorders of the pancreas can include all forms of cell proliferative disorders affecting pancreatic cells. Cell proliferative disorders of the pancreas can include pancreas cancer, a precancer or precancerous condition of the pancreas, hyperplasia of the pancreas, and dysaplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas. Pancreatic cancer can include ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. Pancreatic cancer can also include pancreatic neoplasms having histologic and ultrastructural heterogeneity (e.g., mixed cell types).

A “cell proliferative disorder of the prostate” is a cell proliferative disorder involving cells of the prostate. Cell proliferative disorders of the prostate can include all forms of cell proliferative disorders affecting prostate cells. Cell proliferative disorders of the prostate can include prostate cancer, a precancer or precancerous condition of the prostate, benign growths or lesions of the prostate, malignant growths or lesions of the prostate and metastatic lesions in tissue and organs in the body other than the prostate. Cell proliferative disorders of the prostate can include hyperplasia, metaplasia, and dysplasia of the prostate.

A “cell proliferative disorder of the skin” is a cell proliferative disorder involving cells of the skin. Cell proliferative disorders of the skin can include all forms of cell proliferative disorders affecting skin cells. Cell proliferative disorders of the skin can include a precancer or precancerous condition of the skin, benign growths or lesions of the skin, melanoma, malignant melanoma and other malignant growths or lesions of the skin, and metastatic lesions in tissue and organs in the body other than the skin. Cell proliferative disorders of the skin can include hyperplasia, metaplasia, and dysplasia of the skin.

A “cell proliferative disorder of the ovary” is a cell proliferative disorder involving cells of the ovary. Cell proliferative disorders of the ovary can include all forms of cell proliferative disorders affecting cells of the ovary. Cell proliferative disorders of the ovary can include a precancer or precancerous condition of the ovary, benign growths or lesions of the ovary, ovarian cancer, malignant growths or lesions of the ovary, and metastatic lesions in tissue and organs in the body other than the ovary. Cell proliferative disorders of the ovary can include hyperplasia, metaplasia, and dysplasia of cells of the ovary.

A “cell proliferative disorder of the breast” is a cell proliferative disorder involving cells of the breast. Cell proliferative disorders of the breast can include all forms of cell proliferative disorders affecting breast cells. Cell proliferative disorders of the breast can include breast cancer, a precancer or precancerous condition of the breast, benign growths or lesions of the breast, and malignant growths or lesions of the breast, and metastatic lesions in tissue and organs in the body other than the breast. Cell proliferative disorders of the breast can include hyperplasia, metaplasia, and dysplasia of the breast.

A cell proliferative disorder of the breast can be a precancerous condition of the breast. Compositions of the disclosure may be used to treat a precancerous condition of the breast. A precancerous condition of the breast can include atypical hyperplasia of the breast, ductal carcinoma in situ (DCIS), intraductal carcinoma, lobular carcinoma in situ (LCIS), lobular neoplasia, and stage 0 or grade 0 growth or lesion of the breast (e.g., stage 0 or grade 0 breast cancer, or carcinoma in situ). A precancerous condition of the breast can be staged according to the TNM classification scheme as accepted by the American Joint Committee on Cancer (AJCC), where the primary tumor (T) has been assigned a stage of T0 or Tis; and where the regional lymph nodes (N) have been assigned a stage of N0; and where distant metastasis (M) has been assigned a stage of M0.

The cell proliferative disorder of the breast can be breast cancer. Preferably, compositions of the disclosure may be used to treat breast cancer. Breast cancer includes all forms of cancer of the breast. Breast cancer can include primary epithelial breast cancers. Breast cancer can include cancers in which the breast is involved by other tumors such as lymphoma, sarcoma or melanoma. Breast cancer can include carcinoma of the breast, ductal carcinoma of the breast, lobular carcinoma of the breast, undifferentiated carcinoma of the breast, cystosarcoma phyllodes of the breast, angiosarcoma of the breast, and primary lymphoma of the breast. Breast cancer can include Stage I, II, IIIA, IIIB, IIIC and IV breast cancer. Ductal carcinoma of the breast can include invasive carcinoma, invasive carcinoma in situ with predominant intraductal component, inflammatory breast cancer, and a ductal carcinoma of the breast with a histologic type selected from the group consisting of comedo, mucinous (colloid), medullary, medullary with lymphocytic infiltrate, papillary, scirrhous, and tubular. Lobular carcinoma of the breast can include invasive lobular carcinoma with predominant in situ component, invasive lobular carcinoma, and infiltrating lobular carcinoma. Breast cancer can include Paget's disease, Paget's disease with intraductal carcinoma, and Paget's disease with invasive ductal carcinoma. Breast cancer can include breast neoplasms having histologic and ultrastructural heterogeneity (e.g., mixed cell types).

Preferably, compound of the disclosure, or a pharmaceutically acceptable salt or solvate thereof, may be used to treat breast cancer. A breast cancer that is to be treated can include familial breast cancer. A breast cancer that is to be treated can include sporadic breast cancer. A breast cancer that is to be treated can arise in a male subject. A breast cancer that is to be treated can arise in a female subject. A breast cancer that is to be treated can arise in a premenopausal female subject or a postmenopausal female subject. A breast cancer that is to be treated can arise in a subject equal to or older than 30 years old, or a subject younger than 30 years old. A breast cancer that is to be treated has arisen in a subject equal to or older than 50 years old, or a subject younger than 50 years old. A breast cancer that is to be treated can arise in a subject equal to or older than 70 years old, or a subject younger than 70 years old.

A breast cancer that is to be treated can be typed to identify a familial or spontaneous mutation in BRCA1, BRCA2, or p53. A breast cancer that is to be treated can be typed as having a HER2/neu gene amplification, as overexpressing HER2/neu, or as having a low, intermediate or high level of HER2/neu expression. A breast cancer that is to be treated can be typed for a marker selected from the group consisting of estrogen receptor (ER), progesterone receptor (PR), human epidermal growth factor receptor-2, Ki-67, CA15-3, CA 27-29, and c-Met. A breast cancer that is to be treated can be typed as ER-unknown, ER-rich or ER-poor. A breast cancer that is to be treated can be typed as ER-negative or ER-positive. ER-typing of a breast cancer may be performed by any reproducible means. ER-typing of a breast cancer may be performed as set forth in Onkologie 27: 175-179 (2004). A breast cancer that is to be treated can be typed as PR-unknown, PR-rich, or PR-poor. A breast cancer that is to be treated can be typed as PR-negative or PR-positive. A breast cancer that is to be treated can be typed as receptor positive or receptor negative. A breast cancer that is to be treated can be typed as being associated with elevated blood levels of CA 15-3, or CA 27-29, or both.

A breast cancer that is to be treated can include a localized tumor of the breast. A breast cancer that is to be treated can include a tumor of the breast that is associated with a negative sentinel lymph node (SLN) biopsy. A breast cancer that is to be treated can include a tumor of the breast that is associated with a positive sentinel lymph node (SLN) biopsy. A breast cancer that is to be treated can include a tumor of the breast that is associated with one or more positive axillary lymph nodes, where the axillary lymph nodes have been staged by any applicable method. A breast cancer that is to be treated can include a tumor of the breast that has been typed as having nodal negative status (e.g., node-negative) or nodal positive status (e.g., node-positive). A breast cancer that is to be treated can include a tumor of the breast that has metastasized to other locations in the body. A breast cancer that is to be treated can be classified as having metastasized to a location selected from the group consisting of bone, lung, liver, or brain. A breast cancer that is to be treated can be classified according to a characteristic selected from the group consisting of metastatic, localized, regional, local-regional, locally advanced, distant, multicentric, bilateral, ipsilateral, contralateral, newly diagnosed, recurrent, and inoperable.

A compound of the disclosure, or a pharmaceutically acceptable salt or solvate thereof, may be used to treat or prevent a cell proliferative disorder of the breast, or to treat or prevent breast cancer, in a subject having an increased risk of developing breast cancer relative to the population at large. A subject with an increased risk of developing breast cancer relative to the population at large is a female subject with a family history or personal history of breast cancer. A subject with an increased risk of developing breast cancer relative to the population at large is a female subject having a germ-line or spontaneous mutation in BRCA1 or BRCA2, or both. A subject with an increased risk of developing breast cancer relative to the population at large is a female subject with a family history of breast cancer and a germ-line or spontaneous mutation in BRCA1 or BRCA2, or both. A subject with an increased risk of developing breast cancer relative to the population at large is a female who is greater than 30 years old, greater than 40 years old, greater than 50 years old, greater than 60 years old, greater than 70 years old, greater than 80 years old, or greater than 90 years old. A subject with an increased risk of developing breast cancer relative to the population at large is a subject with atypical hyperplasia of the breast, ductal carcinoma in situ (DCIS), intraductal carcinoma, lobular carcinoma in situ (LCIS), lobular neoplasia, or a stage 0 growth or lesion of the breast (e.g., stage 0 or grade 0 breast cancer, or carcinoma in situ).

A breast cancer that is to be treated can histologically graded according to the Scarff-Bloom-Richardson system, wherein a breast tumor has been assigned a mitosis count score of 1, 2, or 3; a nuclear pleiomorphism score of 1, 2, or 3; a tubule formation score of 1, 2, or 3; and a total Scarff-Bloom-Richardson score of between 3 and 9. A breast cancer that is to be treated can be assigned a tumor grade according to the International Consensus Panel on the Treatment of Breast Cancer selected from the group consisting of grade 1, grade 1-2, grade 2, grade 2-3, or grade 3.

A cancer that is to be treated can be staged according to the American Joint Committee on Cancer (AJCC) TNM classification system, where the tumor (T) has been assigned a stage of TX, Ti, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol−), PN0 (mol+), PN1, PN1(mi), PN1a, PN1b, PN1c, pN2, pN2a, pN2b, pN3, pN3a, pN3b, or pN3c.

A cancer that is to be treated can include a tumor that has been determined to be less than or equal to about 2 centimeters in diameter. A cancer that is to be treated can include a tumor that has been determined to be from about 2 to about 5 centimeters in diameter. A cancer that is to be treated can include a tumor that has been determined to be greater than or equal to about 3 centimeters in diameter. A cancer that is to be treated can include a tumor that has been determined to be greater than 5 centimeters in diameter. A cancer that is to be treated can be classified by microscopic appearance as well differentiated, moderately differentiated, poorly differentiated, or undifferentiated. A cancer that is to be treated can be classified by microscopic appearance with respect to mitosis count (e.g., amount of cell division) or nuclear pleiomorphism (e.g., change in cells). A cancer that is to be treated can be classified by microscopic appearance as being associated with areas of necrosis (e.g., areas of dying or degenerating cells). A cancer that is to be treated can be classified as having an abnormal karyotype, having an abnormal number of chromosomes, or having one or more chromosomes that are abnormal in appearance. A cancer that is to be treated can be classified as being aneuploid, triploid, tetraploid, or as having an altered ploidy. A cancer that is to be treated can be classified as having a chromosomal translocation, or a deletion or duplication of an entire chromosome, or a region of deletion, duplication or amplification of a portion of a chromosome.

In some embodiments, a cancer that is to be treated is a cancer in which a member of the SWI/SNF complex, e.g., SMARCA4, is mutated, deleted and/or exhibits a loss of function (e.g., a decrease of enzymatic activity). For example, a cancer to be treated may be a cancer in which SMARCA4 is mutated. Non limiting examples of cancers in which SMARCA4 mutations occur include small cell carcinoma of the ovary of the hypercalcemic type (SCCOHT), bladder cancer, stomach cancer, lung cancer (e.g., non-small cell lung cancer), glioblastoma brain tumors (glioma, GBM), head and neck cancer, kidney cancer, uterine cancer, cervical cancer, and pancreatic cancer.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer can result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the disclosure, or a pharmaceutically acceptable salt, solvate, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the disclosure, or a pharmaceutically acceptable salt, solvate, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

As used herein, the term “selectively” means tending to occur at a higher frequency in one population than in another population. The compared populations can be cell populations. Preferably, a compound of the disclosure, or a pharmaceutically acceptable salt or solvate thereof, acts selectively on a cancer or precancerous cell but not on a normal cell. Preferably, a compound of the disclosure, or a pharmaceutically acceptable salt or solvate thereof, acts selectively to modulate one molecular target (e.g., a target helicase, such as SMARCA2) but does not significantly modulate another molecular target (e.g., a different helicase, or a non-helicase enzyme, e.g., in the case of a SMARCA2 ATPase inhibitor, the ATPase activity of a different helicase, or a different protein having ATPase activity). A composition of the disclosure, e.g., a composition comprising SMARCA2 inhibitor, and one or more other therapeutic agents, such as prednisone, can modulate the activity of a molecular target (e.g., a target helicase). Modulating refers to stimulating or inhibiting an activity of a molecular target. Preferably, a compound of the disclosure, or a pharmaceutically acceptable salt or solvate thereof, modulates the activity of a molecular target if it stimulates or inhibits the activity of the molecular target by at least 2-fold relative to the activity of the molecular target under the same conditions but lacking only the presence of said compound. More preferably, a compound of the disclosure, or a pharmaceutically acceptable salt or solvate thereof, modulates the activity of a molecular target if it stimulates or inhibits the activity of the molecular target by at least 5-fold, at least 10-fold, at least 20-fold, at least 50-fold, at least 100-fold relative to the activity of the molecular target under the same conditions but lacking only the presence of said compound. The activity of a molecular target may be measured by any reproducible means. The activity of a molecular target may be measured in vitro or in vivo. For example, the activity of a molecular target may be measured in vitro by an enzymatic activity assay or a DNA binding assay, or the activity of a molecular target may be measured in vivo by assaying for expression of a reporter gene.

A composition of the disclosure, e.g., a composition comprising SMARCA2 inhibitor, and one or more other therapeutic agents, such as prednisone, can modulate the activity of a molecular target (e.g., a target helicase). Modulating refers to stimulating or inhibiting an activity of a molecular target. Preferably, a compound of the disclosure, or a pharmaceutically acceptable salt or solvate thereof, modulates the activity of a molecular target if it stimulates or inhibits the activity of the molecular target by at least 2-fold relative to the activity of the molecular target under the same conditions but lacking only the presence of said compound. More preferably, a compound of the disclosure, or a pharmaceutically acceptable salt or solvate thereof, modulates the activity of a molecular target if it stimulates or inhibits the activity of the molecular target by at least 5-fold, at least 10-fold, at least 20-fold, at least 50-fold, at least 100-fold relative to the activity of the molecular target under the same conditions but lacking only the presence of said compound. The activity of a molecular target may be measured by any reproducible means. The activity of a molecular target may be measured in vitro or in vivo. For example, the activity of a molecular target may be measured in vitro by an enzymatic activity assay or a DNA binding assay, or the activity of a molecular target may be measured in vivo by assaying for expression of a reporter gene.

A composition of the disclosure does not significantly modulate the activity of a molecular target if the addition of the compound does not stimulate or inhibit the activity of the molecular target by greater than 10% relative to the activity of the molecular target under the same conditions but lacking only the presence of said compound.

Administering a composition of the disclosure to a cell or a subject in need thereof can result in modulation (i.e., stimulation or inhibition) of an activity of a helicase of interest.

Administering a compound of the disclosure, e.g., a composition comprising aSMARCA2 inhibitor, and one or more other therapeutic agents, such as prednisone, to a cell or a subject in need thereof results in modulation (i.e., stimulation or inhibition) of an activity of an intracellular target (e.g., substrate). Several intracellular targets can be modulated with the compounds of the disclosure, including, but not limited to, helicases.

Activating refers to placing a composition of matter (e.g., protein or nucleic acid) in a state suitable for carrying out a desired biological function. A composition of matter capable of being activated also has an unactivated state. An activated composition of matter may have an inhibitory or stimulatory biological function, or both.

Elevation refers to an increase in a desired biological activity of a composition of matter (e.g., a protein or a nucleic acid). Elevation may occur through an increase in concentration of a composition of matter.

As used herein, “a cell cycle checkpoint pathway” refers to a biochemical pathway that is involved in modulation of a cell cycle checkpoint. A cell cycle checkpoint pathway may have stimulatory or inhibitory effects, or both, on one or more functions comprising a cell cycle checkpoint. A cell cycle checkpoint pathway is comprised of at least two compositions of matter, preferably proteins, both of which contribute to modulation of a cell cycle checkpoint. A cell cycle checkpoint pathway may be activated through an activation of one or more members of the cell cycle checkpoint pathway. Preferably, a cell cycle checkpoint pathway is a biochemical signaling pathway.

As used herein, “cell cycle checkpoint regulator” refers to a composition of matter that can function, at least in part, in modulation of a cell cycle checkpoint. A cell cycle checkpoint regulator may have stimulatory or inhibitory effects, or both, on one or more functions comprising a cell cycle checkpoint. A cell cycle checkpoint regulator can be a protein or not a protein.

Treating cancer or a cell proliferative disorder can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100(5): 2674-8, 2003. In some aspects, cell death occurs by apoptosis.

Preferably, an effective amount of a composition of the disclosure, or a pharmaceutically acceptable salt or solvate thereof, is not significantly cytotoxic to normal cells. A therapeutically effective amount of a compound is not significantly cytotoxic to normal cells if administration of the compound in a therapeutically effective amount does not induce cell death in greater than 10% of normal cells. A therapeutically effective amount of a compound does not significantly affect the viability of normal cells if administration of the compound in a therapeutically effective amount does not induce cell death in greater than 10% of normal cells. In some aspects, cell death occurs by apoptosis.

Contacting a cell with a composition of the disclosure, or a pharmaceutically acceptable salt or solvate thereof, can induce or activate cell death selectively in cancer cells. Administering to a subject in need thereof a compound of the disclosure, or a pharmaceutically acceptable salt or solvate thereof, can induce or activate cell death selectively in cancer cells. Contacting a cell with a composition of the disclosure, or a pharmaceutically acceptable salt or solvate thereof, can induce cell death selectively in one or more cells affected by a cell proliferative disorder. Preferably, administering to a subject in need thereof a composition of the disclosure, or a pharmaceutically acceptable salt or solvate thereof, induces cell death selectively in one or more cells affected by a cell proliferative disorder.

The disclosure relates to a method of treating or preventing cancer by administering a composition of the disclosure, or a pharmaceutically acceptable salt or solvate thereof, to a subject in need thereof, where administration of the composition of the disclosure, or a pharmaceutically acceptable salt or solvate thereof, results in one or more of the following: prevention of cancer cell proliferation by accumulation of cells in one or more phases of the cell cycle (e.g. G1, G1/S, G2/M), or induction of cell senescence, or promotion of tumor cell differentiation; promotion of cell death in cancer cells via cytotoxicity, necrosis or apoptosis, without a significant amount of cell death in normal cells, antitumor activity in animals with a therapeutic index of at least 2. As used herein, “therapeutic index” is the maximum tolerated dose divided by the efficacious dose.

The present disclosure provides methods for the synthesis of the compounds of any of the Formulae described herein. The present disclosure also provides detailed methods for the synthesis of various disclosed compounds of the present disclosure according to the following schemes as well as those shown in the Examples.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

The synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

Compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art. The following descriptions of synthetic methods are designed to illustrate, but not to limit, general procedures for the preparation of compounds of the present disclosure.

Compounds of the present disclosure can be conveniently prepared by a variety of methods familiar to those skilled in the art. The compounds of this disclosure having any of the Formulae described herein may be prepared according to the procedures illustrated in Schemes 1-6 below, from commercially available starting materials or starting materials which can be prepared using literature procedures. Certain variables (such as R₁, R₂, R₅ and A) in Schemes 1-6 are as defined in any Formula described herein, unless otherwise specified.

One of ordinary skill in the art will note that, during the reaction sequences and synthetic schemes described herein, the order of certain steps may be changed, such as the introduction and removal of protecting groups.

One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

Preferred protecting groups include, but are not limited to:

For a hydroxyl moiety: TBS, benzyl, THP, Ac

For carboxylic acids: benzyl ester, methyl ester, ethyl ester, allyl ester

For amines: Cbz, BOC, DMB

For diols: Ac (×2) TBS (×2), or when taken together acetonides

For thiols: Ac

For benzimidazoles: SEM, benzyl, PMB, DMB

For aldehydes: di-alkyl acetals such as dimethoxy acetal or diethyl acetyl.

In the reaction schemes described herein, multiple stereoisomers may be produced. When no particular stereoisomer is indicated, it is understood to mean all possible stereoisomers that could be produced from the reaction. A person of ordinary skill in the art will recognize that the reactions can be optimized to give one isomer preferentially, or new schemes may be devised to produce a single isomer. If mixtures are produced, techniques such as preparative thin layer chromatography, preparative HPLC, preparative chiral HPLC, or preparative SFC may be used to separate the isomers.

The following abbreviations are used throughout the specification and are defined below:
ACN acetonitrile
Ac acetyl
AcOH acetic acid
AlCl₃ aluminum chloride
BINAP (2,2′-bis(diphenylphosphino)-1,1′-binaphthyl)
t-BuOK potassium t-butoxide
tBuONa or t-BuONa sodium t-butoxide
br broad
BOC tert-butoxy carbonyl
Cbz benzyloxy carbonyl
CDCl₃CHCl₃ chloroform
CH₂Cl₂ dichloromethane
CH₃CN acetonitrile
CsCO₃ cesium carbonate
CH₃NO₃ nitromethane
d doublet
dd doublet of doublets
dq doublet of quartets
DCE 1,2 dichloroethane
DCM dichloromethane
Δ heat
δ chemical shift
DIEA N,N-diisopropylethylamine (Hunig's base)
DMB 2,4 dimethoxy benzyl
DMF N,N-Dimethylformamide
DMSO Dimethyl sulfoxide
DMSO-d6 deuterated dimethyl sulfoxide
EA or EtOAc Ethyl acetate
ES electrospray
Et₃N triethylamine
equiv equivalents
g grams
g relative centrifugal force (RCF) expressed in units of gravity
h hours
HATU Hexafluorophosphate azabenzotriazole tetramethyl uronium (1-[Bis(dimethylamino)methylene]-1H-1,2,3-triazolo[4,5-b]pyridinium 3-oxid hexafluorophosphate)
H₂O water
HCl hydrogen chloride or hydrochloric acid
HPLC High performance liquid chromatography
Hz Hertz
IPA isopropyl alcohol
i-PrOH isopropyl alcohol
J NMR coupling constant
K₂CO₃ potassium carbonate
HI potassium iodide
KCN potassium cyanide
LCMS or LC-MS Liquid chromatography mass spectrum
M molar
m multiplet
mg milligram
MHz megahertz
mL milliliter
mm millimeter
mmol millimole
mol mole
[M+1] molecular ion plus one mass unit
m/z mass/charge ratio
m-CPBA meta-chloroperbenzoic acid
MeCN Acetonitrile
MeOH methanol
Mel Methyl iodide
min minutes
μm micron
MsCl Mesyl chloride
MW microwave irradiation
N normal
Na₂SO₄ sodium sulfate
NH₃ ammonia
NaBH(AcO)₃ sodium triacetoxyborohydride
NaI sodium iodide
Na₂SO₄ sodium sulfate
NH₄C₁ ammonium chloride
NH₄HCO₃ ammonium bicarbonate
nm nanometer
NBS N-bromosuccinimide
NMP N-methylpyrrolidinone
NMR Nuclear Magnetic Resonance
Pd(OAc)₂ palladium (II) acetate
Pd/C Palladium on carbon
Pd₂(dba)₃ Tris(dibenzylideneacetone)dipalladium(0)
PMB para methoxybenzyl
ppm parts per million
POCl₃ phosphoryl chloride
prep-HPLC preparative High Performance Liquid Chromatography
PTSA para-toluenesulfonic acid
p-TsOH para-toluenesulfonic acid
RT retention time
rt room temperature
s singlet
t triplet
t-BuXPhos 2-Di-tert-butylphosphino-2′,4′,6′-triisopropylbiphenyl
TEA Triethylamine
TFA trifluoroacetic acid
TfO triflate
THP tetrahydropyran
TsOH tosic acid
UV ultraviolet

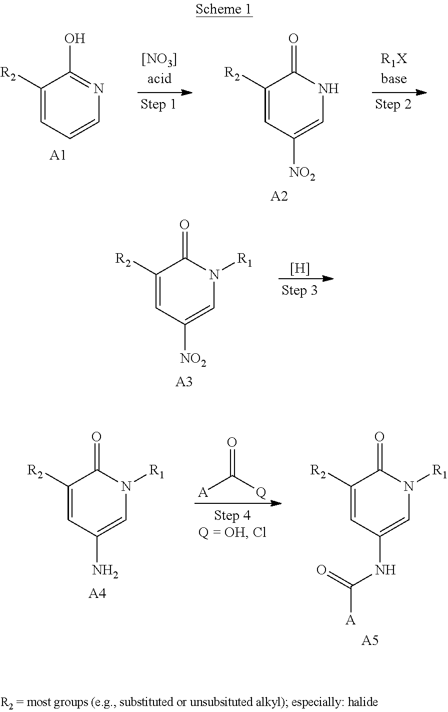

Scheme 1 shows a synthesis of the pyridone-carboxamide portion of the compounds disclosed herein following a general route. A pyridin-2-ol (A1) is converted to a 5-nitropyridin-2(1H)-one (A2) under standard nitration conditions, e.g., using a mixture of nitric acid (HNO₃) and sulfuric acid (H₂SO₄), followed by alkylation in the presence of a base, (e.g., NaH, DMF) to give N-alkylated 5-nitropyridin-2(1H)-one (A3). A3 is reduced to a 5-amino-pyridin-2(1H)-one (A4) using standard reduction reagents (e.g., Fe/NH₄Cl/MeOH—H₂O). Amide coupling, using e.g., a carbonyl chloride (Q=Cl) in the presence of a tertiary amine base (e.g. triethylamine, TEA; N,N-diisopropylethylamine, DIEA), or a carboxylic acid (Q=OH) in the presence of a coupling reagent (e.g., hexafluorophosphate azabenzotriazole tetramethyl uranium, HATU) yields the compound (A5).

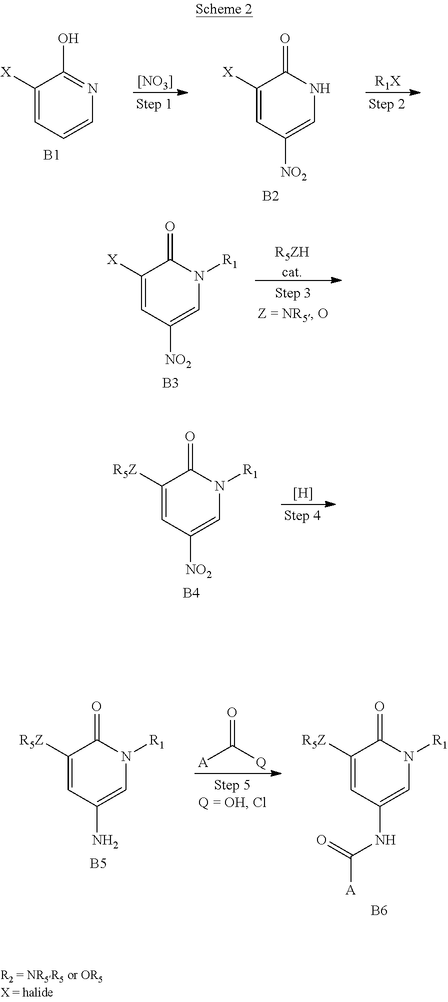

Scheme 2 shows a synthesis of the pyridone-carboxamide portion of the compounds when R₂ is NR_{5′}R₅ or OR₅. The 5-nitropyridin-2(1H)-one B3 is obtained following step 1 and step 2 as described in Scheme 1. Reaction of B3 with an amine in the presence of a catalyst (e.g., Pd(OAc)₂/Xantphos/Cs₂CO₃/dioxane) or with alcohol (R₅ZH) in the presence of a catalyst (e.g., CuI/t-BuOLi) yields intermediate B4. Step 4 and step 5 are the same as step 3 and step 4 in Scheme 1.

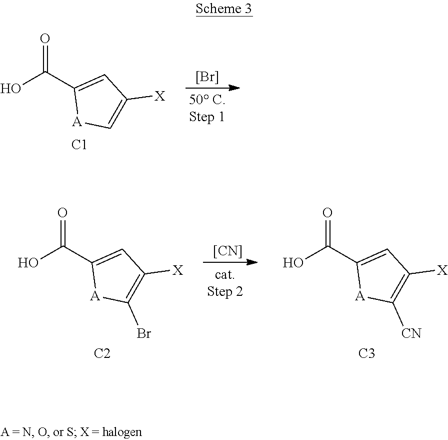

Scheme 3 shows an exemplary synthesis of a A-COOH intermediate containing a cyano group following a general route. For example, a 4-haloheteroaryl-2-carboxylic acid (C1) is brominated using a bromination reagent (e.g. N-bromosuccinimide, NBS) in a suitable solvent (e.g. dimethylformamide, DMF). The resulting 5-bromo-4-haloheteroaryl-2-carboxylic acid (C2) is reacted with zinc cyanide (Zn(CN)₂) using a coupling catalyst (e.g., Pd(PPh₃)₄) in an appropriate solvent (e.g., DMF) to give a 5-cyano-4-haloheteroaryl-2-carboxylic acid (C3).

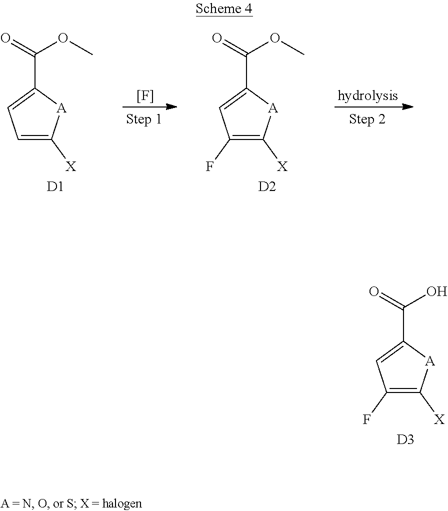

Scheme 4 shows a synthesis of a A-COOH intermediate following a general route. For example, 5-haloheteroaryl-2-carboxylate (D1) is fluorinated using a fluorination reagent (e.g. 1-chloromethyl-4-fluoro-1,4-diazoniabicyclo[2.2.2]octane bis(tetrafluoroborate), Selectfluor™) in a suitable solvent (e.g. CH₃CN). The resulting 5-halo-4-fluoroheteroaryl-2-carboxylate (D2) is hydrolized to 5-halo-4-fluoroheteroaryl-2-carboxylic acid (D3) using suitable reagent (e.g. lithium hydroxide) in an appropriate solvent (e.g., THF/H₂O).

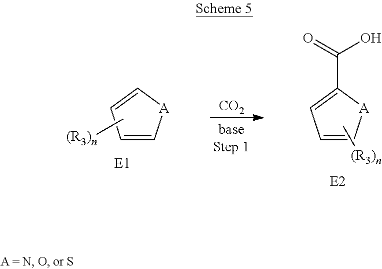

Scheme 5 shows a synthesis of a A-COOH intermediate following a general route. For example, a heteroaryl (E1) is carboxylated using CO₂ and a suitable base (e.g., lithium diisopropylamide, LDA) to yield the heteroaryl carboxylic acid (E2).

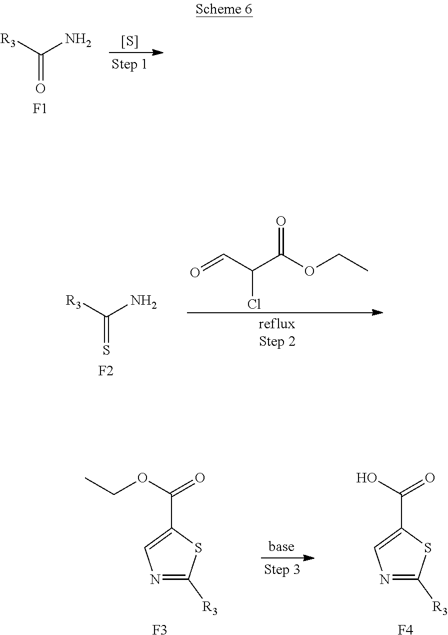

Scheme 6 shows a synthesis of a A-COOH intermediate when A is thiazole. For example, an amide (F1) is converted to a thioamide (F2) using an appropriate thionation reagent (e.g., P₂S₅). F2 is then reacted with ethyl 2-chloro-3-oxopropanoate in an appropriate solvent (e.g. tert-butanol) to yield a ethyl thiazole-5-carboxylate (F3). F3 is hydrolized to give a thiazole-5-carboxylic acid (E4) using suitable base (e.g. sodium hydroxide) in an appropriate solvent (e.g., ethanol).

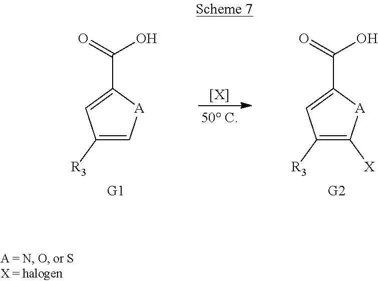

Scheme 7 shows a method for introducing a halogen substituent at the A-COOH intermediate. For example a heteroaryl-2-carboxylic acid (G1) is halogenated using an appropriate agent (e.g. N-chlorosuccinimide, NCS) in an appropriate solvent (e.g. dimethylformamide, DMF) to give a 5-halo-heteroaryl-2-carboxylic acid (G2).

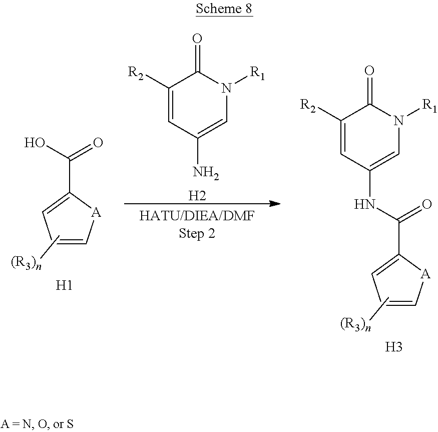

Scheme 8 shows a method for coupling the A-COOH intermediate to the pyridone carboxamide portion of the compounds herein, following a general route. For example, a heteroaryl-2-carboxylic acid (H1) is reacted with a 5-aminopyridin-2(1H)-one (H2) in the presence of a coupling reagent (e.g., hexafluorophosphate azabenzotriazole tetramethyl uranium, HATU) and an appropriate base (e.g. triethylamine, TEA; N,N-diisopropylethylamine, DIEA) in an appropriate solvent (e.g. dimethylformamide, DMF) to give the desired compound (H3).

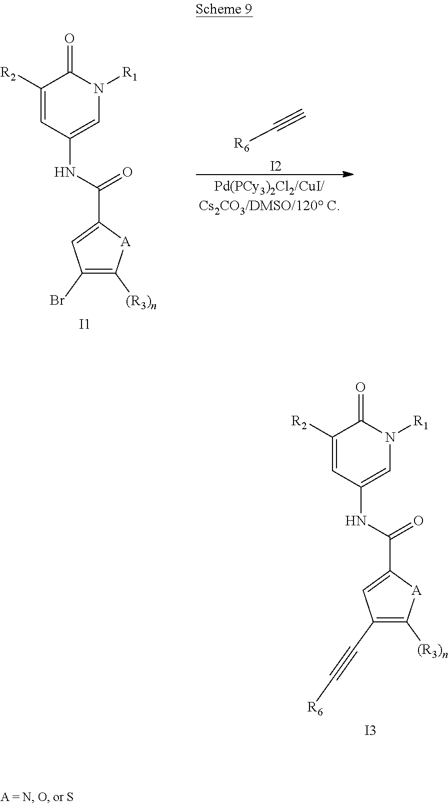

Scheme 9 shows a method for attaching an alkynyl linked group to the compounds herein. For example, a 4-bromo-N-(6-oxo-1,6-dihydropyridin-3-yl)heteroaryl-2-carboxamide (I1) is reacted with an ethynyl compound (I2) via a standard cross-coupling reaction (e.g., Sonogashira coupling) using appropriate catalysts (e.g., a palladium catalyst, e.g., dichlorobis(tricyclohexylphosphine)palladium and a copper catalyst, e.g., CuI) in the presence of a base (e.g., caesium carbonate) in an appropriate solvent (e.g., dimethyl sulfoxide, DMSO) (I3).

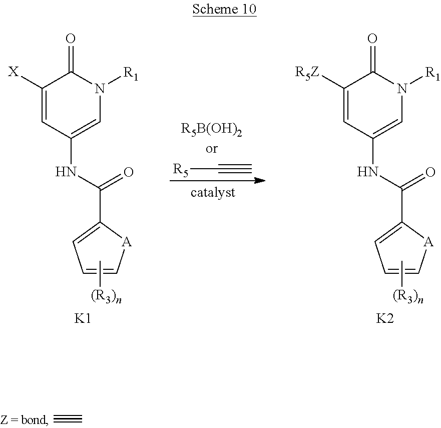

Scheme 10 shows a method for attaching an aryl or alkynyl linked group to the compounds herein. For example, a N-(5-halo-6-oxo-1,6-dihydropyridin-3-yl)heteroaryl-2-carboxamide (K1) is reacted with an alkynyl compound or aryl boronate via a standard cross-coupling reaction (e.g., Sonogashira or Suzuki coupling) using appropriate catalysts (e.g., a palladium catalyst, e.g., dichlorobis(tricyclohexylphosphine)palladium and a copper catalyst, e.g., CuI) in the presence of a base (e.g., caesium carbonate) in an appropriate solvent (e.g., dimethyl sulfoxide, DMSO) to give the desired compound (K2).

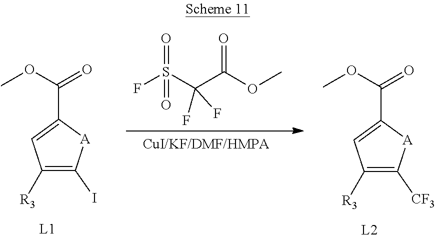

Scheme 11 shows a method for attaching a trifluoromethyl group to the compounds herein. For example, a 5-iodoheteroaryl-2-carboxylate (Li) is reacted with a trifluoromethylating agent (e.g., methyl 2,2-difluoro-2-(fluorodimethylidene-lambda6-sulfanyl)acetate) using appropriate catalysts (e.g., a copper catalyst, e.g., CuI) in an appropriate solvent (e.g., DMF/HMPA) to give the desired compound (L2).

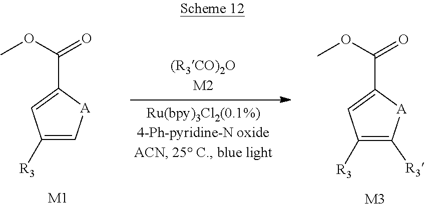

Scheme 11 shows a method for attaching a substituted alkyl group to the compounds herein. For example, a heteroaryl-2-carboxylate (M1) is reacted with an anhydride (M2) using appropriate catalysts (e.g., a ruthenium catalyst, e.g., tris(bipyridine)ruthenium(II) chloride) and an N-oxide (e.g., 4-phenylpyridine N-oxide) and blue light in an appropriate solvent (e.g., acetonitrile, ACN) to give the desired compound (M3).

Example 1: The compounds listed in Table 2, 2a, 2b, 2c, and 2d were synthesized by reaction schemes depicted in the general schemes above or by methods described below.

### Synthesis of Compound 82c: 4-cyano-N-[1-(2,2-difluoroethyl)-5-fluoro-6-oxopyridin-3-yl]-5-(trifluoromethyl)thiophene-2-carboxamide

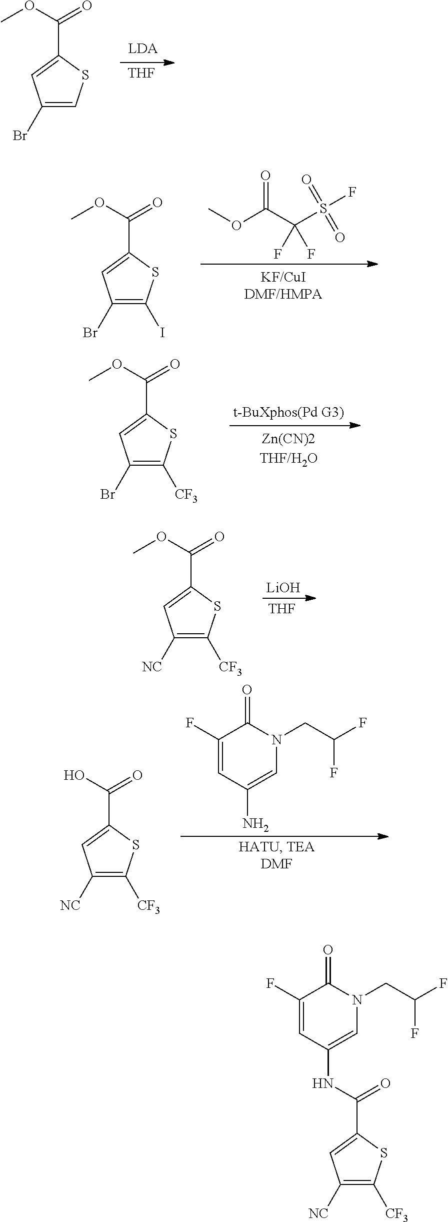

### Step 1: Synthesis of methyl 4-bromo-5-iodothiophene-2-carboxylate

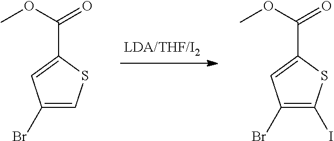

Into a 100-mL 3-necked round-bottom flask purged and maintained with an inert atmosphere of nitrogen, was placed THF (20.00 mL), methyl 4-bromothiophene-2-carboxylate (6.00 g, 27.14 mmol, 1.00 equiv), LDA (2 mol/L) (30.05 mL, 60.10 mmol, 2.21 equiv) was added by dropwise at −78° C. after 40 min at this temperature, 12 (7.00 g, 27.58 mmol, 1.02 equiv) was added by dropwise in THF (5 mL). The resulting solution was stirred for 2 hr at −78° C. in a liquid nitrogen bath. The reaction progress was monitored by GCMS. The reaction was quenched by 5 mL of H₂O, The resulting mixture was concentrated. And the residue was dissolved by ethyl acetate (40 mL), washed by 20% NaHSO₃ aqueous (3×15 ml) to remove excess iodine. The mixture was dried over anhydrous sodium sulfate and concentrated under vacuum. The residue was applied onto a silica gel column with ethyl acetate/petroleum ether (1/10). This resulted in 6.4 g (68%) of methyl 4-bromo-5-iodothiophene-2-carboxylate as a yellow solid. ¹H NMR (300 MHz, DMSO-d₆.ppm) δ 7.69 (s, 1H), 3.83 (s, 3H).

### Step 2: Synthesis of methyl 4-bromo-5-(trifluoromethyl)thiophene-2-carboxylate

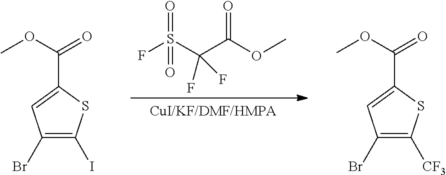

Into a 100-mL vial purged and maintained with an inert atmosphere of nitrogen, was placed DMF (9.00 mL), HMPA (9.00 mL), methyl 4-bromo-5-iodothiophene-2-carboxylate (2.00 g, 5.76 mmol, 1.00 equiv), methyl 2,2-difluoro-2-(fluorodimethylidene-lambda6-sulfanyl)acetate (2.36 g, 12.54 mmol, 2.18 equiv), CuI (570.00 mg, 2.99 mmol, 0.52 equiv), KF (1.00 g, 17.21 mmol, 2.99 equiv). The resulting solution was stirred for 2 hr at 70° C. in an oil bath. The reaction progress was monitored by GCMS. The reaction was quenched by 30 mL of H₂O, The resulting solution was extracted with 3×30 mL of ethyl acetate and the organic layers combined. The resulting mixture was washed with 4×10 mL of saturated sodium chloride. The mixture was dried over anhydrous sodium sulfate and concentrated under vacuum. The residue was applied onto a silica gel column with ethyl acetate/petroleum ether (1/10). This resulted in 1.4 g (90%) of methyl 4-bromo-5-(trifluoromethyl)thiophene-2-carboxylate as yellow oil. ¹H NMR (300 MHz, DMSO-d₆, ppm) δ 7.77 (s, 1H), 3.83 (s, 3H)

### Step 3: Synthesis of methyl 4-cyano-5-(trifluoromethyl)thiophene-2-carboxylate

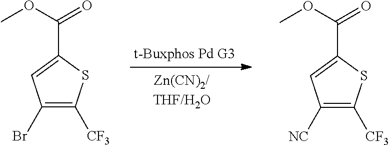

Into a 100-mL vial purged and maintained with an inert atmosphere of nitrogen, was placed THE (5.00 mL), H₂O (5.00 mL), methyl 4-bromo-5-(trifluoromethyl)thiophene-2-carboxylate (1.00 g, 3.45 mmol, 1.00 equiv), Zn(CN)₂ (1.20 g, 10.56 mmol, 3.00 equiv), t-Buxphos Pd G3 (562.00 mg, 0.70 mmol, 0.20 equiv). The resulting solution was stirred for 2 hr at 80° C. in an oil bath. The reaction progress was monitored by GCMS. The resulting mixture was concentrated. The residue was applied onto a silica gel column with ethyl acetate/petroleum ether (1/5). This resulted in 800 mg (98%) of methyl 4-cyano-5-(trifluoromethyl)thiophene-2-carboxylate as a yellow solid. ¹H NMR (300 MHz, Methanol-d₄, ppm) δ 8.12 (s, 1H), 3.84 (s, 3H).

### Step 4: Synthesis of 4-cyano-5-(trifluoromethyl)thiophene-2-carboxylic acid

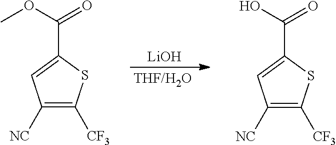

Into a 100-mL 3-necked round-bottom flask, was placed THE (5.00 mL), H₂O (1.00 mL), methyl 4-cyano-5-(trifluoromethyl)thiophene-2-carboxylate (1.00 g, 4.25 mmol, 1.00 equiv), lithium hydroxide (140.00 mg, 5.84 mmol, 1.37 equiv). The resulting solution was stirred for 2 hr at 0° C. in a water/ice bath. The reaction progress was monitored by LCMS. The resulting mixture was concentrated. The pH was adjusted to 5-6 with HCl (1 mol/L). The resulting solution was extracted with 3×30 mL of ethyl acetate and the organic layers combined. The mixture was dried over anhydrous sodium sulfate and concentrated under vacuum. The residue was applied onto a silica gel column with ethyl acetate/petroleum ether (1/5). This resulted in 600 mg (63%) of 4-cyano-5-(trifluoromethyl)thiophene-2-carboxylic acid as a yellow solid. LCMS (ESI): RT=0.71 min, m z=220 [M−H]⁻;

### Step 5: Synthesis of 4-cyano-N-[1-(2,2-difluoroethyl)-5-fluoro-6-oxopyridin-3-yl]-5-(trifluoromethyl)thiophene-2-carboxamide

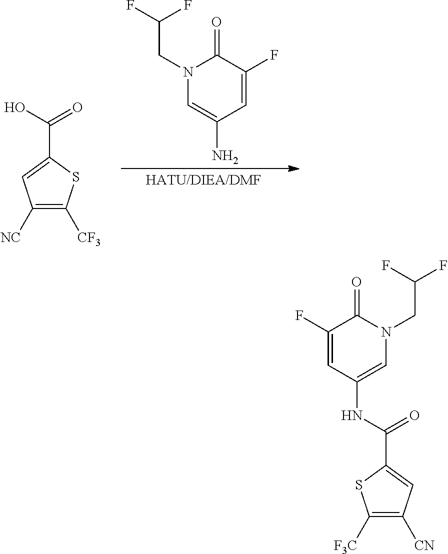

Into a 100-mL round-bottom flask, was placed DMF (5.00 mL), 4-cyano-5-(trifluoromethyl)thiophene-2-carboxylic acid (464.00 mg, 2.09 mmol, 1.00 equiv), 5-amino-1-(2,2-difluoroethyl)-3-fluoropyridin-2-one (490.00 mg, 2.55 mmol, 1.22 equiv), DIEA (812.00 mg, 6.28 mmol, 2.99 equiv), HATU (957.00 mg, 2.51 mmol, 1.20 equiv), The resulting solution was stirred for 2 hr at 25° C. The reaction progress was monitored by LCMS, The reaction was quenched by 20 mL of H₂O, The resulting solution was extracted with 3×15 mL of ethyl acetate and the organic layers combined. The resulting mixture was washed with 3×10 mL of saturated sodium chloride. The mixture was dried over anhydrous sodium sulfate and concentrated under vacuum. The crude residue was purified by C18 column with acetonitrile/water (40%). This resulted in 233.4 mg (28%) of 4-cyano-N-[1-(2,2-difluoroethyl)-5-fluoro-6-oxopyridin-3-yl]-5-(trifluoromethyl)thiophene-2-carboxamide as a white solid.

LCMS (ESI): RT=1.77 min, m z=396.1 [M+H]⁺; ¹H NMR (300 MHz, DMSO-d₆, ppm) δ 10.71 (s, 1H), 8.37 (s, 1H), 8.00 (d, J=18.0 Hz, 1H), 7.71 (dd, J=11.4, 2.1 Hz, 1H), 6.54-6.18 (m, 1H), 4.58 (m, 2H).

### Synthesis of Compound 125: 4-Chloro-N-(5-chloro-1-(2-fluoroethyl)-6-oxo-1,6-dihydropyridin-3-yl)-5-cyanothiophene-2-carboxamide

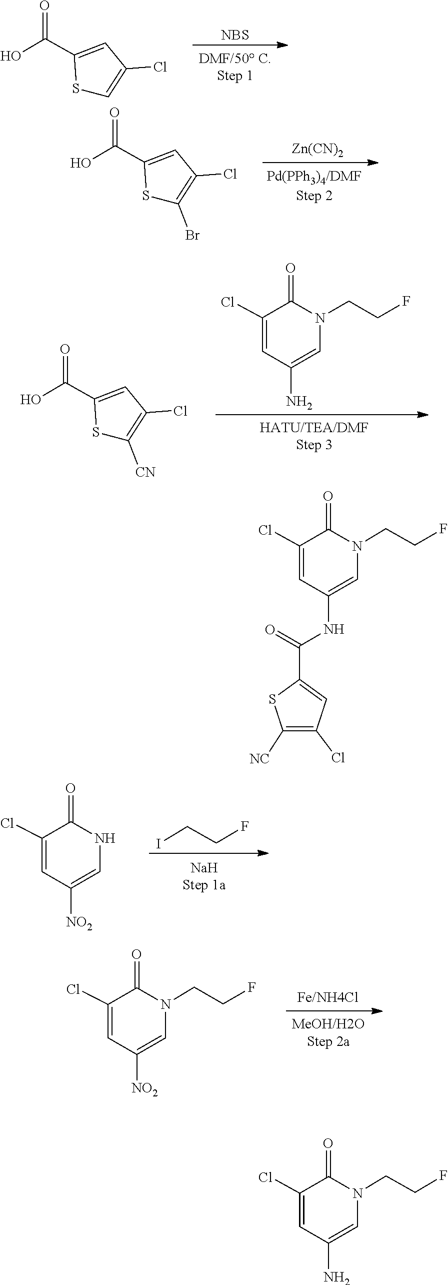

### Step 1: 5-Bromo-4-chlorothiophene-2-carboxylic acid

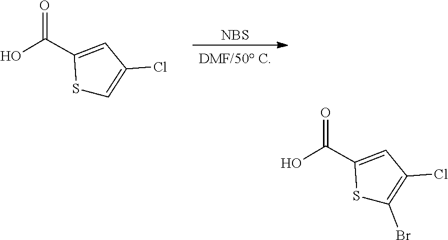 [SMILES: O=C(O)c1cc(Cl)cs1 ; "NBS" ; O=C(O)c1cc(Cl)c(Br)s1]

Into a 10-mL round-bottom flask was placed 4-chlorothiophene-2-carboxylic acid (5 g, 30.75 mmol, 1.00 equiv), NBS (10 g, 101.69 mmol, 1.80 equiv), and N,N-dimethylformamide (10 ml). The resulting solution was stirred for 12 h at 50° C. in an oil bath. The resulting mixture was poured into water and extracted by ethyl acetate (3×50 ml), the organic layer was concentrated under vacuum. The residue was applied onto a silica gel column (mobile phase: ethyl acetate/petroleum ether (1:1)), and 6 g (81% yield) of 5-bromo-4-chlorothiophene-2-carboxylic acid.

LCMS (ESI): RT=0.49 min, m/z=241[M+1]⁺; 1H NMR (300 MHz, DMSO-d6, ppm) δ 13.8 (br, 1H), 7.71 (s, 1H).

### Step 2: 4-Chloro-5-cyanothiophene-2-carboxylic acid

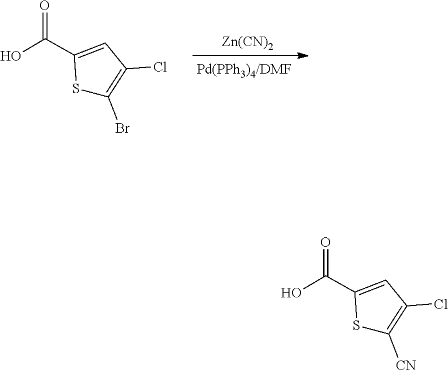

Into a 20-mL round-bottom flask was placed 5-bromo-4-chlorothiophene-2-carboxylic acid (1 g, 4.14 mmol, 1.00 equiv), Zn(CN)₂ (2.5 g, 20.6 mmol, 5.00 equiv), N,N-dimethylformamide (5 mL), and Pd(PPh₃)₄ (1.25 g, 1.03 mmol, 0.25 equiv). The resulting solution was stirred for 2 h at 80° C. in an oil bath. The reaction progress was monitored by LCMS. The resulting mixture was poured into water and extract by ethyl acetate (3×20 ml). The organic layer was concentrated under vacuum to give crude product. The residue was applied onto a silica gel column (mobile phase: ethyl acetate/petroleum ether (1:1)) to give 0.6 g (77% yield) of 4-chloro-5-cyanothiophene-2-carboxylic acid as a white solid.

LCMS (ESI): RT=0.87 min, m/z=188[M+1]⁺

### Step 1a: 3-Chloro-1-(2-fluoroethyl)-5-nitropyridin-2(1H)-one

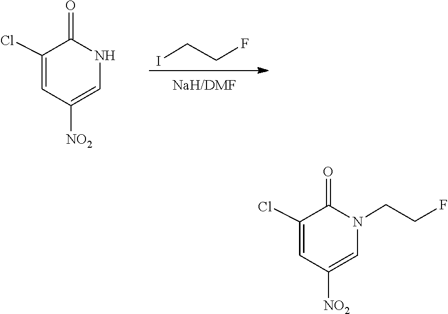

Into a 10-mL round-bottom flask was placed 3-chloro-5-nitro-1,2-dihydropyridin-2-one (5 g, 28.5 mmol, 1.00 equiv), NaH (2.2 g, 57 mmol, 2.00 equiv), 1-fluoro-2-iodoethane (10 g, 57 mmol, 2.00 equiv), and N,N-dimethylformamide (20 mL). The resulting solution was stirred for 12 h at 25° C. The reaction progress was monitored by LCMS. Then the mixture was poured into water and extracted by ethyl acetate (3×40 ml). The organic layers combined and concentrated. The residue was applied onto a silica gel column (mobile phase: ethyl acetate/petroleum ether (1:1)) to give 2 g (46% yield) of 5-amino-3-chloro-1-(2-fluoroethyl)-1,2-dihydropyridin-2-one as a white solid.

LCMS (ESI): RT=0.26 min, m/z=221[M+1]⁺ ¹H NMR (300 MHz, DMSO-d₆, ppm) δ 7.44-7.45 (m, 1H), 6.91-6.92 (m, 1H), 4.74 (t, J=4.7 Hz, 1H), 4.59 (t, J=4.6 Hz, 1H), 4.22 (t, J=4.7 Hz, 1H), 4.13 (t, J=4.7 Hz, 1H).

### Step 2a: 5-Amino-3-chloro-1-(2-fluoroethyl)pyridin-2(1H)-one

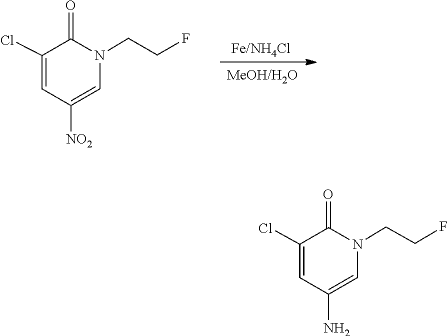 [SMILES: O=c1c(Cl)cc([N+](=O)[O-])cn1CCF ; Nc1cc(Cl)c(=O)n(CCF)c1]

Into a 10-mL round-bottom flask was placed 3-chloro-1-(2-fluoroethyl)-5-nitro-1,2-dihydropyridin-2-one (5 g, 22.67 mmol, 1.00 equiv), Fe (6.3 g, 113.35 mmol, 5.00 equiv), NH₄Cl (6.3 g, 113.35 mmol, 5.00 equiv), methanol (2 mL), and water (1 mL). The resulting solution was stirred for 12 h at 60° C. in an oil bath. The reaction progress was monitored by LCMS. The resulting mixture was cooled to room temperature, filtered, and the filtrate was concentrated under vacuum. The residue was applied onto a silica gel column (mobile phase: MeOH/DCM (1:10)). This resulted in 2 g (46% yield) of 5-amino-3-chloro-1-(2-fluoroethyl)-1,2-dihydropyridin-2-one as a white solid.

LCMS (ESI): (ES, m/z): RT=0.21 min, m/z=191[M+1]⁺; ¹H NMR (300 MHz, DMSO-d₆, ppm) δ7.34-7.35 (m, 1H), 6.81-6.82 (m, 1H), 4.86-4.87 (m, 2H), 4.71 (t, J=4.7 Hz, 1H), 4.51 (t, J=4.6 Hz, 1H), 4.16 (t, J=4.7 Hz, 1H), 4.06 (t, J=4.7 Hz, 1H).

### Step 3: 4-Chloro-N-(5-chloro-1-(2-fluoroethyl)-6-oxo-1,6-dihydropyridin-3-yl)-5-cyanothiophene-2-carboxamide (Compound 125)

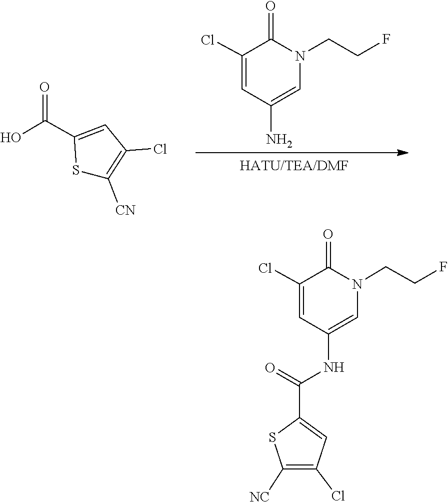 [SMILES: N#Cc1sc(C(=O)O)cc1Cl ; Nc1cc(Cl)c(=O)n(CCF)c1 ; N#Cc1sc(C(=O)Nc2cc(Cl)c(=O)n(CCF)c2)cc1Cl]

Into a 10-mL round-bottom flask was placed 4-chloro-5-cyanothiophene-2-carboxylic acid (5 g, 26.10 mmol, 1.00 equiv), 5-amino-3-chloro-1-(2-fluoroethyl)-1,2-dihydropyridin-2-one (4.98 g, 26.10 mmol, 1.00 equiv), HATU (11.8 g, 31.31 mmol, 1.20 equiv), DIEA (16.8 g, 130.5 mmol, 5.00 equiv), and N,N-dimethylformamide (10 mL). The resulting solution was stirred for 2 h at 25° C. The reaction progress was monitored by LCMS. The resulting solution was extracted with 5×50 mL of ethyl acetate. The organic layer was concentrated under vacuum and the crude product 2.5 g (89%) was purified by Flash-Prep-HPLC with the following conditions (IntelFlash-1): Column, C18 silica gel; mobile phase, CH₃CN/H₂O=20.0% increasing to CH₃CN/H₂O (0.05%)=50.0% within 10 min; Detector, UV 254 nm. This resulted in 695 mg (26% yield) of 4-chloro-N-[5-chloro-1-(2-fluoroethyl)-6-oxo-1,6-dihydropyridin-3-yl]-5-cyanothiophene-2-carboxamide as a white solid.

LCMS (ESI): RT=0.49 min, m/z=359.8[M+1]⁺; ¹HNMR (300 MHz, Methanol-d₄, ppm) δ 8.20 (d, J=2.6 Hz, 1H), 8.00 (d, J=2.7 Hz, 1H), 7.86 (s, 1H), 4.87-4.78 (m, 1H), 4.72-4.63 (m, 1H), 4.49-4.40 (m, 1H), 4.40-4.31 (m, 1H).

### Synthesis of Compound 151: 2-Cyano-N-(5-fluoro-6-oxo-1-(2,2,2-trifluoroethyl)-1,6-dihydropyridin-3-yl)thiazole-5-carboxamide

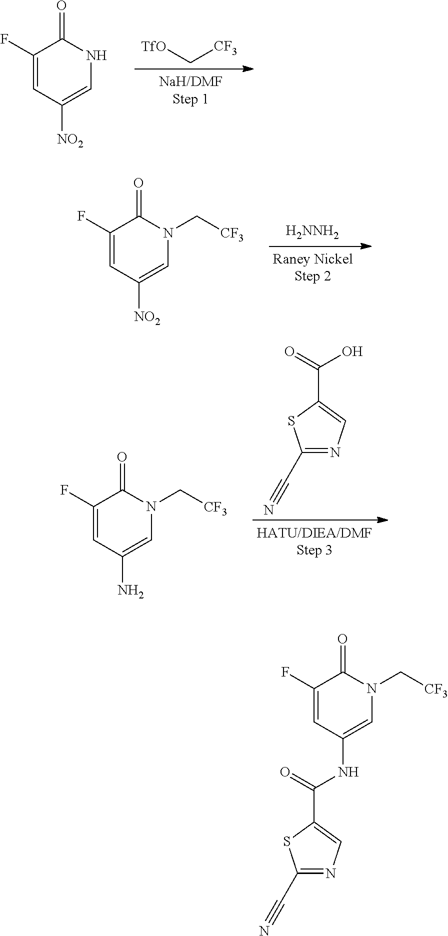

### Step 1: 3-Fluoro-5-nitro-1-(2,2,2-trifluoroethyl)pyridin-2(1H)-one

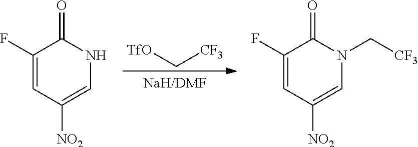 [SMILES: O=c1[nH]cc([N+](=O)[O-])cc1F ; O=S(=O)(OCC(F)(F)F)C(F)(F)F ; O=c1c(F)cc([N+](=O)[O-])cn1CC(F)(F)F]

Into a 100-mL round-bottom flask, was placed 3-fluoro-5-nitro-1,2-dihydropyridin-2-one (1.5 g, 9.49 mmol, 1.00 equiv), Cs₂CO₃ (6.2 g, 19.03 mmol, 2.01 equiv), and N,N-dimethylformamide (20 mL), followed by the dropwise addition of 2,2,2-trifluoroethyl trifluoromethanesulfonate (11 g, 47.39 mmol, 4.99 equiv). The resulting solution was stirred for 3 h at 25° C. The reaction progress was monitored by LCMS. The reaction was quenched by the addition of 50 mL of water. The resulting solution was extracted with 4×50 mL of dichloromethane. The organic layers combined were applied onto a silica gel column with ethyl acetate/petroleum ether (1:9). This resulted in 1.3 g (57% yield) of 3-fluoro-5-nitro-1-(2,2,2-trifluoroethyl)-1,2-dihydropyridin-2-one as a yellow solid.

LCMS (ESI): RT=1.74 min, m z=241.0 [M+H]⁺;

### Step 2: 5-Amino-3-fluoro-1-(2,2,2-trifluoroethyl)pyridin-2(1H)-one

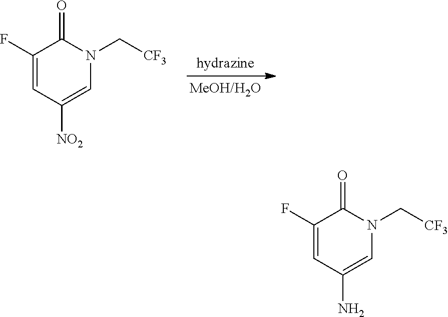 [SMILES: O=c1c(F)cc([N+](=O)[O-])cn1CC(F)(F)F ; Nc1cc(F)c(=O)n(CC(F)(F)F)c1]

Into a 100-mL 3-necked round-bottom flask was placed 3-fluoro-5-nitro-1-(2,2,2-trifluoroethyl)-1,2-dihydropyridin-2-one (400 mg, 1.67 mmol, 1.00 equiv), methanol (2.5 mL), and Raney Ni (300 mg). This was followed by the dropwise addition of hydrazine hydrate (1 mL) while stirring at 0° C. The resulting solution was stirred for 30 min at 0° C. The reaction progress was monitored by LCMS. The solids were filtered out. The resulting mixture was concentrated under vacuum. This resulted in 180 mg (51% yield) of 5-amino-3-fluoro-1-(2,2,2-trifluoroethyl)-1,2-dihydropyridin-2-one as a black solid.

LCMS (ESI): RT=0.80 min, m z=211.0 [M+H]⁺.

### Step 3: 2-Cyano-N-(5-fluoro-6-oxo-1-(2,2,2-trifluoroethyl)-1,6-dihydropyridin-3-yl)thiazole-5-carboxamide (Compound 151)

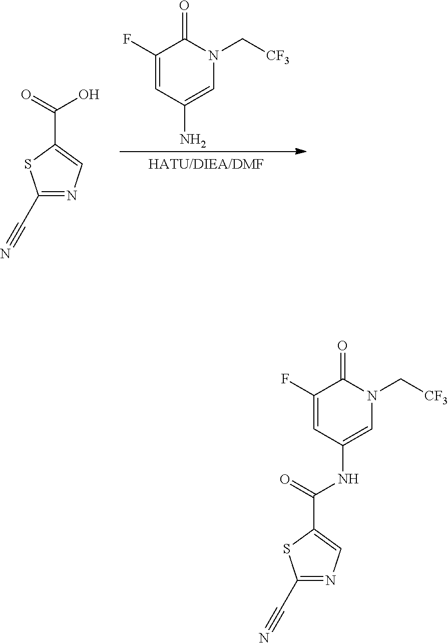 [SMILES: N#Cc1ncc(C(=O)O)s1 ; Nc1cc(F)c(=O)n(CC(F)(F)F)c1 ; N#Cc1ncc(C(=O)Nc2cc(F)c(=O)n(CC(F)(F)F)c2)s1]

Into a 8-mL vial was placed 5-amino-3-fluoro-1-(2,2,2-trifluoroethyl)-1,2-dihydropyridin-2-one (150 mg, 0.71 mmol, 1.00 equiv), 2-cyano-1,3-thiazole-5-carboxylic acid (100 mg, 0.71 mmol, 1.00 equiv), HATU (370 mg, 1.06 mmol, 1.50 equiv), DIEA (254 mg, 2.13 mmol, 3.00 equiv), and N,N-dimethylformamide (2 mL). The resulting solution was stirred for 3 h at 25° C. The reaction progress was monitored by LCMS. The resulting mixture was quenched by 5 ml of ice/water and extracted by dichloromethane (3×10 mL). The organic layers were concentrated under vacuum. The crude product 120 mg (85%) was purified by Prep-HPLC with the following conditions: Column, X-bridge Shield RP 18, 5 um, 19*150 mm; mobile phase, water with 10 mmol NH₄HCO₃ and CH₃CN (10.0% CH₃CN up to 28.0% in 2 min, up to 46.0% in 10 min, up to 100.0% in 1 min, down to 10.0% in 1 min); Detector, UV 254 nm. This resulted in 28.5 mg (24% yield) of 2-cyano-N-[5-fluoro-6-oxo-1-(2,2,2-trifluoroethyl)-1,6-dihydropyridin-3-yl]-LCMS (ESI): RT=1.54 min, m z=346.9 [M+H]⁺; ¹H NMR (300 MHz, DMSO-d₆) δ 10.81 (s, 1H), 8.78 (s, 1H), 8.03 (s, 1H), 7.71 (dd, J=11.2 Hz, 2.5 Hz, 1H), 5.04 (q, J=9.5 Hz, 2H) ppm.

### Synthesis of Compound 297: N-(1-(2,2-difluoroethyl)-5-fluoro-6-oxo-1,6-dihydropyridin-3-yl)-2-(trifluoromethyl)thiazole-5-carboxamide

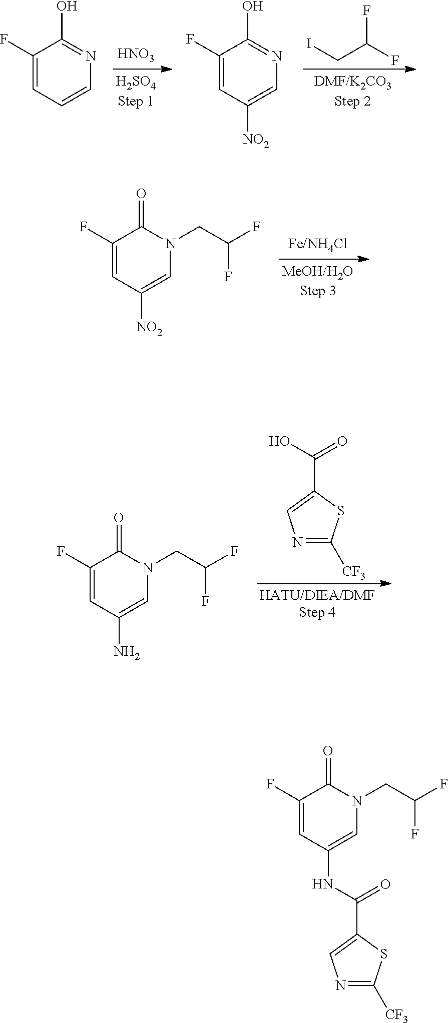

### Step 1: 3-Fluoro-5-nitropyridin-2-ol

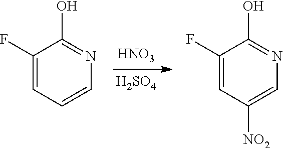

Into a 2000-mL round-bottom flask was placed 3-fluoro-1,2-dihydropyridin-2-one (100 g, 884.22 mmol, 1.00 equiv), and con. H₂SO₄ (700 mL), by the dropwise addition of fuming HNO₃ (150 mL) while stirring at 80° C. The resulting solution was stirred for 2 h at 25° C. The reaction progress was monitored by LCMS. The reaction was poured into 5000 mL of water/ice. The resulting solution was extracted with 3×2000 mL of ethyl acetate. The resulting mixture was concentrated under vacuum. The crude product was crystallized from ethyl acetate to give 40 g (30% yield) of 3-fluoro-5-nitro-1,2-dihydropyridin-2-one as a yellow solid.

LCMS (ESI): RT=0.49 min, m z=159 [M+H]⁺; ¹H NMR (300 MHz, Methanol-d₄ ppm) δ 8.52 (dd, J=2.8, 1.1 Hz, 1H), 8.09 (dd, J=10.0, 2.8 Hz, 1H)

### Step 2: 1-(2,2-Difluoroethyl)-3-fluoro-5-nitropyridin-2(1H)-one

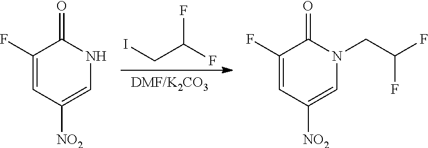

Into a 250-mL round-bottom flask was placed 3-fluoro-5-nitro-1,2-dihydropyridin-2-one (7 g, 12.60 mmol, 1.00 equiv), potassium carbonate (18.3 g, 37.61 mmol, 3.00 equiv), N,N-dimethylformamide (80 mL), and 1,1-difluoro-2-iodoethane (24.5 g, 37.61 mmol, 3.00 equiv). The resulting solution was stirred for 8 h at 80° C. The resulting solution was extracted with 3×500 mL of ethyl acetate. The resulting mixture was concentrated under vacuum. The residue was applied onto a silica gel column with ethyl acetate/petroleum ether (1:5). This resulted in 4.7 g (71% yield) of 1-(2,2-difluoroethyl)-3-fluoro-5-nitro-1,2-dihydropyridin-2-one as yellow oil.

LCMS (ESI): RT=1.05 min, m z=223.0 [M+H]⁺; ¹H NMR (300 MHz, DMSO-d₆, ppm) δ 9.13 (dd, J=2.9, 1.3 Hz, 1H), 8.27 (dd, J=9.8, 2.8 Hz, 1H), 6.60-6.15 (m, 1H), 4.71-4.57 (m, 2H)

### Step 3: 5-Amino-1-(2,2-difluoroethyl)-3-fluoropyridin-2(1H)-one

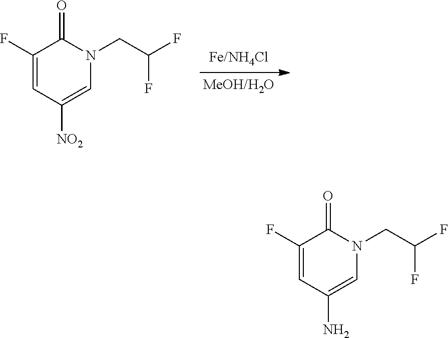 [SMILES: O=c1c(F)cc([N+](=O)[O-])cn1CC(F)F ; "MeOH/H2O" ; Nc1cc(F)c(=O)n(CC(F)F)c1]

Into a 50-mL round-bottom flask was placed 1-(2,2-difluoroethyl)-3-fluoro-5-nitro-1,2-dihydropyridin-2-one (1.7 g, 7.65 mmol, 1.00 equiv), Fe (4.2 g, 76.65 mmol, 10.00 equiv), NH₄C₁ (4.1 g, 76.65 mmol, 10.00 equiv), methanol (10 mL), and water (10 mL). The resulting solution was stirred for 2 h at 60° C., cooled to 25° C. and filtered. The filtrate was concentrated under vacuum. The residue was applied onto a silica gel column with MeOH/DCM (1:10). This resulted in 700 mg (48% yield) of 5-amino-1-(2,2-difluoroethyl)-3-fluoro-1,2-dihydropyridin-2-one as a brown oil.

LCMS (ESI): RT=0.46 min, m/z=193.1 [M+H]⁺; ¹H NMR (300 MHz, DMSO-d₆, ppm) δ 7.42-7.04 (m, 3H), 6.72 (s, 1H), 6.50-6.06 (m, 1H), 4.34-4.26 (m, 2H).

### Step 4: N-(1-(2,2-difluoroethyl)-5-fluoro-6-oxo-1,6-dihydropyridin-3-yl)-2-(trifluoromethyl)thiazole-5-carboxamide (Compound 297)

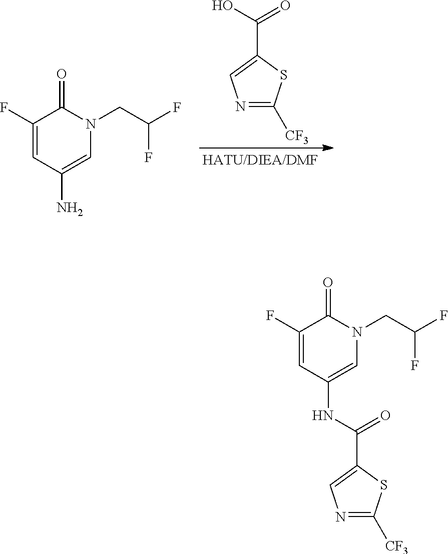 [SMILES: Nc1cc(F)c(=O)n(CC(F)F)c1 ; O=C(O)c1cnc(C(F)(F)F)s1 ; O=C(Nc1cc(F)c(=O)n(CC(F)F)c1)c1cnc(C(F)(F)F)s1]

Into a 25-mL round-bottom flask was placed 5-amino-1-(2,2-difluoroethyl)-3-fluoro-1,2-dihydropyridin-2-one (159 mg, 0.83 mmol, 1.20 equiv), HATU (210 mg, 0.56 mmol, 1.10 equiv), DIEA (190 mg, 1.53 mmol, 3.00 equiv), N,N-dimethylformamide (3 mL), and 2-(trifluoromethyl)-1,3-thiazole-5-carboxylic acid (100 mg, 0.51 mmol, 1.00 equiv). The resulting solution was stirred for 2 h at 25° C. The reaction progress was monitored by LCMS. The resulting solution was extracted with 3×50 mL of ethyl acetate. The resulting mixture was concentrated under vacuum. The crude product 170 mg (90%) was purified by Flash-Prep-HPLC with the following conditions: Column, C18 silica gel; mobile phase, CH₃CN/H₂O (0.05% TFA)=20.0% increasing to CH₃CN/H₂O (0.05% TFA)=30.0% within 8 min; Detector, UV 254 nm. This resulted in 108.6 mg (44% yield) of N-[1-(2,2-difluoroethyl)-5-fluoro-6-oxo-1,6-dihydropyridin-3-yl]-2-(trifluoromethyl)-1,3-thiazole-5-carboxamide as a light yellow solid.

LCMS (ESI): RT=1.66 min, m z=372.0 [M+H]⁺; ¹H NMR (300 MHz, DMSO-d₆, ppm) δ 10.71 (s, 1H), 8.74 (d, J=1.3 Hz, 1H), 8.00 (d, J=2.4 Hz, 1H), 7.67 (dd, J=11.3, 2.6 Hz, 1H), 6.53 (t, J=3.7 Hz, 1H), 4.57-4.46 (m, 2H).

### Synthesis of Compound 298: 4-Chloro-N-(1-(2,2-difluoroethyl)-5-fluoro-6-oxo-1,6-dihydropyridin-3-yl)-5-(trifluoromethyl)thiophene-2-carboxamide

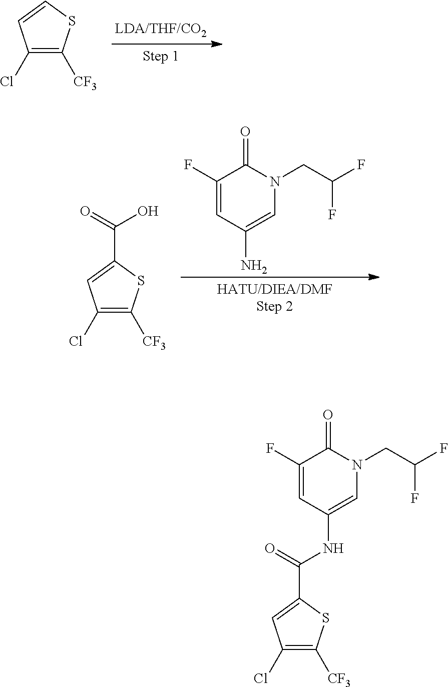 [SMILES: FC(F)(F)c1sccc1Cl ; O=C(O)c1cc(Cl)c(C(F)(F)F)s1 ; Nc1cc(F)c(=O)n(CC(F)F)c1 ; O=C(Nc1cc(F)c(=O)n(CC(F)F)c1)c1cc(Cl)c(C(F)(F)F)s1]

### Step 1: 4-Chloro-5-(trifluoromethyl)thiophene-2-carboxylic acid

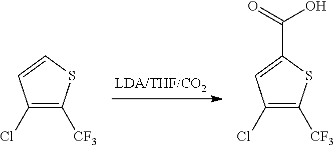 [SMILES: FC(F)(F)c1sccc1Cl ; O=C(O)c1cc(Cl)c(C(F)(F)F)s1]

Into a 50-mL 3-necked round-bottom flask, purged and maintained with an inert atmosphere of nitrogen, was placed 1-chloro-2-(trifluoromethyl)cyclopentane (1 g, 5.794 mmol, 1.00 equiv), THE (5 mL), and LDA (0.31 mL, 6.95 mmol, 1.20 equiv) (2.5 mol/L in THF) at −78° C. (liquid nitrogen/ethanol). The resulting solution was stirred for 30 min at −78° C., then solid CO₂ (excess) was added slowly and in batches. The resulting solution was stirred for an additional 2 h at 25° C., and the reaction was then quenched by the addition of 4 mL of HCl (2 mol/L). The resulting solution was extracted with 3×20 mL of dichloromethane and washed with 3×10 mL saturated NaCl solution. The organic layers combined and concentrated, and the solids were collected.

LCMS (ESI): RT=1.13 min, m/z=229 [M−H]⁺; ¹H NMR (400 MHz, DMSO-d₆ ppm) δ 8.88 (bar, 1H), 7.31 (s, 1H).

### Step 2: 4-Chloro-N-(1-(2,2-difluoroethyl)-5-fluoro-6-oxo-1,6-dihydropyridin-3-yl)-5-(trifluoromethyl)thiophene-2-carboxamide (Compound 298)

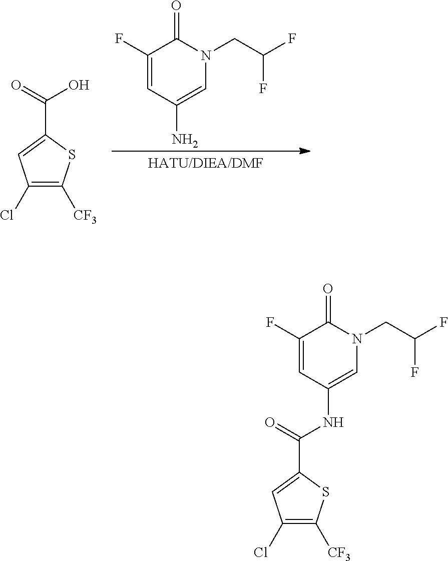 [SMILES: O=C(O)c1cc(Cl)c(C(F)(F)F)s1 ; Nc1cc(F)c(=O)n(CC(F)F)c1 ; O=C(Nc1cc(F)c(=O)n(CC(F)F)c1)c1cc(Cl)c(C(F)(F)F)s1]

Into a 50-mL round-bottom flask was placed 4-chloro-5-(trifluoromethyl)thiophene-2-carboxylic acid (200 mg, 0.86 mmol, 1.00 equiv), 5-amino-1-(2,2-difluoroethyl)-3-fluoro-1,2-dihydropyridin-2-one (199.98 mg, 1.04 mmol, 1.2 equiv), HATU (428.73 mg, 1.12 mmol, 1.30 equiv), DIEA (336.29 mg, 2.60 mmol, 3.00 equiv), and N,N-dimethylformamide (2 mL). The resulting solution was stirred for 2 h at 25° C., and the reaction progress was monitored by LCMS. The resulting mixture was quenched by 10 ml of ice/water. The resulting solution was extracted with 3×15 mL of ethyl acetate. The resulting mixture was concentrated under vacuum. The crude product (160 mg; 85%) was purified by Prep-HPLC with the following conditions: Column, XSelect CSH Prep C18 OBD Column, 5 um, 19*150 mm; mobile phase, Water (0.05% NH₄HCO₃) and ACN (40% Phase B up to 65% in 7 min); Detector, UV. This resulted in 72 mg (36% yield) of 4-chloro-N-(1-(2,2-difluoroethyl)-5-fluoro-6-oxo-1,6-dihydropyridin-3-yl)-5-(trifluoromethyl)thiophene-2-carboxamide as a white solid.

LCMS (ESI): RT=2.439 min, m/z=405 [M+H]⁺; ¹H NMR (400 MHz, DMSO-d₆ ppm) δ 10.59 (s, 1H), 8.09 (d, J=1.5 Hz, 1H), 8.02 (t, J=2.1 Hz, 1H), 7.68 (dd, J=11.2, 2.6 Hz, 1H), 6.36 (t, J=3.7 Hz, 1H), 4.53 (td, J=15.1, 3.7 Hz, 2H).

### Alternative Synthesis of Compound 298: 4-Chloro-N-(1-(2,2-difluoroethyl)-5-fluoro-6-oxo-1,6-dihydropyridin-3-yl)-5-(trifluoromethyl)thiophene-2-carboxamide

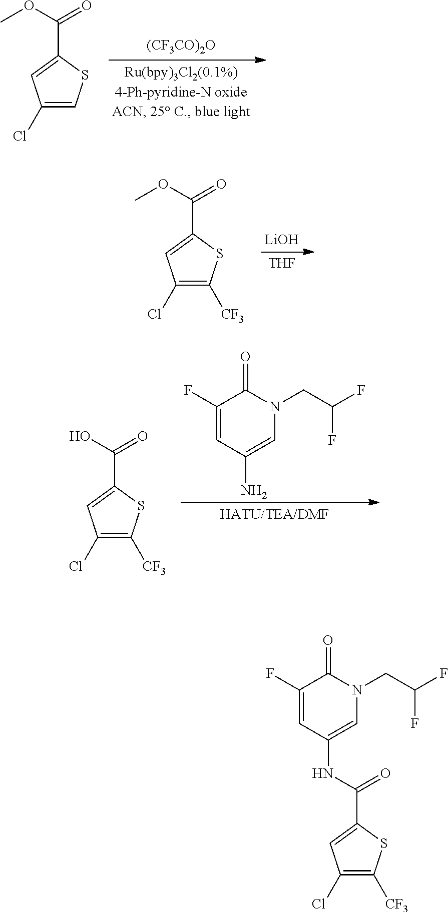

### Step 1: Methyl 4-Chloro-5-(trifluoromethyl)thiophene-2-carboxylate

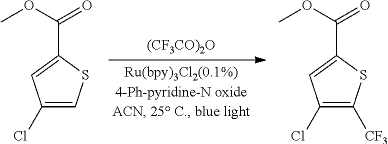

Into a 1-L 3-necked round-bottom flask purged and maintained with an inert atmosphere of nitrogen, was placed 4-phenylpyridine N-oxide (58.43 g, 339.732 mmol, 2.00 equiv), methyl 4-chlorothiophene-2-carboxylate (30.00 g, 169.87 mmol, 1.00 equiv), tris(bipyridine)ruthenium(II) chloride (108.79 mg, 0.170 mmol, 0.001 equiv), ACN (300 mL), trifluoroacetic anhydride (78.49 g, 373.705 mmol, 2.20 equiv). The resulting solution was stirred for 8 hr at 25° C. under blue light. The solids were filtered out. The resulting solution was extracted with 4×300 mL of petroleum ether and concentrated. This resulted in 64 g (51%) of methyl 4-chloro-5-(trifluoromethyl)thiophene-2-carboxylate as red oil.

### Step 2: 4-Chloro-5-(trifluoromethyl)thiophene-2-carboxylic acid

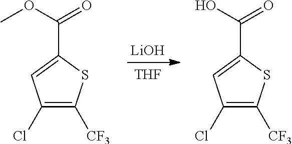

Into a 1-L 3-necked round-bottom flask, was placed methyl 4-chloro-5-(trifluoromethyl)thiophene-2-carboxylate (64.00 g, 261.64 mmol, 1.00 equiv) and tetrahydrofuran (600 mL), and the resulting solution was stirred at 0° C. Then lithium hydroxide (18.80 g, 784.92 mmol, 3.00 equiv) and H₂O (200 mL) was added for 20 min. The resulting solution was allowed to react, with stirring, for an additional 4 hr at 25° C. The resulting solution was extracted with 50 mL of ethyl acetate The pH value of the solution was adjusted to 4-5 with HCl. The resulting solution was extracted with 3×150 mL of ethyl acetate, and the organic layer was evaporated. This resulted in 42 g (69%) of 4-chloro-5-(trifluoromethyl)thiophene-2-carboxylic acid as a dark red solid.

### Step 3: 4-Chloro-N-(1-(2,2-difluoroethyl)-5-fluoro-6-oxo-1,6-dihydropyridin-3-yl)-5-(trifluoromethyl)thiophene-2-carboxamide

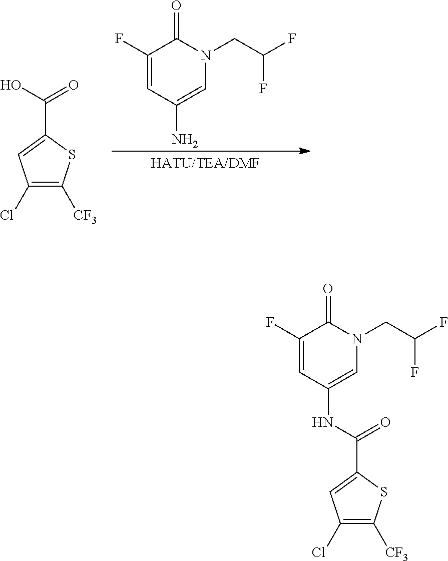 [SMILES: O=C(O)c1cc(Cl)c(C(F)(F)F)s1 ; Nc1cc(F)c(=O)n(CC(F)F)c1 ; O=C(Nc1cc(F)c(=O)n(CC(F)F)c1)c1cc(Cl)c(C(F)(F)F)s1]

Into a 1-L round-bottom flask, was placed 4-chloro-5-(trifluoromethyl)thiophene-2-carboxylic acid (30.00 g, 130.10 mmol, 1.00 equiv), 5-amino-1-(2,2-difluoroethyl)-3-fluoropyridin-2-one (30.00 g, 156.12 mmol, 1.20 equiv), HATU (59.36 g, 156.12 mmol, 1.20 equiv), DIEA (50.44 g, 390.30 mmol, 3.00 equiv), DMF (288.46 mL). The resulting solution was stirred for 2 hr at 25° C. The resulting solution was extracted with 3×1.2 L of ethyl acetate The resulting mixture was washed with 2×1.2 L of Water. The resulting mixture was concentrated. The residue was applied onto a silica gel column with water:ACN (2:3). This resulted in 28.14 g (53%) of 4-chloro-N-[1-(2,2-difluoroethyl)-5-fluoro-6-oxopyridin-3-yl]-5-(trifluoromethyl)thiophene-2-carboxamide as a yellow solid.

LCMS: (ES, m z): RT=2.618 min, m z 404.95[M+H]⁺; H-NMR: ¹H NMR (400 MHz, DMSO-d₆) δ 10.56 (s, 1H), 8.05 (d, J=1.5 Hz, 1H), 8.00 (t, J=2.0 Hz, 1H), 7.65 (dd, J=11.2, 2.5 Hz, 1H), 7.56 (s, 1H), 6.34 (t, J=3.7 Hz, 1H), 4.51 (td, J=15.1, 3.8 Hz, 2H).

### Example 2

### SMARCA2 ATPase and Chromatin Remodeling Assays

General Materials. Adenosine 5′-triphosphate disodium salt hydrate (ATP), Bicine, bovine skin gelatin (BSG), dimethylsulfoxide (DMSO), doxorubicin, glycerol, HEPES, NP-40, phenylmethanesulfonyl fluoride (PMSF), Tris-HCl, Tris(2-carboxyethyl)phosphine hydrochloride solution (TCEP) and Tween 20 were commercially available.

Substrates. Chicken erythrocyte mononucleosomes were prepared from fresh chicken blood collected into anticoagulant and filtered through cheesecloth. The filtered chicken blood was diluted in buffer A (10 mM Tris-HCl, pH 7.4, 10 mM NaCl, 3 mM MgCl₂) and centrifuged at 500 g for 15 minutes to collect the erythrocytes. Erythrocyte nuclei were harvested by resuspending the pellets in buffer A containing 0.3% NP-40 detergent. The mixture was stirred with a magnetic bar for 10 mins and centrifuged at 4000 g for 10 minutes. Resuspension, stirring and centrifugation was repeated twice more before the final nuclei pellet was resuspended in buffer A+0.3% NP-40 and stored at −80° C. until purification. For nucleosome purification, nuclei aliquots were rapidly thawed and washed five times with buffer B (10 mM Tris-HCl, pH 7.4, 1 mM CaCl₂), 1 mM PMSF) and collected by centrifugation at 2800 g for 10 minutes. Pellets were resuspended in buffer B and treated with 100 U/mL micrococcal nuclease for 40 minutes at 37° C. The reaction was terminated by the addition of 2 mM EDTA and was centrifuged at 2800 g for 10 minutes. The nuclei were then lysed by resuspending the pellet in 1 mM EDTA and 1 mM PMSF and passing the mixture through a 22G needle 4 to 5 times. The solution was then centrifuged at 15,000 g for 20 minutes and the supernatant was collected. The remaining pellet was lysed through the syringe twice more and the supernatants were pooled after centrifugation. The pooled supernatants were further purified by size exclusion chromatography (GE Sephacryl S300 HR 50/100) in buffer C (10 mM HEPES, pH 7.5, 10 mM KCl, 1 mM EDTA, 1 mM PMSF, 10% glycerol). Fractions were analyzed by agarose gel electrophoresis and SDS-PAGE and fractions containing mononucleosomes were pooled and stored at −80° C.

Molecular Biology: Full-length human SMARCA2 isoform 1 (P51531) transcript clone was amplified from a cDNA library incorporating an N-terminal HIS tag (MGSHHHHHHHHSG) fused directly to Ser2 of SMARCA2 and a C-FLAG tag (YKDDDDK) fused directly to Glu1590 of SMARCA2. The amplified gene was subcloned into pFastBacI (Life Technologies).

Protein Expression: Recombinant baculovirus were generated and protein over-expression was accomplished by infecting exponentially growing Sf21 insect cell culture at 1.24×10⁶ cell/ml with an MOI of 0.1. Infections were carried out for 76 hours, harvested by centrifugation, and stored at −80° C. for purification.

Protein Purification:

Expressed full-length SMARCA2 was purified from cell paste by FLAG affinity chromatography followed by anion exchange chromatography. The protein was dialyzed into a storage buffer containing 25 mM HEPES, 300 mM KCl, 10% Glycerol, pH 7.9, 1 mM TCEP and 0.01% Tween-20. The purity of the protein was measured as 74% using micro-capillary based gel electrophoresis.

The predicted translation for the HIS-SMARCA2-FL-FLAG is set forth in SEQ ID NO: 1.

### General Procedure for SMARCA2 and SMARCA4 ATPase Activity Assay

The SMARCA2 or SMARCA4 ATPase assays were identical. The SMARCA2 or SMARCA4 ATPase assays were performed in a buffer consisting of 20 mM Bicine (pH 7.5), 10 mM KCl, 1 mM MgCl₂, 1 mM TCEP, 0.005% BSG, and 0.002% Tween 20, prepared on the day of use. Compounds in 100% DMSO were 3-fold serially diluted to produce a 10 point curve for IC₅₀ determination and 0.2 uL were transferred into polypropylene 384-well V-bottom plates (Greiner) using an Echo liquid handler (Labcyte). DMSO (0.2 uL) was added to Columns 11, 12, 23, 24, rows A-H for the maximum signal control and 0.2 uL doxorubicin, a known DNA intercalator, was added to columns 11, 12, 23, 24, rows I-P for the minimum signal control. The SMARCA2 or SMARCA4 enzyme (10 uL) was added to the compounds and allowed to incubate with the compounds for 30 min at room temperature. The SMARCA2 or SMARCA4 ATPase assay was initiated by the addition of 10 uL chicken mononucleosome and ATP mixture (final volume=20 uL). The final concentrations of the assay components were as follows: SMARCA2 or SMARCA4 was 5 nM, ATP was 250 uM, chicken mononucleosome was 10 nM, doxorubicin in the minimum signal control wells was 50 uM, and the DMSO concentration was 1%. 5 uL of the reaction mixture was transferred from the 384-well polypropylene plate to a white opaque polystyrene 384-well plate. At 60 minutes, the assays were terminated by the addition of 5 uL ADP-Glo™ Reagent (Promega). After 40 minutes, the luciferase reaction was initiated by the addition of 10 uL of Kinase Detection Reagent (Promega) and incubated for 1 hour. The ADP product generated from the SMARCA2 or SMARCA4 ATPase reaction was determined via luminescence intensity. The IC₅₀ values for compounds of the disclosure are listed in Tables 3.1, 3a.1 and 3b.1 below (“A” means IC₅₀<100 nM; “B” means IC₅₀ ranging between 100 nM and 1 μM; “C” means IC₅₀ ranging between >1 μM and 10 μM; “D” means IC₅₀ ranging between >10 μM and 50 μM; “E” means IC₅₀>50 μM; “-” or “ND” means not determined).

TABLE 3.1
SMARCA2 inhibition by compounds of the disclosure.

| Compd. | FL-SMARCA2 |
|---|---|
| No. | IC₅₀ |
| 1 | 2.724 |
| 2 | 33.25 |
| 3 | >50 |
| 4 | >50 |
| 5 | >50 |
| 6 | >50 |
| 7 | >50 |
| 8 | >50 |
| 9 | >50 |
| 10 | >50 |
| 11 | >50 |
| 12 | >50 |
| 13 | >50 |
| 14 | >50 |
| 15 | >50 |
| 16 | >50 |
| 17 | 4.058 |
| 18 | 19.53 |
| 19 | >50 |
| 20 | >50 |
| 21 | 2.037 |
| 22 | ND |
| 23 | ND |
| 24 | ND |
| 25 | ND |
| 26 | 19.6 |
| 27 | ND |
| 28 | ND |
| 29 | ND |
| 30 | ND |
| 31 | ND |
| 32 | >50 |
| 33 | >50 |
| 34 | 28.5 |
| 35 | >50 |
| 36 | >50 |
| 37 | >50 |
| 38 | ND |
| 39 | ND |
| 40 | 2.63 |
| 41 | >50 |
| 42 | 12.6 |
| 43 | 1.261 |
| 44 | >50 |
| 45 | 43.14 |
| 46 | 11.14 |
| 47 | >50 |
| 48 | 14.08 |
| 49 | >50 |
| 50 | >50 |
| 51 | 46.4 |
| 52 | >50 |
| 53 | 1.478 |
| 54 | >50 |
| 55 | >50 |
| 56 | >50 |
| 57 | >50 |
| 58 | 1.305 |
| 59 | >50 |
| 60 | 16.7 |
| 61 | >50 |
| 62 | >50 |
| 63 | >50 |
| 64 | >50 |
| 65 | 28.4 |
| 66 | >50 |
| 67 | >50 |
| 68 | 8.552 |
| 69 | 2.517 |
| 70 | 16.7 |
| 71 | >50 |
| 72 | 33.2 |
| 73 | 11.5 |
| 74 | 17.24 |
| 75 | 0.4365 |
| 76 | >50 |
| 77 | >50 |
| 78 | >50 |
| 79 | >50 |
| 80 | 0.2091 |
| 81 | 0.2229 |
| 82 | 14.79 |
| 83 | 20.8 |
| 84 | 0.1969 |
| 85 | 11.98 |
| 86 | 0.3646 |
| 87 | >50 |
| 88 | >50 |
| 89 | >50 |
| 90 | >50 |
| 91 | >50 |
| 92 | >50 |
| 93 | >50 |
| 94 | 3.153 |
| 95 | >50 |
| 96 | >50 |
| 97 | >50 |
| 98 | >50 |
| 99 | >50 |
| 100 | >50 |
| 101 | 0.07411 |
| 102 | 4.124 |
| 103 | 0.356 |
| 104 | 0.192 |
| 105 | 27.83 |
| 106 | 0.4172 |
| 107 | 36.72 |
| 108 | >50 |
| 109 | 0.1733 |
| 110 | >50 |
| 111 | >50 |
| 112 | >50 |
| 113 | 14.07 |
| 114 | 9.415 |
| 115 | >50 |
| 116 | 39.3 |
| 117 | >50 |
| 118 | >50 |
| 119 | >50 |
| 120 | >50 |
| 121 | >50 |
| 122 | >50 |
| 123 | 1.195 |
| 124 | >50 |
| 125 | 0.03086 |
| 126 | 46.8 |
| 127 | 0.2516 |
| 128 | 0.07867 |
| 129 | 0.05117 |
| 130 | 0.147 |
| 131 | 0.1127 |
| 132 | 0.1425 |
| 133 | 0.3426 |
| 134 | 0.1485 |
| 135 | 0.2933 |
| 136 | 0.05772 |
| 137 | 0.02599 |
| 138 | 0.00642 |
| 139 | 0.01964 |
| 140 | 0.04671 |
| 141 | 0.09409 |
| 142 | 0.04745 |
| 143 | 0.1214 |
| 144 | 0.2452 |
| 145 | 3.014 |
| 146 | 1.017 |
| 147 | 17.13 |
| 148 | 0.01266 |
| 149 | >50 |
| 150 | 0.02662 |
| 151 | 0.02043 |
| 152 | 0.1656 |
| 153 | 0.1472 |
| 154 | 1.93 |
| 155 | 0.1627 |
| 156 | 0.2346 |
| 157 | 1.5 |
| 158 | 1.85 |
| 159 | 0.1936 |
| 160 | 0.224 |
| 161 | 0.5535 |
| 162 | 0.1936 |
| 163 | 0.3094 |
| 164 | 0.3105 |
| 165 | 0.3434 |
| 166 | 2.723 |
| 167 | 0.6095 |
| 168 | 0.2755 |
| 169 | 0.3749 |
| 170 | 0.376 |
| 171 | >50 |
| 172 | 0.238 |
| 173 | 0.03917 |
| 174 | 0.3883 |
| 175 | 2.04 |
| 176 | 0.0552 |
| 177 | >50 |
| 178 | 30.83 |
| 179 | >50 |
| 180 | 0.08455 |
| 181 | 0.1139 |
| 182 | 0.03553 |
| 183 | >50 |
| 184 | >50 |
| 185 | >50 |
| 186 | 15.64 |
| 187 | 0.1584 |
| 188 | 0.0305 |
| 189 | 0.00909 |
| 190 | 0.0201 |
| 191 | 0.04589 |
| 192 | 0.01706 |
| 193 | 1.229 |
| 194 | 3.116 |
| 195 | 7.59 |
| 196 | 0.03824 |
| 197 | 0.2815 |
| 198 | 0.1073 |
| 199 | >50 |
| 200 | 48.28 |
| 201 | >50 |
| 202 | >50 |
| 203 | 0.04068 |
| 204 | 1.26 |
| 205 | 3.489 |
| 206 | 0.3174 |
| 207 | 0.07293 |
| 208 | 0.123 |
| 209 | >50 |
| 210 | 0.2066 |
| 211 | 2.309 |
| 212 | 8.522 |
| 213 | 17.87 |
| 214 | 29.9 |
| 215 | 0.07099 |
| 216 | >50 |
| 217 | >50 |
| 218 | >50 |
| 219 | >50 |
| 220 | 0.4877 |
| 221 | 1.452 |
| 222 | 1.31 |
| 223 | 18.38 |
| 224 | >50 |
| 225 | >50 |
| 226 | >50 |
| 227 | >50 |
| 228 | >50 |
| 229 | 0.6015 |
| 230 | >50 |
| 231 | 0.1417 |
| 232 | 0.02489 |
| 233 | 7.629 |
| 234 | 0.4783 |
| 235 | 0.05915 |
| 236 | 0.1247 |
| 237 | 0.201 |
| 238 | 0.2324 |
| 239 | >50 |
| 240 | 0.6257 |
| 241 | 0.1119 |
| 242 | 0.03887 |
| 243 | 1.643 |
| 244 | >50 |
| 245 | 2 |
| 246 | 1.349 |
| 247 | 2.027 |
| 248 | 21.01 |
| 249 | 0.03584 |
| 250 | 25.4 |
| 251 | 0.1306 |
| 252 | >50 |
| 253 | >50 |
| 254 | 5.813 |
| 255 | 16.73 |
| 256 | 6.339 |
| 257 | 19.12 |
| 258 | >50 |
| 259 | 0.05115 |
| 260 | 0.01528 |
| 261 | 7.375 |
| 262 | 15.32 |
| 263 | 21.6 |
| 264 | 6.227 |
| 265 | 0.08147 |
| 266 | >50 |
| 267 | >50 |
| 268 | 16.65 |
| 269 | 5.187 |
| 270 | >50 |
| 271 | >50 |
| 272 | >50 |
| 273 | >50 |
| 274 | >50 |
| 275 | 0.3813 |
| 276 | 28.68 |
| 277 | >50 |
| 278 | >50 |
| 279 | 24 |
| 280 | >50 |
| 281 | >50 |
| 282 | >50 |
| 283 | 33.18 |
| 284 | 3.214 |
| 285 | 0.02722 |
| 286 | 9.485 |
| 287 | 1.277 |
| 288 | >50 |
| 289 | 10.56 |
| 290 | 0.1359 |
| 291 | >50 |
| 292 | 6.83 |
| 293 | 0.1268 |
| 294 | 0.1171 |
| 295 | 0.173 |
| 296 | 0.09398 |
| 297 | 0.07568 |
| 298 | 0.02411 |
| 299 | >50 |
| 300 | 2.735 |
| 301 | 16.71 |
| 302 | >50 |
| 303 | >50 |
| 304 | >50 |
| 305 | >50 |
| 306 | >50 |
| 307 | 0.9403 |
| 308 | 23.3 |
| 309 | 29.6 |
| 310 | >50 |
| 311 | >50 |
| 312 | >50 |
| 313 | >50 |
| 314 | >50 |
| 315 | >50 |
| 316 | >50 |
| 317 | >50 |
| 318 | 2.883 |
| 319 | 1.317 |
| 320 | 0.1514 |
| 321 | 0.1 > 505 |
| 322 | >50 |
| 323 | 0.01923 |
| 324 | 0.03494 |
| 325 | 0.0355 |
| 326 | 3.778 |
| 327 | 0.8459 |
| 328 | 0.03836 |
| 329 | 0.03122 |
| 330 | 0.04223 |
| 331 | 0.1715 |
| 332 | 0.3313 |
| 333 | 0.4169 |
| 334 | 0.1817 |
| 335 | 7.464 |
| 336 | 1.241 |
| 337 | 0.03369 |
| 338 | 0.08896 |
| 339 | 0.9429 |
| 340 | 0.6948 |
| 341 | 0.6682 |
| 342 | 0.2777 |
| 343 | 0.02304 |
| 345 | 0.0165 |
| 346 | 0.1807 |

TABLE 3.2
SMARCA4 inhibition by compounds of the disclosure.

| Compd. | FL-SMARCA4 |
|---|---|
| No. | IC₅₀ |
| 1 | 39.58 |
| 2 | >50 |
| 3 | >50 |
| 4 | >50 |
| 5 | >50 |
| 6 | >50 |
| 7 | >50 |
| 8 | >50 |
| 9 | >50 |
| 10 | >50 |
| 11 | >50 |
| 12 | >50 |
| 13 | >50 |
| 14 | >50 |
| 15 | >50 |
| 16 | >50 |
| 17 | 26.59 |
| 18 | >50 |
| 19 | >50 |
| 20 | >50 |
| 21 | 8.998 |
| 22 | >50 |
| 23 | >50 |
| 24 | >50 |
| 25 | >50 |
| 26 | >50 |
| 27 | >50 |
| 28 | >50 |
| 29 | >50 |
| 30 | >50 |
| 31 | >50 |
| 32 | >50 |
| 33 | >50 |
| 34 | >50 |
| 35 | >50 |
| 36 | >50 |
| 37 | >50 |
| 38 | ND |
| 39 | ND |
| 40 | 11.6 |
| 41 | ND |
| 42 | ND |
| 43 | ND |
| 44 | >50 |
| 45 | ND |
| 46 | ND |
| 47 | ND |
| 48 | >50 |
| 49 | ND |
| 50 | ND |
| 51 | >50 |
| 52 | >50 |
| 53 | 16.7 |
| 54 | >50 |
| 55 | >50 |
| 56 | >50 |
| 57 | >50 |
| 58 | 13.57 |
| 59 | >50 |
| 60 | 34.69 |
| 61 | >50 |
| 62 | >50 |
| 63 | >50 |
| 64 | >50 |
| 65 | >50 |
| 66 | >50 |
| 67 | >50 |
| 68 | >50 |
| 69 | 11.69 |
| 70 | >50 |
| 71 | >50 |
| 72 | >50 |
| 73 | 35.6 |
| 74 | 31.5 |
| 75 | 1.515 |
| 76 | >50 |
| 77 | >50 |
| 78 | >50 |
| 79 | >50 |
| 80 | 0.7578 |
| 81 | 0.7228 |
| 82 | 28.9 |
| 83 | >50 |
| 84 | 1.119 |
| 85 | 34.69 |
| 86 | 0.6685 |
| 87 | >50 |
| 88 | >50 |
| 89 | >50 |
| 90 | >50 |
| 91 | >50 |
| 92 | >50 |
| 93 | >50 |
| 94 | 11.5 |
| 95 | >50 |
| 96 | >50 |
| 97 | >50 |
| 98 | >50 |
| 99 | ND |
| 100 | ND |
| 101 | 0.2374 |
| 102 | 18.6 |
| 103 | 0.531 |
| 104 | 0.229 |
| 105 | >50 |
| 106 | 0.8384 |
| 107 | >50 |
| 108 | >50 |
| 109 | 0.5589 |
| 110 | >50 |
| 111 | >50 |
| 112 | >50 |
| 113 | 16.64 |
| 114 | 14.35 |
| 115 | 16.7 |
| 116 | >50 |
| 117 | >50 |
| 118 | >50 |
| 119 | >50 |
| 120 | >50 |
| 121 | >50 |
| 122 | >50 |
| 123 | 11.6 |
| 124 | >50 |
| 125 | 0.1082 |
| 126 | >50 |
| 127 | 0.5268 |
| 128 | 0.1609 |
| 129 | 0.1959 |
| 130 | 0.4434 |
| 131 | 0.3033 |
| 132 | 0.2996 |
| 133 | 2.18 |
| 134 | 0.6305 |
| 135 | 1.996 |
| 136 | 0.1843 |
| 137 | 0.1113 |
| 138 | 0.01606 |
| 139 | 0.05408 |
| 140 | 0.1466 |
| 141 | 0.352 |
| 142 | 0.1626 |
| 143 | 0.3307 |
| 144 | 1.174 |
| 145 | 11.99 |
| 146 | 3.588 |
| 147 | >50 |
| 148 | 0.0 > 5075 |
| 149 | >50 |
| 150 | 0.09482 |
| 151 | 0.06741 |
| 152 | 0.6164 |
| 153 | 0.7145 |
| 154 | 8.08 |
| 155 | 0.5289 |
| 156 | 0.8838 |
| 157 | 11.05 |
| 158 | 13.73 |
| 159 | 0.7589 |
| 160 | 0.864 |
| 161 | 4.031 |
| 162 | 0.9398 |
| 163 | 1.168 |
| 164 | 1.343 |
| 165 | 1.074 |
| 166 | 16.64 |
| 167 | 2.863 |
| 168 | 1.27 |
| 169 | 1.415 |
| 170 | 2.428 |
| 171 | >50 |
| 172 | 1.071 |
| 173 | 0.2159 |
| 174 | 1.475 |
| 175 | 5.603 |
| 176 | 0.2669 |
| 177 | >50 |
| 178 | 37.7 |
| 179 | >50 |
| 180 | 0.3828 |
| 181 | 0.3135 |
| 182 | 0.1265 |
| 183 | >50 |
| 184 | >50 |
| 185 | >50 |
| 186 | >50 |
| 187 | 0.616 |
| 188 | 0.1187 |
| 189 | 0.03368 |
| 190 | 0.08514 |
| 191 | 0.184 |
| 192 | 0.08741 |
| 193 | 8.933 |
| 194 | 21.88 |
| 195 | 29.8 |
| 196 | 0.1808 |
| 197 | 0.8309 |
| 198 | 0.283 |
| 199 | >50 |
| 200 | 45.3 |
| 201 | >50 |
| 202 | >50 |
| 203 | 0.118 |
| 204 | 5.777 |
| 205 | 22.64 |
| 206 | 0.6487 |
| 207 | 0.3418 |
| 208 | 0.6322 |
| 209 | >50 |
| 210 | 0.6936 |
| 211 | 27.51 |
| 212 | >50 |
| 213 | >50 |
| 214 | >50 |
| 215 | 0.4915 |
| 216 | >50 |
| 217 | >50 |
| 218 | >50 |
| 219 | >50 |
| 220 | 1.932 |
| 221 | 6.277 |
| 222 | 10.16 |
| 223 | >50 |
| 224 | >50 |
| 225 | >50 |
| 226 | >50 |
| 227 | >50 |
| 228 | >50 |
| 229 | 1.884 |
| 230 | >50 |
| 231 | 0.544 |
| 232 | 0.1109 |
| 233 | 36.4 |
| 234 | 1.741 |
| 235 | 0.2723 |
| 236 | 0.555 |
| 237 | 0.8631 |
| 238 | 0.8791 |
| 239 | >50 |
| 240 | 2.391 |
| 241 | 0.6532 |
| 242 | 0.1677 |
| 243 | 11.72 |
| 244 | >50 |
| 245 | 12.1 |
| 246 | 6.37 |
| 247 | 10.7 |
| 248 | >50 |
| 249 | 0.2599 |
| 250 | >50 |
| 251 | 0.6231 |
| 252 | >50 |
| 253 | >50 |
| 254 | 47.1 |
| 255 | >50 |
| 256 | 21.35 |
| 257 | 23.74 |
| 258 | >50 |
| 259 | 0.2278 |
| 260 | 0.06542 |
| 261 | 24.44 |
| 262 | 25.94 |
| 263 | 27.36 |
| 264 | 38.5 |
| 265 | 0.5903 |
| 266 | >50 |
| 267 | >50 |
| 268 | 41.79 |
| 269 | 24.85 |
| 270 | >50 |
| 271 | >50 |
| 272 | >50 |
| 273 | >50 |
| 274 | >50 |
| 275 | 2.098 |
| 276 | 47.1 |
| 277 | >50 |
| 278 | >50 |
| 279 | >50 |
| 280 | >50 |
| 281 | >50 |
| 282 | >50 |
| 283 | >50 |
| 284 | 30.65 |
| 285 | 0.08683 |
| 286 | 16.68 |
| 287 | 3.664 |
| 288 | >50 |
| 289 | 42.6 |
| 290 | 0.6796 |
| 291 | >50 |
| 292 | 8.482 |
| 293 | 0.2997 |
| 294 | 0.3865 |
| 295 | 0.7355 |
| 296 | 0.5195 |
| 297 | 0.448 |
| 298 | 0.1647 |
| 299 | >50 |
| 300 | 5.961 |
| 301 | >50 |
| 302 | >50 |
| 303 | >50 |
| 304 | >50 |
| 305 | >50 |
| 306 | >50 |
| 307 | 3.273 |
| 308 | >50 |
| 309 | >50 |
| 310 | >50 |
| 311 | >50 |
| 312 | >50 |
| 313 | >50 |
| 314 | >50 |
| 315 | >50 |
| 316 | >50 |
| 317 | >50 |
| 318 | 13.48 |
| 319 | 16.62 |
| 320 | 0.5621 |
| 321 | 0.6345 |
| 322 | >50 |
| 323 | 0.06831 |
| 324 | 0.1374 |
| 325 | 0.1626 |
| 326 | 17.17 |
| 327 | 1.511 |
| 328 | 0.108 |
| 329 | 0.1113 |
| 330 | 0.1169 |
| 331 | 0.9243 |
| 332 | 2.2 |
| 333 | 0.8413 |
| 334 | 0.3374 |
| 335 | 45.1 |
| 336 | 5.866 |
| 337 | 0.1042 |
| 338 | 0.3631 |
| 339 | 2.318 |
| 340 | 4.892 |
| 341 | 9.282 |
| 342 | 0.7385 |
| 343 | 0.1075 |
| 345 | 0.03789 |
| 346 | 0.6057 |

TABLE 3a.1
SMARCA2 inhibition by compounds of the disclosure.

| Compd. | FL-SMARCA2 |
|---|---|
| No. | IC₅₀ |
| 1a | >50 |
| 2a | >50 |
| 3a | >50 |
| 4a | >50 |
| 5a | >50 |
| 6a | >50 |
| 7a | >50 |
| 8a | >50 |
| 9a | >50 |
| 10a | >50 |
| 11a | >50 |
| 12a | >50 |
| 13a | >50 |
| 14a | >50 |
| 15a | >50 |
| 16a | 26.8 |
| 17a | >50 |
| 18a | >50 |
| 19a | >50 |
| 20a | >50 |
| 21a | >50 |
| 22a | >50 |
| 23a | >50 |
| 24a | >50 |
| 25a | >50 |
| 26a | >50 |
| 27a | >50 |
| 28a | >50 |
| 29a | >50 |
| 30a | >50 |
| 31a | >50 |
| 32a | >50 |
| 33a | >50 |
| 34a | >50 |
| 35a | >50 |
| 36a | >50 |
| 37a | >50 |
| 38a | >50 |
| 39a | >50 |
| 40a | >50 |
| 41a | >50 |
| 42a | 13.17 |
| 43a | >50 |
| 44a | 42.5 |
| 45a | >50 |
| 46a | 42.7 |
| 47a | >50 |
| 48a | >50 |
| 49a | >50 |
| 50a | >50 |
| 51a | >50 |
| 52a | >50 |
| 53a | >50 |
| 54a | >50 |
| 55a | >50 |
| 56a | >50 |
| 57a | >50 |
| 58a | >50 |
| 59a | >50 |
| 60a | 14.26 |
| 61a | >50 |
| 62a | 44 |
| 63a | >50 |
| 64a | >50 |
| 65a | 4.91 |
| 66a | 32.1 |
| 67a | >50 |
| 68a | >50 |
| 69a | 1.533 |
| 70a | 8.001 |
| 71a | 2.72 |

TABLE 3a.2
SMARCA4 inhibition by compounds of the disclosure.

| Compd. | FL-SMARCA4 |
|---|---|
| No. | IC₅₀ |
| 1a | >50 |
| 2a | >50 |
| 3a | >50 |
| 4a | >50 |
| 5a | >50 |
| 6a | >50 |
| 7a | >50 |
| 8a | >50 |
| 9a | >50 |
| 10a | >50 |
| 11a | >50 |
| 12a | >50 |
| 13a | >50 |
| 14a | >50 |
| 15a | >50 |
| 16a | >50 |
| 17a | >50 |
| 18a | >50 |
| 19a | ND |
| 20a | >50 |
| 21a | >50 |
| 22a | >50 |
| 23a | >50 |
| 24a | >50 |
| 25a | >50 |
| 26a | >50 |
| 27a | >50 |
| 28a | >50 |
| 29a | >50 |
| 30a | >50 |
| 31a | >50 |
| 32a | >50 |
| 33a | >50 |
| 34a | >50 |
| 35a | >50 |
| 36a | >50 |
| 37a | >50 |
| 38a | >50 |
| 39a | >50 |
| 40a | >50 |
| 41a | >50 |
| 42a | >50 |
| 43a | >50 |
| 44a | >50 |
| 45a | >50 |
| 46a | 41.1 |
| 47a | >50 |
| 48a | >50 |
| 49a | >50 |
| 50a | >50 |
| 51a | >50 |
| 52a | >50 |
| 53a | >50 |
| 54a | >50 |
| 55a | >50 |
| 56a | >50 |
| 57a | >50 |
| 58a | >50 |
| 59a | >50 |
| 60a | 46.7 |
| 61a | >50 |
| 62a | 48.2 |
| 63a | >50 |
| 64a | >50 |
| 65a | 27.02 |
| 66a | >50 |
| 67a | >50 |
| 68a | >50 |
| 69a | 8.485 |
| 70a | 25.09 |
| 71a | 39.58 |

TABLE 3b.1
SMARCA2 inhibition by compounds of the disclosure.

| Compd. | FL-SMARCA2 |
|---|---|
| No. | IC₅₀ |
| 1b | >50 |
| 2b | >50 |
| 3b | >50 |
| 4b | 42 |
| 5b | >50 |
| 6b | >50 |
| 7b | >50 |
| 8b | >50 |
| 9b | 43.7 |
| 10b | >50 |
| 11b | >50 |
| 12b | >50 |
| 13b | >50 |
| 14b | >50 |
| 15b | >50 |
| 16b | >50 |
| 17b | 0.6448 |
| 18b | 0.9172 |
| 19b | 2.024 |
| 20b | 2.456 |
| 21b | >50 |
| 22b | >50 |
| 23b | >50 |

TABLE 3b.2
SMARCA4 inhibition by compounds of the disclosure.

| Compd. | FL-SMARCA4 |
|---|---|
| No. | IC₅₀ |
| 1b | >50 |
| 2b | >50 |
| 3b | >50 |
| 4b | >50 |
| 5b | >50 |
| 6b | >50 |
| 7b | >50 |
| 8b | >50 |
| 9b | >50 |
| 10b | >50 |
| 11b | >50 |
| 12b | >50 |
| 13b | >50 |
| 14b | >50 |
| 15b | >50 |
| 16b | >50 |
| 17b | 4.783 |
| 18b | 5.343 |
| 19b | 12.62 |
| 20b | 11.46 |
| 21b | >50 |
| 22b | >50 |
| 23b | >50 |

TABLE 3c.1
SMARCA2 inhibition by compounds of the disclosure.

| Compd. | FL-SMARCA2 |
|---|---|
| No. | IC₅₀ |
| 1c | 0.402 |
| 2c | 24.57 |
| 3c | 40 |
| 4c | 6.982 |
| 5c | 0.02851 |
| 6c | 0.9006 |
| 7c | >50 |
| 8c | >50 |
| 9c | 2.88 |
| 10c | 3.351 |
| 11c | >50 |
| 12c | 34.8 |
| 13c | >50 |
| 14c | >50 |
| 15c | >50 |
| 16c | >50 |
| 17c | >50 |
| 18c | >50 |
| 19c | >50 |
| 20c | >50 |
| 21c | 0.2676 |
| 22c | 0.03012 |
| 23c | 0.1728 |
| 24c | 11.28 |
| 25c | 0.0142 |
| 26c | 5.413 |
| 27c | >50 |
| 28c | >50 |
| 29c | 25.4 |
| 30c | >50 |
| 31c | >50 |
| 32c | 5.668 |
| 33c | 8.902 |
| 34c | 0.0938 |
| 35c | 0.6984 |
| 36c | >50 |
| 37c | 0.4822 |
| 38c | >50 |
| 39c | 0.07798 |
| 40c | 0.09954 |
| 41c | 0.08268 |
| 42c | 0.2033 |
| 43c | 0.1304 |
| 44c | 1.04 |
| 45c | 0.09806 |
| 46c | >50 |
| 47c | 0.06495 |
| 48c | 25.3 |
| 49c | 16.78 |
| 50c | 2.615 |
| 51c | >50 |
| 52c | >50 |
| 53c | >50 |
| 54c | 0.04523 |
| 55c | 0.07784 |
| 56c | 0.2968 |
| 57c | 0.02089 |
| 58c | 0.04472 |
| 59c | 0.1321 |
| 60c | 0.2515 |
| 61c | 0.02665 |
| 62c | 0.008047 |
| 63c | 0.03335 |
| 64c | 0.008785 |
| 65c | 0.03367 |
| 66c | 0.2362 |
| 67c | 0.3706 |
| 68c | 0.06714 |
| 69c | 1.004 |
| 70c | 0.2601 |
| 71c | 0.09202 |
| 72c | 0.21 |
| 73c | 0.8471 |
| 74c | 0.06751 |
| 75c | 0.01997 |
| 76c | 24.1 |
| 77c | 6.555 |
| 78c | 0.09829 |
| 79c | 0.0568 |
| 80c | 0.1614 |
| 81c | 0.07982 |
| 82c | 0.009882 |
| 83c | 0.01943 |
| 84c | 1.85 |
| 85c | 0.08113 |
| 86c | 0.01722 |
| 87c | >50 |
| 88c | >50 |
| 89c | >50 |
| 90c | >50 |
| 91c | >50 |
| 92c | 0.1198 |
| 93c | >50 |
| 94c | >50 |
| 95c | 0.603 |
| 96c | 10.53 |
| 97c | 0.0607 |
| 98c | 0.1085 |
| 99c | 0.04064 |
| 100c | 6.813 |
| 101c | >50 |
| 102c | >50 |
| 103c | 31.28 |
| 104c | 22.4 |
| 105c | 0.04069 |
| 106c | 0.028 |
| 107c | 0.05078 |
| 108c | 0.08179 |
| 109c | 0.06249 |
| 110c | >50 |
| 111c | 0.01048 |
| 112c | >50 |
| 113c | 0.03448 |
| 114c | 30.54 |
| 115c | >50 |
| 116c | 0.2063 |
| 117c | 0.00522 |
| 118c | 40.27 |
| 119c | 6.172 |
| 120c | 0.09094 |
| 121c | 0.1911 |
| 122c | 0.05203 |
| 123c | 0.471 |
| 124c | 0.01085 |
| 125c | 0.05223 |
| 126c | 0.235 |
| 127c | 0.3518 |
| 128c | 0.2108 |
| 129c | 0.05131 |
| 130c | 0.01657 |
| 131c | 0.008677 |
| 132c | 5. > 503 |
| 133c | 0.01385 |
| 134c | 0.00812 |
| 135c | 39.5 |
| 136c | 0.02625 |
| 137c | 0.3004 |
| 138c | 0.0288 |
| 139c | 0.05994 |
| 140c | 0.03397 |
| 141c | 0.02796 |
| 142c | 0.04993 |
| 143c | 0.006077 |
| 144c | 0.02011 |
| 145c | 0.08168 |
| 146c | 9.545 |
| 147c | 5.24 |
| 148c | 0.1464 |
| 149c | 8.034 |
| 1 > 50c | 0.008977 |
| 151c | 0.02962 |
| 152c | 4.688 |
| 153c | 0.2113 |
| 154c | 0.07113 |
| 155c | 30.77 |
| 156c | 0.3467 |
| 157c | 0.4815 |
| 158c | 2.519 |
| 159c | 0.01672 |
| 160c | 0.1911 |
| 161c | ND |
| 162c | ND |
| 163c | ND |
| 164c | ND |
| 165c | ND |
| 166c | ND |
| 167c | ND |
| 168c | ND |
| 169c | ND |
| 170c | ND |
| 171c | ND |
| 172c | ND |
| 173c | ND |
| 174c | ND |
| 175c | ND |
| 176c | 9.19 |
| 177c | 0.0127 |
| 178c | 0.02145 |
| 179c | ND |

TABLE 3c.2
SMARCA4 inhibition by compounds of the disclosure.

| Compd. | FL-SMARCA4 |
|---|---|
| No. | IC_{>50} |
| 1c | >50 |
| 2c | >50 |
| 3c | >50 |
| 4c | 18.07 |
| 5c | 0.07592 |
| 6c | 0.679 |
| 7c | >50 |
| 8c | >50 |
| 9c | 7.82 |
| 10c | 7.129 |
| 11c | >50 |
| 12c | >50 |
| 13c | >50 |
| 14c | >50 |
| 15c | >50 |
| 16c | >50 |
| 17c | >50 |
| 18c | >50 |
| 19c | >50 |
| 20c | >50 |
| 21c | 0.5732 |
| 22c | 0.06075 |
| 23c | 0.2192 |
| 24c | 38.7 |
| 25c | 0.08 > 508 |
| 26c | 6.274 |
| 27c | >50 |
| 28c | >50 |
| 29c | 41.4 |
| 30c | >50 |
| 31c | >50 |
| 32c | 12.46 |
| 33c | 8.155 |
| 34c | 0.2326 |
| 35c | 0.9047 |
| 36c | >50 |
| 37c | 2.492 |
| 38c | >50 |
| 39c | 0.1432 |
| 40c | 0.2586 |
| 41c | 0.2499 |
| 42c | 0.4773 |
| 43c | 0.3291 |
| 44c | 2.191 |
| 45c | 0.2322 |
| 46c | >50 |
| 47c | 0.177 |
| 48c | >50 |
| 49c | 16.8 |
| 50c | 22.1 |
| 51c | >50 |
| 52c | >50 |
| 53c | >50 |
| 54c | 0.1656 |
| 55c | 0.269 |
| 56c | 1.036 |
| 57c | 0.0999 |
| 58c | 0.178 |
| 59c | 0.3786 |
| 60c | 0.492 |
| 61c | 0.08666 |
| 62c | 0.02684 |
| 63c | 0.1004 |
| 64c | 0.02972 |
| 65c | 0.1542 |
| 66c | 0.772 |
| 67c | 0.8067 |
| 68c | 0.1212 |
| 69c | 3.766 |
| 70c | 0.3854 |
| 71c | 0.3036 |
| 72c | 1.547 |
| 73c | 2.93 |
| 74c | 0.2449 |
| 75c | 0.0545 |
| 76c | >50 |
| 77c | 30.51 |
| 78c | 0.2565 |
| 79c | 0.1427 |
| 80c | 0.4941 |
| 81c | 0.1984 |
| 82c | 0.06615 |
| 83c | 0.06295 |
| 84c | 28.9 |
| 85c | 0.2334 |
| 86c | 0.04513 |
| 87c | >50 |
| 88c | >50 |
| 89c | >50 |
| 90c | >50 |
| 91c | >50 |
| 92c | 0.346 |
| 93c | >50 |
| 94c | >50 |
| 95c | 5.708 |
| 96c | 29.31 |
| 97c | 0.1907 |
| 98c | 0.2873 |
| 99c | 0.08012 |
| 100c | 27.6 |
| 101c | >50 |
| 102c | >50 |
| 103c | 26 |
| 104c | 29.6 |
| 105c | 0.1715 |
| 106c | 0.05335 |
| 107c | 0.1416 |
| 108c | 0.1997 |
| 109c | 0.1586 |
| 110c | >50 |
| 111c | 0.02651 |
| 112c | >50 |
| 113c | 0.1109 |
| 114c | >50 |
| 115c | >50 |
| 116c | 0.5475 |
| 117c | 0.00699 |
| 118c | >50 |
| 119c | 16.09 |
| 120c | 0.2391 |
| 121c | 0.3841 |
| 122c | 0.1295 |
| 123c | 1.074 |
| 124c | 0.01774 |
| 125c | 0.1655 |
| 126c | 0.9569 |
| 127c | 0.9 > 501 |
| 128c | 0.823 |
| 129c | 0.1432 |
| 130c | 0.04404 |
| 131c | 0.01374 |
| 132c | 11.35 |
| 133c | 0.0607 |
| 134c | 0.0128 |
| 135c | 35.8 |
| 136c | 0.1174 |
| 137c | 0.5582 |
| 138c | 0.06568 |
| 139c | 0.1861 |
| 140c | 0.06326 |
| 141c | 0.05981 |
| 142c | 0.119 |
| 143c | 0.008168 |
| 144c | 0.06911 |
| 145c | 0.2084 |
| 146c | 15.3 |
| 147c | 7.956 |
| 148c | 0.4324 |
| 149c | 16.08 |
| 150c | 0.01452 |
| 151c | 0.1108 |
| 152c | 37.5 |
| 153c | 0.4391 |
| 154c | 0.1468 |
| 155c | 36.16 |
| 156c | 0.6595 |
| 157c | 4.728 |
| 158c | 8.236 |
| 159c | 0.1053 |
| 160c | 0.3841 |
| 161c | ND |
| 162c | ND |
| 163c | ND |
| 164c | ND |
| 165c | ND |
| 166c | ND |
| 167c | ND |
| 168c | ND |
| 169c | ND |
| 170c | ND |
| 171c | ND |
| 172c | ND |
| 173c | ND |
| 174c | ND |
| 175c | ND |
| 176c | 9.19 |
| 177c | 0.07973 |
| 178c | 0.06674 |
| 179c | ND |

TABLE 3d.1
SMARCA2 inhibition by compounds of the disclosure.

| Compd. | FL-SMARCA2 |
|---|---|
| No. | IC_{>50} |
| 1d | >50 |
| 2d | >50 |
| 3d | >50 |
| 4d | 32 |
| 5d | >50 |
| 6d | 22.37 |
| 7d | >50 |
| 8d | >50 |
| 9d | >50 |
| 16d | 16.7 |
| 17d | >50 |
| 18d | >50 |
| 19d | >50 |
| 20d | 7.373 |
| 21d | 16.67 |
| 22d | 6.38 |
| 23d | 12.46 |
| 24d | 18.41 |
| 25d | >50 |
| 26d | >50 |
| 27d | >50 |
| 28d | >50 |
| 29d | >50 |
| 30d | >50 |
| 31d | 48.1 |
| 32d | >50 |
| 33d | >50 |
| 34d | >50 |
| 35d | >50 |
| 36d | 36.5 |
| 37d | >50 |
| 38d | >50 |
| 39d | >50 |
| 40d | >50 |
| 41d | >50 |
| 42d | 27.62 |
| 43d | >50 |
| 44d | 29.55 |
| 45d | >50 |
| 46d | >50 |
| 47d | 25.1 |
| 48d | >50 |
| 49d | >50 |
| 50d | 34.55 |
| 51d | >50 |
| 52d | 34 |
| 53d | >50 |
| 54d | 9.192 |
| 55d | 0.0926 |
| 56d | 0.09838 |
| 57d | 0.05157 |
| 58d | 0.06189 |
| 59d | 0.03406 |
| 60d | 0.018 |
| 61d | 0.197 |
| 62d | 0.02246 |
| 63d | 0.1649 |
| 64d | 0.1651 |
| 65d | 2.361 |
| 66d | 0. > 5053 |
| 67d | 0.01463 |
| 68d | 0.07249 |
| 69d | 0.02658 |
| 70d | 1.689 |
| 71d | 0.5359 |
| 72d | 0.4806 |
| 73d | 0.0109 |
| 74d | 0.4625 |
| 75d | 0.9478 |
| 76d | 1.314 |
| 77d | 0.1597 |
| 78d | 0.05755 |
| 79d | 0.04318 |
| 80d | 0.2235 |
| 81d | 0.06701 |
| 82d | 1.363 |
| 83d | 0.465 |
| 84d | 8.575 |
| 85d | 0.104 |
| 86d | 0.1007 |
| 87d | 0.9784 |
| 88d | 0.5765 |
| 89d | 0.1869 |
| 90d | 1.387 |
| 91d | 0.0229 |
| 92d | 0.03135 |
| 93d | 0.04308 |
| 94d | 1.768 |
| 95d | 0.3339 |
| 96d | 0.03815 |
| 97d | 2.695 |
| 98d | 0.03064 |
| 99d | 0.1948 |
| 100d | 0.00606 |
| 101d | 0.166 |
| 102d | 0.01936 |
| 103d | >50 |
| 104d | 0.02997 |
| 105d | 0.03141 |
| 106d | 2.185 |
| 107d | 0.5723 |
| 108d | 0.5421 |
| 109d | 1.064 |
| 110d | 1.466 |
| 111d | 1.704 |
| 112d | 0.3449 |
| 113d | 0.967 |
| 114d | 0.1116 |
| 115d | 0.03584 |
| 116d | 0.2519 |
| 117d | 22.4 |
| 118d | 0.2446 |
| 119d | 0.9615 |
| 120d | 0.07005 |
| 121d | 16.8 |
| 122d | 0.2528 |
| 123d | 0.5199 |
| 124d | >50 |
| 125d | 0.03566 |
| 126d | 1.578 |
| 127d | 0.5862 |
| 128d | >50 |
| 129d | 0.05838 |
| 130d | 0.0309 |
| 131d | >50 |
| 132d | >50 |
| 133d | 0.0461 |
| 134d | 0.0188 |
| 135d | 0.16 |
| 136d | 0.0419 |
| 137d | >50 |
| 138d | 1.68 |
| 139d | 3.1 |
| 140d | 0.772 |

TABLE 3d.2
SMARCA4 inhibition by compounds of the disclosure.

| Compd. | FL-SMARCA4 |
|---|---|
| No. | IC₅₀ |
| 1d | >50 |
| 2d | >50 |
| 3d | >50 |
| 4d | >50 |
| 5d | >50 |
| 6d | >50 |
| 7d | >50 |
| 8d | >50 |
| 9d | >50 |
| 16d | >50 |
| 17d | >50 |
| 18d | >50 |
| 19d | >50 |
| 20d | 28.9 |
| 21d | >50 |
| 22d | 28.9 |
| 23d | 15.29 |
| 24d | >50 |
| 25d | >50 |
| 26d | >50 |
| 27d | >50 |
| 28d | >50 |
| 29d | >50 |
| 30d | >50 |
| 31d | >50 |
| 32d | >50 |
| 33d | >50 |
| 34d | >50 |
| 35d | >50 |
| 36d | >50 |
| 37d | >50 |
| 38d | >50 |
| 39d | >50 |
| 40d | >50 |
| 41d | >50 |
| 42d | >50 |
| 43d | >50 |
| 44d | >50 |
| 45d | >50 |
| 46d | >50 |
| 47d | >50 |
| 48d | >50 |
| 49d | >50 |
| 50d | >50 |
| 51d | >50 |
| 52d | >50 |
| 53d | >50 |
| 54d | 28.44 |
| 55d | 0.246 |
| 56d | 0.5353 |
| 57d | 0.1906 |
| 58d | 0.102 |
| 59d | 0.2676 |
| 60d | 0.03627 |
| 61d | 0.6545 |
| 62d | 0.05706 |
| 63d | 0.5178 |
| 64d | 0.498 |
| 65d | 7.794 |
| 66d | 1.29 |
| 67d | 0.03715 |
| 68d | 0.2048 |
| 69d | 0.1201 |
| 70d | 4.751 |
| 71d | 1.256 |
| 72d | 1.308 |
| 73d | 0.0327 |
| 74d | 1.014 |
| 75d | 11.25 |
| 76d | 10.18 |
| 77d | 0.4631 |
| 78d | 0.3773 |
| 79d | 0.3096 |
| 80d | 1.138 |
| 81d | 0.2414 |
| 82d | 8.091 |
| 83d | 2.393 |
| 84d | >50 |
| 85d | 0.5962 |
| 86d | 0.5118 |
| 87d | 4.986 |
| 88d | 3.966 |
| 89d | 2.068 |
| 90d | 2.713 |
| 91d | 0.06983 |
| 92d | 0.1553 |
| 93d | 0.1473 |
| 94d | 17.92 |
| 95d | 1.06 |
| 96d | 0.09733 |
| 97d | 3.38 |
| 98d | 0.1213 |
| 99d | 0.2443 |
| 100d | 0.008526 |
| 101d | 0.2846 |
| 102d | 0.04635 |
| 103d | >50 |
| 104d | 0.08204 |
| 105d | 0.09055 |
| 106d | 5.629 |
| 107d | 1.537 |
| 108d | 1.778 |
| 109d | 3.161 |
| 110d | 4.615 |
| 111d | 5.102 |
| 112d | 2.136 |
| 113d | 2.882 |
| 114d | 0.3723 |
| 115d | 0.09928 |
| 116d | 0.6734 |
| 117d | >50 |
| 118d | 0.5439 |
| 119d | 2.224 |
| 120d | 0.2012 |
| 121d | 17.25 |
| 122d | 0.8346 |
| 123d | 1.578 |
| 124d | >50 |
| 125d | 0.09292 |
| 126d | 4.373 |
| 127d | 1.446 |
| 128d | >50 |
| 129d | 0.161 |
| 130d | 0.132 |
| 131d | >50 |
| 132d | >50 |
| 133d | 0.165 |
| 134d | 0.0587 |
| 135d | 0.334 |
| 136d | 0.0852 |
| 137d | >50 |
| 138d | 4.61 |
| 139d | 6.7 |
| 140d | 1.77 |

### General Procedure for SMARCA2 FRET Nucleosome Remodeling Activity Assay

The SMARCA2 FRET nucleosome remodeling assay was performed in a buffer consisting of 20 mM Bicine (pH 7.5), 10 mM KCl, 0.15 mM MgCl₂, 1 mM TCEP, 0.005% BSG, and 0.002% Tween 20, prepared on the day of use. Compounds in 100% DMSO (0.2 uL) were transferred into black polystyrene low volume 384-well plates, producing a 10 point curve for IC₅₀ determination. DMSO (0.2 uL) was added to Columns 11, 12, 23, 24, rows A-H for the maximum signal control and 0.2 uL SMARCA2 control compound (Compound 138) was added to columns 11, 12, 23, 24, rows I-P for the minimum signal control. The SMARCA2 enzyme (8 uL) was added to the compounds by an electronic multichannel pipettor and allowed to incubate with the compounds for 30 min at room temperature. The SMARCA2 remodeling assay was initiated by the addition of 2 uL of a nucleosome remodeling assay substrate and ATP mixture (final volume=10 uL). The final concentrations of the assay components were as follows: SMARCA2 was 5 nM, ATP was 85 uM, nucleosome remodeling assay substrate was 6 nM, control compound (Compound 138) in the minimum signal control wells was 1 uM, and the DMSO concentration was 2%. The assays were terminated after 15 minutes by the addition of 2 uL EDTA (final concentration of 25 mM). The SMARCA2 remodeling activity was determined by measuring the fluorescence ratio of the Cy3 and Cy5 labels (□em=531 nm, □ex=579 and 685 nm). The remodeling of the nucleosome substrate causes an increase in the spacing between the Cy3 and Cy5 labels and therefore a reduction in FRET and an increase in Cy3/Cy5 fluorescence ratio.

### Percent Inhibition Calculation

$$\% inh = 100 - ( \frac{{signal}_{cmpd} - {signal}_{min}}{{signal}_{max} - {signal}_{min}} ) \times 100$$

Where signal is luminescence intensity or Cy3/Cy5 ratio in the assay well, and min and max are the respective minimum and maximum signal controls.

### Four-Parameter IC₅₀ Fit

$$Y = Bottom + \frac{( Top - Bottom )}{( 1 + {( \frac{X}{{IC}_{50}} )}^{Hill Coefficient} )}$$

Where Y is the % inhibition and X is the compound concentration and the top and bottom plateaus may occasionally be fixed at 100 or 0 respectively in a 3-parameter fit. The results are shown in Table 4 (“A” means IC₅₀<100 nM; “B” means IC₅₀ ranging between 100 nM and 1 μM; “C” means IC₅₀ ranging between >1 μM and 10 μM; “D” means IC₅₀ ranging between >10 μM and 50 μM; “E” means IC₅₀>50 μM; “-” or “ND” means not determined).

TABLE 4
Results of remodeling assay

| Cmpd. | SMARCA2 |
|---|---|
| No. | Remodeling IC₅₀ |
| 1 | 7.115 |
| 101 | 0.314 |
| 125 | 0.06608 |
| 128 | 0.223 |
| 129 | 0.1141 |
| 131 | 0.171 |
| 136 | 0.0288 |
| 137 | 0.02904 |
| 138 | 0.01428 |
| 139 | 0.0253 |
| 140 | 0.089 |
| 141 | 0.399 |
| 142 | 0.06 |
| 143 | 0.168 |
| 144 | 1.269 |
| 145 | 5.059 |
| 146 | 2.573 |
| 148 | 0.02224 |
| 150 | 0.02174 |
| 151 | 0.02875 |
| 173 | 0.0758 |
| 176 | 0.0818 |
| 188 | 0.0222 |
| 189 | 0.0234 |
| 190 | 0.035 |
| 191 | 0.0653 |
| 192 | 0.0219 |
| 196 | 0.0181 |
| 249 | 0.07 |
| 260 | 0.0182 |
| 265 | 0.143 |
| 285 | 0.00922 |
| 290 | 0.407 |
| 295 | 0.499 |
| 298 | 0.0471 |

### Example 3-Long Term Proliferation (LTP) Assay

### Analysis of Cell Proliferation and Viability: Suspension Cell Lines

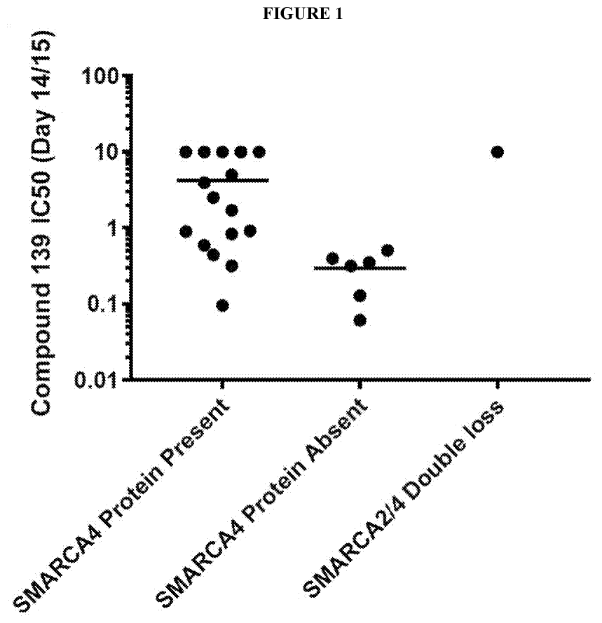

For assessment of cell proliferation and viability in cell lines cultured in suspension, exponentially growing cells were plated, in triplicate, in 96-well plates at a density previously determined to result in log linear phase growth throughout the assay period in a final volume of 150 μl. Cells were incubated in the presence of increasing concentrations of Compound 139 up to 10 μM. Viable cell number was determined every 3-4 days for up to 14 days via laser scanning imaging cytometry. On days of cell counts, growth media and Compound 139 were replaced and cells split back to the starting density. Total cell number is expressed as split-adjusted viable cells per well. For each cell line, IC₅₀ values were determined from concentration-dependence curves at each time point. The IC₅₀ values for the various lung cancer cell lines are summarized in FIG. 1, and Table below. The day 15 IC₅₀ of compounds of the disclosure in A549 cells are summarized in Table 6. In both tables, “A” means IC₅₀<100 nM; “B” means IC₅₀ ranging between 100 nM and 1 μM; “C” means IC₅₀ ranging between >1 μM and 10 μM; “D” means IC₅₀ ranging between >10 μM and 50 μM; “E” means IC₅₀>50 μM; “-” or “ND” means not determined).

TABLE 5
Summary of Inhibition of SMARCA2 in various cancer cell lines by compounds of the disclosure

| | Compd. | Compd. | Compd. | SMARCA4 | | |
|---|---|---|---|---|---|---|
| Cell line | 139 IC₅₀ | 298 IC₅₀ | 82c IC₅₀ | Protein | Cancer Indication | Cancer Subtype |
| A549 | 0.316 | 0.142 | 0.259 | Absent | Lung NSCLC | adenocarcinoma |
| CALU6 | 1.21 | 1.4 | ND | Present | Lung NSCLC | undifferentiated |
| | | | | | | carcinoma |
| CORL105 | 0.095 | 0.715 | 0.17 | Present | Lung NSCLC | adenocarcinoma |
| CORL23 | 1.7 | 1.4 | ND | Present | Lung NSCLC | large cell |
| | | | | | | carcinoma |
| COV644 | ND | 5.6 | ND | ND | Ovarian Cancer | NS |
| DLD1 | ND | 1.4 | ND | Present | Colon Cancer | adenocarcinoma |
| HCC15 | 0.677 | 0.569 | ND | Absent | Lung NSCLC | squamous cell |
| | | | | | | carcinoma |
| HCC44 | 1.266 | 3.3 | ND | Present | Lung NSCLC | adenocarcinoma |
| HCT116 | 0.169 | 0.836 | 0.142 | Present | Colon Cancer | NS |
| HS766T | 1 | 10 | ND | ND | Pancreatic Cancer | ductal carcinoma |
| HT1376 | ND | 0.385 | ND | ND | Bladder Cancer | transitional cell |
| | | | | | | carcinoma |
| MIAPACA2 | ND | 0.871 | ND | ND | Pancreatic Cancer | ductal carcinoma |
| NCIH1299 | 0.857 | 0.548 | 1.7 | ND | Lung NSCLC | non small cell |
| | | | | | | carcinoma |
| NCIH1437 | 1.5 | 1.4 | ND | Present | Lung NSCLC | adenocarcinoma |
| NCIH1563 | 2.3 | 1.1 | ND | Present | Lung NSCLC | adenocarcinoma |
| NCIH1568 | 0.395 | 0.509 | 10 | Absent | Lung NSCLC | adenocarcinoma |
| NCIH1573 | 0.504 | 0.701 | ND | Absent | Lung NSCLC | adenocarcinoma |
| NCIH1581 | ND | ND | 10 | Absent | Lung NSCLC | large cell |
| | | | | | | carcinoma |
| NCIH1693 | 0.101 | 0.202 | ND | Absent | Lung NSCLC | adenocarcinoma |
| NCIH1975 | 10 | 10 | ND | Present | Lung NSCLC | non small cell |
| | | | | | | carcinoma |
| NCIH2030 | 1.145 | 1.1 | ND | Absent | Lung NSCLC | non small cell |
| | | | | | | carcinoma |
| NCIH2085 | 0.314 | ND | 10 | Present | Lung NSCLC | adenocarcinoma |
| NCIH2122 | 2.8 | 3.9 | ND | Present | Lung NSCLC | adenocarcinoma |
| NCIH23 | 1.1 | 0.891 | ND | Absent | Lung NSCLC | non small cell |
| | | | | | | carcinoma |
| NCIH358 | 10 | 10 | 1.7 | Present | Lung NSCLC | bronchioloalveolar |
| | | | | | | adenocarcinoma |
| NCIH358 | 10 | 5.1 | ND | Present | Lung NSCLC | bronchioloalveolar |
| SMARCA2 | | | | | | adenocarcinoma |
| NCIH358 | 0.347 | 0.312 | 0.383 | Absent | Lung NSCLC | bronchioloalveolar |
| SMARCA4 | | | | | | adenocarcinoma |
| NCIH441 | 5.2 | 5 | ND | Present | Lung NSCLC | adenocarcinoma |
| NCIH460 | 10 | 10 | 10 | Present | Lung NSCLC | large cell |
| | | | | | | carcinoma |
| NCIH522 | 10 | 10 | 9.7 | Absent | Lung NSCLC | adenocarcinoma |
| NCIH596 | 8.8 | 10 | ND | ND | Lung NSCLC | mixed |
| | | | | | | adenosquamous |
| | | | | | | carcinoma |
| NCIH810 | 0.174 | 0.946 | ND | Present | Lung NSCLC | large cell |
| | | | | | | carcinoma |
| OV7 | ND | 0.154 | ND | ND | Ovarian Cancer | NS |
| PANC1 | 10 | 0.223 | ND | ND | Pancreatic Cancer | ductal carcinoma |
| RERFLCAI | 0.061 | 0.135 | 0.026 | Absent | Lung NSCLC | squamous cell |
| | | | | | | carcinoma |
| SW900 | 10 | 10 | ND | Present | Lung NSCLC | squamous cell |
| | | | | | | carcinoma |
| HCC1588 | 0.906 | ND | ND | Present | Lung NSCLC | squamous cell |
| | | | | | | carcinoma |
| NCIH1395 | 10 | ND | ND | Present | Lung NSCLC | adenocarcinoma |
| NCIH1435 | 0.443 | ND | ND | Present | Lung NSCLC | adenocarcinoma |
| NCIH1650 | 5 | ND | ND | Present | Lung NSCLC | bronchioalveolar |
| | | | | | | carcinoma |
| NCIH1792 | 10 | ND | ND | Present | Lung NSCLC | adenocarcinoma |
| NCIH1793 | 0.89 | ND | ND | Present | Lung NSCLC | adenocarcinoma |
| NCIH1838 | 0.587 | ND | ND | Present | Lung NSCLC | non-small cell lung |
| | | | | | | cancer |
| NCIH2126 | 5.5 | ND | ND | Present | Lung NSCLC | adenocarcinoma |
| NCIH2342 | 2.5 | ND | ND | Present | Lung NSCLC | non-small cell lung |
| | | | | | | cancer |
| NCIH838 | 10 | ND | ND | Present | Lung NSCLC | adenocarcinoma |
| HCC827 | 10 | ND | ND | Present | Lung NSCLC | adenocarcinoma |
| | | | | | | (bronchioalveolar |
| | | | | | | features) |

TABLE 6
Day 15 IC₅₀ of compounds of the disclosure in A549 cells

| Cmpd. | Day 15 |
|---|---|
| No. | IC₅₀ |
| 43 | 15.2 |
| 47 | 30 |
| 80 | 6.252 |
| 101 | 0.356 |
| 125 | 0.484 |
| 138 | 20 |
| 139 | 0.316 |
| 142 | 0.534 |
| 173 | 3.9 |
| 176 | 20 |
| 188 | 9.9 |
| 190 | 3.9 |
| 191 | 8.3 |
| 192 | 4.2 |
| 249 | 0.323 |
| 259 | 0.177 |
| 260 | 0.093 |
| 265 | 0.851 |
| 267 | 10 |
| 298 | 0.142 |
| 323 | 0.106 |
| 324 | 0.282 |
| 325 | 0.312 |
| 345 | 0.058 |
| 62c | 0.089 |
| 64c | 0.088 |
| 82c | 0.259 |
| 96c | 10 |
| 18d | 30 |

Analysis of Cell Proliferation and Viability: Adherent Cell Lines Protocol 1

The following cell lines were evaluated in an adherent cell line proliferation assay described herein: A549, HCC15, COV434, NCIH460, NCIH358, NCIH358-SMARCA2, NCIH358-SMARCA, NCIH1703, NCIH838, NCIH322, NCIH2122, NCIH2023, NCIH1355, NCIH1693, NCIH441, NCIH2030, NCIH1573, NCIH1373, NCIH1650, and NCIH1693. Plating densities for each cell line were determined based on growth curves (measured by ATP viability) and density over a 7 day timecourse. On the day before compound treatment, cells were plated in either 96-well plates in triplicate (for the day 0-7 timecourse) or 6-well plates (for replating on day 7 for the remainder of the timecourse). On Day 0, cells were either untreated, DMSO-treated, or treated with increasing concentrations of Compound 139 up to 10 μM. Plates were read on Day 0, Day 4, and Day 7 using a luminescent cell viability assay, with compound/media being replenished on Day 4. On Day 7, the 6-well plates were trypsinized, centrifuged, and resuspended in fresh media for counting by Vi-Cell. Cells from each treatment were replated at the original density in 96-well plates in triplicate. Cells were allowed to adhere to the plate overnight, and cells were treated as on Day 0. On Day 7, 11 and 14, plates were read using a luminescent cell viability assay, with compound/media being replenished on Day 11. Averages of triplicates were used to plot proliferation over the timecourse, and calculate IC₅₀ values.

### Analysis of Cell Proliferation and Viability: Adherent Cell Lines Protocol 2

The following cell lines were evaluated in an adherent cell line proliferation assay described herein: NCIH522, CORL23, NCIH1693, NCIH1838, HCC1588, NCIH1435, NCIH2085, HCC827, NCIH1792, NCIH596, NCIH1568, NCIH1793, NCIH2126, CORL105, NCIH1573, NCIH1693, NCIH1395, HCC44, NCIH2126, NCIH520, NCIH1373, NCIH2172, NCIH23, HCT116, RERFLCAI, NCIH2347, NCIH2110, NCIH647, NCIH1437, AGS, KMS11, BT549, HUPT4, NCIH1048, TE10, and TE14. Cell densities were determined by growth curve as per protocol 1. For the proliferation assay, on Day 0, cells were either untreated, DMSO-treated, or treated with increasing concentrations of Compound 139 up to 10 μM. Plates were read on Day 0, Day 4, and Day 7 using luminescent cell viability assay. On Day 7, the 6-well plates were trypsinized, centrifuged, and resuspended in fresh media for counting by Vi-Cell. Cells from each treatment were replated at the original density in 96-well plates in triplicate. Cells were treated with compound as on Day 0. On Day 7, 11 and 14, plates were read using a luminescent cell viability assay. Averages of triplicates were used to plot proliferation over the timecourse, and calculate IC₅₀ values.

### Example 4—High-Throughput Proliferation (HTP) Assay for A549 and H383 Cells

Assay for A549 Cells

Thawing vials: A549 cells (ATCC #CCL-185™) were thawed from a cryovial containing approximately 1.6×10\hat{ }6 cells frozen in 70% F12K medium, 20% HI-FBS and 10% DMSO. Cells were thawed by submerging the bottom half of the vial in a 37° C. water bath and gently flicked until almost thawed. The cells were transferred into a 15 mL conical tube containing 9 mL of F12K medium+10% HI-FBS and then centrifuged at 200×g for 4 min before drawing off the media without disturbing the cell pellet in order to remove the DMSO. The cells were resuspended again in 15 mL of F12K fresh complete media+10% HI-FBS and grown up in a T75 flask at 37° C. with 5% CO₂ Incubator. Viability was measured to obtain baseline.

Frozen stock vials preparation: The cell culture was transferred to a 50 mL conical tube, spun down to remove medium, and resuspended at 1-2×10\hat{ }6 cells/mL in complete medium containing 10% DMSO. The culture was then stored for 24 hours at −80 degree C., (with the initial rate of freezing is −1 degree C. per minute in a freezing container), then transferred and stored in liquid nitrogen.

Routine subculture: Sub-confluent cultures (70-80%) were split every 4 days, seeding out at 1-2×10\hat{ }6 cells/75 cm².

Day 0: Assay ready plates were prepared by dispensing 50 nL of compound or DMSO in the appropriate wells of 384-well white CulturPlates using Echo550. Using the Multiflo, A549 cells were plated at 50 μL/well in F12K complete media+10% HI-FBS+1% pen/strep at cell density determined [1,250 cells/mL (62.5 cells/well)] from growth curve.

Treated cells were incubated at 37° C., 5% CO₂, relative humidity >90% for 7 days. CellTiter-Glo (CTG) ATP detection reagent was prepared according to manufacturer's specifications. Cell assay plates were removed from the incubator and brought to room temperature (RT). Using the MultiDrop liquid handler, 30 μL of CTG were added per well. Plates were placed on the multidrop dispenser shake for 5 seconds at room temperature (RT). Plates were incubated for 30 minutes in the dark at RT before measuring luminescence using the Envison2104.

Data analysis: Using the DMSO control wells as the 0% inhibition (high signal) control and the 10 μM Doxorubicin control wells as the 100% inhibition (low signal) control, percent inhibition EC₅₀s (inflection point) and IC₅₀ s were calculated for the compound dose responses.

Assay for H358 Cells

Thawing vials: H358 cells (ATCC #CRL-5807™) were thawed from a cryovial containing approximately 1.6×10\hat{ }6 cells frozen in 70% RPMI 1640 medium, 20% HI-FBS and 10% DMSO. Cells were thawed by submerging the bottom half of the vial in a 37° C. water bath and gently flicked until almost thawed. The cells were removed and transferred into a 15 mL conical tube containing 9 mL RPMI 1640 medium+10% HI-FBS and then centrifuged 200×g for 4 min before drawing off the media without disturbing the cell pellet to remove the DMSO. The cells were resuspended again in 15 mL of RPMI 1640 fresh complete media+10% HI-FBS and grown up in a T75 flask at 37° C. with 5% CO₂ Incubator. Viability was measured to obtain baseline.

Preparing frozen stock vials: Cell density was counted in a stock flask. The culture was transferred to a 50 mL conical tube and spun down to remove medium, and then resuspend at 1-2×10\hat{ }6 cells/mL in complete medium containing 10% DMSO and transferred to a 1 mL per vial.

Routine subculture: Sub-confluent cultures (70-80%) were split every 4 days, seeding out at 1-2×10\hat{ }6 cells/75 cm².

Day 0: Assay ready plates were prepared by dispensing 50 nL of compound stock or DMSO in the appropriate wells of 384-well white CulturPlates using Echo550. Using the Multiflo, H358 cells at 50 μL/well were plated in RPMI 1640 complete media+10% HI-FBS+1% pen/strep at cell density determined [20,000 cells/mL (1,000 cells/well)] from growth curve. Treated cells were inclubated at 37° C., 5% CO₂, relative humidity >90% for 7 days. CellTiter-Glo (CTG) ATP detection reagent was prepared according to manufacturer's specifications. Cell assay plates were removed from incubator and bring to room temperature (RT).

Using the MultiDrop liquid handler, 30 μL of CTG were added per well. Plates were placed on the multidrop dispenser shake for 5 seconds at RT. Plates were incubated for 30 minutes in the dark at RT before measuring luminescence using the Envison2104.

Data analysis: Using the DMSO control wells as the 0% inhibition (high signal) control and the 10 uM Doxorubicin control wells as the 100% inhibition (low signal) control, percent inhibition EC₅₀s (inflection point) and IC₅₀s were calculated for the compound dose responses.

TABLE 7
Inhibition of SMARCA2 by compounds of
the disclosure in A549 and H358 cells

| | A549 | H358 |
|---|---|---|
| Compd. | SMARCA2 | SMARCA2 |
| No. | IC₅₀ | IC₅₀ |
| 80 | 11.48 | ND |
| 101 | 2.272 | ND |
| 125 | 1.16 | ND |
| 129 | 0.9044 | ND |
| 138 | 20 | ND |
| 139 | 0.868 | ND |
| 140 | 1.25 | ND |
| 142 | 1.342 | ND |
| 148 | 20 | ND |
| 173 | *13.1 | ND |
| 176 | 20 | ND |
| 182 | 20 | ND |
| 188 | *18.1 | ND |
| 189 | 9.689 | ND |
| 190 | 11.99 | ND |
| 191 | *13.4 | ND |
| 192 | 20 | ND |
| 196 | 0.597 | ND |
| 203 | 20 | ND |
| 207 | 0.8922 | ND |
| 220 | 20 | ND |
| 222 | 20 | ND |
| 223 | 20 | ND |
| 231 | 20 | ND |
| 232 | *15.53 | ND |
| 234 | 20 | ND |
| 235 | 20 | ND |
| 236 | 20 | ND |
| 237 | 20 | ND |
| 238 | *7.27 | ND |
| 240 | 20 | ND |
| 242 | 17.4 | ND |
| 249 | 0.734 | ND |
| 251 | 5.465 | ND |
| 259 | 2.46 | ND |
| 260 | 0.5281 | ND |
| 265 | 7.302 | ND |
| 275 | 20 | ND |
| 285 | 0.934 | ND |
| 297 | 2.09 | >10 |
| 298 | 0.6816 | >10 |
| 302 | >10 | ND |
| 323 | 0.3127 | ND |
| 330 | >10 | *8.74 |
| 336 | >10 | ND |
| 337 | 0.7693 | ND |
| 338 | 3.209 | ND |
| 339 | >10 | ND |
| 340 | >10 | ND |
| 342 | *9.49 | >10 |
| 343 | 0.4919 | >10 |
| 345 | 0.3549 | >10 |
| 346 | 5.238 | >10 |
| 5c | 1.155 | >10 |
| 22c | 0.664 | >10 |
| 25c | 0.6135 | >10 |
| 34c | 1.456 | >10 |
| 35c | 3.703 | 8.761 |
| 37c | 9.05 | >10 |
| 39c | 1.37 | >10 |
| 40c | 2.833 | >10 |
| 41c | 4.269 | >10 |
| 42c | 5.082 | >10 |
| 43c | 2.938 | >10 |
| 45c | 4.256 | >10 |
| 47c | 1.406 | >10 |
| 48c | >10 | >10 |
| 54c | 1.923 | >10 |
| 55c | 3.777 | >10 |
| 56c | 4.4 | *9.02 |
| 57c | 3.611 | >10 |
| 58c | 1.495 | >10 |
| 59c | 1.187 | 2.03 |
| 60c | >10 | >10 |
| 61c | 1.507 | >10 |
| 62c | 0.5485 | >10 |
| 63c | 2.286 | >10 |
| 64c | 0.3745 | >10 |
| 65c | 4.319 | >10 |
| 66c | >10 | >10 |
| 67c | >10 | >10 |
| 68c | 5.907 | >10 |
| 70c | 6.249 | 9.156 |
| 71c | 1.949 | >10 |
| 72c | 4.495 | >10 |
| 73c | *6.01 | >10 |
| 74c | 3.927 | >10 |
| 75c | 1.78 | >10 |
| 78c | 1.574 | >10 |
| 79c | 1.294 | >10 |
| 80c | 3.976 | >10 |
| 81c | 2.011 | >10 |
| 82c | 0.5889 | >10 |
| 83c | 3.471 | >10 |
| 85c | 2.371 | >10 |
| 86c | 1.727 | >10 |
| 92c | 2.326 | >10 |
| 95c | >10 | >10 |
| 96c | >10 | >10 |
| 97c | 3.781 | >10 |
| 98c | 3.295 | >10 |
| 99c | 1.703 | >10 |
| 111c | 1.948 | >10 |
| 113c | *4.03 | >10 |
| 116c | *3.79 | >10 |
| 117c | 1.823 | 1.489 |
| 120c | >10 | >10 |
| 121c | >10 | >10 |
| 122c | 4.603 | >10 |
| 123c | 3.98 | >10 |
| 124c | 1.075 | 7.229 |
| 125c | 4.456 | >10 |
| 126c | 4.01 | >10 |
| 127c | >10 | >10 |
| 128c | 5.13 | 3.89 |
| 129c | >10 | >10 |
| 130c | 1.211 | >10 |
| 131c | >10 | >10 |
| 134c | 0.909 | 3.887 |
| 136c | 0.7338 | >10 |
| 139c | 0.6393 | 0.5504 |
| 140c | >10 | >10 |
| 141c | 6.017 | >10 |
| 142c | 3.211 | 4.65 |
| 143c | 0.4582 | >10 |
| 144c | 1.679 | >10 |
| 145c | >10 | >10 |
| 146c | >10 | >10 |
| 147c | >10 | >10 |
| 148c | 4.248 | >10 |
| 149c | >10 | >10 |
| 150c | 0.9317 | >10 |
| 151c | 0.8886 | 4.855 |
| 153c | >10 | 6.952 |
| 154c | >10 | >10 |
| 155c | >10 | >10 |
| 157c | 3.35 | 9.72 |
| 158c | 0.6875 | 6.645 |
| 160c | 0.7865 | 7.144 |
| 177c | 1.37 | >10 |
| 178c | >10 | >10 |
| 55d | >10 | >10 |
| 56d | 6.245 | >10 |
| 57d | >10 | >10 |
| 58d | >10 | >10 |
| 59d | 2.08 | >10 |
| 60d | 3.65 | >10 |
| 61d | 4.331 | >10 |
| 62d | >10 | >10 |
| 63d | >10 | >10 |
| 64d | 3.808 | >10 |
| 65d | >10 | >10 |
| 67d | 2.115 | >10 |
| 68d | 2.406 | >10 |
| 69d | 2.042 | >10 |
| 70d | 1.963 | 9.845 |
| 71d | 2.384 | 4.088 |
| 72d | 5.756 | >10 |
| 73d | 1.811 | >10 |
| 74d | 1.649 | 2.44 |
| 75d | >10 | >10 |
| 76d | >10 | >10 |
| 77d | 7.438 | >10 |
| 78d | 6.373 | >10 |
| 79d | 3.649 | >10 |
| 80d | >10 | >10 |
| 81d | 2.37 | >10 |
| 82d | *4.73 | >10 |
| 83d | >10 | >10 |
| 84d | >10 | >10 |
| 85d | 4.171 | >10 |
| 86d | 3.953 | *8.56 |
| 87d | >10 | >10 |
| 88d | >10 | >10 |
| 89d | 5.625 | >10 |
| 90d | >10 | >10 |
| 91d | >10 | >10 |
| 92d | 1.987 | >10 |
| 93d | 3.927 | >10 |
| 95d | >10 | >10 |
| 96d | 4.435 | >10 |
| 97d | >10 | >10 |
| 98d | 2.931 | >10 |
| 99d | 6.684 | >10 |
| 100d | 4.347 | 6.245 |
| 101d | 3.74 | >10 |
| 102d | 1.046 | >10 |
| 103d | >10 | >10 |
| 104d | 4.367 | >10 |
| 105d | 4.295 | >10 |
| 106d | >10 | >10 |
| 107d | >10 | >10 |
| 108d | >10 | >10 |
| 109d | >10 | >10 |
| 110d | >10 | >10 |
| 111d | >10 | >10 |
| 112d | *9.96 | >10 |
| 113d | >10 | >10 |
| 114d | 2.728 | >10 |
| 115d | 2.085 | >10 |
| 116d | *8.92 | >10 |
| 117d | 1.96 | 2.274 |
| 118d | *7.18 | >10 |
| 119d | >10 | >10 |
| 120d | 2.12 | >10 |
| 121d | 5.025 | 7.38 |
| 122d | 8.444 | >10 |
| 123d | >10 | >10 |
| 124d | >10 | >10 |
| 125d | >10 | >10 |
| 126d | >10 | >10 |
| 127d | >10 | >10 |
| 128d | 3.239 | 4.05 |
| 129d | 2.173 | >10 |
| 130d | 3.369 | 6.747 |
| 131d | >10 | >10 |
| 132d | 7.985 | >10 |
| 133d | 2.89 | >10 |
| 134d | 1.33 | >10 |
| 135d | 2.38 | >10 |
| 136d | 2.22 | >10 |
| 137d | >10 | >10 |
| 138d | >10 | >10 |
| 139d | >10 | >10 |
| 140d | 7.18 | >10 |

Unless otherwise defined, all technical and scientific terms used herein have the same meaning as commonly understood by one of ordinary skill in the art to which this invention belongs. In the specification, the singular forms also include the plural unless the context clearly dictates otherwise. Unless specifically stated or obvious from context, as used herein, the terms “a,” “an,” and “the” are understood to be singular or plural. Unless specifically stated or obvious from context, as used herein, the term “or” is understood to be inclusive.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. Where names of cell lines or genes are used, abbreviations and names conform to the nomenclature of the American Type Culture Collection (ATCC) or the National Center for Biotechnology Information (NCBI), unless otherwise noted or evident from the context.

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

### Example 5—Compound 82c Efficacy Study in the Treatment of Subcutaneous A549 Xenograft Model

The efficacy of compound 82c was studied in vivo for the treatment of A549 subcutaneous xenograft model in BALB/c nude mice. Seventy-five BALB/c nude female mice were injected cell A549 at 1×10⁷ per mouse.

Fifty mice were selected and assigned into five groups using randomized block design based upon their tumor volumes and 10 mice per group. The 5 groups were treated p.o. with vehicle (0.5% NaCMC 0.1% Tween pH4), compound 82c 5 mg/kg BIDx21, 12.5 mg/kg BIDx10/QDx11, 25 mg/kg BIDx7/QDx14 and 50 mg/kg QDx10/3 days off/30 mg/kg QDx8.

All mice from 12.5 mg/kg BID were adjusted from BID to QD from treatment Day 11 until to the end of the study. #36˜#40 mice from group 25 mg/kg BID were compound administration holiday due to mice body weight loss. All mice from group 25 mg/kg BID were adjusted from BID to QD from treatment Day 8. All mice from group 50 mg/kg QD were compound administration holiday from Day 11 to Day 13 due to mice body weight loss and this group was adjusted from 50 mg/kg to 30 mg/kg until the end of the study.

Tumor Inoculation. Each mouse was inoculated subcutaneously at the right flank with A549 tumor cells (1×10⁷ cells/mouse) in 0.2 mL of base media (F12K) for tumor development. The treatments were started when the tumor size reached 126.94 mm³ for the tumor efficacy study (Day 14 post inoculation). Fifty tumor-bearing mice were block randomized into 5 groups with 10 mice in each. Test article administration and animal numbers in each group are shown in Table 7.

TABLE 7
Tumor weights, volumes, and TGI of different treatment groups, day 21

| | | Tumor | Weight- | Tumor | Volume- |
|---|---|---|---|---|---|
| | | Weight (g), | based | Volume (mm\hat{ }3), | based |
| Group | Treatment | Day 21 | TGI(%)^{#} | Day 21 | TGI(%)^{†} |
| 1 | Vehicle | 1.465 ± 0.141 | — | 1553.16 ± 161.44 | — |
| | BID×21 | | | | |
| 2 | Compound 82c | 0.773 ± 0.052 | 47.23* | 737.75 ± 78.79 | 57.01* |
| | 5 mg/kg | | | | |
| | BID×21 | | | | |
| 3 | Compound 82c | 0.609 ± 0.063 | 58.44* | 579.14 ± 59.39 | 68.31* |
| | 12.5 mg/kg | | | | |
| | BID×10 QD×11 | | | | |
| 4 | Compound 82c | 0.509 ± 0.077 | 65.26* | 469.53 ± 70.97 | 75.87* |
| | 25 mg/kg | | | | |
| | BID×7 QD×14 | | | | |
| 5 | Compound 82c | 0.496 ± 0.058 | 66.17* | 461.87 ± 60.61 | 76.56* |
| | 50 mg/kg QD×10 | | | | |
| | 3 days off | | | | |
| | 30 mg/kg QD×8 | | | | |

Note:
^{#}TGI(%) = (1 − TWₜᵣₑₐₜₘₑₙₜ/TW_{control)}) × 100%. TGI(%) > 58% was considered to be effective.
^{†}TGI(%) = (1 − (TVtreatment − Dx − TVtreatment − D1)/(TVcontrol − Dx − TVcontrol − D1)) × 100%. TGI(%) > 58% was considered to be effective.
*P < 0.001. P < 0.05 was considered to be statistically significant; P < 0.01 was considered to be statistically extremely significant.

Tumor Measurements and Endpoints. The major endpoint was tumor growth delay or cure. Tumor size was measured twice weekly in two dimensions using a caliper, and the volume was expressed in mm³ using the formula: V=0.5 a×b² where a and b were the long and short diameters of the tumor, respectively. The tumor size was then used for calculations of T/C (%) values. T/C (%) was calculated according to the following equation: T/C (%)=T_{RTV}/C_{RTV}×1000/9 T_{RTV}=TV_{treatment-Dn}/TV_{treatment-D1}, C_{RTV}=TV_{control-Dn}/TV_{control-D1}, T/C (%)<42%, it was considered to be effective. The tumor size was then used for calculations of TGI (%) values. TGI (%) was calculated according to the following equation: TGI (%)=(1−(TV_{Treatment/Dx}−TV_{Treatment/D1})/(TV_{Control/Dx}−TV_{Control/D1}))×100%, TGI□58 means this medicine is effective. The tumor weight was then used for calculations of TGI (%) values. TGI (%) was calculated according to the following equation: TGI (%)=(1−TW_{Treatment}/TW_{Control})×100%

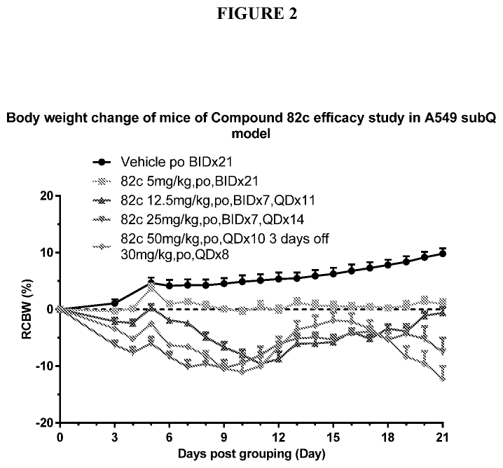

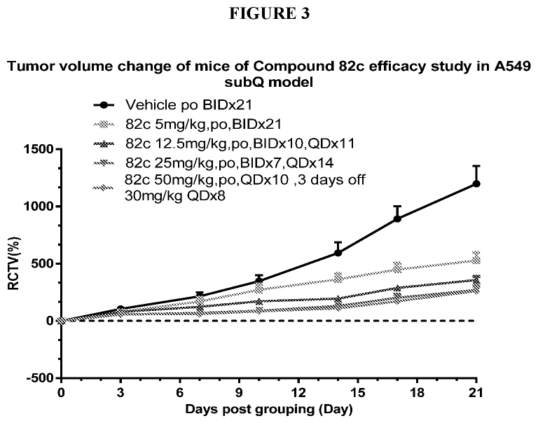

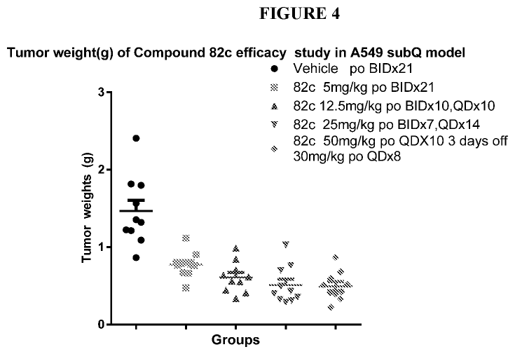

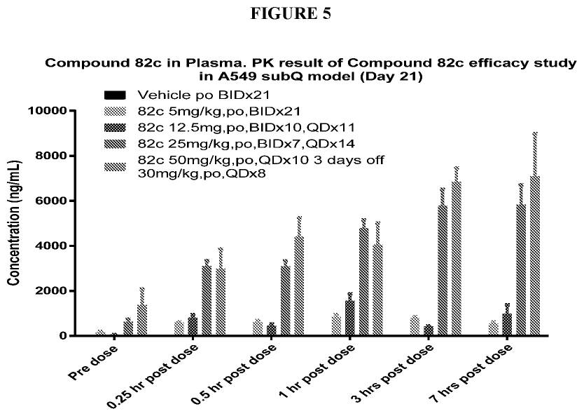

Statistical Analysis. Summary statistics, including mean and the standard error of the mean (SEM), were provided for the tumor volumes of each group at each time point. Statistical analyses of difference in tumor volumes among the groups were conducted on Day 21 after the last dose. Statistical analysis of difference in tumor volume and tumor weight among the groups was conducted. All data was analyzed using GraphPad Prism software. P<0.05 was considered to be statistically significant. A two-way ANOVA combined with Bonferroni post-test was performed to compare tumor volumes among vehicle and all treatment groups. A one-way ANOVA combined with Dunnett's Multiple Comparison Test to compare tumor weight among vehicle and all treatment groups. Results of the experiments are shown in FIGS. 2-5.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects are excluded are not set forth explicitly herein.

### Exemplary Embodiments

Embodiment 0. In some aspects, the present disclosure features a compound of Formula (I):

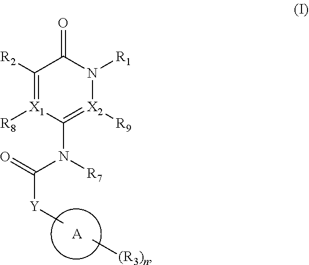

or a pharmaceutically acceptable salt thereof, wherein

A is a heteroaryl, heterocycloalkyl, aryl, or a cycloalkyl;

X₁ and X₂ are each independently selected from —CH and N;

Y is selected from the group consisting of a bond, —NH, —C(O), C₁-C₆ alkyl, —C(CH₃)₂—O—, and —CH₂—NH—CH₂—;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, —OR₅, —C(O)NH₂, —NO₂;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

each R₅ is independently selected from the group consisting of H, cyano, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, C₃-C₆ cycloalkyl, C₃-C₆ heterocycloalkyl or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′}; R₈ and R_{9′} are each independently selected from the group consisting of H, halo, and C₁-C₃ alkyl;

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

Embodiment 1. A compound of Formula (IA):

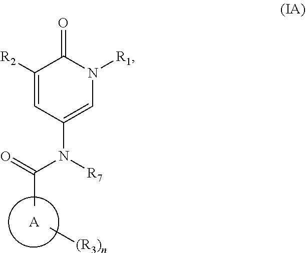

or a pharmaceutically acceptable salt thereof, wherein

A is a heteroaryl, heterocycloalkyl, aryl, or a cycloalkyl;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, C₃-C₆ cycloalkyl, C₃-C₆ heterocycloalkyl or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

Embodiment 2. A compound of compound Formula (IA) or a pharmaceutically acceptable salt thereof, wherein

A is a heteroaryl, heterocycloalkyl, aryl, or a cycloalkyl;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

Embodiment 3. A compound of Formula (IB):

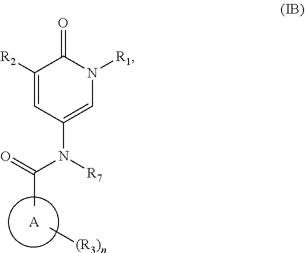

or a pharmaceutically acceptable salt thereof, wherein

A is a heteroaryl, heterocycloalkyl, aryl, or a cycloalkyl;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, C₃-C₆ cycloalkyl, C₃-C₆ heterocycloalkyl or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H and C₁-C₆ alkyl;

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted

Embodiment 4. A compound of Formula (IB) or a pharmaceutically acceptable salt thereof, wherein

A is a heteroaryl, heterocycloalkyl, aryl, or a cycloalkyl;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H and C₁-C₆ alkyl;

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

Embodiment 5. A compound of Formula (IC):

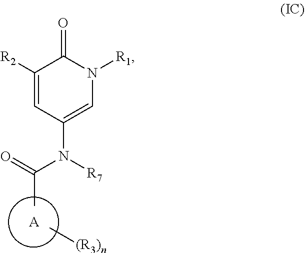

or a pharmaceutically acceptable salt thereof, wherein

A is a 5- or 6-membered heteroaryl having 1 to 4 heteroatoms selected from N, O, and S;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, C₃-C₆ cycloalkyl, C₃-C₆ heterocycloalkyl or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

Embodiment 6. A compound of Formula (IC) or a pharmaceutically acceptable salt thereof, wherein

A is a 5- or 6-membered heteroaryl having 1 to 4 heteroatoms selected from N, O, and S;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

Embodiment 7. A compound of Formula (ID):

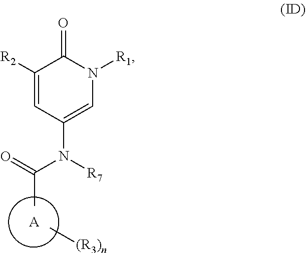

or a pharmaceutically acceptable salt thereof, wherein

A is a heteroaryl, heterocycloalkyl, aryl, or a cycloalkyl;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅;

each Q is independently selected from the group consisting of C₁-C₃ alkyl, C₂-C₆ alkenyl, C₃-C₆ cycloalkyl, C₃-C₆ heterocycloalkyl, and C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

provided that at least one R₃ is QR₆, wherein Q is C₂-C₆ alkynyl.

Embodiment 8. A compound of Formula (IE)

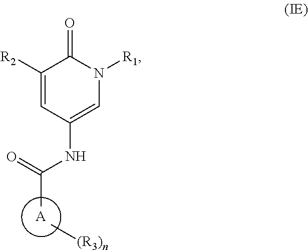

or a pharmaceutically acceptable salt thereof, wherein

A is a 5-membered heteroaryl having 1 to 4 heteroatoms selected from N, O, and S;

R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;

R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅;

R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};

each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};

each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};

each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,

wherein Q is C₁-C₃ alkyl, C₂-C₆ alkenyl, or C₂-C₆ alkynyl;

each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};

m is 1, 2, 3, 4, 5, or 6;

n is 0, 1, 2, 3, or 4; and

each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

Embodiment 9. The compound of any one of Embodiments 0-9, wherein each alkyl, alkoxyl, alkenyl, alkynyl, alkylcarbonyl, or alkylsulfonyl is unsubstituted or substituted with one or more substituents from the group consisting of halo, amino, alkoxyl, cycloalkyl, aryl, heterocycloalkyl, and heteroaryl.

Embodiment 10. The compound of any one of Embodiments 0-9, wherein each cycloalkyl, aryl, aryloxyl, heterocycloalkyl, or heteroaryl is unsubstituted or substituted with one or more substituents from the group consisting of halo, alkyl, haloalkyl, alkoxyl, cycloalkyl, aryl, heterocycloalkyl, and heteroaryl.

Embodiment 11. The compound of any one of Embodiments 0-10, wherein each aminocarbonyl, or aminosulfonyl is unsubstituted or substituted with one or more substituents from the group consisting of halo, alkyl, alkoxyl, cycloalkyl, aryl, heterocycloalkyl, and heteroaryl.

Embodiment 12. The compound of any one of Embodiments 0-11, wherein each cycloalkyl is independently a C₃-C₁₄ cycloalkyl.

Embodiment 13. The compound of any one of Embodiments 0-12, wherein each cycloalkyl is independently a C₃-C₈ cycloalkyl.

Embodiment 14. The compound of any one of Embodiments 0-13, wherein each aryl is independently a C₆-C₁₀ aryl.

Embodiment 15. The compound of any one of Embodiments 0-14, wherein each heteroaryl is independently a 5 to 6 membered heteroaryl.

Embodiment 16. The compound of any one of Embodiments 0-15, wherein each heterocycloalkyl is independently a 3 to 8-membered heterocycloalkyl.

Embodiment 17. The compound of any one of Embodiments 0-15, wherein each heterocycloalkyl is independently a 7 to 12-membered heterocycloalkyl.

Embodiment 17a. The compound of any one of Embodiments 0-17, wherein X₁ and X₂ are each independently selected from —CH and N.

Embodiment 17b. The compound of Embodiment 17a, wherein X₁ is —CH.

Embodiment 17c. The compound of Embodiment 17a, wherein X₁ is N.

Embodiment 17d. The compound of any one of Embodiments 0-17a, wherein X₂ is —CH.

Embodiment 17e. The compound of any one of Embodiments 0-17a, wherein X₂ is —N.

Embodiment 17f The compound of any one of Embodiments 0-17e, wherein Y is selected from the group consisting of a bond, —NH, —C(O), C₁-C₆ alkyl, —C(CH₃)₂—O—, and —CH₂—NH—CH₂—;

Embodiment 17 g. The compound of Embodiment 17f, wherein Y is a bond.

Embodiment 17h. The compound of Embodiment 17f, wherein Y is —NH.

Embodiment 17i. The compound of Embodiment 17f, wherein Y is —C(O).

Embodiment 17j. The compound of Embodiment 17f, wherein Y is a C₁-C₆ alkyl.

Embodiment 17k. The compound of Embodiment 17j, wherein Y is CH₃.

Embodiment 17l. The compound of Embodiment 17j, wherein Y is CH₂—CH₃.

Embodiment 17m. The compound of Embodiment 17f, wherein Y is —C(CH₃)₂—O—,

Embodiment 17n. The compound of Embodiment 17f, wherein Y is —CH₂—NH—CH₂—.

Embodiment 17o. The compound of any one of Embodiments 0-17n, wherein R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′}.

Embodiment 17p. The compound of any one of Embodiments 0-17n, R₈ and R_{9′} are each independently selected from the group consisting of H, halo, and C₁-C₃ alkyl.

Embodiment 18. The compound of any one of Embodiments 0-17p, wherein A is a 6 membered heteroaryl.

Embodiment 19. The compound of any one Embodiments 0-17n, wherein A is a 7-12 membered heteroaryl.

Embodiment 20. The compound of any one Embodiments 0-17n, wherein A is a 3 to 8-membered heterocycloalkyl having 1 to 4 heteroatoms selected from N, O, and S.

Embodiment 21. The compound of Embodiment 17, wherein A is a monocyclic heterocycloalkyl.

Embodiment 22. The compound of any one Embodiments 0-17n, wherein A is a 7 to 12-membered heterocycloalkyl having 1 to 4 heteroatoms selected from N, O, and S.

Embodiment 23. The compound of any one Embodiments 0-17n, wherein A is a 10-membered heterocycloalkyl having 1 to 4 heteroatoms selected from N, O, and S.

Embodiment 24. The compound of Embodiment 22 or 23, wherein A is a bicyclic heterocycloalkyl.

Embodiment 25. The compound of any one Embodiments 0-17n, wherein A is C₃-C₁₄ cycloalkyl.

Embodiment 26. The compound of Embodiment 25, wherein A is C₃-C₈ cycloalkyl.

Embodiment 27. The compound of Embodiment 26, wherein A is a C₃ cycloalkyl.

Embodiment 28. The compound of Embodiment 27, wherein A is cyclopropyl.

Embodiment 29. The compound of Embodiment 26, wherein A is a C₄ cycloalkyl.

Embodiment 30. The compound of Embodiment 26, wherein A is a C₅ cycloalkyl.

Embodiment 31. The compound of Embodiment 26, wherein A is a C₆ cycloalkyl.

Embodiment 32. The compound of any one of Embodiments 0-31, wherein R₃ is selected from the group consisting of halo, hydroxyl, COOH, cyano, nitro, oxo, C₁-C₆ alkyl, C₂-C₆ alkenyl, C₂-C₆ alkynyl, C₁-C₆ haloalkyl, C₃-C₈ cycloalkyl, 4 to 7-membered heterocycloalkyl, 5 to 12-membered heterocycloalkyl, aminocarbonyl, mono-C₁-C₆ alkylaminocarbonyl, di-C₁-C₆ alkylaminocarbonyl, C₁-C₆ alkylcarbonylamino, QR₆, —(CH₂)ₘR₆, —NR₅R_{5′}, and —OR₅.

Embodiment 33. The compound of any one of Embodiments 0-32, wherein R₆ is selected from the group consisting of halo, hydroxyl, COOH, cyano, C₂-C₆ alkenyl, C₂-C₆ alkynyl, C₁-C₆ alkoxyl, 4 to 7-membered heterocycloalkyl, —NR₅R_{5′.}

Embodiment 34. The compound of any one of Embodiments 0-33, wherein A is selected from thiazolyl, isothiazolyl, thiazol-2-onyl, thiophenyl, pyrrolyl, pyrazolyl, imidazolyl, 1,2,3-thiadiazolyl, 1,2,4-thiadiazolyl, furanyl, oxazolyl, isoxazolyl, 1,2,4-triazolyl, and 1,2,3-triazolyl.

Embodiment 35. The compound of any one of Embodiments 0-34, wherein A is selected from thiazolyl, thiophenyl, pyrrolyl, and pyrazolyl.

Embodiment 36. The compound of any one of Embodiments 0-35, wherein A is thiazolyl or thiophenyl.

Embodiment 37. The compound of any one of Embodiments 0-36, wherein A is N-substituted pyrrolyl.

Embodiment 38. The compound of any one of Embodiments 0-37, wherein R₁ is selected from the group consisting of H, C₁-C₆ alkyl, C₁-C₆ haloalkyl, C₆-C₁₀ aryl, C₃-C₈ cycloalkyl, and —(CH₂)ₘR₄.

Embodiment 39. The compound of Embodiment 38, wherein R₁ is selected from the group consisting of H, C₁-C₆ alkyl, or C₁-C₆ haloalkyl.

Embodiment 40. The compound of Embodiment 39, wherein R₁ is methyl, ethyl, halomethyl or haloethyl.

Embodiment 41. The compound of Embodiment 40, wherein R₁ is fluoroalkyl.

Embodiment 42. The compound of Embodiment 41, wherein R₁ is selected from the group consisting of fluoromethyl, difluoromethyl, trifluoromethyl, fluoroethyl, difluoroethyl, and trifluoroethyl.

Embodiment 43. The compound of Embodiment 38, wherein R₁ is C₃-C₈ cycloalkyl.

Embodiment 44. The compound of Embodiment 43, wherein R₁ is cyclopropyl.

Embodiment 45. The compound of Embodiment 38, wherein R₁ is C₆-C₁₀ aryl.

Embodiment 46. The compound of Embodiment 45, wherein R₁ is phenyl.

Embodiment 47. The compound of Embodiment 38, wherein R₁ is —(CH₂)ₘR₄.

Embodiment 48. The compound of Embodiment 47, wherein R₄ is selected from the group consisting of C₁-C₆ alkoxyl, mono-C₁-C₆ alkylamino, and di-C₁-C₆ alkylamino.

Embodiment 49. The compound of Embodiment 47, wherein R₄ is hydroxyl.

Embodiment 50. The compound of Embodiment 48, wherein R₄ is mono-C₁-C₆ alkylamino.

Embodiment 51. The compound of Embodiment 50, wherein R₄ is methylamino.

Embodiment 52. The compound of Embodiment 48, wherein R₄ is di-C₁-C₆ alkylamino.

Embodiment 53. The compound of Embodiment 46, wherein R₄ is dimethylamino.

Embodiment 54. The compound of Embodiment 47, wherein R₄ is C₁-C₆ alkoxyl.

Embodiment 55. The compound of Embodiment 54, wherein R₄ is methoxyl.

Embodiment 56. The compound of Embodiment 47, wherein R₄ is C₆-C₁₀ aryl.

Embodiment 57. The compound of Embodiment 56, wherein R₄ is phenyl.

Embodiment 58. The compound of Embodiment 47, wherein R₄ is C₃-C₈ cycloalkyl.

Embodiment 59. The compound of Embodiment 58, wherein R₄ is cyclopropyl.

Embodiment 60. The compound of Embodiment 47, wherein R₄ is a 5-membered heteroaryl.

Embodiment 61. The compound of Embodiment 60, wherein R₄ is pyrazolyl or imidazolyl.

Embodiment 62. The compound of Embodiment 47, wherein R₄ is a 5-membered heterocycloalkyl.

Embodiment 63. The compound of Embodiment 62, wherein R₄ is pyrrolidinyl.

Embodiment 64. The compound of any one of Embodiments 47-63, wherein m is 1.

Embodiment 65. The compound of any one of Embodiments 47-63, wherein m is 2.

Embodiment 66. The compound of any one of Embodiments 47-63, wherein m is 3, 4, 5, or 6.

Embodiment 67. The compound of any one of Embodiments 0-65, wherein R₂ is selected from the group consisting of H, halo, cyano, C₁-C₆ alkyl, —(CH₂)ₘR₄, —NR₅R_{5′}, —OR₅, —C(O)NH₂, and —NO₂.

Embodiment 68. The compound of Embodiment 67, wherein R₂ is H.

Embodiment 69. The compound of Embodiment 67, wherein R₂ is cyano.

Embodiment 69a. The compound of Embodiment 67, wherein R₂ is —C(O)NH₂.

Embodiment 69b. The compound of Embodiment 67, wherein R₂ is —NO₂.

Embodiment 70. The compound of Embodiment 67, wherein R₂ is halo.

Embodiment 71. The compound of Embodiment 70, wherein R₂ is F, Cl, or Br.

Embodiment 72. The compound of Embodiment 67, wherein R₂ is C₁-C₆ alkyl.

Embodiment 73. The compound of Embodiment 72, wherein R₂ is methyl, ethyl, or propyl.

Embodiment 74. The compound of Embodiment 67, wherein R₂ is —(CH₂)m R₄.

Embodiment 75. The compound of Embodiment 74, wherein R₄ is C₆-C₁₀ aryl.

Embodiment 76. The compound of Embodiment 75, wherein R₄ is phenyl.

Embodiment 77. The compound of Embodiment 74, wherein R₄ is a 5-membered heteroaryl.

Embodiment 78. The compound of Embodiment 77, wherein R₄ is 1-methyl-pyrazolyl.

Embodiment 79. The compound of Embodiment 67, wherein R₂ is —NR₅R_{5′}, R₅ is H and R_{5′} is C₁-C₆ alkyl.

Embodiment 80. The compound of Embodiment 67, wherein R₂ is —NR₅R_{5′}, and R₅ and R_{5′} are both C₁-C₆ alkyl.

Embodiment 81. The compound of Embodiment 67, wherein R₂ is —NR₅R_{5′}, R₅ is H and R_{5′} is —(CH₂)ₘR_{4′}.

Embodiment 82. The compound of Embodiment 56, wherein R₄′ is C₁-C₆ alkoxyl.

Embodiment 83. The compound of Embodiment 82, wherein R₄′ is methoxyl.

Embodiment 84. The compound of Embodiment 81, wherein R₄′ is di-C₁-C₆ alkylamino.

Embodiment 85. The compound of Embodiment 84, wherein R₄′ is dimethylamino.

Embodiment 86. The compound of Embodiment 81, wherein R₄′ is a 6-membered heteroaryl.

Embodiment 87. The compound of Embodiment 86, wherein R₄′ is pyridinyl.

Embodiment 88. The compound of Embodiment 81, wherein R₄′ is a 6-membered heterocycloalkyl.

Embodiment 89. The compound of Embodiment 88, wherein R₄′ is morpholinyl.

Embodiment 90. The compound of Embodiment 81, wherein R₄′ is a 5-membered heteroaryl.

Embodiment 91. The compound of Embodiment 90, wherein R₄′ is 1-methylpyrazolyl.

Embodiment 92. The compound of Embodiment 90, wherein R₄′ is imidazolyl

Embodiment 93. The compound of Embodiment 81, wherein R₄′ is a 5-membered heterocyclyl.

Embodiment 94. The compound of Embodiment 93, wherein R₄′ is pyrrolidinyl.

Embodiment 95. The compound of Embodiment 67, wherein R₂ is —OR₅ and R₅ is —(CH₂)ₘR₄′.

Embodiment 96. The compound of Embodiment 95, wherein R₄′ is selected from the group consisting of C₁-C₆ alkoxyl, mono-C₁-C₆ alkylamino, and di-C₁-C₆ alkylamino.

Embodiment 97. The compound of Embodiment 96, wherein R₄′ is C₁-C₆ alkoxyl.

Embodiment 98. The compound of Embodiment 97, wherein R₄′ is methoxyl.

Embodiment 99. The compound of Embodiment 96, wherein R₄′ is mono-C₁-C₆ alkylamino.

Embodiment 100. The compound of Embodiment 99, wherein R₄′ is methylamino.

Embodiment 101. The compound of Embodiment 96, wherein R₄′ is di-C₁-C₆ alkylamino.

Embodiment 102. The compound of Embodiment 101, wherein R₄′ is dimethylamino.

Embodiment 103. The compound of Embodiment 95, wherein R₄′ is a 6-membered heterocycloalkyl.

Embodiment 104. The compound of Embodiment 103, wherein R₄′ is 1-methylpiperazine or morpholinyl.

Embodiment 105. The compound of any one of Embodiments 74-104, wherein m is 1.

Embodiment 106. The compound of any one of Embodiments 74-104 wherein m is 2.

Embodiment 107. The compound of any one of Embodiments 0-106, wherein R₃ is selected from the group consisting of halo, cyano, nitro, oxo, C₁-C₆ alkyl, C₁-C₆ alkenyl, C₁-C₆ haloalkyl, C₃-C₈ cycloalkyl, C₆-C₁₀ aryl, 5 to 6-membered heteroaryl, 5 to 12-membered heterocycloalkyl, aminocarbonyl, mono-C₁-C₆ alkylaminocarbonyl, di-C₁-C₆ alkylaminocarbonyl, C₁-C₆ alkylsulfonyl, aminosulfonyl, QR₆, —(CH₂)ₘR₆, —NR₅R_{5′}, and —OR₅.

Embodiment 108. The compound of Embodiment 107, wherein R₃ is selected from the group consisting of halo, cyano, nitro, oxo, C₁-C₆ alkyl, C₁-C₆ alkenyl, C₁-C₆ haloalkyl, C₃-C₈ cycloalkyl, 5 to 12-membered heterocycloalkyl, aminocarbonyl, mono-C₁-C₆ alkylaminocarbonyl, di-C₁-C₆ alkylaminocarbonyl, C₁-C₆ alkylsulfonyl, aminosulfonyl, QR₆, —(CH₂)ₘR₆, —NR₅R_{5′}, and —OR₅.

Embodiment 109. The compound of Embodiment 107, wherein R₃ is halo.

Embodiment 110. The compound of Embodiment 107, wherein R₃ is cyano.

Embodiment 111. The compound of Embodiment 107, wherein R₃ is nitro.

Embodiment 112. The compound of Embodiment 107, wherein R₃ is oxo.

Embodiment 113. The compound of Embodiment 107, wherein R₃ is C₁-C₆ alkenyl.

Embodiment 114. The compound of Embodiment 107, wherein R₃ is C₁-C₆ haloalkyl.

Embodiment 115. The compound of Embodiment 114, wherein R₃ is trifluoromethyl.

Embodiment 116. The compound of Embodiment 107, wherein R₃ is aminocarbonyl, mono-C₁-C₆ alkylaminocarbonyl, or di-C₁-C₆ alkylaminocarbonyl.

Embodiment 117. The compound of Embodiment 107, wherein R₃ is methylaminocarbonyl.

Embodiment 118. The compound of Embodiment 107, wherein R₃ is dimethylaminocarbonyl.

Embodiment 119. The compound of Embodiment 107, wherein R₃ is C₁-C₆ alkylsulfonyl.

Embodiment 120. The compound of Embodiment 107, wherein R₃ is aminosulfonyl.

Embodiment 121. The compound of Embodiment 107, wherein R₃ is methylsulfonyl.

Embodiment 122. The compound of Embodiment 107, wherein R₃ is C₆-C₁₀ aryl.

Embodiment 123. The compound of Embodiment 122, wherein R₃ is phenyl.

Embodiment 124. The compound of Embodiment 122 or 123, wherein the C₆-C₁₀ aryl is substituted with one or more C₁-C₆ alkyl, halogen, or C₁-C₆ alkoxyl.

Embodiment 125. The compound of Embodiment 107, wherein R₃ is C₃-C₈ cycloalkyl.

Embodiment 126. The compound of Embodiment 125, wherein R₃ is cyclopropyl.

Embodiment 127. The compound of Embodiment 107, wherein R₃ is a 5 to 6-membered heteroaryl.

Embodiment 128. The compound of Embodiment 127, wherein R₃ is selected from oxazolyl, pyridinyl, furanyl, thiazolyl, pyrrolyl, imidazolyl, and pyrazolyl.

Embodiment 129. The compound of Embodiment 127 or 128, wherein the 5 to 6-membered heteroaryl is substituted with one or more methyl.

Embodiment 130. The compound of Embodiment 127 or 128, wherein the 5 to 6-membered heteroaryl is substituted with one or more C₁-C₆ haloalkyl.

Embodiment 131. The compound of Embodiment 130, wherein the 5 to 6-membered heteroaryl is substituted with trifluoromethyl.

Embodiment 132. The compound of Embodiment 129, wherein R₃ is selected from the group consisting of 2-methylthiazolyl, 1,2-dimethyl-pyrrolyl, 1-methyl-imidazolyl, and 1-methyl-pyrazolyl.

Embodiment 133. The compound of Embodiment 107, wherein R₃ is 5 to 12-membered heterocycloalkyl.

Embodiment 134. The compound of Embodiment 134, wherein R₃ is 2,3-dihydrobenzofuranyl.

Embodiment 135. The compound of Embodiment 107, wherein R₃ is —(CH₂)ₘR₆.

Embodiment 136. The compound of Embodiment 135, wherein R₆ is hydroxyl.

Embodiment 137. The compound of Embodiment 135, wherein R₆ is C₆-C₁₀ aryl.

Embodiment 138. The compound of Embodiment 137, wherein C₆-C₁₀ aryl is substituted with C₁-C₆ alkoxyl.

Embodiment 139. The compound of Embodiment 137 or 138, wherein C₆-C₁₀ aryl is phenyl.

Embodiment 140. The compound of any one of Embodiments 135-139, wherein m is 1.

Embodiment 141. The compound of Embodiment 107, wherein R₃ is QR₆.

Embodiment 142. The compound of Embodiment 141, wherein R₆ is a 4, 5 or 6-membered heterocyclyl.

Embodiment 143. The compound of Embodiment 142, wherein R₆ is oxetanyl, pyrrolidinyl, or morpholinyl.

Embodiment 144. The compound of Embodiment 141, wherein R₆ is a 5 or 6-membered heteroaryl.

Embodiment 145. The compound of Embodiment 144, wherein R₆ is pyridinyl, pyrimidinyl, furanyl, thiazolyl, imidazolyl, or pyrrolyl.

Embodiment 146. The compound of Embodiment 141, wherein R₆ is amino.

Embodiment 147. The compound of Embodiment 141, wherein R₆ is di-C₁-C₆ alkylamino.

Embodiment 148. The compound of Embodiment 147, wherein R₆ is dimethylamino.

Embodiment 149. The compound of Embodiment 141, wherein R₆ is hydroxyl.

Embodiment 150. The compound of Embodiment 142, wherein R₆ is C₁-C₆ haloalkyl.

Embodiment 151. The compound of Embodiment 150, wherein R₆ is trifluoromethyl.

Embodiment 152. The compound of any one of Embodiments 141-151, wherein Q is prop-1-ynyl.

Embodiment 153. The compound of any one of Embodiments 141-151, wherein Q is a C₁-C₃ alkyl.

Embodiment 154. The compound of Embodiment 153, wherein Q is substituted with OH.

Embodiment 155. The compound of Embodiment 153, wherein Q is substituted with halo.

Embodiment 156. The compound of Embodiment 155, wherein Q is substituted with fluoro. Embodiment 157. The compound of Embodiment 153 or 154, wherein Q is methyl.

Embodiment 158. The compound of Embodiment 107, wherein R₃ is —NR₅R_{5′.}

Embodiment 159. The compound of Embodiment 158, wherein R₅ is H and R_{5′} is C₃-C₈ cycloalkyl.

Embodiment 160. The compound of Embodiment 159, wherein R_{5′} is cyclopentyl.

Embodiment 161. The compound of Embodiment 158, wherein R₅ is H and R_{5′} is C₁-C₆ alkyl.

Embodiment 162. The compound of Embodiment 161, wherein R_{5′} is methyl.

Embodiment 163. The compound of Embodiment 161, wherein R_{5′} is i-propyl.

Embodiment 164. The compound of Embodiment 158, wherein R₅ is H and R_{5′} is C₁-C₆ alkylcarbonyl.

Embodiment 165. The compound of Embodiment 164, wherein R_{5′} is ethanoyl.

Embodiment 166. The compound of Embodiment 107, wherein R₃ is OR₅.

Embodiment 167. The compound of Embodiment 166, wherein R₅ is C₁-C₆ alkyl.

Embodiment 168. The compound of Embodiment 167, wherein the C₁-C₆ alkyl is methyl.

Embodiment 169. The compound of any one of the preceding Embodiments, wherein n is 2 or 3.

Embodiment 170. The compound of Embodiment 107, wherein n is 1 and R₃ is cyano.

Embodiment 171. The compound of Embodiment 107, wherein n is 1 or 2 and R₃ is halo.

Embodiment 172. The compound of Embodiment 107, wherein n is 2, one R₃ is halo and the other R₃ is cyano.

Embodiment 173. The compound of any Embodiment 107, 171, or 172, wherein halo is selected from Cl, Br, and I.

Embodiment 174. The compound of Embodiment 1, wherein A is thiazolyl.

Embodiment 175. The compound of Embodiment 1, wherein A is thiophenyl.

Embodiment 176. The compound of Embodiment 174 or 175, wherein n is 1 and R₃ is cyano.

Embodiment 177. The compound of Embodiment 174 or 175, wherein n is 2 and R₃ is selected from halo and cyano.

Embodiment 178. The compound any one of Embodiments 174 or 177, wherein n is 2 and each R₃ is halo.

Embodiment 179. The compound of Embodiment 177 or 178, wherein halo is chloro or fluoro.

Embodiment 180. The compound of Embodiment 9, wherein

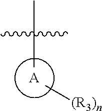

is selected from

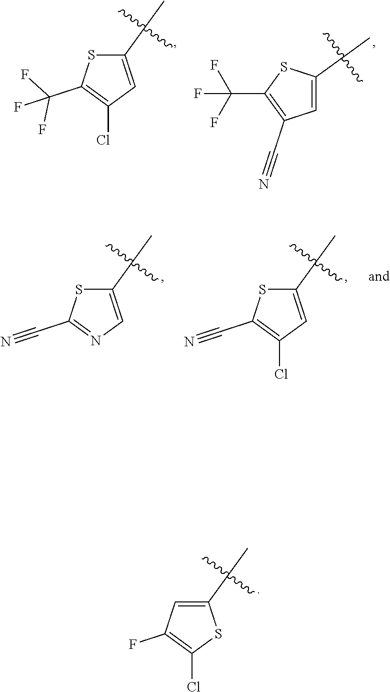 [SMILES: *c1cc(Cl)c(C(F)(F)F)s1 ; *c1cc(C#N)c(C(F)(F)F)s1 ; *c1cnc(C#N)s1 ; *c1cc(Cl)c(C#N)s1 ; *c1cc(F)c(Cl)s1]

Embodiment 181. The compound of any one of Embodiments 174-180, wherein R₁ is haloalkyl.

Embodiment 182. The compound of Embodiment 181, wherein R₁ is fluoroalkyl.

Embodiment 183. The compound of Embodiment 182, wherein R₁ is fluoroethyl or difluoroethyl.

Embodiment 184. The compound of any one of Embodiments 174-183, wherein R₂ is halo.

Embodiment 185. The compound of Embodiment 184, wherein R₂ is fluoro.

Embodiment 186. The compound of any one of Embodiments 0-185, wherein each amino, alkylamino or dialkylamino is unsubstituted or substituted.

Embodiment 187. The compound of any one of Embodiments 0-185, wherein each amino, alkylamino or dialkylamino is unsubstituted.

Embodiment 188. A compound selected from Table 2, Table 2a, Table 2b, Table 2c, Table 2d, and pharmaceutically acceptable salts thereof.

Embodiment 189. A method of treating cancer in a subject in need thereof, comprising administering a therapeutically effective amount of a any one of Embodiments 0-188 to the subject or a cell of the subject.

Embodiment 190. A compound of any one of Embodiments 0-188 for use in the treatment of cancer in a cell or subject.

Embodiment 191. A compound of any one of Embodiments 0-188 for use as a medicament for the treatment of cancer in a cell or subject.

Embodiment 192. The use of a compound of any one of Embodiments 0-188 in the manufacture of a medicament for the treatment of cancer in a cell or subject.

Embodiment 193. The use, compound for use or method of any one of Embodiments 189-192, wherein the subject or cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

Embodiment 194. The use, compound for use or method of any one of Embodiments 189-192, wherein the subject or a cell of the subject comprises a biomarker of sensitivity to a SMARCA2 antagonist.

Embodiment 195. The use, compound for use or method of Embodiment 194, wherein the biomarker is a decreased activity or function of SMARCA4.

Embodiment 196. The use, compound for use or method of Embodiment 194, wherein the biomarker is loss of function of SMARCA4.

Embodiment 197. The use, compound for use or method of any one of Embodiments 189-192, wherein the subject has a cancer characterized by loss of function of SMARCA4.

Embodiment 198. The use, compound for use or method of any one of Embodiments 189-192, wherein said subject or cell of the subject exhibits a decreased activity or function of SMARCA4 when compared to a control level of the activity or the function of SMARCA4. In some embodiments, the subject or cell of the subject exhibits a SMARCA4 mutation as compared to wild-type SMARCA4.

Embodiment 199. The use, compound for use or method of Embodiment 198, wherein the control level is the level of activity or function of SMARCA4 in a subject that does not have cancer.

Embodiment 200. A method of modulating (e.g., inhibiting) an activity of SMARCA2, comprising contacting SMARCA2 enzyme with a compound of any one of Embodiments 1-188.

Embodiment 201. The compound of any one of Embodiments 0-188 for use in inhibiting an activity of SMARCA2, wherein the compound is contacted with a SMARCA2 enzyme.

Embodiment 202. The compound of any one of Embodiments 0-188 for use as a medicament for inhibiting an activity of SMARCA2, wherein the medicament is contacted with a SMARCA2 enzyme.

Embodiment 203. The compound of any one of Embodiments 0-188 for use in the manufacture of a medicament for inhibiting an activity of SMARCA2, wherein the medicament is to be contacted with a SMARCA2 enzyme.

1. A compound of Formula (I): 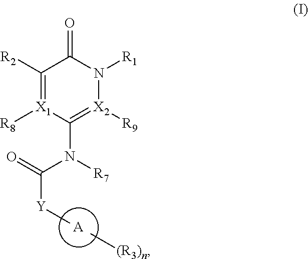
or a pharmaceutically acceptable salt thereof, wherein
A is a heteroaryl, heterocycloalkyl, aryl, or a cycloalkyl;
X₁ and X₂ are each independently selected from —CH and N;
Y is selected from the group consisting of a bond, —NH, —C(O), C₁-C₆ alkyl, —C(CH₃)₂—O—, -, and —CH₂—NH—CH₂—;
R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;
R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, —OR₅, —C(O)NH₂, —NO₂;
R₄ and R₄′ are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};
each R₅ is independently selected from the group consisting of H, cyano, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};
each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};
each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,
wherein Q is C₁-C₃ alkyl, C₂-C₆ alkenyl, C₃-C₆ cycloalkyl, C₃-C₆ heterocycloalkyl or C₂-C₆ alkynyl;
each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};
R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};
R₈ and R_{9′} are each independently selected from the group consisting of H, halo, and C₁-C₃ alkyl;
m is 1, 2, 3, 4, 5, or 6;
n is 0, 1, 2, 3, or 4; and
each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

2. A compound of Formula (IA) 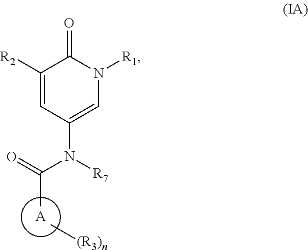
or a pharmaceutically acceptable salt thereof, wherein
A is a heteroaryl, heterocycloalkyl, aryl, or a cycloalkyl;
R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;
R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;
R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};
each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};
each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};
each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,
wherein Q is C₁-C₃ alkyl, C₂-C₆ alkenyl, C₃-C₆ cycloalkyl, C₃-C₆ heterocycloalkyl or C₂-C₆ alkynyl;
each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};
R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};
m is 1, 2, 3, 4, 5, or 6;
n is 0, 1, 2, 3, or 4; and
each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

3. The compound of claim 2, wherein
R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅; and
wherein Q is C₁-C₃ alkyl, C₂-C₆ alkenyl, or C₂-C₆ alkynyl.

4. A compound of Formula (IB): 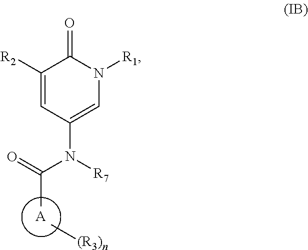
or a pharmaceutically acceptable salt thereof, wherein
A is a heteroaryl, heterocycloalkyl, aryl, or a cycloalkyl;
R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;
R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;
R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};
each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};
each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};
each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,
wherein Q is C₁-C₃ alkyl, C₂-C₆ alkenyl, C₃-C₆ cycloalkyl, C₃-C₆ heterocycloalkyl or C₂-C₆ alkynyl;
each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};
R₇ is selected from the group consisting of H and C₁-C₆ alkyl;
m is 1, 2, 3, 4, 5, or 6;
n is 0, 1, 2, 3, or 4; and
each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

5. The compound of claim 4, wherein
R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅; and
wherein Q is C₁-C₃ alkyl, C₂-C₆ alkenyl, or C₂-C₆ alkynyl.

6. A compound of Formula (IC) 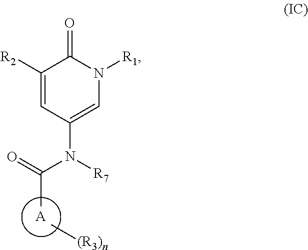
or a pharmaceutically acceptable salt thereof, wherein
A is a 5- or 6-membered heteroaryl having 1 to 4 heteroatoms selected from N, O, and S;
R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;
R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;
R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};
each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};
each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};
each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,
wherein Q is C₁-C₃ alkyl, C₂-C₆ alkenyl, C₃-C₆ cycloalkyl, C₃-C₆ heterocycloalkyl or C₂-C₆ alkynyl;
each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};
R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};
m is 1, 2, 3, 4, 5, or 6;
n is 0, 1, 2, 3, or 4; and
each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

7. The compound of claim 6, wherein
R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅; and
wherein Q is C₁-C₃ alkyl, C₂-C₆ alkenyl, or C₂-C₆ alkynyl.

8. A compound of Formula (ID) 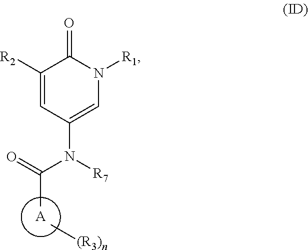
or a pharmaceutically acceptable salt thereof, wherein
A is a heteroaryl, heterocycloalkyl, aryl, or a cycloalkyl;
R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;
R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, —S(O)₀₋₂R₅, and —OR₅;
R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};
each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};
each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};
each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅;
each Q is independently selected from the group consisting of C₁-C₃ alkyl, C₂-C₆ alkenyl, C₃-C₆ cycloalkyl, C₃-C₆ heterocycloalkyl, and C₂-C₆ alkynyl;
each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};
R₇ is selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};
m is 1, 2, 3, 4, 5, or 6;
n is 0, 1, 2, 3, or 4; and
each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted;
provided that at least one R₃ is QR₆, wherein Q is C₂-C₆ alkynyl.

9. A compound of Formula (IE) 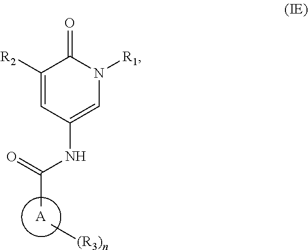
or a pharmaceutically acceptable salt thereof, wherein
A is a 5-membered heteroaryl having 1 to 4 heteroatoms selected from N, O, and S;
R₁ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, and —(CH₂)ₘR₄;
R₂ is selected from the group consisting of H, halo, COOH, cyano, C₁-C₆ alkyl, C₂-C₆ alkenyl, C₂-C₆ alkynyl, C₃-C₈ cycloalkyl, C₆-C₁₀ aryl, heterocycloalkyl, heteroaryl, —(CH₂)ₘR₄, —NR₅R_{5′}, and —OR₅;
R₄ and R_{4′} are each independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, —NR₅R_{5′};
each R₅ is independently selected from the group consisting of H, C₁-C₆ alkyl, C₂-C₆ alkenyl, C₂-C₆ alkynyl, C₁-C₆ alkoxyl, C₁-C₆ alkylcarbonyl, C₃-C₈ cycloalkyl, C₆-C₁₀ aryl, C₆-C₁₀ aryloxyl, heterocycloalkyl, heteroaryl, and —(CH₂)ₘR_{4′};
each R_{5′} is independently selected from the group consisting of H, C₁-C₆ alkyl, C₂-C₆ alkenyl, C₂-C₆ alkynyl, C₁-C₆ alkylcarbonyl, C₃-C₈ cycloalkyl, C₆-C₁₀ aryl, heterocycloalkyl, heteroaryl, and —(CH₂)ₘR_{4′};
each R₃ is independently selected from the group consisting of halo, hydroxyl, COOH, cyano, nitro, oxo, C₁-C₆ alkyl, C₂-C₆ alkenyl, C₂-C₆ alkynyl, C₃-C₈ cycloalkyl, C₆-C₁₀ aryl, C₆-C₁₀ aryloxyl, heterocycloalkyl, heteroaryl, aminocarbonyl, C₁-C₆ alkylsulfonyl, aminosulfonyl, QR₆, —(CH₂)ₘR₆, —NR₅R_{5′}, and —OR₅,
wherein Q is C₁-C₃ alkyl, C₂-C₆ alkenyl, or C₂-C₆ alkynyl;
each R₆ is independently selected from the group consisting of halo, hydroxyl, COOH, cyano, C₂-C₆ alkenyl, C₂-C₆ alkynyl, C₁-C₆ alkoxyl, C₃-C₈ cycloalkyl, C₆-C₁₀ aryl, C₆-C₁₀ aryloxyl, heterocycloalkyl, heteroaryl, and —NR₅R_{5′};
m is 1, 2, 3, 4, 5, or 6;
n is 0, 1, 2, 3, or 4; and
each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted.

10. The compound of any one of claims 1-9, wherein each alkyl, alkenyl, alkynyl, alkoxy, alkylcarbonyl, alkylsulfonyl, aminocarbonyl, aminosulfonyl, cycloalkyl, aryl, aryloxyl, heterocycloalkyl, or heteroaryl is unsubstituted or substituted with one or more substituents selected from the group consisting of an alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino, alkylamino, dialkylamino, arylamino, diarylamino and alkylarylamino, acylamino, alkylcarbonylamino, arylcarbonylamino, carbamoyl and ureido, amidino, imino, sulfhydryl, alkylthio, arylthio, thiocarboxylate, sulfates, alkylsulfinyl, sulfonato, aminosulfonyl, alkylsulfonyl, sulfonamido, nitro, trifluoromethyl, cyano, azido, cycloalkyl, heterocyclyl, alkylaryl, aromatic and heteroaromatic substituent.

11. The compound of any one of claims 1-10, wherein each alkyl, alkoxyl, alkenyl, alkynyl, alkylcarbonyl, or alkylsulfonyl is unsubstituted or substituted with one or more substituents from the group consisting of halo, amino, alkoxyl, cycloalkyl, aryl, heterocycloalkyl, and heteroaryl.

12. The compound of any one of claims 1-11, wherein each cycloalkyl, aryl, aryloxyl, heterocycloalkyl, or heteroaryl is unsubstituted or substituted with one or more substituents from the group consisting of halo, alkyl, haloalkyl, alkoxyl, cycloalkyl, aryl, heterocycloalkyl, and heteroaryl. In some embodiments, each cycloalkyl, aryl, aryloxyl, heterocycloalkyl, or heteroaryl is unsubstituted or substituted with one or more substituents from the group consisting of halo, alkyl, haloalkyl, and alkoxyl.

13. The compound of any one of claims 1-12, wherein each aminocarbonyl, or aminosulfonyl is unsubstituted or substituted with one or more substituents from the group consisting of halo, alkyl, alkoxyl, cycloalkyl, aryl, heterocycloalkyl, and heteroaryl.

14. The compound of any one of claims 1-13, wherein R₃ is selected from the group consisting of halo, hydroxyl, COOH, cyano, nitro, oxo, C₁-C₆ alkyl, C₂-C₆ alkenyl, C₂-C₆ alkynyl, C₁-C₆ haloalkyl, C₃-C₈ cycloalkyl, 4 to 7-membered heterocycloalkyl, 5 to 12-membered heterocycloalkyl, aminocarbonyl, mono-C₁-C₆ alkylaminocarbonyl, di-C₁-C₆ alkylaminocarbonyl, C₁-C₆ alkylcarbonylamino, QR₆, —(CH₂)ₘR₆, —NR₅R_{5′}, and —OR₅.

15. The compound of any one of claims 1-14, wherein R₆ is selected from the group consisting of halo, hydroxyl, COOH, cyano, C₂-C₆ alkenyl, C₂-C₆ alkynyl, C₁-C₆ alkoxyl, 4 to 7-membered heterocycloalkyl, —NR₅R_{5′}.

16. The compound of any one of claims 1-15, wherein A is selected from thiazolyl, isothiazolyl, thiazol-2-onyl, thiophenyl, pyrrolyl, pyrazolyl, imidazolyl, 1,2,3-thiadiazolyl, 1,2,4-thiadiazolyl, furanyl, oxazolyl, isoxazolyl, 1,2,4-triazolyl, and 1,2,3-triazolyl.

17. The compound of any one of claims 1-16, wherein A is selected from thiazolyl, thiophenyl, pyrrolyl, and pyrazolyl.

18. The compound of any one of claims 1-17, wherein A is thiazolyl or thiophenyl.

19. The compound of any one of claims 1-17, wherein A is N-substituted pyrrolyl.

20. The compound of any one of claims 1-19, wherein R₁ is selected from the group consisting of H, C₁-C₆ alkyl, C₁-C₆ haloalkyl, C₆-C₁₀ aryl, C₃-C₈ cycloalkyl, and —(CH₂)ₘR₄.

21. The compound of any one of claims 1-20, wherein R₁ is selected from the group consisting of unsubstituted or substituted C₁-C₆ alkyl, of unsubstituted or substituted C₁-C₆ haloalkyl, of unsubstituted or substituted C₆-C₁₀ aryl, of unsubstituted or substituted C₃-C₈ cycloalkyl, and —(CH₂)ₘR₄.

22. The compound of claim 20, wherein R₁ is selected from the group consisting of H, C₁-C₆ alkyl, or C₁-C₆ haloalkyl.

23. The compound of claim 22, wherein R₁ is methyl, ethyl, halomethyl or haloethyl.

24. The compound of claim 22, wherein R₁ is fluoroalkyl.

25. The compound of claim 23, wherein R₁ is selected from the group consisting of fluoromethyl, difluoromethyl, trifluoromethyl, fluoroethyl, difluoroethyl, and trifluoroethyl.

26. The compound of claim 20, wherein R₁ is C₃-C₈ cycloalkyl.

27. The compound of claim 20, wherein R₁ is an unsubstituted or substituted C₃-C₈ cycloalkyl.

28. The compound of claim 26, wherein R₁ is cyclopropyl.

29. The compound of claim 20, wherein R₁ is C₆-C₁₀ aryl.

30. The compound of claim 29, wherein R₁ is phenyl.

31. The compound of claim 20, wherein R₁ is —(CH₂)ₘR₄.

32. The compound of claim 31, wherein R₄ is selected from the group consisting of C₁-C₆ alkoxyl, mono-C₁-C₆ alkylamino, and di-C₁-C₆ alkylamino.

33. The compound of claim 31, wherein R₄ is hydroxyl.

34. The compound of claim 32, wherein R₄ is mono-C₁-C₆ alkylamino.

35. The compound of claim 34, wherein R₄ is methylamino.

36. The compound of claim 32, wherein R₄ is di-C₁-C₆ alkylamino.

37. The compound of claim 36, wherein R₄ is dimethylamino.

38. The compound of claim 31, wherein R₄ is C₁-C₆ alkoxyl.

39. The compound of claim 38, wherein R₄ is methoxyl.

40. The compound of claim 31, wherein R₄ is C₆-C₁₀ aryl.

41. The compound of claim 40, wherein R₄ is phenyl.

42. The compound of claim 31, wherein R₄ is C₃-C₈ cycloalkyl

43. The compound of claim 42, wherein R₄ is cyclopropyl.

44. The compound of claim 31, wherein R₄ is a 5-membered heteroaryl.

45. The compound of claim 44, wherein R₄ is pyrazolyl or imidazolyl.

46. The compound of claim 31, wherein R₄ is a 5-membered heterocycloalkyl.

47. The compound of claim 46, wherein R₄ is pyrrolidinyl.

48. The compound of any one of claims 31-47, wherein m is 1.

49. The compound of any one of claims 31-47, wherein m is 2.

50. The compound of any one of claims 31-47, wherein m is 3, 4, 5, or 6.

51. The compound of any one of claims 1-50, wherein R₂ is selected from the group consisting of H, halo, cyano, C₁-C₆ alkyl, —(CH₂)ₘR₄, —NR₅R_{5′}, and —OR₅.

52. The compound of claim 51, wherein R₂ is H.

53. The compound of claim 51, wherein R₂ is cyano.

54. The compound of claim 51, wherein R₂ is halo.

55. The compound of claim 54, wherein R₂ is F, Cl, or Br.

56. The compound of claim 51, wherein R₂ is C₁-C₆ alkyl.

57. The compound of claim 56, wherein R₂ is methyl, ethyl, or propyl.

58. The compound of claim 51, wherein R₂ is —(CH₂)ₘR₄.

59. The compound of claim 58, wherein R₄ is C₆-C₁₀ aryl.

60. The compound of claim 59, wherein R₄ is phenyl.

61. The compound of claim 58, wherein R₄ is a 5-membered heteroaryl.

62. The compound of claim 61, wherein R₄ is 1-methyl-pyrazolyl.

63. The compound of claim 51, wherein R₂ is —NR₅R_{5′}, R₅ is H and R_{5′} is C₁-C₆ alkyl.

64. The compound of claim 51, wherein R₂ is —NR₅R_{5′}, and R₅ and R_{5′} are both C₁-C₆ alkyl.

65. The compound of claim 51, wherein R₂ is —NR₅R_{5′}, R₅ is H and R_{5′}—(CH₂)ₘR_{4′}.

66. The compound of claim 64, wherein R_{4′} is C₁-C₆ alkoxyl.

67. The compound of claim 66, wherein R_{4′} is methoxyl.

68. The compound of claim 65, wherein R_{4′} is di-C₁-C₆ alkylamino.

69. The compound of claim 68, wherein R_{4′} is dimethylamino.

70. The compound of claim 65, wherein R_{4′} is a 6-membered heteroaryl.

71. The compound of claim 70, wherein R_{4′} is pyridinyl.

72. The compound of claim 65, wherein R_{4′} is a 6-membered heterocycloalkyl.

73. The compound of claim 72, wherein R_{4′} is morpholinyl.

74. The compound of claim 65, wherein R_{4′} is a 5-membered heteroaryl.

75. The compound of claim 74, wherein R_{4′} is 1-methylpyrazolyl.

76. The compound of claim 74, wherein R_{4′} is imidazolyl

77. The compound of claim 65, wherein R_{4′} is a 5-membered heterocyclyl.

78. The compound of claim 77, wherein R_{4′} is pyrrolidinyl.

79. The compound of claim 51, wherein R₂ is —OR₅ and R₅ is —(CH₂)ₘR_{4′}.

80. The compound of claim 79, wherein R_{4′} is selected from the group consisting of C₁-C₆ alkoxyl, mono-C₁-C₆ alkylamino, and di-C₁-C₆ alkylamino.

81. The compound of claim 80, wherein R_{4′} is C₁-C₆ alkoxyl.

82. The compound of claim 81, wherein R_{4′} is methoxyl.

83. The compound of claim 80, wherein R_{4′} is mono-C₁-C₆ alkylamino.

84. The compound of claim 83, wherein R_{4′} is methylamino.

85. The compound of claim 80, wherein R_{4′} is di-C₁-C₆ alkylamino.

86. The compound of claim 85, wherein R_{4′} is dimethylamino.

87. The compound of claim 79, wherein R_{4′} is a 6-membered heterocycloalkyl.

88. The compound of claim 87, wherein R_{4′} is 1-methylpiperazine or morpholinyl.

89. The compound of any one of claims 58-88, wherein m is 1.

90. The compound of any one of claims 58-88, wherein m is 2.

91. The compound of any one of claims 1-90, wherein each R₃ is selected from the group consisting of halo, cyano, nitro, oxo, C₁-C₆ alkyl, C₁-C₆ alkenyl, C₁-C₆ haloalkyl, C₃-C₈ cycloalkyl, C₆-C₁₀ aryl, 5 to 6-membered heteroaryl, 5 to 12-membered heterocycloalkyl, aminocarbonyl, mono-C₁-C₆ alkylaminocarbonyl, di-C₁-C₆ alkylaminocarbonyl, C₁-C₆ alkylsulfonyl, aminosulfonyl, -QR₆, —(CH₂)ₘR₆, —NR₅R_{5′}, and —OR₅.

92. The compound of claim 91, wherein each R₃ is selected from the group consisting of halo, cyano, nitro, oxo, C₁-C₆ alkyl, C₁-C₆ alkenyl, C₁-C₆ haloalkyl, C₃-C₈ cycloalkyl, 5 to 12-membered heterocycloalkyl, aminocarbonyl, mono-C₁-C₆ alkylaminocarbonyl, di-C₁-C₆ alkylaminocarbonyl, C₁-C₆ alkylsulfonyl, aminosulfonyl, -QR₆, —(CH₂)ₘR₆, —NR₅R_{5′}, and —OR₅.

93. The compound of claim 91, wherein R₃ is halo.

94. The compound of claim 91, wherein R₃ is cyano.

95. The compound of claim 91, wherein R₃ is nitro.

96. The compound of claim 91, wherein R₃ is oxo.

97. The compound of claim 91, wherein R₃ is C₁-C₆ alkenyl.

98. The compound of claim 91, wherein R₃ is C₁-C₆ haloalkyl.

99. The compound of claim 98, wherein R₃ is trifluoromethyl.

100. The compound of claim 91, wherein R₃ is aminocarbonyl, mono-C₁-C₆ alkylaminocarbonyl, or di-C₁-C₆ alkylaminocarbonyl.

101. The compound of claim 91, wherein R₃ is methylaminocarbonyl.

102. The compound of claim 91, wherein R₃ is dimethylaminocarbonyl.

103. The compound of claim 91, wherein R₃ is C₁-C₆ alkylsulfonyl.

104. The compound of claim 91, wherein R₃ is aminosulfonyl.

105. The compound of claim 91, wherein R₃ is methylsulfonyl.

106. The compound of claim 91, wherein R₃ is C₆-C₁₀ aryl.

107. The compound of claim 106, wherein R₃ is phenyl.

108. The compound of claim 106 or 107, wherein the C₆-C₁₀ aryl is substituted with one or more C₁-C₆ alkyl, halogen, or C₁-C₆ alkoxyl.

109. The compound of claim 91, wherein R₃ is C₃-C₈ cycloalkyl.

110. The compound of claim 109, wherein R₃ is cyclopropyl.

111. The compound of claim 91, wherein R₃ is a 5 to 6-membered heteroaryl.

112. The compound of claim 111, wherein R₃ is selected from oxazolyl, pyridinyl, furanyl, thiazolyl, pyrrolyl, imidazolyl, and pyrazolyl.

113. The compound of claim 111 or 112, wherein the 5 to 6-membered heteroaryl is substituted with one or more methyl.

114. The compound of claim 111 or 112, wherein the 5 to 6-membered heteroaryl is substituted with one or more C₁-C₆ haloalkyl.

115. The compound of claim 114, wherein the 5 to 6-membered heteroaryl is substituted with trifluoromethyl.

116. The compound of claim 113, wherein R₃ is selected from the group consisting of 2-methylthiazolyl, 1,2-dimethyl-pyrrolyl, 1-methyl-imidazolyl, and 1-methyl-pyrazolyl.

117. The compound of claim 91, wherein R₃ is 5 to 12-membered heterocycloalkyl.

118. The compound of claim 117, wherein R₃ is 2,3-dihydrobenzofuranyl.

119. The compound of claim 91, wherein R₃ is —(CH₂)ₘR₆.

120. The compound of claim 119, wherein R₆ is hydroxyl.

121. The compound of claim 119, wherein R₆ is C₆-C₁₀ aryl.

122. The compound of claim 121, wherein C₆-C₁₀ aryl is substituted with C₁-C₆ alkoxyl.

123. The compound of claim 121 or 122, wherein C₆-C₁₀ aryl is phenyl.

124. The compound of any one of claims 119-123, wherein m is 1.

125. The compound of claim 91, wherein R₃ is QR₆.

126. The compound of claim 125, wherein R₆ is a 5-membered heterocyclyl.

127. The compound of claim 126, wherein R₆ is pyrrolidine.

128. The compound of claim 125, wherein R₆ is a 6-membered heteroaryl.

129. The compound of claim 128, wherein R₆ is pyridinyl.

130. The compound of claim 125, wherein R₆ is amino.

131. The compound of claim 125, wherein R₆ is di-C₁-C₆ alkylamino.

132. The compound of claim 131, wherein R₆ is dimethylamino.

133. The compound of claim 125, wherein R₆ is hydroxyl.

134. The compound of claim 126, wherein R₆ is C₁-C₆ haloalkyl.

135. The compound of claim 134, wherein R₆ is trifluoromethyl.

136. The compound of any one of claims 125-135, wherein Q is prop-1-ynyl.

137. The compound of any one of claims 125-135, wherein Q is a C₁-C₃ alkyl.

138. The compound of claim 137, wherein Q is substituted with OH.

139. The compound of claim 137, wherein Q is substituted with halo.

140. The compound of claim 139, wherein Q is substituted with fluoro.

141. The compound of claim 137 or 138, wherein Q is methyl.

142. The compound of claim 91, wherein R₃ is —NR₅R_{5′}.

143. The compound of claim 142, wherein R₅ is H and R_{5′} is C₃-C₈ cycloalkyl.

144. The compound of claim 143, wherein R_{5′} is cyclopentyl.

145. The compound of claim 142, wherein R₅ is H and R_{5′} is C₁-C₆ alkyl.

146. The compound of claim 145, wherein R_{5′} is methyl.

147. The compound of claim 145, wherein R_{5′} is i-propyl.

148. The compound of claim 142, wherein R₅ is H and R_{5′} is C₁-C₆ alkylcarbonyl.

149. The compound of claim 148, wherein R_{5′} is ethanoyl.

150. The compound of claim 91, wherein R₃ is OR₅.

151. The compound of claim 150, wherein R₅ is C₁-C₆ alkyl

152. The compound of claim 151, wherein the C₁-C₆ alkyl is methyl.

153. The compound of any one of claims 1-152, wherein n is 2 or 3.

154. The compound of claim 91, wherein n is 1 and R₃ is cyano.

155. The compound of claim 91, wherein n is 1 or 2 and R₃ is halo.

156. The compound of claim 91, wherein n is 2, one R₃ is halo and the other R₃ is cyano.

157. The compound of any claim 91, 155, or 156, wherein halo is selected from Cl, Br, and I.

158. The compound of claim 1, wherein A is thiazolyl.

159. The compound of claim 1, wherein A is thiophenyl.

160. The compound of claim 158 or 159, wherein n is 1 and R₃ is cyano.

161. The compound of claim 158 or 159, wherein n is 2 and R₃ is selected from halo and cyano.

162. The compound any one of claim 158 or 161, wherein n is 2 and each R₃ is halo.

163. The compound of claim 161 or 162, wherein halo is chloro or fluoro.

164. The compound of claim 1, wherein 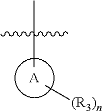
is selected from

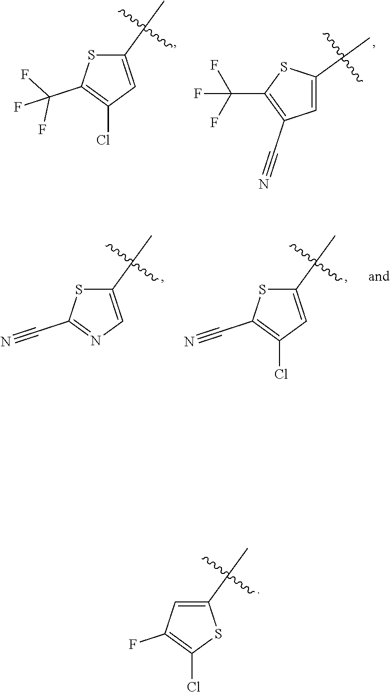 [SMILES: *c1cc(Cl)c(C(F)(F)F)s1 ; *c1cc(C#N)c(C(F)(F)F)s1 ; *c1cnc(C#N)s1 ; *c1cc(Cl)c(C#N)s1 ; *c1cc(F)c(Cl)s1]

165. The compound of claim 1, wherein 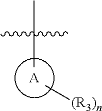
is selected from

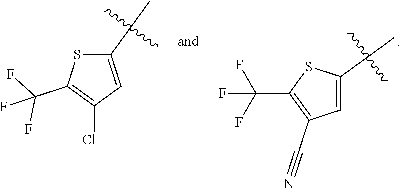 [SMILES: *c1cc(Cl)c(C(F)(F)F)s1 ; *c1cc(C#N)c(C(F)(F)F)s1]

166. The compound of any one of claims 158-164, wherein R₁ is haloalkyl.

167. The compound of claim 166, wherein R₁ is fluoroalkyl.

168. The compound of claim 167, wherein R₁ is fluoroethyl or difluoroethyl.

169. The compound of any one of claims 158-168, wherein R₂ is halo.

170. The compound of claim 169, wherein R₂ is fluoro.

171. The compound of any one of claims 1-170, wherein each amino, alkylamino or dialkylamino is unsubstituted or substituted.

172. The compound of any one of claims 1-170, wherein each amino, alkylamino or dialkylamino is unsubstituted.

173. The compound of any one of claims 1-172, wherein at least one R₃ is QR₆ and wherein Q is C₂-C₆ alkynyl.

174. The compound of any one of claims 1-173, wherein 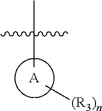
is selected from

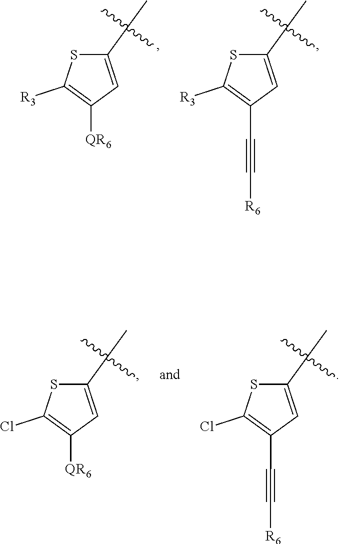

175. The compound of claim 171, wherein n is 2 and at least one R₃ is selected from halo and cyano.

176. A compound selected from Table 2, Table 2a, Table 2b, Table 2c, Table 2d, pharmaceutically acceptable salts thereof.

177. A method of treating cancer in a subject in need thereof, comprising administering a therapeutically effective amount of a compound of any one of claims 1-176 to the subject or a cell of the subject.

178. A compound of any one of claims 1-175 for use in the treatment of cancer in a cell or subject

179. A compound of any one of claims 1-175 for use as a medicament for the treatment of cancer in a cell or subject.

180. The use of a compound of any one of claims 1-175 in the manufacture of a medicament for the treatment of cancer in a cell or subject.

181. The use, compound for use or method of any one of claims 177-180, wherein the subject or cell of the subject exhibits a decreased activity or function of SMARCA4 when compared to a control level of the activity or the function of SMARCA4.

182. The use, compound for use or method of any one of claims 177-180, wherein the subject or a cell of the subject comprises a biomarker of sensitivity to a SMARCA2 antagonist.

183. The use, compound for use or method of claim 182, wherein the biomarker is a decreased activity or function of SMARCA4.

184. The use, compound for use or method of claim 182, wherein the biomarker is loss of function of SMARCA4.

185. The use, compound for use or method of any one of claims 177-180, wherein the subject has a cancer characterized by loss of function of SMARCA4.

186. The use, compound for use or method of any one of claims 177-180, wherein said subject or cell of the subject exhibits a decreased activity or function of SMARCA4 when compared to a control level of the activity or the function of SMARCA4.

187. The use, compound for use or method of claim 186, wherein the control level is the level of activity or function of SMARCA4 in a subject or cell from a subject that does not have cancer.

188. The use, compound for use or method of any one of claims 177-180, wherein said subject or cell of the subject exhibits a SMARCA4 mutation as compared to wild-type SMARCA4.

189. The use, compound for use or method of any one of claims 177-180, wherein said subject or cell of the subject exhibits a loss of SMARCA4 protein expression as compared to a control level of SMARCA4 protein expression.

190. The use, compound for use or method of claim 189, wherein the control level is the level of SMARCA4 protein expression in a subject or cell from a subject that does not have cancer.

191. The use, compound for use or method of any one of claims 177-180, wherein said subject or cell of the subject exhibits a loss of SMARCA4 mRNA expression as compared to a control level of SMARCA4 mRNA expression.

192. The use, compound for use or method of claim 191, wherein the control level is the level of SMARCA4 mRNA expression in a subject or cell from a subject that does not have cancer.

193. A method of modulating (e.g., inhibiting) an activity of SMARCA2, comprising contacting SMARCA2 enzyme with a compound of any one of claims 1-175.

194. The compound of any one of claims 1-175 for use in inhibiting an activity of SMARCA2, wherein the compound is contacted with a SMARCA2 enzyme.

195. The compound of any one of claims 1-175 for use as a medicament for inhibiting an activity of SMARCA2, wherein the medicament is contacted with a SMARCA2 enzyme.

196. The compound of any one of claims 1-175 for use in the manufacture of a medicament for inhibiting an activity of SMARCA2, wherein the medicament is to be contacted with a SMARCA2 enzyme.


SEQUENCE LISTING (1 sequence)

SEQ ID NO: 1 - protein, 1610 aa, Artificial Sequence
  predicted translation for the HIS-SMARCA2-FL-FLAG
MGSHHHHHHHHSGSTPTDPGAMPHPGPSPGPGPSPGPILGPSPGPGPSPGSVHSMMGPSPGPPSVSHPMPTMGSTDFPQEGMHQMHKPIDGIHDKGIVEDIHCGSMKGTGMRPPHPGMGPPQSPMDQHSQGYMSPHPSPLGAPEHVSSPMSGGGPTPPQMPPSQPGALIPGDPQAMSQPNRGPSPFSPVQLHQLRAQILAYKMLARGQPLPETLQLAVQGKRTLPGLQQQQQQQQQQQQQQQQQQQQQQQPQQQPPQPQTQQQQQPALVNYNRPSGPGPELSGPSTPQKLPVPAPGGRPSPAPPAAAQPPAAAVPGPSVPQPAPGQPSPVLQLQQKQSRISPIQKPQGLDPVEILQEREYRLQARIAHRIQELENLPGSLPPDLRTKATVELKALRLLNFQRQLRQEVVACMRRDTTLETALNSKAYKRSKRQTLREARMTEKLEKQQKIEQERKRRQKHQEYLNSILQHAKDFKEYHRSVAGKIQKLSKAVATWHANTEREQKKETERIEKERMRRLMAEDEEGYRKLIDQKKDRRLAYLLQQTDEYVANLTNLVWEHKQAQAAKEKKKRRRRKKKAEENAEGGESALGPDGEPIDESSQMSDLPVKVTHTETGKVLFGPEAPKASQLDAWLEMNPGYEVAPRSDSEESDSDYEEEDEEEESSRQETEEKILLDPNSEEVSEKDAKQIIETAKQDVDDEYSMQYSARGSQSYYTVAHAISERVEKQSALLINGTLKHYQLQGLEWMVSLYNNNLNGILADEMGLGKTIQTIALITYLMEHKRLNGPYLIIVPLSTLSNWTYEFDKWAPSVVKISYKGTPAMRRSLVPQLRSGKFNVLLTTYEYIIKDKHILAKIRWKYMIVDEGHRMKNHHCKLTQVLNTHYVAPRRILLTGTPLQNKLPELWALLNFLLPTIFKSCSTFEQWFNAPFAMTGERVDLNEEETILIIRRLHKVLRPFLLRRLKKEVESQLPEKVEYVIKCDMSALQKILYRHMQAKGILLTDGSEKDKKGKGGAKTLMNTIMQLRKICNHPYMFQHIEESFAEHLGYSNGVINGAELYRASGKFELLDRILPKLRATNHRVLLFCQMTSLMTIMEDYFAFRNFLYLRLDGTTKSEDRAALLKKFNEPGSQYFIFLLSTRAGGLGLNLQAADTVVIFDSDWNPHQDLQAQDRAHRIGQQNEVRVLRLCTVNSVEEKILAAAKYKLNVDQKVIQAGMFDQKSSSHERRAFLQAILEHEEENEEEDEVPDDETLNQMIARREEEFDLFMRMDMDRRREDARNPKRKPRLMEEDELPSWIIKDDAEVERLTCEEEEEKIFGRGSRQRRDVDYSDALTEKQWLRAIEDGNLEEMEEEVRLKKRKRRRNVDKDPAKEDVEKAKKRRGRPPAEKLSPNPPKLTKQMNAIIDTVINYKDRCNVEKVPSNSQLEIEGNSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDEDYKDDDDK